(19) United States
(12) Patent Application Publication
(10) Pub. No.: US 2025/0137026 A1
(43) Pub. Date: May 1, 2025

(54) METHODS FOR RECOMBINANT PROTEIN EXPRESSION IN EUKARYOTIC CELLS

(71) Applicant: Wageningen Universiteit, Wageningen (NL)

(72) Inventors: Christian Andreas Klaus Birger SÜDFELD, Wageningen (NL); Sarah D'ADAMO, Wageningen (NL); Maria João GANTE DE VASCONCELOS BARBOSA, Wageningen (NL); René Hubertus WIJFFELS, Wageningen (NL)

(21) Appl. No.: 18/282,782

(22) PCT Filed: Mar. 18, 2022
(86) PCT No.: PCT/NL2022/050146

§ 371 Date: Sep. 18, 2023

(30) Foreign Application Priority Data
Mar. 19, 2021 (EP) 21163793.9

(51) Int. Cl. C12P 21/00; C12N 9/22; C12N 15/11; C12N 15/90

(52) U.S. Cl. CPC C12P 21/00; C12N 9/22; C12N 15/11; C12N 15/902; C12N 2310/20

(57) ABSTRACT

Methods and means are provided for efficient recombinant protein expression in eukaryotic cells. More specifically, methods, nucleic acids, and cells are provided for high protein expression, employing a polymerase I promoter and expression from a genomic region that promotes high expression from polymerase I promoters.

### CROSS REFERENCE TO RELATED APPLICATIONS

This application is a 371 National Stage application of International Application No. PCT/NL2022/050146, filed Mar. 18, 2022, which claims the benefit of European Patent Application 21163793.9, filed Mar. 19, 2021, each of which is incorporated herein by reference in its entirety.

### INCORPORATION OF SEQUENCE LISTING

The sequence listing that is contained in the file named VONL031US_revised_ST25.txt, which is 124 kilobytes (measured in MS-WINDOWS) and created on Apr. 18, 2024, is filed herewith by electronic submission and incorporated herein by reference.

### FIELD

The invention is directed to efficient recombinant protein expression in eukaryotic cells.

### BACKGROUND OF THE INVENTION

Biotechnological processes revolve around the use of biological systems to produce commercially interesting compounds. Often, these biological systems are engineered to increase productivities or robustness of a production process or to facilitate the formation of products that are not naturally produced by the organism. This practice, called genetic engineering often aims at heterologous or homologous expression of protein-coding genes to drive the formation of a protein of interest (POI) in a target organism for miscellaneous purposes. In eukaryotic cells, this is almost exclusively achieved by transfecting cells with a DNA molecule that harbours the respective gene of interest (GOI) flanked by a transcriptional promoter, 5′-UTR, 3′-UTR and transcriptional terminator, together forming the expression cassette (EC). Depending on the target organism, the expression cassette can remain in an episomal plasmid/chromosome or integrate into an existing chromosome at random or in a targeted way.

In all cases, the expression of the GOI is achieved by utilising a transcriptional promoter that recruits eukaryotic DNA-directed RNA polymerase II (Pol II) which transcribes the GOI including 5′-UTR and 3′-UTR into a pre-mRNA molecule. This nascent primary transcript is co- and post-transcriptionally modified to yield a mature messenger RNA (mRNA) molecule. The processing mechanisms importantly include addition of a 7-methylguanosine cap (5′-cap), polyadenylation i.e. the addition of a poly-A tail, and alternative splicing. The mRNA is exported to the cytoplasm where it is decoded by ribosomes to give rise to a polypeptide chain in a process called translation. Translation initiation is an intricate procedure that typically involves the recognition of the 5′-cap and poly-A tail of an mRNA by a series of eukaryotic translation initiation factors (eIF) which recruit the 40S ribosomal subunit to the transcript, forming the 43S preinitiation complex (43S PIC). The 43S PIC scans the 5′-UTR of the mRNA until it reaches the AUG start codon. Upon binding of the 60S ribosomal subunit some eIFs dissociate and translation commences, giving rise to the POI.

Usually the rate limiting step for gene expression is the initiation of transcription, which is highly dependent on the DNA sequence of the transcriptional promoter element used for transfection. Eukaryotic cells have evolved complex regulatory mechanisms that allow them to finetune the expression of all protein-coding genes precisely tailored to their physiological state. Cis- and trans-acting elements are part of this regulatory machinery and they influence the level of transcription of protein-coding genes in ways that are often difficult to predict. Depending on the insertion site of an EC within the genome, GOIs can be expressed at strongly varying levels. The chromatin structure and adjacent cis acting elements or lack thereof can also cause complete silencing of a transgene, in certain cases.

A typical eukaryotic cell can have between 10,000 and 30,000 protein-coding genes, which are all transcribed by the same enzyme, Pol II. The amount of RNA that can be transcribed from a single copy of a GOI is therefore very low and usually the rate-limiting factor for gene expression. Genetic engineers have tried to address this problem by selecting strong promoters, e.g. of highly transcribed endogenous genes or viral promoters or by optimising DNA sequences of synthetic promoters. The aim hereby is to increase the affinity of transcriptional initiation factor proteins and Pol II for the promoter region to favour the expression of the GOI over that of all other protein-coding genes.

An alternative approach is to insert multiple copies of an EC to increase the level of transcription. These strategies often utilise random or transposon-mediated insertion of ECs that can result in heterochromatinization of the transcriptional promoter (Soimi et al., 2000, RNA 11 (7), p 1004-1011) and further in unpredictable recombination events between different copies of ECs.

Another strategy for heterologous protein expression is by targeting an expression construct to an rDNA locus. This has been described in a range of yeast species including S. cerevisiae (Lopes et al., 1989, Gene, July 15; 79 (2): 199-206; Lopes et al., 1996, Yeast, April; 12 (5): 467-77; Lopes et al., Gene 1991 Aug. 30; 105 (1): 83-90) K. lactis (Bergkamp et al. 1992, Curr Genet pr; 21 (4-5): 365-70), Y. lipolytica (Le Dal et al., 1994, Curr Genet. 26, 38-44), A. adeninivorans (Wartmann et al., 1998, Yeast 14, 1017-1025; Steinborn et al., 2005, FEMS Yeast Research 5 (11): 1047-54), H. polymorpha (Klabunde et al., 2002 Appl. Microbiol. Biotechnol. 58, 797-805; Steinborn et al., 2006, Microbial Cell Factories 5 (1): 33), Pichia stipitis (Klabunde et al., 2003 FEMS Yeast Res November; 4 (2): 185-93) and Pichia pastoris (Marx et al., 2009, FEMS Yeast Res, December; 9 (8): 1260-70; Steinborn et al., 2006, supra; Guo et al., 2015, Sci Rep. 5:11730; Song et al., 2019, BMC Biotechnology, 19 (1), 54). To achieve high expression, these approaches typically use constitutive promoters and often aim for high EC copy numbers.

Palmer et al. (Nucleic Acids Res. 1993 Jul. 25; 21 (15): 3451-7) and U.S. Pat. No. 6,368,862B1 describe recombinant protein expression in fibroblasts from a vector comprising a RNA polymerase I (Pol I) promoter, where translation block is overcome by insertion of an Internal Ribosome Entry Site (IRES) into the 5′ leader of an RNA polymerase I transcript. Protein production from the RNA pol I transcript is enhanced by the addition of an SV40 polyadenylation signal. It is concluded that protein production reaches levels comparable to that produced from RNA polymerase II driven expression vectors when RNA Polymerase I driven expression vectors contain both elements.

U.S. Pat. No. 5,910,628 (Miller) discloses a method of increasing the production of a protein translated from an uncapped eukaryotic messenger ribonucleic acid (mRNA) and construct for use therein comprising a 5′ untranslated region including a 5′ translation enhancing segment.

U.S. Pat. No. 5,994,526 (Meulewater) discloses chimeric genes that comprise a first promoter recognized by a DNA-dependent RNA polymerase different from a eukaryotic RNA polymerase II; a DNA region encoding a chimeric RNA which comprises a 5′ UTR, an AU-rich heterologous coding sequence, a 3′ UTR; and optionally a terminator sequence recognized by said RNA polymerase, such that upon transcription by the RNA polymerase an uncapped RNA species is produced which comprises a first translation enhancing sequence derived from the 5′ region of genomic or subgenomic RNA of a positive stranded RNA plant virus; a heterologous RNA coding sequence encoding a polypeptide or protein of interest; and a second translation enhancing sequence derived from the 3′ region of genomic or subgenomic RNA of a positive-stranded RNA plant virus.

Wen et al. (Biochem Biophys Res Commun. 2008 Mar. 21; 367 (4): 846-51) describe a vector system for gene therapy, wherein a protein is expressed from a Polymerase I promoter in human cells using an internal ribosome entry site (IRES). In this system, expression of the FIX protein was found to be much lower than those observed with a moderate Pol II promoter construct.

US2019/0225973 A describes a Saccharomyces cerevisiae expression system and a construction method and application thereof, wherein the gene expression cassette includes from upstream to downstream an rDNA promoter, an internal ribosome entry site (IRES) sequence, an exogenous gene expression cassette, a poly (T) sequence, and an rDNA terminator.

Accordingly, there remains a need for alternative methods for protein expression systems that ensure high levels of recombinant protein expression and that do not suffer from the above drawbacks.

### SUMMARY OF THE INVENTION

In one aspect, a method is described for producing or expressing one or more proteins of interest in a eukaryotic cell by
a. introducing into a eukaryotic cell a nucleic acid molecule comprising a polynucleotide encoding a protein of interest (POI)
   wherein said nucleic acid molecule is targeted to the nucleolar DNA, preferably to a nucleolar organizer region (NOR), of said organism to form or insert upon integration of said nucleic acid molecule a chimeric gene comprising the following operably-linked elements:
      i. a polymerase I promoter;
      ii. a polynucleotide encoding an internal ribosomal entry site (IRES);
      iii. said polynucleotide encoding said POI
      iv. optionally, a 3′ end region/transcription terminator

Prior to introduction, said nucleic acid molecule may already comprise said polymerase I promoter, preferably the nucleic acid molecule already comprises the chimeric gene, such that the chimeric gene is preformed and inserted as a whole. Alternatively, the nucleic acid molecule may also not comprise a Poll promoter and be inserted downstream of an existing pol I promoter, such that the polynucleotide encoding an internal ribosomal entry site (IRES) and polynucleotide encoding said POI become operably linked, thereby forming the chimeric gene.

For targeting to the nucleolar DNA, the nucleic acid molecule may be flanked with one or more flanking sequences for allowing integration of said nucleic acid molecule at a predefined site in said nucleolar DNA by (one-sided or two-sided) homologous recombination. Alternatively or additionally, a DNA break may be induced at a predefined site in said nucleolar DNA, thereby allowing integration/insertion of said nucleic acid molecule at said predefined site. The flanking sequence(s) may be at least 15 nt in length and has (have) at least 80% sequence identity to the DNA at said predefined site in the nucleolar DNA where said chimeric gene is to be integrated. The DNA break can be induced at said predefined site by providing the cell with or expressing in said cell a sequence specific nuclease (SSN), such as an RNA-guided nuclease, that recognizes a DNA sequence at and introduces a DNA break at the predefined site.

The nucleic acid molecule may be integrated in or in the vicinity of an rRNA cistron, preferably within 10 kb of an rRNA cistron. The chimeric gene may also be inserted outside an existing rDNA cistron, i.e. inserted in such a way so as not to interrupt an existing rDNA cistron or not significantly interfere with the function or expression of the rDNA cistron, such as in the intergenic region between two rDNA cistrons.

The chimeric gene may further comprise a polynucleotide encoding a translational enhancer (TE) or a cap-independent translation enhancer (CITE) element. The chimeric gene may also comprise a terminator functional in the cell of the eukaryotic organisms, such as a pol I or pol II terminator, preferably from the same or a related species. Preferably, the terminator is the alpha tubulin terminator, preferably of the same (or a related) species. The chimeric gene may further comprise a polynucleotide encoding a second IRES (and optionally a second TE/CITE) operably-linked to a second polynucleotide encoding a second protein of interest (POI), so as to express multiple POIs from a single transcript.

The cell can be selected from a (non-human) animal cell, plant cell, a protist cell and fungal cell. The cell can also be a (unicellular) plant cell, algal cell or yeast cell, preferably wherein said cell is selected from a Nannochloropsis sp., a Chlorella sp., a Saccharomyces sp. or Pichia sp, even more preferably Nannochloropsis oceanica.

The cell may have a copy nr of rDNA cistrons of less than 200, less than 150, less than 100, preferably less than 70, such as less than 60 copies, less than 50 copies, less than 45 copies, less than 40 copies, less than 35 copies, less than 30 copies, less than 25 copies, less than 20 copies, less than 15 copies, less than 10 copies preferably less than 5, such as 4.

Expression of said POI can be enhanced compared to when said chimeric gene is inserted into non-NOR genomic DNA. Expression of said POI can also be enhanced compared to expression driven by an average pol II promoter, preferably enhanced compared to a strong pol II promoter

The method may comprise the further step of isolating and optionally purifying said one or more POIs.

In a further aspect, a chimeric gene is described for producing/expressing one or more proteins of interest (POI) as described in any one of the method embodiments, i.e. a chimeric gene comprising the following operably-linked elements:
i. a polymerase I promoter;
ii. a polynucleotide encoding an internal ribosomal entry site (IRES);
iii. said polynucleotide encoding said POI
iv. optionally, a 3′ end region/transcription terminator

The chimeric gene may further comprise a polynucleotide encoding a translational enhancer (TE) or a cap-independent translation enhancer (CITE) element. The chimeric gene may further comprise a polynucleotide encoding a second IRES (and optionally a second TE/CITE) operably-linked to a second polynucleotide encoding a second protein of interest (POI), so as to express multiple POIs from a single transcript

In another aspect, a (transgenic/cis-genic) eukaryotic cell is described for producing/expressing one or more proteins of interest (POI) as described in any of the method embodiments, the cell comprising a chimeric gene as described herein, i.e. a chimeric gene comprising the following operably-linked elements:
i. a polymerase I promoter;
ii. a polynucleotide encoding an internal ribosomal entry site (IRES);
iii. said polynucleotide encoding said POI
iv. optionally, a 3′ end region/transcription terminator
   wherein the chimeric gene has been inserted into (is located in) the nucleolar DNA of said cell, preferably into a nucleolar organiser region (NOR).

The chimeric gene can be located in or in the vicinity of an rDNA cistron, preferably within 10 kb of an rDNA cistron. The chimeric gene may also be located outside an existing rDNA cistron, i.e. inserted in such a way so as not to interrupt and existing rDNA cistron or not significantly interfere with the function or expression of the rDNA cistron, such as in the intergenic region between two rDNA cistrons.

The chimeric gene may further comprise a polynucleotide encoding a translational enhancer (TE) or a cap-independent translation enhancer (CITE) element. The chimeric gene may also comprise a terminator functional in the cell of the eukaryotic organisms, such as a pol I or pol II terminator, preferably from the same or a related species. Preferably, the terminator is the alpha tubulin terminator, preferably of the same (or a related) species. The chimeric gene may further comprise a polynucleotide encoding a second IRES (and optionally a second TE/CITE) operably-linked to a second polynucleotide encoding a second protein of interest (POI), so as to express multiple POIs from a single transcript.

The cell can be selected from a (non-human) animal cell, plant cell, a protist cell and fungal cell. The cell can also be a (unicellular) plant cell, algal cell or yeast cell, preferably wherein said cell is selected from a Nannochloropsis sp., a Chlorella sp., a Saccharomyces sp. or Pichia sp, more preferably a Nannochloropsis sp, even more preferably Nannochloropsis oceanica.

The cell may have a copy nr of rDNA cistrons of less than 200, less than 150, less than 100, preferably less than 70, such as less than 60 copies, less than 50 copies, less than 45 copies, less than 40 copies, less than 35 copies, less than 30 copies, less than 25 copies, less than 20 copies, less than 15 copies, less than 10 copies preferably less than 5, such as 4.

Expression of said POI can be enhanced compared to when said chimeric gene is inserted into non-NOR genomic DNA. Expression of said POI can also be enhanced compared to expression driven by an average pol II promoter, preferably enhanced compared to a strong pol II promoter

In another aspect, a nucleic acid molecule or vector is described for expressing one or more proteins of interest (POI) in a eukaryotic cell, said nucleic acid molecule or vector comprising a polynucleotide encoding said at least one (POI), wherein upon integration into the nucleolar DNA, preferably into a nucleolar organizer region (NOR), of said eukaryotic cell a chimeric gene is formed as herein described. Thus, upon integration a chimeric gene is formed comprising the following operably-linked elements:
i. a polymerase I promoter;
ii. a polynucleotide encoding an internal ribosomal entry site (IRES);
iii. said polynucleotide encoding said POI
iv. optionally, a 3′ end region/transcription terminator

Also described is a kit for expressing one or more proteins of interest (POIs) in a eukaryotic cell, said kit comprising one or more containers comprising the vector or nucleic acid molecule as herein described

The polynucleotide encoding said at least one (POI) can be flanked with one or more flanking sequences that allow insertion of said polynucleotide encoding said POI into a predefined site in a nucleolar organizer region (NOR) of said eukaryotic cell by (one-sided or two-sided) homologous recombination to form or insert the herein described chimeric gene. Alternatively or additionally, the nucleic acid molecule or vector or kit further comprises an expression cassette for expressing a sequence specific nuclease capable of recognizing a DNA sequence at and inducing a DNA break at a predefined site of the nucleolar DNA (e.g. NOR) of said eukaryotic cell for allowing integration of said polynucleotide encoding said POI at said predefined site to form or insert said chimeric gene.

The nucleic acid molecule or vector or kit may already comprise said polymerase I promoter, preferably the nucleic acid molecule or vector or kit already comprises said chimeric gene. Alternatively, nucleic acid molecule or vector or kit may also not comprise a Pol I promoter and be inserted downstream of an existing pol I promoter, such that the polynucleotide encoding an internal ribosomal entry site (IRES) and polynucleotide encoding said POI become operably linked, thereby forming the chimeric gene.

The chimeric gene may further comprise a polynucleotide encoding a translational enhancer (TE) or a cap-independent translation enhancer (CITE) element. The chimeric gene may also comprise a terminator functional in the cell of the eukaryotic organisms, such as a pol I or pol II terminator, preferably from the same or a related species. Preferably, the terminator is the alpha tubulin terminator, preferably of the same (or a related) species. The chimeric gene may further comprise a polynucleotide encoding a second IRES (and optionally a second TE/CITE) operably-linked to a second polynucleotide encoding a second protein of interest (POI), so as to express multiple POIs from a single transcript.

In yet another aspect, a method is described for producing one or more proteins or polypeptide of interest (POI), comprising the steps of
a. providing a cell as described herein comprising a chimeric gene as described herein; and optionally
b. isolating and/or purifying said one or more proteins or polypeptides.

Thus, the cell comprises a chimeric gene comprising the following operably-linked elements:
i. a polymerase I promoter;
ii. a polynucleotide encoding an internal ribosomal entry site (IRES);
iii. said polynucleotide encoding said POI
iv. optionally, a 3′ end region/transcription terminator
   wherein the chimeric gene has been inserted into the nucleolar DNA of said cell, preferably into a nucleolar organiser region (NOR).

The chimeric gene can be located in or in the vicinity of an rDNA cistron, preferably within 10 kb of an rRNA cistron. The chimeric gene may also be located outside an existing rDNA cistron, i.e. inserted in such a way so as not to interrupt an existing rDNA cistron or not significantly interfere with the function or expression of the rDNA cistron, such as in the intergenic region between two rDNA cistrons.

The chimeric gene may further comprise a polynucleotide encoding a translational enhancer (TE) or a cap-independent translation enhancer (CITE) element. The chimeric gene may also comprise a terminator functional in the cell of the eukaryotic organisms, such as a pol I or pol II terminator, preferably from the same or a related species. Preferably, the terminator is the alpha tubulin terminator, preferably of the same (or a related) species. The chimeric gene may further comprise a polynucleotide encoding second IRES (and optionally a second TE/CITE) operably-linked to a second polynucleotide encoding a second protein of interest (POI), so as to express multiple POIs from a single transcript.

The cell can be selected from a (non-human) animal cell, plant cell, a protist cell and fungal cell. The cell can also be a (unicellular) plant cell, algal cell or yeast cell, preferably wherein said cell is selected from a Nannochloropsis sp., a Chlorella sp., a Saccharomyces sp. or Pichia sp, more preferably a Nannochloropsis sp, even more preferably Nannochloropsis oceanica.

The cell may have a copy nr of rDNA cistrons of less than 200, less than 150, less than 100, preferably less than 70, such as less than 60 copies, less than 50 copies, less than 45 copies, less than 40 copies, less than 35 copies, less than 30 copies, less than 25 copies, less than 20 copies, less than 15 copies, less than 10 copies preferably less than 5, such as 4.

Expression of said POI in can be enhanced compared to when said chimeric gene is inserted into non-NOR genomic DNA. Expression of said POI can also be enhanced compared to expression driven by an average pol II promoter, preferably enhanced compared to a strong pol II promoter

### FIGURE LEGENDS

FIG. 1: Schematic of the trapping construct (TC) and control construct (CC) that were used to transform N. oceanica cells. The TC is a promotorless cassette that relies on insertion into an actively transcribed gene for expression of EGFP and zeo^{R} by “trapping” of upstream exons. If the construct is inserted into an intron, the splice acceptor (SA) sequence ensures that the transgenes are ligated onto upstream exons during RNA splicing. In the CC, transcription of the transgene is driven by the endogenous promoter and terminator of the VCP1 gene and the α-tubulin gene respectively. The P2A sequence encodes a viral peptide that facilitates synthesis of 2 independent proteins from a single transcript, yielding free EGFP and zeo^{R}. The TC is flanked by recognition sites for the type IIS restriction endonuclease MmeI which was an essential part for tracing the insertion sites in transformant strains.

FIG. 2: EGFP fluorescence of TC transformant strains. The boxplot represents results of flow cytometry analysis for 50,000 cells plotted with the ordinate on a logarithmic scale. Hinges of the boxes reach to the first and third quartiles of the distributions, whereas whiskers extend to an additional 1.5×IQR. Multiple strains were found with single cell fluorescence levels comparable to the control strain. TC strain #17 showed strongly increased fluorescence compared to all other strains. The wild type and a representative CC strain are highlighted grey.

FIG. 3: Analysis of transgene expression in TC #17 compared to a strain carrying the CC and to the wild type using different methodologies. (A) Fluorescence microscopy images using transillumination and GFP channels. (B) Quantification of single cell GFP fluorescence intensity using flow cytometry. Dot and error bars show the mean±SD (N=6) of the median of fluorescence emission distributions. Asterisks denote significant difference compared to the CC, assessed by Tukey's HSD test. (C) Quantification of EGFP transcript abundance relative to the control construct measured by RTq-PCR using Actin as a reference gene. TC #17 displayed a ˜135-fold increase compared to transcript levels of transformants carrying the CC. (D) Western blot with a GFP-binding antibody on 30 μg of soluble protein separated by SDS-PAGE. Multiple bands of different sizes correspond to EGFP-zeo^{R} fusion protein (43.6 kDa) and free EGFP (29.4 kDa). No difference in size of the protein was detected between TC #17 and CC strains, indicating that translation initiation likely occurs on the EGFP AUG codon in TC #17.

FIG. 4: Schematic of the bicistronic reporter construct EC-BRA including mechanism of mRNA translation. Transcription of the reporter cassette was driven by an endogenous promoter belonging to the lipid droplet surface protein (P_{LDSP}) and terminated at the α-tubulin terminator (T_{α-tub}). The polycistronic mRNA facilitated translation of the fluorescent reporter tdTomato and zeo^{R} in the regular cap-dependent manner. After translating the first 2 genes, ribosomal subunits (ellipses) would dissocicate at the 3′-end of zeo^{R} through 3 consecutive translational STOP codons. Translation of the NanoLuciferase (NLuc) gene was possible only through cap-independent translation, i.e. by ribosomal binding to the sequence upstream of the NLuc AUG codon. This sequence (represented as I) was different for the 4 EC-BRA constructs. EC-BRA-Noc-IRES contained the putative N. oceanica IRES consisting of 255 nucleotides upstream of EGFP ATG codon in TC #17. EC-BRA-CrPV-IRES carried the well-documented cricket paralysis virus IRES and EC-BRA-crTMV-IRES carried the IRES (CP,148) (CR) of crucifer-infecting tobamovirus. EC-BRA-NC contained no additional sequence in between zeo^{R} and NLuc and served as a negative control. All constructs had a single nucleotide insertion after zeo^{R} to prevent production of functional luciferase in the case of ribosomal read-through at the 3 translational STOP codons.

FIG. 5: Luminescence readings for transformants carrying different versions of the bicistronic reporter construct EC-BRA. Transformant strains for all 4 constructs were analysed for expression of tdTomato via flow cytometry. 9-10 independent strains with comparable tdTomato fluorescence levels were selected and subjected to luciferase assays (N=4 technical replicates). The values are normalized to the optical densities of samples. Transformants carrying the construct EC-BRA-Noc-IRES (putative N. oceanica IRES) displayed substantially increased luminescence levels compared to the wild type. Strains carrying constructs with the CrPV, the crTMV IRES and the negative control (NC) did not show an increase in luminescence compared to the wild type. (*): p<0.05; (**): p<0.01; (***): p<0.001

FIG. 6: Schematics of TC insertion in TC #17. (A) Representation of the insertion of the TC into chromosome 3 in strain TC #17 (top). A double-strand break (DSB) in the 25S rRNA gene probably caused integration of the cassette via NHEJ. Nucleotide sequence conservation of 25S rRNA genes from H. sapiens (HS) and S. cerevisiae (SC) compared to N. oceanica (NO) (bottom). The alignment is represented by grey bars for identities, black bars for mismatches and horizontal lines for gaps. Helices 64-71 (H64-71) lie proximately upstream of the TC insertion site in TC #17 and they are conserved on the nucleotide sequence level between different phyla. (B) Schematic of the secondary structure within domain IV of the 25S rRNA, modified from (Leshin et al., 2011, RNA Biology, 8 (3), 478-487). The nucleotide sequence shown corresponds to the S. cerevisiae rRNA but the secondary structure is highly conserved and likely identical in N. oceanica. The helices with the highest degree of nucleotide sequence conservation are enclosed in a rectangle. TC insertion in TC #17 occurred in the loop of helix 71. Helices that have been reported to interact with the SSU or ITAF proteins are marked with an asterisk.

FIG. 7: Schematic representation of EC1-5. EC1 was amplified from chromosome 3 in TC #17. Different parts of the native rDNA cistron were removed in EC2-5 to ascertain which elements have importance for gene expression.

FIG. 8: Quantification of transgene expression in EC1-5 strains by flow cytometry. The boxplot illustrates the levels of single cell fluorescence for 10 representative EC1-5 strains. All constructs gave rise to transformant strains with fluorescence intensities comparable to that of TC #17. This suggests that the rDNA elements between the Pol I promoter and the 25S rRNA gene are dispensable for transgene expression.

FIG. 9: Schematic representation of EC6-7. With EC5 as the shortest previously tested and fully functional construct, EC6-7 were constructed by removing parts of the 25S rRNA gene to narrow down elements important for transgene expression.

FIG. 10: Quantification of transgene expression in EC5-7 strains by flow cytometry. The boxplot illustrates the levels of single cell fluorescence for 10 representative EC5-7 strains. All constructs gave rise to colonies with fluorescence intensity comparable to that of TC #17. However, for construct EC7, most colonies were non-fluorescent whereas only a small fraction showed strong fluorescence emission.

FIG. 11: Putative HR-mediated insertion schematics and genetic characterization of EC1 and EC7 transformant strains. (A) Possible insertion of EC1 and EC7 into chromosome 3 via HR. The linear EC (top) can homologously recombine with the genomic DNA (center) via double crossovers at the ends of the cassette. Genotyping PCRs were carried out on genomic DNA of transformant strains (bottom) to check whether the ECs had been inserted via HR. (B) Genetic characterization for independent EC1 (left) and EC7 (right) strains. PCR reactions were carried out using primers illustrated in (A) to check for integration of the EC inside the NOR of chromosome 3. Arrows on the right sides of gels indicate the expected size for successful HR. Almost all EC1 strains had the same genotype as TC strain #17 indicating high efficiency of HR with this construct. For EC7 strains, the cassette was inserted inside this NOR only in strains with strong fluorescence emission, whereas no HR-mediated insertion was observed for non-fluorescent strains. For some of the strong fluorescent strains the amplicons were larger than expected. In these transformants HR had occurred between sequences of the 25S rRNA gene instead of the Pol I promoter or terminator.

FIG. 12: (A) Schematic representation of CRISPR-Cas-mediated insertion of EC7 into the genome. EC7 was enclosed between homology flanks facilitating HDR-mediated insertion adjacent to the rDNA cistron on chromosome 3 (EC7-CRISPR-NOR). (B) Fluorescence emission levels of EC7 transformants created by CRISPR-Cas technique. Strains carrying EC7 adjacent to or inside of the rDNA cistron show comparable transgene expression levels, whereas EC7 inserted into random genomic loci by non-homologous end joining (EC7-NHEJ) does not induce strong reporter expression. Correct HDR-mediated insertion of the cassettes into the genome was verified by PCR.

FIG. 13: Design of ECT1-5 and fluorescence screening of transformant strains. (A) ECT1 carried the same elements between the α-tubulin terminator and the Pol I terminator as EC7, except for the 25S rDNA sequence which was removed in ECT1. ECT2-5 were based on ECT1 and carried deletions of either the Poll terminator (ECT2) or the α-tubulin terminator (ECT3-5). In ECT4, the α-tubulin terminator was replaced by the endogenous LDSP gene terminator. ECT5 was modified to encode an A107 tract flanked by an HDV ribozyme sequence to facilitate formation of a free 3′-poly (A) tail on the EGFP cassette upon transcription of this construct. All cassettes were targeted to replace the rDNA cistron of chromosome 3 through HR using CRISPR/Cas technique. (B) Single cell fluorescence levels of representative transformants. The mean±SD (N=6) of the median of the single cell green fluorescence are shown together with changes relative to the mean of EC7. Significance levels were calculated by Tukey's HSD test. Removal of the 25S rDNA sequence downstream of the α-tubulin terminator did not impair transgene expression in ECT 1 transformants compared to a representative EC7 strain. Similarly, removal of the Pol I terminator did not interfere with expression, indicating that another DNA element in these transformants can substitute for transcriptional termination. Removal of the α-tubulin terminator however substantially decreased gene expression efficiency in ECT3 strains. Expression levels were not restored by substitution with the LDSP terminator sequence in ECT4. Addition of a sequence that facilitates formation of a free poly (A) tail on the transcript increased gene expression levels of transformants slightly compared to ECT3 strains but did not restore the high fluorescence levels of EC7. (*): p<. 05; (**): p<. 01; (***): p<. 001.

FIG. 14: EGFP transcript abundance in representative ECT1, ECT2 and ECT3 transformants. EGFP transcript abundance was quantified by RTq-PCR using the standard curve method with correction for differences in starting template using Actin as a reference gene. The mean±SD (N=3) of EGFP transcript normalized to the abundance in the ECT1 transformant are given together with relative changes compared to the mean of ECT1 and significance levels. (*): p<. 05; (***): p<. 001.

FIG. 15: Partial IRES deletions causes a substantial decrease or loss of fluorescence in transformant strains. (A) Schematic of ECs with partial IRES deletions, compared to ECT2. The ECs were designed to integrate into the rDNA locus of chromosome 3 by HDR. (B) EGFP fluorescence emission levels of representative strains quantified by flow cytometry. The mean±SD (N=6) of the medians of single cell green fluorescence distributions is shown. Relative changes of the wild type-corrected average fluorescence compared to ECT2 are shown together with significance levels above each sample group. (***): p<. 001.

FIG. 16: Investigation of Pol I-based transcription. (A) Design of constructs ECP- and ECPL was based on ECT2. The Pol I promoter sequence was removed in ECP-, and substituted for the medium-high strength Pol II promoter of the LDSP gene in ECPL. Both cassettes were targeted to replace the rDNA cistron of chromosome 3 through HR using CRISPR/Cas technique. (B) Single cell green fluorescence of representative transformants. The mean±SD (N=6) of the median single cell green fluorescence is shown together with relative changes of the wild type-corrected average fluorescence compared to ECT2. Significance levels are given above each sample group. ECP-transformants are viable but do not display increased green fluorescence over the wild type. Transformant strains carrying the cassette under control of the LDSP promoter display fluorescence levels comparable to levels observed for random genomic insertion of this construct (data not shown), a decrease of ˜95% compared to ECT2 transformants. Together with previous experiments these data suggest that Pol II is not responsible for transgene expression in ECT2 transformants. (*): p<. 05; (**): p<. 01; (***): p<. 001.

FIG. 17: Fluorescence quantification in strains carrying different reporter genes. All reporter genes yielded functional protein, including tdTomato which has a molecular weight of ˜54 kDa. The boxplots show the single cell fluorescence distribution of representative samples.

FIG. 18: Expression of luciferase and camelid antibody genes using the novel expression system. (A) Schematic of ECs transformed into N. oceanica to test expression of Nanoluc luciferase and anti-GFP V_{H}H. Both ECs were based on ECT1 and integrated into the NOR of chromosome 3 via HR and CRISPR/Cas technique. ECVHH-NLuc carried a fusion of an anti-GFP V_{H}H and the Nanoluc luciferase gene (NLuc) which replaced the EGFP-P2A-bleo^{R} cassette of ECT1. Downstream of the TE element (T_{α-tub}) we inserted an antibiotic resistance cassette (P_{LDSP}-blast^{R}-T_{35S}) for selection of transformant strains. In ECVHH-his, NLuc had been replaced with a his-tag coding sequence. (B) Luminescence activity in 3 ECVHH-NLuc transformant strains compared to strains carrying control constructs. Luminescence signal was on average ˜12× and 43× higher in ECVHH-NLuc transformants compared to strains in which expression of NLuc was driven by the nitrate reductase promoter (P(NR)-NLuc) and the Ribi promoter (P(Ribi)-NLuc) respectively. Numbers above boxes indicate the median of 4 technical replicates (8 for wild type, WT). Values for the 3 ECVHH-NLuc transformant strains were pooled and evaluated together. (C) Indirect ELISA with protein extracts of ECVHH-NLuc and ECVHH-his strains. Soluble extracts were analysed for their capacity to bind to GFP, which had been immobilized on a microtiter plate. Bound antibodies were quantified by using a secondary anti-camelid antibody coupled to HRP for colorimetric detection with TMB. Bars represent the mean±SE of technical triplicates for 3 strains per EC pooled together. Signals produced by extracts of both ECs were higher than those produced by a conventional mammalian anti-GFP IgG (PC). Signals of ECVHH-his samples were on average 24% higher compared to signals of ECVHH-NLuc samples. (D) SDS-PAGE analysis of V_{H}H-his purification. Protein extract from an ECVHH-his transformant was subjected to immobilized metal affinity chromatography for nanobody purification. The different fractions were analysed by SDS-PAGE. Raw extract and flowthrough were loaded onto the gel at the same protein concentration. The arrow indicates the expected position of V_{H}H-his. Remarkably, the V_{H}H-his band was visible for the raw extract with Coomassie blue staining and did not require more sensitive detection methods. This band was not present in wild type extracts (data not shown) and it was not visible in the flowthrough, which shows that V_{H}H-his was effectively bound to the chromatography column. Elution fractions that did not show any signal other than the expected band were pooled and used in a subsequent ELISA for V_{H}H quantification in raw extracts. (E) Calibration curve of quantitative indirect ELISA for V_{H}H quantification in microalgal raw extracts. Purified V_{H}H-his at 0-4 μg ml⁻¹ was added to immobilized GFP. Bound antibody was quantified as explained above. Dependency of signal strength on standard concentration was well explained with a Michaelis-Menten fit (dashed line, R²>0.995). (F) ELISA signal of soluble extracts from 3 ECVHH-his transformant strains. Serial dilutions between 1-100 μg soluble protein ml⁻¹ were added to immobilized GFP and analysed in the same assay as described under (e). Signal strength depended on protein concentration following a Michaelis-Menten relation, which was also observed for pure V_{H}H-his. Based on the data presented here, V_{H}H-his concentrations in soluble extracts were calculated using nonlinear regression with the Michaelis-Menten model described under (e). Signals observed for wild type controls are shown as white triangles, the 3 ECVHH-his transformant strains are shown as grey diamonds, grey squares and black circles. Data are represented as the mean±SE of technical duplicates.

FIG. 19: (A) Design of yEC1 and yEC2 for Pol I based expression in yeast NOR. The linear ECs were designed to integrate into 25S rRNA genes of S. cerevisiae via HR. They carry an EGFP-P2A-URA3 reporter gene fusion flanked by the previously discussed IRES which is a fusion of 25S rDNA helices H64-H71 and the SA element of N. oceanica VCP1 intron 1. The H64-H71 nucleotide sequence was identical to the N. oceanica version and to the S. cerevisiae version in yEC1 and yEC2 respectively. Homology arms direct the constructs to H71 of the 25S rRNA gene. (B) Fluorescence microscopy images of yEC1 and yEC2 transformant strains compared to the parental strain and transformants carrying a control construct. Reporter fluorescence is slightly enhanced for yEC2 compared to yEC1, indicating that activity of the employed IRES is organism-specific and might depend on high sequence similarity to the endogenous rRNA.

FIG. 20: Schematic of ECs used to transform P. pastoris. The linear ECs were designed to integrate into either the 26S rDNA or the AOX1 locus by homologous recombination. Integration into the 26S rDNA cistron was designed to cause a functional combination of Pol I-based transcription and IRES-mediated translation of the reporter genes in PPEC-TEV-26S strains. In strains carrying the control construct PPEC-GAP-26S, transcription of reporter genes was mediated by the Pol II promoter of the glyceraldehyde-3-phosphate dehydrogenase (GAP) gene. The reporter gene EGFP was polycistronically linked to the antibiotic resistance gene zeo^{R} using a P2A linker peptide. The coding sequence is highlighted in grey. Homology flanks (HF) of 1 kb length were added to facilitate insertion at the target site of the genome (light grey) by HR. Insertion of PPEC-TEV-AOX1 and PPEC-GAP-AOX1 was designed to occur at the AOX1 locus in the opposite orientation of the AOX1 gene.

FIG. 21: EGFP fluorescence analysis for PPEC transformant strains. The boxplot represents single cell fluorescence emission of ten transformant strains for PPEC-TEV-26S, PPEC-GAP-26S and PPEC-GAP-AOX1. Hinges of the boxes reach to the first and third quartiles of the distributions, whereas whiskers extend to an additional 1.5×IQR. Gene expression is much more variable for PPEC-TEV-26S strains compared to the control constructs.

FIG. 22: Schematic of ECs designed to investigate the α-tubulin terminator as a potential TE in P. pastoris. The ECs were based on PPEC-TEV-26S and facilitated integration into the 26S rDNA locus in the same fashion. PPEC-Noc-TEV-TE carries the Noc-IRES and α-tubulin terminator on the 5′- and 3′-side of the TEV IRES and the coding sequence (grey) respectively. In PPEC-Noc-TE, the TEV IRES was deleted to evaluate the potential of the Noc-IRES sequence as an IRES in P. pastoris.

FIG. 23: EGFP fluorescence analysis for P. pastoris transformant strains carrying translational elements from N. oceanica. The boxplot represents single cell fluorescence emission of ten transformant strains for PPEC-Noc-TEV-TE and PPEC-Noc-TE. Hinges of the boxes reach to the first and third quartiles of the distributions, whereas whiskers extend to an additional 1.5×IQR. No improvement of EGFP fluorescence was seen for either construct compared to best performers among PPEC-TEV-26S transformants. PPEC-Noc-TEV-TE strains showed moderate fluorescence emission which was significantly higher than that of the wild type, suggesting that the Noc-IRES is functionally active in P. pastoris.

FIG. 24: Quantification of single cell EGFP fluorescence of ECT1-5 strains. Single cell green fluorescence emission of transformant strains was quantified by flow cytometry. The scatter plot presents median fluorescences of three colonies per construct compared to the fluorescence of the wild type (WT) and a representative EC7 strain. Fluorescence levels within constructs were similar between different colonies. Correct insertion was verified by PCR and sequencing for three colonies per construct. One colony per construct was chosen for quantification of fluorescence emission shown in FIG. 1.15. Different colours represent different colonies for ECT1-5. For wild type and EC7, the mean±SD (N=3) are shown.

FIG. 25: Quantification of single cell EGFP fluorescence of IRES-deletion construct strains. Single cell green fluorescence emission of ECi1, ECi2 and ECi3 transformant strains was quantified by flow cytometry. The scatter plot presents median fluorescences for duplicate cultures of six colonies compared to the fluorescence of the wild type (WT) and a representative ECT2 strain. Fluorescence levels within constructs were similar between different colonies. Correct insertion was verified by PCR and sequencing for three colonies per construct. One colony per construct was chosen for quantification of fluorescence emission shown in FIG. 15. Different colours represent different colonies for ECi1-3. For wild type and ECT2, the mean±SD (N=3) are shown.

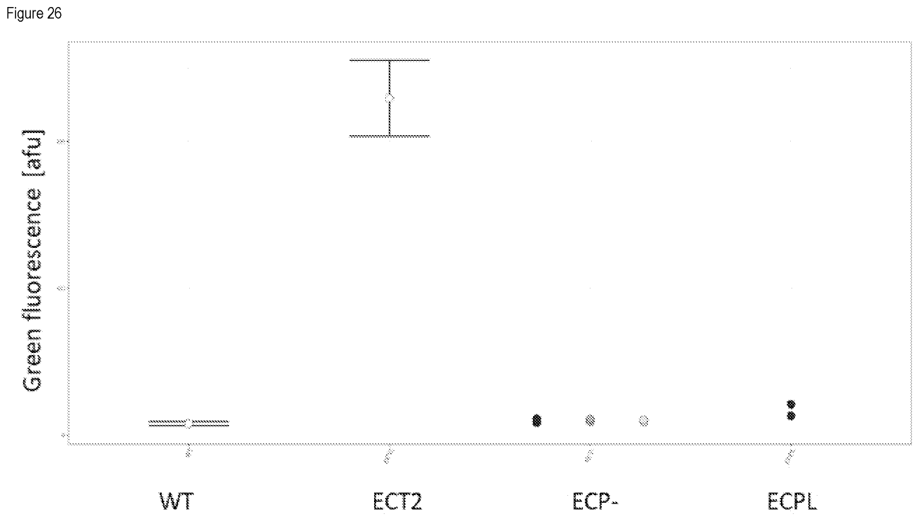

FIG. 26: Quantification of single cell EGFP fluorescence of ECP- and ECPL construct strains. Single cell green fluorescence emission of three ECP- and one ECPL transformant strains was quantified by flow cytometry. The scatter plot presents median fluorescences for duplicate cultures compared to the fluorescence of the wild type (WT) and a representative ECT2 strain. Fluorescence levels within constructs were similar between different colonies. Correct insertion was verified by PCR and sequencing. One colony per construct was chosen for quantification of fluorescence emission shown in FIG. 16. Different colours represent different ECP-colonies. For wild type and ECT2, the mean±SD (N=3) are shown.

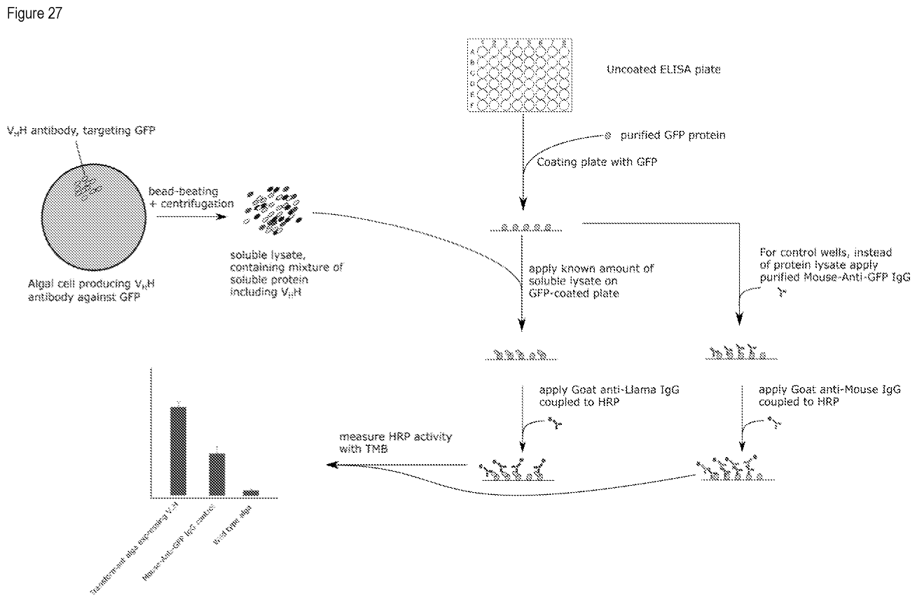

FIG. 27: Schematic representation of ELISA procedure for qualitative examination of V_{H}H activity in N. oceanica lysate. HRP: Horseradish peroxidase; IgG: Immunoglobulin G; TMB: 3,3′,5,5′-Tetramethylbenzidine.

FIG. 28: Schematic representation of ELISA procedure for quantitation of V_{H}H activity in N. oceanica lysates. HRP: Horseradish peroxidase; IgG: Immunoglobulin G; TMB: 3,3′,5,5′-Tetramethylbenzidine.

FIG. 29: Schematic representation of the expression constructs for transfection of mammalian cells.

FIG. 30: Schematic representation of the transfection procedure. The procedure includes methods and means to ensure delivery of ECs to the nucleolus of mammalian cells, in order to trigger EC integration by HR/HDR.

### DETAILED DESCRIPTION

Although RNA polymerase II (Pol II) transcribes the entirety of the protein-coding genes in eukaryotic cells, only ˜5% of the total RNA is mRNA (Lodish et al., Molecular Cell Biology, 4th edition, Section 11.6). The vast majority of RNA molecules (50-80% in mammalian cells, Lodish et al, supra; Russel and Zomerdijk, Biochem Soc Symp. 2006; (73): 203-16). are rRNA molecules. 4 different types of rRNA exist in eukaryotes: 18S, 5.8S, 25S/28S and 5S rRNA. The first 3 kinds constitute the bulk of rRNA and they are all expressed from 1 transcriptional unit, termed an rDNA cistron. rDNA cistrons are arranged the same way in all eukaryotes and they often occur in tandem arrays of sometimes hundreds of rDNA cistrons separated by non-transcribed spacer regions. A single rDNA cistron consists of a transcriptional promoter for eukaryotic DNA-directed RNA polymerase I (Pol I), the rRNA genes in order of 18S, 5.8S and 25S/28S separated by internal transcribed spacers ITS1 and ITS2 and demarcated by a Pol I terminator at the 3′ boundary

Remarkably, Pol I is a dedicated enzyme for the transcription of only the rRNA genes, but it is responsible for the synthesis of almost the entire cellular RNA. Several reasons contribute to the strong transcriptional activity of Pol I. (i) rDNA cistrons are organised in a designated area of the nucleus called nucleolus that concentrates all necessary machinery for transcription of the rDNA cistrons (high concentration of Pol I and related transcription factors), as well as for co/post-transcriptional processing of pre-rRNA. (ii) Pol I is a highly efficient while comparably simple enzyme that has an increased transcription speed compared to Pol II. It passes through nucleosomes (Merkl et al., Biorxiv Oct. 2, 2018) and does not seem to pause during elongation, like its Pol II counterpart. (iii) When Pol I escapes the promoter region, the transcription factors remain bound to the promoter DNA, recruiting the next Pol I enzyme, facilitating simultaneous transcription of a single rDNA cistron by multiple Pol I complexes.

The inventors have developed a gene expression system that employs Pol I for expression of a GOI and targeting this to the nucleolar DNA, thereby facilitating tremendously improved levels of gene expression. In N. oceanica for example, both mRNA and protein levels were found to be significantly higher than when using a promoter for Pol II. Naturally, RNA molecules synthesized by Pol I are not undergoing the same post-transcriptional processing like mRNAs so they lack a 5′-cap and poly-A tail. We found that by introducing elements for cap-independent translation around the GOI, translation can occur despite the lack of mRNA features. Said elements are an internal ribosome entry sites (IRES) and optionally a (cap-independent) translation enhancer (TE). Presence of a TE was found to further enhance gene expression. This strategy is an improvement of the traditional, Pol II-based way of genetically engineering eukaryotes and it has the potential to completely replace it in cases where high levels of a POI are desired.

The IRES in our construct offers the additional advantage of creating polycistronic transcripts, which is not normally possible in eukaryotic systems. Thus, depending on the length of the GOIs and efficiency of the transformation system, multiple GOIs or even entire metabolic pathways could potentially be overexpressed at high levels from a single construct. This would greatly simplify metabolic engineering of eukaryotic cells. First, the DNA construct can be much shorter in a polycistronic expression system because only 1 transcriptional promoter and terminator are required. Second, in eukaryotic transformants, transgenes are often silenced by mechanisms such as e.g. RNA interference (RNAi) or heterochromatinization. When utilising monocistronic constructs for expressing multiple GOIs, each transcriptional unit can be the target of such transcriptional down-regulation. In a polycistronic cassette, all POIs can theoretically be expressed at the same level because they all have the same level of transcript abundance. Furthermore, neither RNAi nor heterochromatinization have ever been reported to affect the activity of Pol I.

This promoter concept can be used for the expression of recombinant protein or genes in a variegate range of organisms. Our solution offers the advantage to use one single expression cassette (EC) and achieving high expression level already with a single integration event (e.g. in N. oceanica), thus avoiding gene silencing and deletion of other genes often linked with multicopy random transformation.

Thus, in a first aspect, a method is provided for producing/expressing one or more proteins of interest in a eukaryotic cell comprising the step of:
introducing into a eukaryotic cell a nucleic acid molecule comprising a polynucleotide encoding a protein of interest (POI)
wherein said nucleic acid molecule is targeted to the nucleolar DNA (i.e. the nucleolar genome), preferably to a nucleolar organizer region (NOR), of said organism, to form upon integration of said nucleic acid molecule a chimeric gene comprising the following operably-linked elements (in the 5′ to 3′ direction):
   i. a polymerase I promoter
   ii. a polynucleotide encoding an internal ribosomal entry site (IRES);
   iii. said polynucleotide encoding said POI
   iv. optionally a 3′ end region/transcription terminator

Thus, upon integration into the nucleolar DNA of said nucleic acid molecule, the chimeric gene thus encodes an (chimeric) mRNA molecule (also referred to as a fusion RNA or fuRNA) comprising an IRES and a polynucleotide encoding said POI.

The nucleolar DNA of an organism is the genomic DNA of an organism that is organized in nucleoli, i.e. specific structures within the nucleus that are the site of ribosome biogenesis. Nucleoli are made of proteins, DNA and RNA and form around specific chromosomal regions called nucleolar organizing regions (NORs). A nucleolar organizer region (NOR) is a part of the genome that contains ribosomal DNA (rDNA) cistrons and any additional sequences which form the DNA constituent of the nucleolus. The genome architecture regarding NORs is comparable between different eukaryotic organisms. NORs usually exist as clusters of rDNA tandem repeats which are distributed typically over 1-6 chromosomes (McStay & Grummt, 2008, Annual Review of Cell and Developmental Biology, 24 (1), 131-157). The number of cistron copies in a single tandem repeat however varies significantly between different species and usually ranges from 70-140 (McStay & Grummt, 2008, Annual Rev Cell Developm Biology, 24 (1), 131-157; Petes, 1979, PNAS, 76 (1), 410-414; Sáez-Vásquez & Gadal, 2010, Molecular Plant, 3 (4), 678-690)

An “rDNA cistron” or “rDNA gene” or “rRNA encoding gene”, as used herein, is a transcriptional unit encoding one or more rRNAs. The 18S, the 5.8S, and the 25/28S RNA molecules are expressed from one cistron, where the respective coding sequences are interlaced with two internal transcribed spacers, ITS1 and ITS2, and flanked upstream by a 5′ external transcribed spacer and a downstream 3′ external transcribed spacer (Zentner et al., 2011 Nucleic Acid Res 39 (12) 4949-4960); Edger et al., 2014, PLOS ONE 9 (7) e101341. These components are transcribed together to form the 45S pre-rRNA. The 45S pre-rRNA is then post-transcriptionally cleaved by C/D box and H/ACA box snoRNAs (Watkins et al., 2012, RNA. 3 (3): 397-414), removing the two spacers and resulting in the three rRNAs by a complex series of steps (Venema et al., 1999. Anual Rev Genet 33 (1) 261-311.

Thus, a NOR can e.g. be identified by the presence of rDNA cistrons, i.e. rRNA encoding genes. The boundaries of a NOR can be defined by the proximal and distal junctions (DJs and PJs) that act as anchor points and between which the rDNA arrays are located. See e.g. Mangal et al., 2017 (FEBS J, December; 284 (23): 3977-3985), Floutsakou at al., 2013 (Genome Res. 2013 December; 23 (12): 2003-2012) Schoffer et al., 2018 (Histochem Cell Biol. 2018; 150 (3): 209-225) and McStay & Grummt (supra).

In humans, the NORs are located on the short arms of the acrocentric chromosomes 13, 14, 15, 21 and 22, the genes RNR1, RNR2, RNR3, RNR4, and RNR5 respectively. These regions code for 5.8S, 18S, and 28S ribosomal RNA. The NORs are “sandwiched” between the repetitive, heterochromatic DNA sequences of the centromeres and telomeres (McStay B, 2016 Genes & Development. 30 (14): 1598-610). NOR sequences for the short arms of chromosomes 13, 14, 15, 21, and 22 are described in The Genome Reference Consortium. (“GRCh38.p13 has been released” GenomeRef. Retrieved 16 Aug. 2019). Some sequences of flanking sequences proximal and distal to NORs have been reported (Floutsakou et al., 2013, Genome Research. 23 (12): 2003-12). Coding regions of rDNA are highly conserved among species. Thus, conserved sequences at coding regions of rDNA allow comparisons of remote species, even between yeast and human. Human 5.8S rRNA has 75% identity with yeast 5.8S rRNA. In yeast, a NOR exists on chromosome XII (Petes et al., PNAS Jan. 1, 1979 76 (1) 410-414). In Komagataella phaffii (P. pastoris), NORs are located at the 3′ end of each of the 4 chromosomes (Küberl et al., J Biotechnol 2011 Jul. 20; 154 (4): 312-20). In Arabidosis thaliana, A. thaliana NORs are located close to the telomeres on the top or north arms of chromosomes 2 and 4 (NOR2 and NOR4, respectively) (Copenhaver et al., Plant J. 9 (2) 1996, p 259-272). In Drosophila melanogaster, NORs are located on the short arm of the entirely heterochromatic Y chromosome and in the centric heterochromatin of the X chromosome (Ritossa et al., 1966. Natl Cancer Inst Monogr. December; 23:449-72).

As used herein, a “promoter” or a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase.

As used herein, a polymerase I (pol I) promoter, is a promoter that can drive transcription by RNA polymerase I, the polymerase that transcribes ribosomal RNA (except the 5S ribosomal RNA, which is operated by an RNA polymerase III promoter). By contrast, mRNA genes (i.e. encoding proteins) are transcribed by RNA polymerase II (Pol II), while RNA polymerase III (pol III) transcribes DNA to synthesize ribosomal 5S rRNA, tRNA and other small RNAs. Pol I does not require a TATA box in the promoter, but instead relies on an upstream control element (UCE) located between −200 and −107, and a core element located between −45 and +20. When Pol I escapes and clears the promoter during transcription, UBF and SL1 remain-promoter bound, ready to recruit another Pol I. Thus, contrary to Pol II-transcribed genes, which associate with only one complex at a time, each active rDNA gene can be transcribed multiple times simultaneously, making it the fastest polymerase. The term polymerase I promoter is sometimes interchangeably used with the term rDNA promoter, although the 5S ribosomal RNA is driven by an RNA polymerase III promoter (the latter being excluded from the present scope)

As used herein, an internal ribosomal entry site (IRES) is an RNA element that allows for translation initiation in a cap-independent manner. IRESs are often located in the 5′ UTR but can also be located elsewhere in a transcript to allow initiation of translation of a downstream open reading frame. Many viral genes employ IRESs but also eukaryotic IRESs exists (sometimes referred to as viral and cellular IRESs respectively). IRESs are often used to allow expression of two or more proteins from a single vector under the control of a single promoter. IRESs are well known in the art and are for example described in Yamamoto et al. (Trends Biochem Sci 2017 August; 42 (8): 655-668) Viral IRESs are e.g. described in Justine Mailliot et al. (Wiley Interdiscip Rev RNA 2018 March; 9 (2)) and cellular IRESs, e.g in Komar et al. (Cell Cycle. 2011; 10 (2): 229-240). Thus, the presence of an IRES in the chimeric gene enables translation and thus expression of the POI.

Some IRESs like HCV-like IRESs directly bind the 40S ribosomal subunit to position their initiator codons in such a way that they are located in the ribosomal P-site without mRNA scanning. These IRESs still use the eukaryotic initiation factors (eIFs) elF2, elF3, elF5, and elF5B, but do not require the factors elF1, elF1A, and the elF4F complex. Others, such as picornavirus IRESs, do not bind the 40S subunit directly, which is recruited instead through interaction with elF4G (Hellen et al., Genes Dev 2001, 15 (13): 1593-1612). Many viral IRESs (and cellular IRESs) require additional proteins to mediate their function, known as IRES trans-acting factors (ITAFs).

IRES elements vary in length from less than 100 to >1000 nucleotides (Baird et al., 2006) with popular studied IRESs typically being between 200-440 nucleotides long (Bochkov & Palmenberg, 2006, Bio Techniques, 41 (3), 283-292; Pestova et al., 1998, Genes and Development, 12 (1), 67-83; Wilson et al., 2000), Type I viral IRESs are located up to >150 nucleotides upstream of the open reading frame (ORF) under their control (Jackson, et. al., 2014, EMBO Journal, 33(1), 76-92) whereas types II-IV are usually located immediately upstream of the ORF and position the ribosome directly onto the initiation codon (Baird et al., 2006).

A functional IRES can be identified for example by testing the putative IRES sequence in a polycistronic, (e.g. bicistronic) reporter construct. When an IRES segment is located between two reporter open reading frames in a bicistronic mRNA molecule, it can drive translation of the downstream protein coding region independently of the 5′-cap structure bound to the 5′ end of the mRNA molecule. If the IRES is functional, both proteins are produced in the cell; the first reporter protein is produced by the cap-dependent initiation, while translation initiation of the second protein is directed by the IRES element located between the two reporter protein coding regions.

A 3′ end region or transcription terminator, as used herein, refers a section of nucleic acid sequence that marks the end of a gene or operon in genomic DNA during transcription. This sequence mediates transcriptional termination by providing signals in the newly synthesized transcript RNA that trigger processes which release the transcript RNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs.

In one embodiment, the nucleic acid molecule comprising the polynucleotide encoding the POI can be targeted to be integrated downstream of an endogenous polymerase I promoter that is naturally already present in the genome of said organism, such that the endogenous promoter at its endogenous location becomes operably-linked to and capable of directing transcription of the polynucleotide encoding the POI. For example, any of the endogenous rDNA cistron promoters can be used to drive transcription of the integrated polynucleotide encoding the POI. In this embodiment, the IRES may also already be endogenously present at the site of integration of the nucleic acid molecule such that upon integration the polynucleotide encoding the POI becomes operably linked to the endogenous IRES and Pol I promoter, or the nucleic acid molecule comprising the polynucleotide encoding the POI already comprises the IRES and is inserted downstream of the endogenous pol I promoter.

In a preferred embodiment, the nucleic acid to be integrated already comprises a polymerase I promoter operably linked to the IRES and the polynucleotide encoding the POI, which are together targeted to be integrated into the nucleolar DNA (e.g. NOR).

Similarly, the nucleic acid molecule comprising the polynucleotide encoding the POI can be targeted to the nucleolar DNA such that upon integration it becomes operably-linked to an endogenous 3′ end region/transcription terminator naturally already present in the nucleolar genome of said organism. For example, the terminator of one of the endogenous rDNA cistrons can be used. Alternatively, the nucleic acid molecule comprising the polynucleotide encoding the POI can already comprise a 3′ end region/transcription terminator, which are together targeted to be integrated into the nucleolar DNA (e.g. NOR).

Thus, also provided is a method for expressing or producing one or more proteins of interest (POI) in a eukaryotic cell, comprising the steps of:
a. introducing into a eukaryotic cell a nucleic acid molecule comprising a chimeric gene comprising the following operably-linked elements:
   i. a polymerase I promoter
   ii. a polynucleotide encoding an internal ribosomal entry site (IRES).
   iii. A polynucleotide encoding a protein of interest (POI)
   iv. Optionally a 3′ end region/transcription terminator

wherein said chimeric gene is integrated into the nucleolar DNA of said organism, such as into the nucleolar organizer region (NOR).

Not using an endogenously present promoter and/or terminator (and/or IRES), but starting with a nucleic acid molecule already comprising the promoter, terminator and IRES (i.e. the chimeric gene) has the advantage of more flexibility in the choice of the promoter, terminator and IRES to optimize expression of the POI and in addition widens the range where the gene can be targeted within the nucleolar DNA. Thus, using a nucleic acid molecule already comprising the chimeric gene for targeting to the nucleolar DNA additionally simplifies the targeting in terms of choice of flanking regions and/or use of sequence specific nucleases for targeted insertion (see further below). Furthermore, in this way the chimeric gene can be targeted to a location where it is expected to be least disruptive (does not negatively affect the function of the endogenous rDNA genes) or to a location that is suspected or known to be suitable for high expression (e.g. an actively transcribed region of the nucleolus).

To target the nucleic acid molecule into the nucleolar DNA, e.g. into the NOR, the nucleic acid molecule can be flanked with one or more flanking sequences for allowing integration of said nucleic acid molecule at a predefined site in said nucleolar DNA by (one-sided or two-sided) homologous recombination. As will be known to a person skilled in the art, such flanking sequence or sequences need to have sufficient homology over a sufficient length to the genomic region or regions flanking the predefined site for allowing targeted integration by homologous recombination.

Another way in which the nucleic acid molecule can be integrated into the nucleolar DNA, e.g. into the NOR, is by inducing a targeted DNA break or nick at a predefined site in said nucleolar DNA, upon which the nucleic acid molecule can be inserted at or near the break site, i.e. the predefined site. This can occur without flanking sequences having homology to the genomic region or regions flanking the predefined site, but also in the presence of such flanking region or regions (which will then aid in the targeting at the desired position). Preferably, such a predefined site is a location that is suspected or known to be suitable for high expression (e.g. an actively transcribed region of the nucleolus, such as the present NOR locus on chromosome 3 in N. oceanica).

A targeted DNA break, e.g. a single stranded break (a nick) or a double stranded break, can be induced at the predefined site by any method know in the art, e.g. by providing the cell with or expressing in the cell a sequence specific nuclease (SSN). The nucleic acid molecule according to the invention can be used as a template to repair the DNA break and as such be inserted or integrated at the break site.

SSNs can be designed or programmed to recognize and cleave basically any desired target sequence. Examples of SSNs include e.g. meganucleases (MGNs), zinc-finger nucleases (ZNFs), TAL effector nucleases TALENs) or a nucleic acid-guided nuclease, such as DNA-guided-nucleases or RNA-guided nucleases. Examples of RNA-guided nucleases include e.g. Cas9, Cas12a/Cpf1, C2C12, Mad8, Cas-Phi, as e.g. described in Gaj et al. (Cold Spring Harb Perspect Biol. 2016; 8 (12): a023754) and Makarova et al (Nature Reviews Microbiology volume 18, pages 67-83 (2020).

SSNs can be expressed in the cell by transforming or transfecting the cell with an expression cassette encoding the nuclease, optionally together with (an expression cassette encoding) a guide nucleic acid (e.g. guide DNA or guide RNA) in case of a nucleic acid-guided nuclease, wherein the guide polynucleotide is capable of directing the nuclease to the desired location/sequence in the nucleolar DNA. Alternatively, the SSN can be provided to the cell as a protein, e.g. by electroporation, optionally together with (an expression cassette encoding) a guide nucleic acid (e.g. guide DNA or guide RNA) in case of a nucleic acid-guided nuclease, or with a ribonucleoprotein complex comprising the nuclease and its guide.

In one embodiment, the flanking sequence(s) flanking the nucleic acid molecule or chimeric gene may be at least 10, 15, 20, 30, 40, 50, 100, 150, 200, 300, 400, 500, 600, 700, 800, 900 nt, 1 kb or more in length, such as about 1.1, 1.2, 1.3, 1.5, 1.5, 1.6, 1.7, 1.8, 1.9, 2 kb, or even more such as about 3 kb, 4 kb, 5 kb more in length and/or may have at least 70%, 75%, 80%, 85%, 90%, 91%, 92%, 93%, 94%, 95%, 96%, 97%, 98%, 99% or 100% sequence identity to the nucleolar DNA at said predefined site in the nucleolar DNA where said chimeric gene is to be integrated. The higher the % sequence identity (homology) of the flanking regions to the DNA at the predefined site (and the longer the flanking sequences), the more precise and seamlessly the integration will be.

Preferably, the nucleic acid molecule of the invention is integrated into a transcriptionally active NOR to obtain a high level of gene expression. Such transcriptionally active chromosomal regions are also referred to as euchromatic regions or euchromatin. In terms of nucleolar DNA, actively transcribed regions are often found where rDNA genes, i.e. rRNA encoding genes, are located, usually in so-called rDNA cistrons. Transcriptionally active or euchromatic NORs can e.g. be identified as undercondensed regions of the chromosome e.g. by DAPI staining (Pontvianne et al., Volume 16, Issue 6, 9 Aug. 2016, Pages 1574-1587) or electron tomography (Heliot et al., Molecular Biology of the Cell 2017, Vol. 8, No. 11) or by silver staining (Goodpasture et al., Chromosoma volume 53, pages 37-50, 1975). By contrast, inactive copies within a cell can be identified through psoralen crosslinking, restriction digestion and electrophoretic separation analysis (Conconi et al., Cell, VOLUME 57, ISSUE 5, P753-761, Jun. 2, 1989). Optionally, the method comprises the further step of detecting expression of the POI to verify that the chimeric gene is indeed located in a transcriptionally active region of the NOR. Additionally or alternatively, a NOR can be selected that is suspected to or known to be transcriptionally active, such as the present NOR locus on chromosome 3 in N. oceanica.

Thus, in a preferred embodiment, the nucleic acid molecule or the chimeric gene is inserted in the vicinity of or in an rDNA cistron (but still within the nucleolar DNA, preferably the NOR). In the vicinity can be for example within 20 kb, 15 kb 10 kb, 5 kb, 4 kb, 3 kb, 2 kb, 1.5 kb, 1 kb, 750 bp, 500 bp, 400 bp, 300 bp, 250 bp, 200 bp, 150 bp, 100 bp or 50 bp of an rDNA cistron. Preferably, the chimeric gene is inserted not within but in the vicinity of an rRNA cistron, so as not to interfere with rRNA expression and not to negatively affect ribosomal function, such as in the intergenic regions, preferably between 2 tandem repeats (adjacent rDNA cistrons). However, since many organisms have multiple cistron copies, it is expected that even when disrupting a single cistron, this will mostly not negatively impact ribosomal function. This can e.g. be within 20 kb, 15 kb 10 kb, 5 kb, 4 kb, 3 kb, 2 kb, 1.5 kb, 1 kb, 750 bp, 500 bp, 400 bp, 300 bp, 250 bp, 200 bp, 150 bp, 100 bp or 50 bp upstream of the endogenous promoter of the rDNA or upstream of the (RNA) coding sequence of the rDNA gene. This can also e.g. be within 20 kb, 15 kb 10 kb, 5 kb, 4 kb, 3 kb, 2 kb, 1.5 kb, 1 kb, 750 bp, 500 bp, 400 bp, 300 bp, 250 bp, 200 bp, 150 bp, 100 bp or 50 bp downstream of the endogenous terminator or of the (RNA) coding sequence of the rDNA gene. Preferably, the insertion is not made within the junction sequences (DJ and PJ), which are responsible for anchoring the rDNA arrays.

In a preferred embodiment, the chimeric gene is targeted to be located in or in the vicinity of a 25S rDNA gene or 28S rDNA gene, depending on the organism, i.e. in the coding sequence of the 25S/28S gene or promoter region. In another preferred embodiment, the chimeric gene is targeted to the present NOR locus on chromosome 3 in N. oceanica.

It was presently found that expression of the POI with the pol I promoter was significantly enhanced when the chimeric gene was inserted in the nucleolus compared to elsewhere in the genome. It has also been found that with the use of a pol I promoter expression of the POI was significantly higher compared to a pol II promoter, when targeted to the nucleolus, whereas expression from that pol II promoter was found to be similar when not targeted to the nucleolus. Without wishing to be bound by theory, by targeting the nucleic acid molecule of the invention in the vicinity of or within an rDNA gene/cistron, it is believed that it is inserted in a transcriptionally active genomic region favourable for pol I mediated transcription and hence that the chimeric gene will also be actively transcribed (to a similar extent), since it can make use of the transcription enabling and enhancing factors associated with the endogenous rRNA expression machinery.

Thus, in one embodiment, expression of the POI is enhanced compared to when the chimeric gene would be present in the genome of the eukaryotic cell outside the nucleolus (outside the NOR), i.e. expression is higher when the chimeric gene is present in the nucleolar genome compare to the nucleoplasmic genome genome. Expression can be enhanced, such as about 1.5 fold, about 2 fold, about 3 fold, about 4 fold, about 5 fold, about 6 fold, about 7 fold, about 8 fold, about 9 fold, about 10 fold or even more, such as about 12 fold, about 15 fold, about 20 fold, about 25 fold, about 230 fold, about 35 fold, about 40 fold, about 45 fold, about 50 fold, or even more, e.g. about 12-43 fold, or expression can go from undetectable to detectable. In another embodiment, expression of the POI is enhanced compared to a pol II promoter, such as an average pol II promoter, or enhanced compared to a strong or constitutive pol II promoter (a pol II promoter having average or strong or constitutive activity in the eukaryotic organism where the POI is to be expressed). For example, expression can be enhanced at least about 1.5 fold, about 2 fold, about 3 fold, about 4 fold, about 5 fold, about 10 fold, about 15 fold or about 20 fold or even more with respect to an average pol II promoter, such as LDSP promoter (in N. oceanica), e.g. about 20 fold. In another embodiment, expression of the POI is enhanced compared to a strong or constitutive pol II promoter. For example, expression can be enhanced at least about 1.5 fold, about 2 fold, about 3 fold, about 4 fold, about 5 fold, about 6 fold, about 7 fold, about 8 fold, about 9 fold, about 10 fold or even higher with respect to a strong or constitutive pol II promoter, such as the VCP promoter (in N. oceanica), e.g. about 8 fold. Further, examples of strong or constitutive pol II promoters include e.g. the TEF promoter (Maury et al., 2016, PLOS ONE, 11 (3), e0150394) and the promoter of the GAP gene ((Marx et al., 2009, FEMS Yeast Research, 9 (8), 1260-1270; Song et al., 2019, BMC Biotechnology, 19 (1), 54; Várnai et al., 2014, Microbial Cell Factories, 13 (1), 57; Zhang et al., 2009, Molecular Biology Reports, 36 (6), 1611-1619)

On the mRNA level, expression of the transcript can be even further enhanced compare to pol II driven mRNA expression. For example, transcript abundance can be enhanced compared to an average or strong pol II promoter at least by about 1.5 fold, about 2 fold, about 3 fold, about 4 fold, about 5 fold, about 6 fold, about 7 fold, about 8 fold, about 9 fold, about 10 fold, about 15 fold, about 20 fold or even more, such as about 25 fold, about 50 fold, about 75 fold, about 100 fold, about 125 fold, about 135 fold, about 150 fold, about 175 fold, or about 200 fold. The comparison can each time be with respect to a pol II promoter present in the nucleoplasmic genome or in the nucleolar genome.

(Quantitative) protein expression can be measured using any technique available in the art, such as western blotting, Elisa, enzymatic assays, fluorescence measurement.

It was presently found that the presence of a translational enhancer (TE) can further enhance expression of the POI, e.g. by as about 1.5 fold, about 2 fold, about 3 fold, about 4 fold, about 5 fold, about 6 fold, about 7 fold, about 8 fold, about 9 fold, about 10 fold or even higher. Thus, in any of the embodiments described herein, the chimeric gene preferably further comprises a sequence encoding a translation enhancer (TE), e.g. a cap-independent translation enhancer (CITE). A TE, as used herein, refers to an RNA element in a transcript that is capable of enhancing translation). A CITE, as used herein, refers to a translation enhancer (a TE) that is capable of enhancing translation in a Cap-independent manner. CITE elements are typically found in the 3′ UTR in positive strand RNA plant viruses (3′ CITE), where they substitute for the absence of a 5′-cap and poly (A) tail by either recruiting eukaryotic translation initiation factors (eIFs) or the ribosomal subunits to the viral genome. (Gao et al., 2012, Journal of Virology, 86 (18), 9828-9842; Nicholson et al., 2010, Rna, 16 (7), 1402-1419; Stupina et al., 2008, Rna, 14 (11), 2379-2393; Wang et al., 2009, Journal of Biological Chemistry, 284 (21), 14189-14202). To induce translation initiation, the recruited trans-acting factors need to be brought into proximity with the 5′-UTR. In most viruses that contain 3′-CITE elements, this is achieved through circularization of the RNA molecule by long distance RNA-RNA interactions between complementary bases in the 5′-UTR and the 3′-UTR of the viral genome or through recruitment of a protein bridge by designated elements in both UTRs (Bradrick et al., 2006, Nucleic Acids Research, 34 (4), 1293-1303; Gazo et al., 2004, Journal of Biological Chemistry, 279 (14), 13584-13592; Souii et al., 2015, Current Microbiology, 71 (3), 387-395). RNA circularization is also observed for mRNA molecules, where the 5′-cap and poly (A) tail can be connected through a protein bridge consisting of elF4E, elF4G and poly (A) binding protein (PABP) (Svitkin & Sonenberg, 2006; Wells et al., 1998, Molecular Cell, 2 (1), 135-140). In addition to being a necessary requisite for ribosome entry, mRNA circularization is thought to further function in guiding the translational machinery back to the 5′-UTR after termination, thereby minimizing eIF dissociation rates and maximising translation efficiency. This might also be the case for circularized RNA plant viruses. Whereas most 3′-CITEs do not require an IRES element in the 5′-UTR of an RNA to facilitate cap-independent translation, interactions between 3′-TEs and 5′-IRESs are able to increase IRES-mediated translation initiation levels, as was reported for foot-and-mouth disease virus (García-Nuñez et al., 2014, Virology, 448, 303-313; Serrano et al., 2006, Journal of General Virology, 87 (10), 3013-3022).

It was presently found that in the absence of an IRES (e.g. in ECi3 transformants), no reporter gene expression was observed, suggesting that the present TE element (in the alpha tubulin terminator) is not capable of driving cap-independent translation initiation independently of an IRES. However, the TE (in the alpha tubulin terminator) did increase expression, which could not be solely attributed to transcription termination or the presence or absence of a poly (A) tail. Thus, without wishing to be bound by theory, it is believed that the TE element may stimulate IRES-mediated translation by recruiting trans-acting factors such as eIFs or ITAFs to the RNA which might increase the chance of ribosome recruitment to the IRES. Alternatively, the TE element may simply enhance translation by facilitating circularization of the fuRNA, which could help to re-recruit ribosomes or translation-associated proteins back to the IRES after translation termination.

The TE can be located anywhere in the transcript, but may advantageously be located between the POI encoding polynucleotide and the 3′ end region/transcription terminator or within the terminator. Preferably, a TE from the same (endogenous) or a related species is used of the eukaryotic cell in which the POI is to be expressed or a viral TE active in the eukaryotic cell is used. For example, the TE can be selected from any of the above describe CITEs/TEs or any of the following:

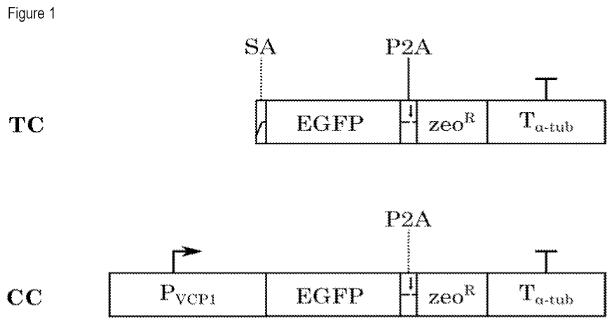

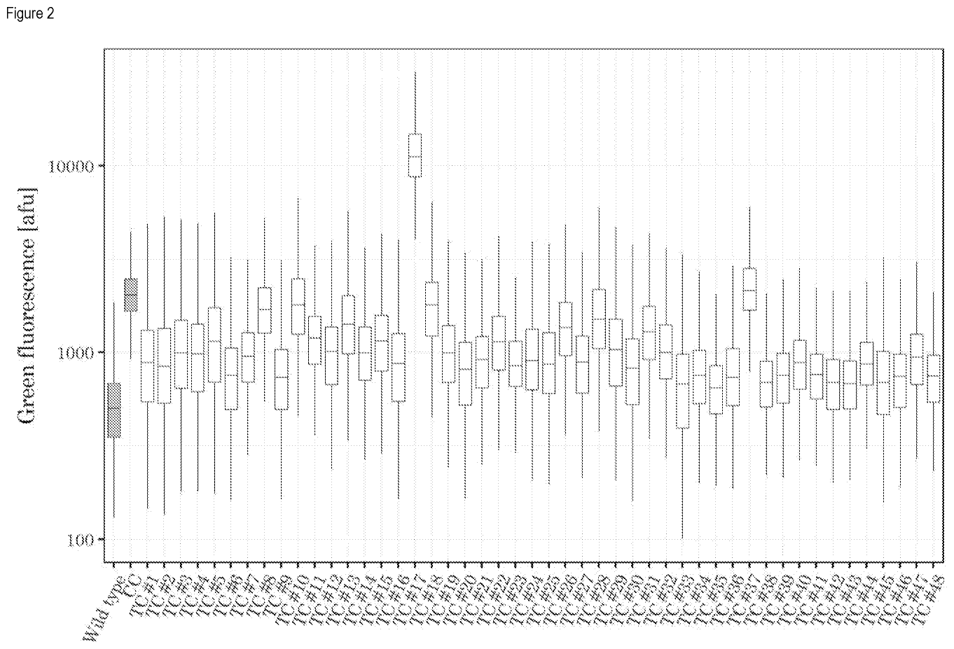

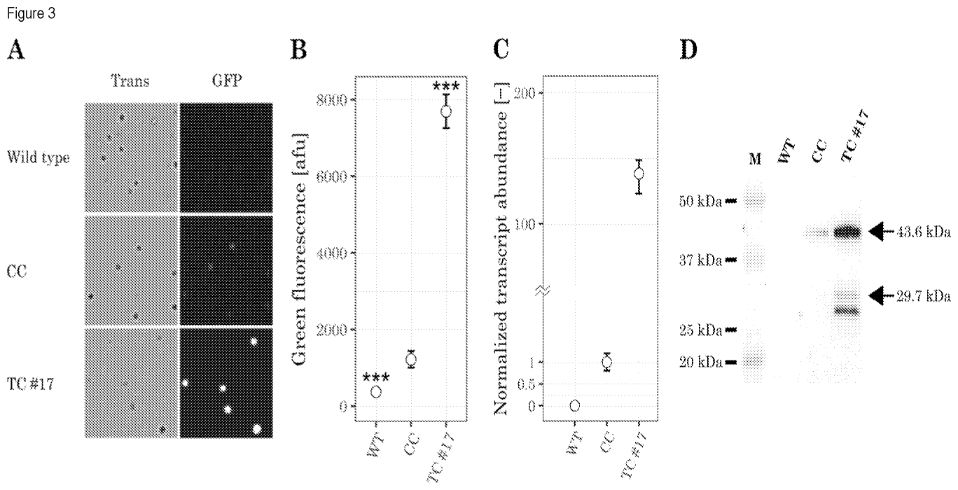

Members of the BTE (Barley yellow dwarf virus or BYDV-like element) class of CITEs, such as from BYDV itself, e.g. as shown in Truniger (Front. Plant Sci., 29 Nov. 2017) FIG. 1 or Wang et al., Virology. 2010 Jun. 20; 402 (1): 177-86). Members of the TED (translation enhancer domain) class, such as from Satellite tobacco necrosis virus, e.g. as shown in Truniger et al., (2017, supra) FIG. 2A or in U.S. Pat. No. 5,994,526. Members of the PTE (Panicum mosaic virus or PMV-like translational enhancer) class, such as from Panicum mosaic virus, as e.g. shown in Truniger (2017, supra) FIG. 2B or in Batten et al., (FEBS Lett 2006 May 15; 580 (11): 2591-7). Members of the ISS (I-shaped structure) class, such as from Melon necrotic spot virus (MNSV), e.g. as shown in Truniger (2017, supra) FIG. 2C or in Truniger et al. (2008, Plant J, December; 56 (5): 716-27). Members of the YSS (Y-shaped structure) class, such as from Tomato bushy stunt virus (TBSV), as e.g. shown in Truniger (2017, supra) FIG. 2D or in Fabian et al. (2006, RNA July; 12 (7): 1304-14). Members belonging to the TSS (T-shaped structure) class, such as from Turnip crinkle virus (TCV), as e.g. shown in Truniger (2017, supra) FIG. 3A,B or in McCormack et al. (2008, J Virol, 2008 September; 82 (17): 8706-20). Members of the CXTE (Cucurbit aphid-borne yellows virus or CABYV-Xinjiang-like translation element) class, such as from Cucurbit aphid-borne yellows virus, as e.g. shown in Truniger (2017, supra) FIG. 3C or in Miras et al., (2014, New Phytol, 2014 April; 202 (1): 233-46). The X region of the 3′UTR of Hepatitis C virus (HCV), which was reported to functionally interact with the IRES in the 5′UTR, enhancing translation 3-5 fold (Ito et al., J Virol. 1998 November; 72 (11): 8789-8796). More recently, it was shown that the RBP IG2FBP1 can interact with both, 5′ and 3′ UTR of HCV RNA (Weinlich et al., 2009, RNA August; 15 (8): 1528-42). It was suggested that IG2FBP1 then recruits elF3 and thereby enhances IRES mediated translation. Duck Hepatitis A virus also contains a 3′ TE element that increases IRE-mediated translation (Chen et al., 2018, Front Microbiol September 25; 9:2250). CU-rich elements (CUREs) may recruit PTB to the RNA (Matoulkova et al., 2-12, RNA Biol, May; 9 (5): 563-76), which can act as an ITAF to increase IRES dependent translation initiation (Sawicka et al., 2008, Biochem Soc Trans, 36 (4): 641-647). Sindbis virus (SINV) contains 3′ TEs shown to enhance translation in insect, but not mammalian cells, also when introduced into another, normally non-infectious alphavirus (Garcia-Moreno et al., 2016, Sci Rep January 12; 6:19217). Foot and mouth disease virus (FMDV) contains a 3′ TE that enhances translation mediated by a 5′ IRES through long distance interactions between conserved nucleotides (Lopez de Quinto et al., 2002 October 15; 30 (20): 4398-405; Garcia-Nunez et al., 2014 January 5; 448:303-13). Serrano et al. (2006, J Gen virol October; 87 (Pt 10): 3013-3022) describes interacting regions and Diaz-Toledano (2017 Nucleic Acid Res, February 17; 45 (3): 1416-1432) describes specific nucleotides.

In a specific embodiment, the TE may be the TE as present in the alpha tubulin terminator, preferably the alpha tubulin of the same (i.e. endogenous) or a related species is of the eukaryotic cell in which the POI is to be expressed. The TE may comprise or be comprised in the sequence of the alpha tubulin terminator from N. oceanica, e.g. the sequence of SEQ ID NO. 6, or a functional fragment of any one thereof.

In any of the embodiments described herein, the chimeric gene may further comprise a (polynucleotide encoding) a poly-adenylation (polyA) signal sequence. The terms “PolyA”, “Poly A element,” “Poly A region,” and “Poly A signal” and “Poly A sequence” are used interchangeably herein and is to mean nucleotide sequences capable of directing “polyadenylation” at the 3′ end of an RNA, e.g., by chemical reactions involving addition of multiple adenosine residues. “Poly A element,” “Poly A region,” and “Poly A signal” and “poly A sequence” are used interchangeably herein. A representative example of a Poly A element is provided by the SV40 polyA region. The pol I promoter can be any pol I promoter functional in the eukaryotic cell, i.e. that can drive expression of the operably linked coding region in the eukaryotic cell to the desired level. Similarly, it could be advantageous to use a pol I promoter from the same species or a related species of the eukaryotic cell wherein it is desired to express the POI. For example a human Pol I promoter is described in Russel et al. (2005, Trends Biochem Sci Volume 30, Issue 2, February, p 87-96) and Financsek et al. (1892, PNAS May; 79 (10): 3092-6), several primate pol II promoter as described in Agrawal et al., (2018, PloS One, December 5; 13 (12): e0207531), especially supplemental figure S3, a yeast pol I promoter is described in Kulkens et al. (1991 Nucleic Acid Res, Volume 19, Issue 19, 11 Oct. 1991, p 5363-5370) and RDN37-1 (https://www.yeastgenome.org/locus/S000006486), Arabidopsis thaliana pol I promoter is described in (Doelling et al. (1995, Plant J vol 8 (5), a Drosophila pol I promoter is described in Kohorn et al. (1983 Nature 304, p 179-181) In a specific embodiment, the Pol I promoter may comprise the sequence of SEQ ID NO. 1, SEQ ID NO. 2, SEQ ID NO. 3 or SEQ ID NO. 4, or nt 1-924 of SEQ ID NO. 11 (EC7), nt 1-924 of SEQ ID NO. 13 (EC7tdTomato), nt 1144-2067 of SEQ ID NO. 15 (ECT2-tdTomato) or nt 1144-2067 of SEQ ID NO. 16 (EC-VHH), or a functional fragment of any one thereof.

In any of the embodiments described herein, the 3′end region/transcription terminator can be any such element functional in said cell. It could be advantageous to use a terminator from the same species or a related species of the eukaryotic cell wherein it is desired to express the POI. It could also be advantageous to use corresponding pol I promoter-terminator pairs from the same organism or even the same gene. For example, primate terminators are described in Agrawal et al. (2018, supra), especially figure S3, yeast terminators are described in Reeder et al., (1999 Mol Cel Biol November; 19 (11): 7369-76). In a preferred embodiment, a pol II terminator can be used, such as the 3′ end region/terminator of the alpha-tubulin gene, preferably originating from the same or a related species of the eukaryotic cell. A pol II terminator can also be used in addition to a Pol I terminator. In a specific embodiment, the alpha-tubulin terminator can comprise SEQ ID NO. 6 or a functional fragment thereof. In another embodiment, the pol II terminator can be the CaMV 35S terminator, e.g. comprising nt 4714-4950 of SEQ ID NO. 16 (EE-VHH), or the AOX1 terminator, such as from Pichia pastoris, e.g. nt 2293-2539 of SEQ ID NO. 18 (PPEC-TEV-26S), or the URA3 RNA polymerase II transcriptional terminator, such as from S. cerevisiae, e.g. nt 1327-2130 of SEQ ID NO. 20 (yEC2) or a functional fragment of any one thereof. The pol I termination can comprise the sequence of SEQ ID NO. 7, SEQ ID NO. 8, SEQ ID NO. 9 or SEQ ID NO. 10 or nt 3579-4265 of SEQ ID NO. 11 (EC7), nt 4293-4979 of SEQ ID NO. 13 (EC7-tdTomato, or a functional fragment of any one thereof.

Preferably, the chimeric gene in any of the embodiments described herein comprises a terminator and a TE/CITE element, or the terminator comprises a TE/CITE element. For example, the chimeric gene comprises a Pol I and/or Pol II terminator and a TE/CITE element, or a Pol I and/or pol II terminator comprising a TE/CITE element, such as the alpha tubulin terminator.

In any of the embodiments described herein, the IRES can be any IRES functional in said cell and capable of initiation translation of the POI. It could be advantageous to use an IRES from the same species or a related species of the eukaryotic cell wherein it is desired to express the POI. Also viral IRESs can advantageously be used if they function in the cell where the POI is to be expressed. For example, the IRES can be selected from human elF4G homologue DAP5 or Mnt genes, the encephalomyocarditis virus (EMCV) IRES, the hepatitis C virus IRES, the Gtx IRES, the dicistrovirus intergenic region (IGR) IRES. Other IRESs that can be used are e.g. the Tobamovirus (TMV) IRES (Dorokhov et al., J Gen Virol, September; 87 (Pt 9): 2693-2697), the Turnip crinkle virus (TCV) IRES (May et al., J Virol March 29; 91 (8): e02421-16), the pelargonium flower break virus (PFBV) IRES (Fernández-Miragall et al., PLOS One, 2011; 6 (7): e22617), IRESs from picornavirus such as the poliovirus IRES (Pelletier et al., 1988 Nature 334, p 320-325) or foot and mouth disease virus IRES (Belsham, 1992, EMBO J 11:1105-1110), the tobacco etch virus (TEV) IRES (Zeenko et al., 2005, J Biol Chem July 22; 280 (29): 26813-24), the Swine fever virus IRES (Fletcher et al., 2002, J Virol, May; 76 (10): 5024-33), an IRES from or active in Pichia pastoris (Huang et al., 2019, Biotechnol Biofuels, December 27; 12:300). Further IRESs can be found in the IRES database (http://iresite.org) as described in Mokrejs et al (2006 Nucleic Acid Res 2006 Jan. 1; 34). In a specific embodiment, the IRES may comprise the sequence of SEQ ID NO. 5, or comprise SEQ ID 19 or the IRES as present in PPEC-TEV-26S (nt 1001-1146 SEQ ID NO. 15), or comprise SEQ ID NO. 21 or the IRES as present in yEC2 (nt 301-555 of SEQ ID NO. 16), or a functional fragment of any one thereof.

Other sequences that can beneficially be included in the chimeric gene to further enhance expression are (polynucleotides encoding) leader sequences, such as the omega-leader sequence of TMV (Mandeles 1968, J Biol chem 243 (13) 10 p 3671-3674). A leader sequence or 5′ untranslated region (5′ UTR), as used herein, refers to the region in an mRNA preceding (5′ of) the start codon where translation is initiated and begins at the transcription start site and ends one nucleotide (nt) before the start codon (usually AUG) of the coding region. A leader sequence/5′UTR is involved in regulation of translation of the coding sequence in the mRNA, e.g. by interaction with the translational machinery

The methods and chimeric genes described herein may be used to express multiple POIs from the same chimeric gene. Thus, in another embodiment, the chimeric gene may further comprise a polynucleotide encoding a second IRES (and optionally a second CITE/TE) operably-linked to a second polynucleotide encoding a second protein of interest. The chimeric gene may also comprise a third, fourth and fifth etc IRES operably linked to a third, fourth and fifth etc POI coding region (and optionally a third, fourth and fifth etc CITE/TE). For each additional POI the same or a different IRES may be used. This will result in a polycistronic mRNA from which the various POIs can be translated via each respective IRES.

The eukaryotic cell can be any cell wherein it is desired to express or produce a POI. Preferably such a cell can be cultured at the desired scale and is susceptible to transformation, i.e. taking up the nucleic acid molecule encoding the POI and integrating the nucleic acid molecule into its nucleolar DNA so as to express the POI.

The eukaryotic cell can be selected from an animal call, plant cell, a protist cell and fungal cell.

Animal cells can be mammalian cells, such as mouse, rat or human cells, or can be insect cells, e.g. lepidopteran cells. Examples of suitable cells include Chines Hamster Ovary Cells (CHO), Baby Hamster Kidney Cells (BHK), Human Embryonic Kidney Cells (HEK 293), PER.C6® Cells, and derivatives thereof.

In some embodiments, the cell (e.g. animal or human cell), is in vitro or ex vivo. In some embodiments, the cell is not a human embryonic stem cell. In some embodiments, the method is not a method performed on the human (or animal) body, i.e. the cell is not a cell in the human (or animal) body (but can be a cell of the human or animal body ex vivo or in vitro).

In one embodiment, the cell is a plant cell, such as from a higher plant, or from an alga or seaweed (pluricellular or unicellular).

In one embodiment, the eukaryotic cell can be a lower eukaryotic cell or a unicellular eukaryotic cell, such as a protist, algal, yeast or fungal cell.

The eukaryotic cell can also be a fungal cell, e.g, a yeast cell. For example, it can be selected from an Aspergillus species including, but not limited to, Aspergillus nidulans, Aspergillus niger, Aspergillus terreus, Aspergillus oryzae and Aspergillus terreus; more preferably the Aspergillus species is Aspergillus nidulans or Aspergillus niger. Alternatively, the fungal species could be a Candida species. Or the yeast or fungal species may be selected from: Aspergillus species including Aspergillus fumigatus, Aspergillus nidulans, Aspergillus terreus, Aspergillus versicolor, Canariomyces species including Canariomyces thermophile; Chaetomium species including Chaetomium mesopotamicum, Chaetomium thermophilum; Candida species including Candida bovina, Candida sloofii, Candida thermophila, Candida tropicalis, Candida krusei (=Issatchenkia orientalis); Cercophora species including Cercophora coronate, Cercophora septentrionalis; Coonemeria species including Coonemeria aegyptiaca; Corynascus species including Corynascus thermophiles; Geotrichum species including Geotrichum candidum; Kluyveromyces species including Kluyveromyces fragilis, Kluyveromyces marxianus; Malbranchea species including Malbranchea cinnamomea, Malbranchea sulfurea; Melanocarpus species including Melanocarpus albomyces; Myceliophtora species including Myceliophthora fergusii, Myceliophthora thermophila; Mycothermus species including Mycothermus thermophiles (=Scytalidium thermophilum/Torula thermophila); Myriococcum species including Myriococcum thermophilum; Paecilomyces species including Paecilomyces thermophila; Remersonia species including Remersonia thermophila; Rhizomucor species including Rhizomucor pusillus, Rhizomucor tauricus; Saccharomyces species including Saccharomyces cerevisiae, Schizosaccharomyces species including Schizosaccharomyces pombe, Scytalidium species including Scytalidium thermophilum; Sordaris species including Sordaria thermophila; Thermoascus species including Thermoascus aurantiacus, Thermoascus thermophiles; Thermomucor species including Thermomucor indicae-seudaticae and Thermomyces species including Thermomyces ibadanensis, Thermomyces lanuginosus, Yarrowia species. The yeast cell or fungal cell can also be selected from a Saccharomyces species, such as Saccharomyces cerevisiae.

In one embodiment, the cell is a plant cell, for example a lower plant cell, such as a algal cell, e.g. a green algal cell or a microalgal cell. Microalgae include inter alia a species of a genus selected from the group consisting of Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricospheera, Crypthecodinium, Cryptomonas, Cyanidioschyzon, Cyclotella, Cylindrotheca, Cymatopleura, Dixoniella, Dunaliella, Ellipsoidon, Emiliania, Entomoneis Eremosphaera, Emodesmius, Euglena, Eustigmatos, Franceia, Fragilaria, Fragilariopsis, Gloeothamnion, Haematococcus, Halocafeteria, Hantzschia, Heterosigma, Hymenomonas, Isochrysis, Lepocinclis, Micractinium, Microchloropsis, Monodus, Monoraphidium, Nannochloris, Nannochioropsis, Navicula, Neochloris, Nephrochloris, Nephroselmis, Nitzschia, Ochromonas, Oedogonium, Oocystis, Ostreococcus, Parachlorella, Parietochioris, Pascheria, Pavlova, Pelagomonas, Phoeodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Schizochytrium, Skeletonema, Spyrogyra, Stichococcus, Tetrachlorella, Tetraselmis, Thalassiosira, Tribonema, Vaucheria, Viridiella, Vischena, and Volvox. For example, the cell may be from a diatom (Bacillariophyte) such as a species of Achnanthes, Amphora, Chaetoceros, Cyclotella, Cylindrotheca, Cymatopleura, Entomoneis, Fragileria, Fragilariopsis, Navicula, Nitzschia, Phoeodactylum, or Thalassiosira, for example, a species of Eustigmatos, Monodus, Nannochloropsis or Vischeria.

In a preferred embodiment, the cell is from a Chlorella species (such as Chlorella vulgaris, Chlorella sorokiniana, Chlorella kessleri, Chlorella luteoviridis, Chlorella desiccata, Chlorella minutissima, Chlorella sp.) or a Microchloropsis species (Microchloropsis gaditana or Microchloropsis salina).

In another preferred embodiment, the cell is of a Nannochloropsis species, such as Nannochloropsis oculata, Nannochloropsis limnetica, Nannochloropsis australis, Nannochloropsis salina, or Nannochloropsis oceanica, preferably Nannochloropsis oceanica. Further Nannochloropsis species are e.g. described in Andersen at al. (Protist, 1998 February; 149 (1): 61-74).

In another preferred embodiment, the cell is of a eukaryotic organism that has a relatively low copy number of rDNA genes. For example, in Nannochloropsis oceanica all 4 rDNA loci contain single cistrons instead of tandem repeats (Li et al., Plant Cell, vol. 26, no. 4, pp. 1645-1665 April 2014). It was presently surprisingly found that when targeting the chimeric gene in the vicinity of an endogenous rDNA gene in Nannochloropsis oceanica, expression was significantly enhanced with when compared to insertion in the non-nucleolar genome, where there was virtually no expression measurable. Without wishing to be bound by any theory, this could be explained by the relatively low copy number of rDNA genes in this organism, whereby there is not much competition or titrating out of transcription factors due to the presence of many copies of such genes in some other species, thus enabling the high expression of the POI.

Other organisms that have relatively low copy number of rDNA genes include e.g. certain algae: 1-2 for Nannochloropsis salina, Ostreococcus tauri and Pelagomonas calceolata, 3-4 for Emiliana huxleyi and Micromonas pusila 10-20 for Bathycoccus prasinos, Mesopedinella arctica and Tetraselmis sp. (Zhu et al., FEMS Microbiol Ecol 2005 Mar. 1; 52 (1): 79-92; Godhe et al., Appl Environ Microbiol, 2008 December; 74 (23): 7174-82), ˜4 for Phaeodactylum tricornutum and ˜12 for Thalassiosira oceanica (Gong et al., Front. Mar. Sci., 26 Apr. 2019). Yeasts or fungi with relatively low copy numbers include e.g. 45 for Aspergillus nidulans (Ganley et al., Genome Res 2007 February; 17 (2): 184-91), 26-63 for Kluyveromyces lactis (Maleszka et al., Mol Gen Genet 1990, 223:342-244) and ˜16 for Pichia pastoris (De Schutter et al., Nat Biotechn 2009, 27, 561-566).

Thus, a relatively low copy number in this respect can be said to be less than 70 copies, less than 60 copies, less than 50 copies, less than 45 copies, less than 40 copies, less than 35 copies, less than 30 copies, less than 25 copies, less than 20 copies, less than 15 copies, less than 10 copies preferably less than 5, such as 4 (in the case of Nannochloropsis oceanica).

Copy number of genes, such as rDNA cistrons, can be determined according to any technique available in the art, e.g. based on (conserved) sequences present in such genes, such as hybridization-based techniques (e.g. Southern blotting, fluorescent in situ hybridisation FISH), CRISPR-based detection assays, PCR based methods (e.g. qPCR, TaqMan), or sequencing based methods (e.g. whole-genome short-read DNA sequencing as described in Gibbons et al., 2014, Nat Commun 2014 Sep. 11; 5:485).

When aiming for multiple copies of the chimeric gene, e.g. to further enhance expression, the nucleic acid can also be targeted to highly homologous sequences between several rDNA cistrons, e.g. by choosing the flanking sequences and/or recognition site of the nuclease accordingly. In this way, enhanced expression may also be achieved in organisms having a relatively high copy number of rDNA cistrons, such as in human cells. For example, for human cells the diploid dosage for 18S, 5.8S and 28S genes was estimated to range between 67-412 (18S, average 217), 9-412 (5.8S, average 164) and 26-282 (28S, average 118), i.e. average haploid 28S dosage being 59 (Gibbons et al., 2014, Nature Communications 5:4850).

The cell can be transformed using any method suitable for said cell type, as will be known to a person skilled in the art and include e.g., viral or bacteriophage infection, transfection, conjugation, protoplast fusion, lipofection, electroporation, calcium phosphate precipitation, polyethyleneimine (PEI)-mediated transfection, DEAE-dextran-mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12) 00283-9. doi: 10.1016/j.addr.2012.09.023), and the like. The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

In a further embodiment, the method comprises the further step of determining/measuring the expression of the POI and/or selecting a cell having a higher expression (e.g. higher than the average, higher than an average Pol II promoter, higher than a strong pol II promoter, higher compared to insertion into the genome outside the nucleolar DNA, higher compared to without a TE/CITE element, or higher compared to an organism with a higher copy nr of rDNA cistrons, or selecting the cell having the highest expression.

In a further embodiment, the methods as herein described contain the further step of isolating and/or purifying said POI.

In a preferred embodiment, a nucleolar locus or NOR region is selected or used that is known or suspected to be transcriptionally active, to insert or form the chimeric gene, such as the present NOR locus on chromosome 3 in N. oceanica.

The protein or polypeptide of interest (POI) may be any protein that is of interest to express or produce, e.g. at large scale. Examples include antibodies, antigens, (e.g. for vaccine production), hormones (e.g. insulin), cytokines, enzymes, such as enzymes for the specific production of molecules (e.g. lipids).

To enhance expression of the POI, the coding sequence may be optimized for expression in the respective eukaryotic cell, e.g. by adapting the codon usage, or sequences may be included that promote targeting of the protein to the desired (sub-) cellular location or to promote export/secretion of the POI.

Also provided is a protein produced and optionally isolated/purified according to the herein described methods

In a second aspect, a chimeric gene is provided as described in any of the herein described embodiments and aspects. Thus, the chimeric gene may comprise the following operably-linked fragments:
i. a polymerase I promoter
ii. a polynucleotide encoding an internal ribosomal entry site (IRES).
iii. A polynucleotide encoding a protein of interest (POI)
iv. Optionally, a 3′ end region/transcription terminator

The chimeric gene may comprise any of the elements as described herein, such as the terminator, CITE/TE element, terminator comprising a CITE/TE element, a leader sequence, as described in any of the other aspects.

In a third aspect, the invention provided a eukaryotic cell (e.g. transgenic or cisgenic) obtainable or obtained by any of the methods described herein. Such a eukaryotic cell comprises the chimeric gene as described in any of the embodiments herein. Thus, provided is a eukaryotic cell comprising a chimeric gene the following operably-linked fragments:
i. a polymerase I promoter
ii. a polynucleotide encoding an internal ribosomal entry site (IRES).
iii. A polynucleotide encoding a protein of interest (POI)
iv. a 3′ end region/transcription terminator

wherein said chimeric gene has been integrated into the nucleolar DNA of said organism, such as into the nucleolar organizer region (NOR).

In one embodiment, the chimeric gene can employ and thus comprise an endogenous polymerase I promoter and/or terminator and/or IRES of said cell (i.e. already naturally present in the nucleolar genome of said cell, e.g. the promoter and/or terminator of an existing rDNA gene), when at least the polynucleotide encoding the POI has been targeted to be operably linked to said endogenous promoter and/or terminator and/or IRES.

In a preferred embodiment, the chimeric gene does not make use of (does not comprise) an endogenous promoter and/or terminator, but a pre-assembled chimeric gene as described is targeted to the nucleolar DNA (e.g. the NOR). Not using an endogenously present promoter and/or terminator, but starting with a nucleic acid molecule already comprising the chimeric gene has the advantage of more flexibility in the choice of the promoter and terminator to optimize expression of the POI, and in addition widens the range where the gene can be targeted within the nucleolar DNA. Thus, using a nucleic acid molecule already comprising a promoter and terminator (i.e. the chimeric gene) additionally simplifies the targeting in terms of choice of flanking regions and/or use of sequence specific nucleases for targeted insertion (as described above). The chimeric gene can be targeted/integrated within an rDNA gene/cistron or in the vicinity thereof.

In a preferred embodiment, the chimeric gene has been targeted/integrated into the nucleolar DNA in the vicinity of but not within an endogenous rDNA gene or cistron (but still within the nucleolar DNA, preferably the NOR). In this way, the chimeric gene does not interrupt the endogenous rDNA gene (especially not the transcript encoding region), and is also not expected to substantially interfere with the function/expression of the endogenous rDNA gene and thus not to negatively affect ribosomal function, such as in the intergenic regions, preferably between 2 tandem repeats (i.e. 2 adjacent rDNA cistrons). The chimeric gene can be located for example within 20 kb, 15 kb 10 kb, 5 kb, 4 kb, 3 kb, 2 kb, 1.5 kb, 1 kb, 750 bp, 500 bp, 400 bp, 300 bp, 250 bp, 200 bp, 150 bp, 100 bp or 50 bp of an rDNA cistron. This can e.g. be within 20 kb, 15 kb 10 kb, 5 kb, 4 kb, 3 kb, 2 kb, 1.5 kb, 1 kb, 750 bp, 500 bp, 400 bp, 300 bp, 250 bp, 200 bp, 150 bp, 100 bp or 50 bp upstream of the endogenous promoter of the rDNA or upstream of the coding sequence of the rDNA gene. This can also e.g. be within 20 kb, 15 kb 10 kb, 5 kb, 4 kb, 3 kb, 2 kb, 1.5 kb, 1 kb, 750 bp, 500 bp, 400 bp, 300 bp, 250 bp, 200 bp, 150 bp, 100 bp or 50 bp downstream of the endogenous terminator or of the coding sequence of the rDNA gene. Preferably, the insertion is not made within the junction sequences (DJ and PJ), which are responsible for anchoring the rDNA arrays.

The chimeric gene in the cell may further comprise any of the promoters, terminators, TE/CITE elements, POIs as described in any of the aspects and embodiments as described herein.

The eukaryotic cell can be any cell wherein it is desired to express a POI. Preferably such a cell can be cultured at the desired scale and is susceptible to transformation, i.e. taking up the nucleic acid molecule encoding the POI and integrating the nucleic acid molecule into its nucleolar DNA.

In such a cell according to the invention, expression of the POI is enhanced compared to when the chimeric gene would be inserted into the genome of the eukaryotic cell outside the nucleolus (outside the NOR), as described in any of the method embodiments described herein

The eukaryotic cell can be any of the cells as described herein, such as for the methods aspect of the invention.

In a preferred embodiment, the cell is from an organism that has a relatively low copy nr of rDNA genes. In another preferred embodiment, the cell is of a Nannochloropsis species, such as Nannochloropsis oculate, Nannochloropsis limnetica, Nannochloropsis australis or Nannochloropsis oceanica, preferably Nannochloropsis oceanica

In a fourth aspect, a nucleic acid molecule or vector is provided for expressing one or more proteins of interest (POI) in a eukaryotic cell or for targeting a polynucleotide encoding a POI to the nucleolar DNA (a NOR) of a eukaryotic cell, said nucleic acid molecule or vector comprising a polynucleotide encoding said at least one (POI), wherein upon integration into the nucleolar DNA, preferably to a nucleolar organizer region (NOR), of said organism a chimeric gene is formed comprising the following operably-linked fragments:
i. a polymerase I promoter
ii. a polynucleotide encoding an internal ribosomal entry site (IRES);
iii. said polynucleotide encoding said POI
iv. a 3′ end region/transcription terminator.

In this aspect or any other aspects or embodiment described herein, the nucleic acid molecule may be flanked with or the vector may comprise one or more flanking sequences that allow insertion of said polynucleotide encoding said POI into a predefined site in the nucleolar DNA of a eukaryotic organism by (one-sided or two-sided) homologous recombination to form said chimeric gene;

Alternatively or additionally, the nucleic acid molecule or vector can further comprise an expression cassette for expressing a sequence specific nuclease capable of inducing a DNA break at a predefined site in the nucleolar DNA of said eukaryotic cell for allowing integration of said polynucleotide encoding said POI at said predefined site to form said chimeric gene.

Thus, in a preferred embodiment, the nucleic acid molecule or vector comprises a chimeric gene comprising the following operably-linked elements:
i. a polymerase I promoter
ii. an internal ribosomal entry site (IRES).
iii. a polynucleotide encoding said protein of interest
iv. A 3′ end region/transcription terminator

optionally wherein said chimeric gene is flanked with one or more flanking sequences that allow insertion of said chimeric gene into a predefined site in the nucleolar DNA of a eukaryotic organism by (one-sided or two-sided) homologous recombination to form said chimeric gene; and/or

optionally wherein nucleic acid molecule or vector further comprises an expression cassette for expressing a sequence specific nuclease (SSN) capable of inducing a DNA break at a predefined site in the nucleolar DNA of said eukaryotic cell for allowing integration of said chimeric gene at said predefined site.

Also provided is a kit for expression of one or more proteins of interest in a eukaryotic cell or for targeting a polynucleotide encoding a POI to the nucleolar DNA (a NOR) of a eukaryotic cell, said kit comprising one or more containers comprising one or more vectors comprising a nucleic acid molecule comprising a polynucleotide encoding said at least one (POI), wherein upon integration into the nucleolar DNA, preferably to a nucleolar organizer region (NOR), of said organism a chimeric gene is formed comprising the following operably-linked fragments:
i. a polymerase I promoter
ii. a polynucleotide encoding an internal ribosomal entry site (IRES);
iii. said polynucleotide encoding said POI
iv. a 3′ end region/transcription terminator.

Also herein, the said nucleic acid molecule may be flanked with one or more flanking sequences that allow insertion of said polynucleotide encoding said POI into a predefined site in the nucleolar DNA (NOR) of a eukaryotic organism by (one-sided or two-sided) homologous recombination to form said chimeric gene to form said chimeric gene;

Alternatively or additionally, the kit may further comprise an expression cassette for expressing a sequence specific nuclease capable of inducing a DNA break at a predefined site in the nucleolar DNA (NOR) of said eukaryotic cell for allowing integration of said polynucleotide encoding said POI at said predefined site to form said chimeric gene.

Thus, in a preferred embodiment, said kit comprises one or more containers comprising one or more vectors comprising a chimeric gene comprising the following operably-linked elements:
i. a polymerase I promoter
ii. an internal ribosomal entry site (IRES).
iii. a polynucleotide encoding said protein of interest
iv. a 3′ end region/transcription terminator

optionally wherein said chimeric gene is flanked with one or more flanking sequences that allow insertion of said chimeric gene into a predefined site in the nucleolar DNA (NOR) of a eukaryotic organism by (one-sided or two-sided) homologous recombination; and/or

optionally wherein said kit further comprises an expression cassette for expressing a sequence specific nuclease capable of inducing a DNA break at a predefined site in the nucleolar DNA (NOR) of said eukaryotic cell for allowing integration of said chimeric gene at said predefined site.

In some embodiments, the nucleic acid molecule or vector or kit may further comprise an expression cassette encoding a guide RNA that is capable of directing the sequence specific nuclease to the predefined site in the nucleolar DNA (NOR) of said eukaryotic cell.

In any of the embodiments of this aspect, the nucleic acid molecule or vector may comprise any of the promoters, terminators, CITE/TE elements, polyA sequences and POIs as described in any of the aspects and embodiments described elsewhere herein.

In any of the embodiments of this aspect, the flanking sequences and SSN and optionally the guide RNA can be as described in any of the aspects and embodiments described elsewhere herein.

The nucleic acid molecules, vectors, chimeric genes and expression cassettes according to this aspect may further comprise any elements necessary or useful for the use in the eukaryotic cell in which the POI is desired to be expressed

In a fifth aspect, a method is provided for expressing or producing a protein or polypeptide of interest (POI), comprising the steps of a. providing a cell as described in any of the embodiments described herein, or produced according to any of the methods described herein, said cell comprising a chimeric gene as in any of the above embodiments described herein; and optionally
b. isolating or purifying said protein or polypeptide.

The cell can be any of the cells as described in any of the method embodiments described herein. In a preferred embodiment, the cell is of a Nannochloropsis species, such as Nannochloropsis oculate, Nannochloropsis limnetica, Nannochloropsis australis, Nannochloropsis salina, or Nannochloropsis oceanica, preferably Nannochloropsis oceanica.

Conveniently, once a cell according to the invention has been produced and confirmed to show the desired level of expression of the POI, it can be used to express further POIs from the same locus, by using the specific nucleolar locus as a landing site (safe harbour) for expressing other or additional POIs from the pol I promoter. More specifically, if at least one of the previously inserted POI encoding sequences is a selectable or screenable marker gene (e.g. a fluorescence gene or antibiotic marker), this can be replaced by a new POI encoding sequence, and loss of the marker function can conveniently be used as a screening tool for cells where the new POI encoding sequence has been inserted at the desired location.

Thus, in a sixth aspect, a method is provided for producing or selecting a cell or a cell strain where a sequence of interest is inserted (by homologous recombination) at a preselected site in the nucleolar genome, comprising the steps of:
a Providing a cell according to the present invention, i.e. a cell comprising a chimeric gene according to the present invention that has been integrated into the nucleolar genome of said organism, wherein said chimeric gene comprises and said cell expresses at least a selectable or screenable marker gene (fluorescence/antibiotic marker)
b. Providing said cell with a nucleic acid encoding a further protein of interest (POI), wherein said nucleic acid encoding said further POI is inserted to inactivate or replace the selectable or screenable marker gene, such that upon insertion the further POI is expressed.
c. Screening for loss of expression of said selectable or screenable marker gene, wherein loss of expression of said marker gene is indicative of insertion of the sequence of interest at said preselected site in the nucleolar genome.

Correct targeting to the preselected site in the nucleolar genome, i.e. the previously inserted/created chimeric gene comprising the selectable or screenable marker gene, can be done by providing said nucleic acid encoding said further POI with the appropriate flanking sequence(s) for homologous recombination and/or by expressing in said cell a SSN capable of inducing a DNA break at said preselected site (essentially as described elsewhere herein). Insertion should be such that such that the nucleic acid encoding the further POI becomes operably linked to the pol I promoter to enable expression of the further POI.

In a further embodiment, said method can comprise the further step of selecting a cell expressing the further POI, optionally selecting a cell having the desired expression level of said further POI (such as the highest expression of the further POI).

Also provided is a cell comprising a chimeric gene according to the present invention that has been integrated into the nucleolar genome of said organism (at the predefined site), wherein said chimeric gene comprises and said cell expresses at least a selectable or screenable marker gene (fluorescence/antibiotic marker).

Such a cell can be used in the above method to replace the selectable or screenable marker gene with a nucleic acid encoding a further POI.

In this aspect, the chimeric gene can comprise any of the elements as described herein (e.g. Pol I promoter, IRES, optionally TE), and the cell can be any of the cells as described herein. In a preferred embodiment of this aspect, the cell is of a Nannochloropsis species, such as Nannochloropsis oculate, Nannochloropsis limnetica, Nannochloropsis australis, or Nannochloropsis salina, Nannochloropsis oceanica, preferably Nannochloropsis oceanica.

In another preferred embodiment of this aspect, the preselected site where the chimeric gene has been inserted or formed is the present NOR locus on chromosome 3 in N. oceanica. In a further preferred embodiment, the cell according to the present invention to be provided with a nucleic acid encoding a further protein of interest (POI) is an N. oceanica cell comprising ECT2-tdTomato (SEQ ID NO. 15) targeted to the NOR locus on chromosome 3 (e.g. strain ECT2-tdTomato-S1) or an N. oceanica cell comprising EC7-tdTomato (SEQ ID NO. 13) targeted to the NOR locus on chromosome 3 (e.g. strain EC7-tdTomato-S1). The selectable or screenable marker gene can be a fluorescent reporter gene such as GFP, YFP, BFP, CFP, Cerulean, mCherry, DsRed, tdTomato, Kusabira Orange, Venus, Emerald, YGPF, EosFP, Ruby, Strawberry, or derivatives thereof, or a luminescent reporter gene such as luciferase enzymes, e.g. firefly luciferase, Renilla luciferase or Nano-luciferase, or a gene that confers a visible morphological characteristic with or without addition of chemicals into growth media, such as the bacterial lacZ gene that encodes β-galactosidase and facilitates blue-white screening of transformant colonies, or a gene that confers resistance against an antibiotic, such as against Ampicillin, Amphotericin B, Carbenicillin, Ciprofloxacin, Chloramphenicol, Erythomycin, Kanamycin, Gentamycin, Neomycin, Nystatin, Rifampicin, Streptomycin, Tetracycline, Blasticidin, Hygromycin, Bleomycin or Zeocin, or a gene that encodes a cytotoxin or a protein that can produce a cytotoxin from an extracellularly added protoxin, such as E. coli relE, chpBK, mqsR, higB, yafQ, or yhaV genes, or Pseudomonas aeruginosa tse2 gene, or Herpes simplex virus type 1 thymidine kinase gene, or Diphteria toxin A fragment gene, when placed under the control of an inducible transcriptional promoter.

### General Definitions

A “polynucleotide” and “nucleic acid” or “nucleic acid molecule”, as used herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. such nucleic acids are at times collectively referred to herein as “constructs,” “plasmids,” or “vectors.”

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones

An “expression cassette”, as used herein, refers to a nucleic acid or polynucleotide comprising a coding sequence operably-linked to a promoter.

A “chimeric gene” as used herein refers to a polynucleotide, e.g. expression cassette, comprising two or more operably-linked elements (e.g. a promoter and a coding sequence) and that is capable of driving expression of an RNA or protein, wherein at least two elements are heterologous with respect to each other. For example, a chimeric gene can comprise a promoter and a coding region which are not naturally associated with each other, such as sequences from different genes and/or from different organisms.

The term “operably-linked” as used herein, refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression.

A DNA sequence that “encodes” a particular RNA is a DNA nucleotide sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

A “protein coding sequence” or a sequence that encodes a particular protein or polypeptide, is a nucleotide sequence that is transcribed into mRNA (in the case of DNA) and is translated (in the case of mRNA) into a polypeptide in vitro or in vivo when placed under the control of appropriate regulatory sequences.

“Heterologous,” as used herein, means a nucleotide or polypeptide sequence that is not found in the native nucleic acid or protein, respectively. A heterologous nucleic acid sequence may be linked to another, e.g. naturally-occurring nucleic acid sequence (or a variant thereof) (e.g., by genetic engineering) to generate a chimeric nucleotide sequence, e.g. a chimeric gene, where such sequences are not naturally associated with each other. The term “heterologous” can also refer to a nucleotide or polypeptide sequence that is not naturally present in a certain organism, i.e. the sequence is heterologous with respect to the organism. Thus, the term heterologous can be used to designate the opposite of endogenous, i.e, the organism, sequence etc. as it occurs in nature.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism that can be isolated from a source in nature is naturally occurring.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. An example of such a case is a DNA (a recombinant) encoding a wild-type protein where the DNA sequence is codon optimized for expression of the protein in a cell (e.g., a eukaryotic cell) in which the protein is not naturally found. A codon-optimized DNA can therefore be recombinant and non-naturally occurring while the protein encoded by the DNA may have a wild type amino acid sequence. Thus, the term “recombinant” polypeptide does not necessarily refer to a polypeptide whose amino acid sequence does not naturally occur. Instead, a “recombinant” polypeptide is encoded by a recombinant non-naturally occurring DNA sequence, but the amino acid sequence of the polypeptide can be naturally occurring (“wild type”) or non-naturally occurring (e.g., a variant, a mutant, etc.). Thus, a “recombinant” polypeptide is the result of human intervention, but may have a naturally occurring amino acid sequence.

The term “purifying the protein”, as is used herein, refers to the purification of one or more proteins, which comprises a series of processes intended to isolate one or more proteins from a complex mixture, usually cells, tissues, and/or growth medium. Various purification strategies can be followed. For example, proteins can be separated based on size, in a method called size exclusion chromatography. Alternatively, proteins can be purified based on charge, e.g. through ion exchange chromatography or free-flow-electrophoresis, or based on hydrophobicity (hydrophobic interaction chromatography). It is also possible to separate proteins based on molecular conformation, for example by affinity chromatography. Said purification may involve the use of a specific tag, for example at the N-terminus and/or C-terminus of the protein. After purification, the proteins may be concentrated. This can for example be carried out with lyophilization or ultrafiltration.

“Transforming” or “transformation” as used herein, refers to introducing an exogenous nucleic acid into the cell in such a way that it becomes integrated into the genome of the cell. Such a cell thus becomes “transformed” or “genetically modified” or “transgenic”

“Transgenic”, as used herein, refers to a cell or an organism in which an exogenous nucleic acid has been integrated into its genome. “Cis-genic” as used herein, refers to cell or organisms wherein a sequence from the same or a related organism has been integrated into the genome, but does not occur in its naturally-occurring sequence context. For example, an endogenous gene can be inserted at a different genomic location, or endogenous genetic element such as coding sequences and promoters can be operably-linked to sequences to which they are not naturally associated (e.g. to create a chimeric gene).

The term “upstream” as used herein, refers to a position in a DNA that is located 5′ to the position specified with regard to the direction of transcription (i.e., located 5′ in the RNA); and the term “downstream” as used herein refers to a position in a DNA that is located 3′ to the position specified (i.e., located 3′ in the RNA).

A polynucleotide or polypeptide has a certain percent “sequence identity” to another polynucleotide or polypeptide, meaning that, when aligned, that percentage of bases or amino acids are the same, and in the same relative position, when comparing the two sequences, preferably over their full length. This is sometimes also referred to as homology, e.g. sequences sharing a certain sequence identity are often referred to as homologous sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

By “hybridizable” or “complementary” or “substantially complementary” it is meant that a nucleic acid (e.g. RNA, DNA) comprises a sequence of nucleotides that enables it to noncovalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible, i.e. they share a certain sequence identity. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

The singular forms “a,” “an,” and “the” as used herein also include plural referents unless the context clearly dictates otherwise.

Unless defined otherwise, all technical and scientific terms used herein have the same meaning as commonly understood by one of ordinary skill in the art to which this invention belongs. Definitions of common terms in cell biology and molecular biology, as are used herein, can be found in The Encyclopedia of Molecular Biology, published by Blackwell Science Ltd., (1994) (ISBN 0-632-02182-9); Benjamin Lewin, (2009) Genes X, published by Jones & Bartlett Publishing, (ISBN-10:0763766321); Kendrew et al. (eds.) (1995), Molecular Biology and Biotechnology: a Comprehensive Desk Reference, published by VCH Publishers, Inc., (ISBN 1-56081-569-8) and Coligan et al., eds., 2009. Current Protocols in Protein Sciences Wiley Intersciences.

All publications mentioned herein are incorporated herein by reference to disclose and describe the methods and/or materials in connection with which the publications are cited.

### EXAMPLES

### Example 1: Construction of a gene-trapped transformant library

Gene trap (GT) constructs have been widely employed for studying gene functions and for defining gene expression patterns (Acosta-García et al., 2004; Blanvillain & Gallois, 2008, Methods in Molecular Biology (Clifton, N.J.), 427, 121-135; Bouché & Bouchez, 2001; Trinh & Fraser, 2013, Development Growth and Differentiation, 55 (4), 434-445). As insertional mutagens they play a central role in creating gene knockout libraries and they are frequently employed for the study of animal cells, plants and bacteria. The heterokont unicellular microalga Nannochloropsis has recently gained attention as a prospective candidate for sustainable production of biofuel feedstocks and high-value compounds (Alves et al., 2018, Scientific Reports, 8 (1); Liu et al., 2017, Renewable and Sustainable Energy Reviews, 72, 154-162; Xu & Boeing, 2014), but our understanding of cellular metabolism in this organism is limited. GT studies may help to demystify the complex regulatory networks involved in the production of value-added compounds and they are rendered a promising choice for this genus because Nannochloropsis integrates foreign DNA into its genome in random positions (Jinkerson et al., 2013, Bioengineered, 4 (1), 37-43).

With the aim of studying gene functions and expression patterns we transformed Nannochloropsis oceanica with a gene trapping construct (TC) displayed in FIG. 1. The cassette encodes EGFP and the antibiotic resistance protein zeo^{R} separated by a viral 2A peptide (P2A) to facilitate synthesis of both proteins from a single transcript (Donnelly et al., 2001, Journal of General Virology, 82 (5), 1013-1025). The absence of a transcriptional promoter prohibits expression of transgenes unless the cassette is inserted into a gene. A splice acceptor (SA) sequence was included to safeguard transgene expression in the event of insertion into an intron. In this case, transgenes would be ligated onto upstream exons during RNA splicing (Acosta-García et al., 2004). A transcriptional terminator of an endogenous gene (T_{α-tub}) was attached to prevent transcription of sequences downstream of the TC. Terminal recognition sequences for the type IIS restriction enzyme MmeI were added to the cassette to allow for a simplified genome walking procedure. This has proven feasible for the identification of disrupted genes during insertional mutagenesis studies with other organisms (Goodman et al., 2009, Cell Host and Microbe, 6 (3), 279-289; Morgan et al., 2008, Nucleic Acids Research, 36 (20), 6558-6570; Zhang et al., 2014, The Plant Cell, 26 (4), 1398-1409). Cells were separately transformed with a control construct (CC) which was designed to drive the expression of the same transgenes under control of an endogenous promoter. VCP1 encodes the major photosynthetic light harvesting complex protein in the microalga and this gene has by far the highest transcript abundance among all cellular mRNAs (Li et al., 2014b, Plant Cell, 26 (4), 1645-1665). Its promoter (P_{VCP1}) is routinely used to transform Nannochloropsis with high efficiency (Kilian et al., 2011, Proceedings of the National Academy of Sciences of the United States of America, 108 (52), 21265-21269; Kilian & Vick, 2013, U.S. patent application Ser. No. 13/915,555; Li et al., 2014a, Bioscience, Biotechnology and Biochemistry, 78 (5), 812-817).

### Example 2: EGFP Screen and Characterization of Transformant Strains

Transformants for both constructs were selected for antibiotic resistance on agar plates and then subjected to a GFP screen using flow cytometry analysis. FIG. 2 shows the single cell fluorescence emission levels for representative cultures of a selection of 48 independent colonies in boxplot representation on a logarithmic scale. Significant differences compared to the wild type (WT) fluorescence emission were found for the majority of transformants, suggesting good prospects for GT-based gene studies in this organism. Importantly, TC strain #17 displayed exceptionally high levels of fluorescence emission, exceeding that of all other transformant strains. In order to understand which promoter was responsible for the high GFP expression in TC #17 we traced the insertion using a customized MmeI-assisted genome walking protocol (Goodman et al., 2009, Cell Host and Microbe, 6 (3), 279-289).

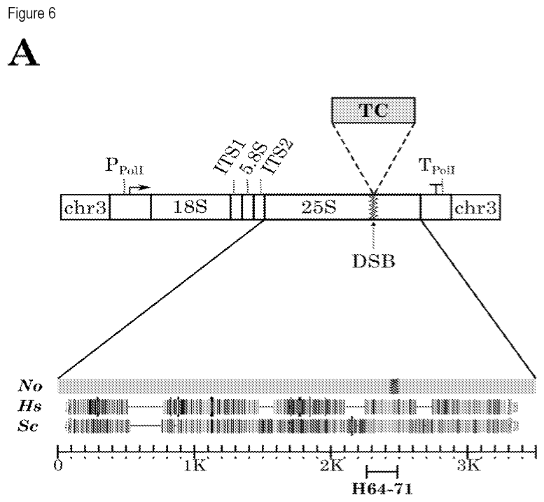

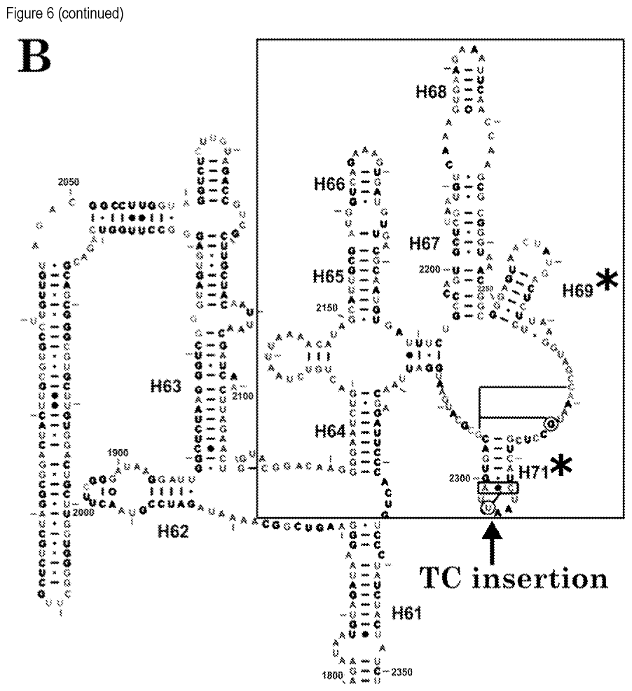

The procedure revealed the sequence of 20 nucleotides of genomic DNA flanking the insertion cassette on both sides. We performed BLAST analyses of these sequences against the genome of the organism and found 3 possible insertion sites with identical sequences. After manually curating the annotations of these loci, they turned out to be ribosomal DNA cistrons. Using PCR analysis and sequencing with unique primers for all potential sites we found that the TC had been inserted in scaffold 341 at nucleotide position 19840 (current genome assembly chromosome 3, nucleotide position 1029753), 2336 nucleotides into a 25S ribosomal RNA (rRNA) gene (FIG. 6). To verify that this was the only insertion of the TC in the genome of TC #17 and the causative mutation for the phenotype, we amplified the full-length ribosomal DNA (rDNA) cistron including the promoter, terminator and the inserted TC from chromosome 3 of this strain (EC1 in FIG. 7) and transformed the wild type strain with this cassette. EC1 transformant strains showed the same strong fluorescence emission that was observed for TC #17, confirming the link between the insertion of the TC into the rDNA cistron and the high level of fluorescence emission.

### Example 3: Transgene Expression is Driven by Polymerase I in TC #17

Because rRNA is not subject to translation, a rRNA-TC fusion transcript should not yield any functional protein. The 18S, 5.8S and 25S/28S rRNA molecules are an essential part of eukaryotic cytosolic ribosomes and they are synthesized in a designated subnuclear organelle called the nucleolus (Brown & Gurdon, 1964, Proceedings of the National Academy of Sciences of the United States of, 51, 139-146; Hadjiolov, 1985; Perry, 1962, Proceedings of the National Academy of Sciences, 48 (12), 2179-2186). The 3 rRNA genes are arranged as a single transcriptional unit which is transcribed by DNA-directed RNA polymerase I (Pol I) to yield a pre-rRNA molecule which undergoes extensive co- and post-transcriptional modification to generate free 18S, 5.8S and 25S/28S rRNA (Lodish et al., 2000). Although rRNA molecules are heavily post-transcriptionally modified, they lack features which are exclusive for protein-encoding mRNAs such as a 5′ cap and a poly (A) tail, which are added during polymerase II transcription. In eukaryotes, these features demarcate RNA molecules that contain protein-encoding information and they are thus a necessary prerequisite for translation initiation (Poulin & Sonenberg, 2000). However, an exception to this is the mechanism of cap-independent translation initiation, which is mediated by a cis-acting RNA element called an internal ribosome entry site (IRES) that is present in the 5′ untranslated region (UTR) of an RNA molecule. IRESs recruit the 40S ribosomal subunit directly to the transcript via non-canonical RNA-protein interactions that do not require a 5′ cap but usually involve binding of a subset of the eukaryotic initiation factors and sometimes additional proteins called IRES trans acting factors (ITAFs) (Semler & Waterman, 2008, Trends in Microbiology, 16 (1), 1-5). IRESs were first discovered in positive-stranded RNA viruses (Jang et al., 1988, Journal of Virology, Pelletier & Sonenberg, 1988, Nature) but they were soon also found in 5′ UTRs of cellular RNAs (Johannes & Sarnow, 1998, Rna, 4 (12), 1500-1513; Macejak & Sarnow, 1991, Nature, 353 (6339), 90-94). For reviews on IRESs see (Thompson, 2012) and (Yamamoto et al., 2017).

### Example 4: Translation of the Transcript is Facilitated by an IRES

Whereas IRES-mediated translation initiation can be favored over cap-dependent translation initiation under certain conditions such as during pathological stress, it is usually much less efficient than translation via the canonical way (Andreev et al., 2009, Nucleic Acids Research, 37 (18), 6135-6147; Bert et al., 2006, RNA, 12 (6), 1074-1083; Gilbert, 2010; Young et al., 2008, Journal of Biological Chemistry, 283 (24), 16309-16319). Assuming an IRES-mediated translation of transgene transcript in TC #17 may therefore seem contradictory, considering that the strain showed ˜8× increased reporter fluorescence compared to CC strains. However, when we quantified EGFP transcript abundance relative to Actin through RTq-PCR we found a ˜135-fold increase in TC #17 compared to the CC strain (FIG. 3B). This vast difference would explain the increased fluorescence even under assumption of an inefficient mode of translation initiation. Moreover, the high transcript abundance is another indication that the transgene is transcribed by Pol I in TC #17. Pol I is a highly efficient enzyme with significantly improved processivity and elongation speed compared to Pol II, which is reflected in the differences in cellular levels of RNA species. Whereas Pol II transcribes 5,000-50,000 distinct protein-encoding genes in a typical eukaryotic cell (Straalen & Roelofs, 2013), only 5% of the total RNA is mRNA (Lodish et al., 2000). 50-80% are transcripts of the 3 different rRNA genes expressed by Pol I (Lodish et al., 2000; Russell & Zomerdijk, 2006). Therefore, the immense increase in transcription observed for TC #17 confirms an involvement of Pol I in transgene expression. Under the hypothesis that the transgenes in TC #17 are transcribed as a 25S rRNA-TC fusion (‘fuRNA’) by Pol I, the presence of an IRES element upstream of EGFP may be an obvious explanation for the high fluorescence emission in this strain. However, at the time of this discovery, transgene expression employing Pol I and an IRES had rarely been reported (Wen et al., 2008, Biochemical and Biophysical Research Communications, 367 (4), 846-851) and never for microalgae. Recently, a combination of a Pol I promoter and IRES element was shown to drive heterologous gene expression in Saccharomyces cerevisiae, substantiating that Pol I-transcribed RNA molecules can undergo cap-independent translation (Bao et al., 2019). In order to apprehend the underlying molecular mechanism responsible for the extraordinarily high transgene expression levels in TC #17, it was crucial to ascertain the presence of an IRES.

IRES elements vary in length from less than 100 to >1000 nucleotides (Baird et al., 2006) with popular studied IRESs typically being between 200-440 nucleotides long (Bochkov & Palmenberg, 2006, Bio Techniques, 41 (3), 283-292; Pestova et al., 1998, Genes and Development, 12 (1), 67-83; Wilson et al., 2000). Type I viral IRESs are located up to >150 nucleotides upstream of the open reading frame (ORF) under their control (Jackson, 2013, Cold Spring Harbor Perspectives in Biology, 5(2); Pestova et al., 1994, Virology, 204(2), 729-737; Sweeny et. al., 2014, EMBO Journal, 33(1), 76-92) whereas types II-IV are usually located immediately upstream of the ORF and position the ribosome directly onto the initiation codon (Baird et al., 2006). The recombinant EGFP protein of TC #17 showed no visible size difference in western blot analysis (FIG. 3D) compared to EGFP from strains carrying the CC, suggesting that translation initiation occurs in the vicinity of the EGFP AUG codon. The presence of an in-frame translational STOP codon 75 nucleotides upstream of the EGFP AUG codon further supports this notion. Because most IRESs are located very close to the site of translation initiation, it is therefore likely that the putative IRES in the fuRNA lies close to the EGFP AUG codon.

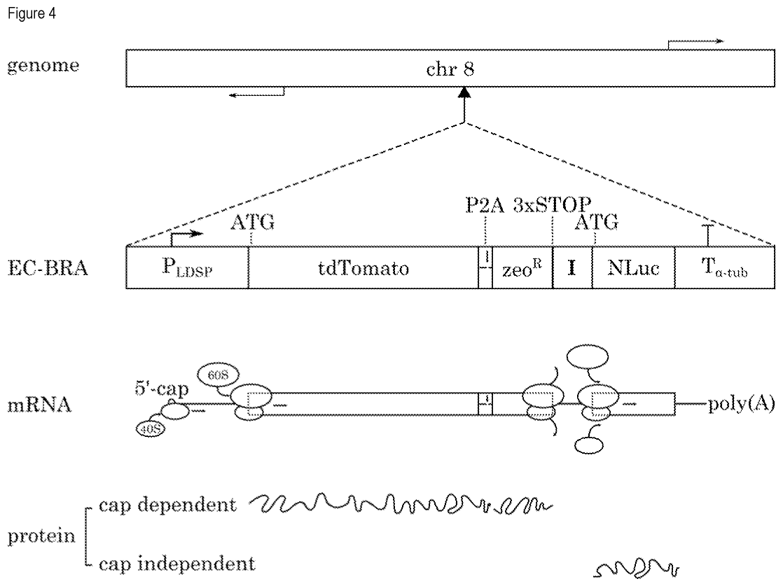

To test this hypothesis, a bicistronic reporter assay was designed, which is a conventional method to quantify cap-independent translation driven by putative IRES sequences (Thompson, 2012). In this type of assay, a eukaryotic organism is transformed with a construct carrying a single transcriptional unit encoding a polycistronic transcript with 2 reporter genes under control of only 1 promoter and terminator. In between the 2 reporter genes lies the putative IRES sequence and several translational STOP codons at the 3′-end of the 5′-located gene. The 5′-located gene will be translated through canonical cap-dependent translation. Cap-dependent translation will terminate at the STOP codons and cause dissociation of the ribosome (see schematic in FIG. 4, “mRNA”). The 3′-located gene can only be translated if the putative IRES sequence indeed promotes cap-independent translation. Thus, activity of the 3′-located reporter protein in transformant strains correlates with IRES activity of the sequence in question. We designed multiple polycistronic reporter constructs to verify IRES activity of the fuRNA. From 5′ to 3′-end the DNA cassettes (FIG. 4) contained the Pol II-recruiting transcriptional promoter of the endogenous lipid droplet surface protein (LDSP) gene (P_{LDSP}), a gene encoding the particularly strong fluorescent reporter tdTomato, linked via a P2A peptide sequence to zeo^{R}, 3 consecutive translational STOP codons, the putative IRES sequence (I), the NanoLuciferase gene (NLuc) as IRES-reporter and the α-tubulin terminator. With regard to the putative IRES element, we decided to consider a sequence of 255 nucleotides (hereafter referred to as Noc-IRES) preceding the EGFP ATG-codon in TC #17, corresponding to 203 nucleotides (2134-2336) of the 25S rRNA gene and the splice acceptor sequence of the TC.

The Noc-IRES sequence was inserted in EC-BRA-Noc-IRES, whereas a negative control construct EC-BRA-NC contained no sequence in between the reporter genes. Because there are no known IRESs with confirmed activity in Nannochloropsis we chose to conduct the experiment with 2 control IRESs that had previously been successfully employed to drive cap-independent translation across all kingdoms. These sequences are the cricket paralysis virus (CrPV) intergenic region (IGR) IRES (Hodgman & Jewett, 2014, New Biotechnology, 31 (5), 499-505; Kieft, 2008) and the crucifer-infecting tobamovirus (crTMV) IRES (CP,148) (Dorokhov et al., 2002, Proceedings of the National Academy of Sciences of the United States of America, 99 (8), 5301-5306; Ivanov et al., 1997, Virology, 232 (1), 32-43; Marom et al., 2009, RNA Biology, 6 (4), which were included in constructs EC-BRA-CrPV-IRES and EC-BRA-crTMV-IRES respectively. All constructs had a single nucleotide insertion in the sequence containing the 3 translational STOP codons to ensure that the NLux gene would not be translated in-frame in the unlikely case of ribosomal read-through.

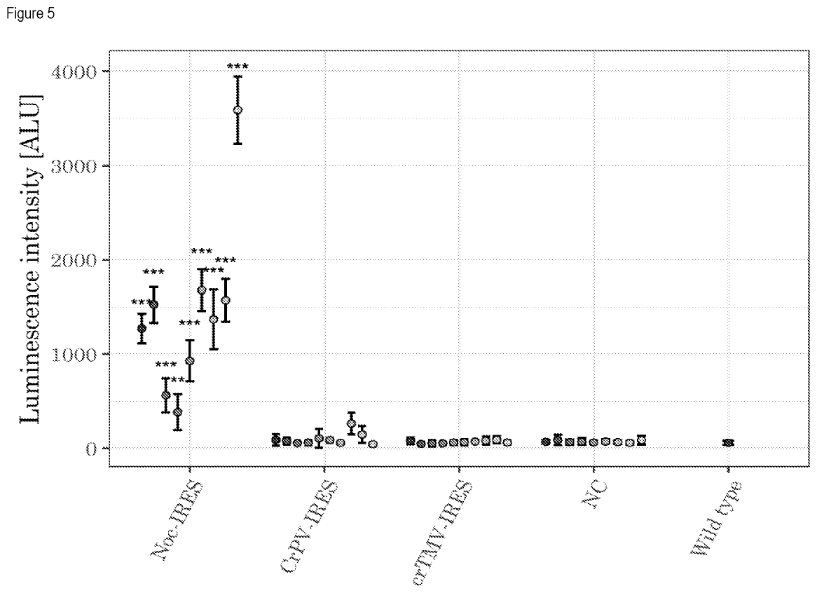

At least 20 transformant strains per construct were screened for presence of transgenes and tdTomato fluorescence levels. 10 strains with similar levels of the fluorescent reporter were selected, subcultured and subjected to luminescence assays in order to quantify activity of the IRES-reporter. FIG. 5 clearly shows that only EC-BRA-Noc-IRES had substantial luciferase activity. The negative control EC-BRA-NC showed the same activity levels as the wild type strain, which rules out the possibility that luciferase synthesis in EC-BRA-Noc-IRES was the consequence of ribosomal read-through. The 2 viral IRESs are not active in N. oceanica as EC-BRA-CrPV-IRES and EC-BRA-crTMV-IRES transformants did not exhibit an improved luminescence signal compared to the negative control. A substantial increase in luminescence signal was observed for all strains carrying EC-BRA-Noc-IRES (p<0.001). This is strong evidence for the ability of Noc-IRES to promote cap-independent recruitment of ribosomes in Nannochloropsis and therefore an explanation for the translation of Pol I-transcribed fuRNA transcripts in TC #17.

### Example 5: Several Elements of the Noc-IRES Sequence May be Responsible for Ribosomal Recruitment

Understanding IRES activity of specific DNA sequences is difficult because IRES elements are versatile in sequence, secondary structure and modus operandi (Thompson, 2012). However, polypyrimidine (poly (Y)) tracts are features that are present in multiple viral and cellular IRESs. In an IRES setting, they recruit poly (Y) tract binding protein (PTB) as an ITAF (Jang & Wimmer, 1990, Genes and Development, 4 (9), 1560-1572; Jaramillo-Mesa et al., 2018, Journal of Virology, 93 (5); Martinez-Salas et al., 2018). Poly (Y) tracts are also conserved features of splice acceptor sequences in gene introns and consequently a poly (Y) tract is present immediately upstream of the EGFP ATG codon in the TC. BLAST analysis of this poly (Y) tract against known IRES sequences revealed similarity to known cellular IRESs, e.g. of the elF4G homologue DAP5 and Mnt genes of humans (Marash & Kimchi, 2005, Cell Death and Differentiation, 12 (6), 554-562; Mitchell et al., 2005, Genes and Development, 19 (13), 1556-1571) and also to an encephalomyocarditis virus (EMCV) IRES (Jang et al., 1988, Journal of Virology). Furthermore, IRESs of e.g. the hepatitis C virus (Angulo et al., 2016, Nucleic Acids Research, 44 (3), 1309-1325; Malygin et al., 2013, Nucleic Acids Research, 41 (18), 8706-8714) or the 5′ UTR of cellular mRNAs like the homeodomain protein Gtx (Chappell et al., 2000, Proceedings of the National Academy of Sciences of the United States of America, 97 (4), 1536-1541) have been shown to directly interact with the 40S ribosomal subunit via base pairing between the IRES and complementary sequences of the 18S rRNA. Thus, similar to the general mechanism of ribosomal recruitment in prokaryotes (Laursen et al., 2005, Microbiology and Molecular Biology Reviews, 69 (1), 101-123), complementarity of an RNA sequence to rRNA genes may promote cap-independent ribosomal binding in eukaryotes (Jaramillo-Mesa et al., 2018, Journal of Virology, 93 (5).

The insertion of the TC in TC #17 occurred at position 2336 of the 25S rRNA gene, which corresponds to nucleotide 2296 of the well-studied S. cerevisiae 25S rDNA (FIG. 6A). (FIG. 6A). The proximal ˜200 nucleotide sequence immediately upstream of this position roughly corresponds to helices 64-71 in domain IV of the 25S rRNA which are highly conserved in nucleotide sequence and structure among ribosomes across all kingdoms of life (FIG. 6B). These helices have a 95.7% and 93.3% nucleotide sequence identity between Nannochloropsis and yeast or human homologues, respectively, whereas other regions of the rDNA are much less conserved with overall identities between the entire 25S rRNA genes in these species being only 75.3% and 42.6%. The characteristics of some of these highly conserved helices are well-studied and they may explain how cap-independent translation can occur in TC #17.

Helix 69 (H69) e.g. is part of the intersubunit bridge B2a which connects the large ribosomal subunit (LSU) to the decoding center of the small subunit (SSU) at the heart of the ribosome during translation (Yusupov et al., 2001, Science, 292 (5518), 883-896). It has been shown that deletion of H69 entirely prohibits subunit joining in the absence of tRNA, indicating that B2a is the most essential connection between the ribosomal subunits (Ali et al., 2006, Molecular Cell, 23 (6), 865-874). H69 further contacts the tRNAs in the A- and P-sites (Hirabayashi et al., 2006, Journal of Biological Chemistry, 281 (25), 17203-17211; Stark et al., 2002, Nature Structural Biology, 9 (11), 849-854) and it has been proposed to serve important functions in signal relay between the decoding site of the SSU and the GTPase-associated elements of the LSU (Bashan et al., 2003, Molecular Cell, 11 (1), 91-102; Cochella & Green, 2005, Science, 308 (5725), 1178-1180; Rodnina et al., 2002, Biochimie, 84 (8), 745-754) although this is subject to debate (Ali et al., 2006, Molecular Cell, 23 (6), 865-874). Finally, experimental evidence revealed that a key function of H69 is its involvement in peptide release and ribosome recycling upon translation termination. The RNA helix interacts with release factor (RF) (Weixlbaumer et al., 2008, Science, 322 (5903), 953-956) and ribosome recycling factor (RFF) proteins (Ali et al., 2006, Molecular Cell, 23 (6), 865-874; Pai et al., 2008, Journal of Molecular Biology, 376 (5), 1334-1347; Wilson et al., 2005, EMBO Journal, 24 (2), 251-260) and further with proteins involved in stalled ribosome rescue in bacteria (Gagnon et al., 2012, Science, 335 (6074), 1370-1372). Given the strong interaction of H69 with helix 44 of the 18S rRNA in the intersubunit bridge B2a, the H69 hairpin structure in the fuRNA may directly be involved in recruiting the SSU to the transcript via base-pairing with the 18S rRNA similar to the interaction during subunit joining. Analogous mechanisms for SSU recruitment have been shown for several IRESs (Angulo et al., 2016, Nucleic Acids Research, 44 (3), 1309-1325; Chappell et al., 2000, Proceedings of the National Academy of Sciences of the United States of America, 97 (4), 1536-1541; Malygin et al., 2013, Nucleic Acids Research, 41 (18), 8706-8714). Alternatively, the involvement of H69 in RNA-protein interactions during peptide release, ribosome recycling and ribosome rescue suggests an implication of this RNA structure in recruitment of ITAF proteins in an IRES setting.

The nucleotide sequence connecting H69 and the SA element in the fuRNA are helix 70 (H70) and helix 71 (H71) partially (see FIG. 6). These highly conserved regions are part of the intersubunit surface and they promote ribosome stability and binding of ribosomal proteins (RPs) (Gigova et al., 2014, RNA, 20 (10), 1632-1644). H71 was found to directly interact with the RP Rpl23, which in turn binds other RPs and a weakening of the H71-Rpl23 interaction is concomitant with depletion of several other RPs from LSUs in yeast (Gigova et al., 2014, RNA, 20 (10), 1632-1644). Intriguingly, a potential Rpl23 interaction partner known as Rpl26 has been shown to function as an ITAF which binds to the IRES-containing 5′-UTR of p53 mRNA in human cells and recruits polysomes, thereby significantly increasing translation levels (Chen et al., 2012, Botanical Studies, 53 (1), 125-133; Takagi et al., 2005, Cell, 123 (1), 49-63). Next to that, Rpl26 directly binds the 3′-UTR of p73 mRNA and induces its translation by recruiting the cap-binding protein elF4E which is also implicated in cap-independent translation in picornavirus IRES elements (Avanzino et al., 2017, Proceedings of the National Academy of Sciences of the United States of America, 114 (36), 9611-9616). Rpl38, another potential interaction partner of the H71-binding Rpl23, is likely another ITAF that regulates the assembly of ribosomes on the IRES of Hox mRNAs in animal cells (Xue et al., 2015, Metabolic Engineering, 27, 1-9). If the H70-H71 tract of the fuRNA in N. oceanica associates with the same RPs that have been shown to directly or indirectly bind to this region in functional yeast ribosomes, these proteins could act as ITAFs that recruit eIFs or ribosomes to the IRES.

In conclusion, several features in the ORF-proximal 5′ UTR of the fuRNA either resemble IRES elements or have been suggested to bind the 18S rRNA or ITAF proteins which can facilitate ribosome recruitment, suggesting that transgene expression in TC #17 is driven by Pol I in combination with cap-independent translation. We decided to further investigate the potential of exploiting this mechanism by learning more about the genetic elements that are involved in this process and in order to simplify the genetic construct employed for transformation.

### Example 6: Defining the Minimal Elements for Pol I-Based Gene Expression

Ribosome biogenesis in the nucleolus of eukaryotic cells is a complex and highly controlled process (Pederson, 2011, Cold Spring Harbor Perspectives in Biology, 3 (3), a000638-a000638). The primary Pol I transcript undergoes multiple endo- and exonucleolytic cleavages and other modifications to yield free 18S, 5.8S and 25/28S rRNA molecules (Lodish et al., 2000). During this, the intermediates are continuously screened by an RNA surveillance machinery that recognizes aberrant or unstable RNA supposedly via interaction with tertiary structures (Hamill et al., 2010, Proceedings of the National Academy of Sciences of the United States of America, 107 (34), 15045-15050; Rammelt et al., 2011, RNA, 17 (9), 1737-1746; Schmidt & Butler, 2013; Wong et al., 2015, Research and Reports in Biochemistry, 111). In a process reminiscent of protein ubiquitinylation, erroneous RNA molecules are tagged for degradation via polyadenylation and subsequently nucleolytically degraded by a polyprotein called the nuclear exosome (Allmang, 2000, Nucleic Acids Research, 28 (8), 1684-1691; Houseley et al., 2006; Vanacova & Stef, 2007). This complex of 3′->5′ exonucleases is not only an intricate quality control machinery involved in the degradation of defective RNAs but it is also involved in rRNA maturation. Surprisingly, the fuRNA which carries an insertion of the entire TC inside of the 25S rRNA in TC #17 is not degraded but exported to the cytosol instead. Unfortunately, whereas quality control mechanisms are relatively well understood for mRNAs (Doma & Parker, 2007), knowledge about the surveillance mechanisms for rRNA is lacking.

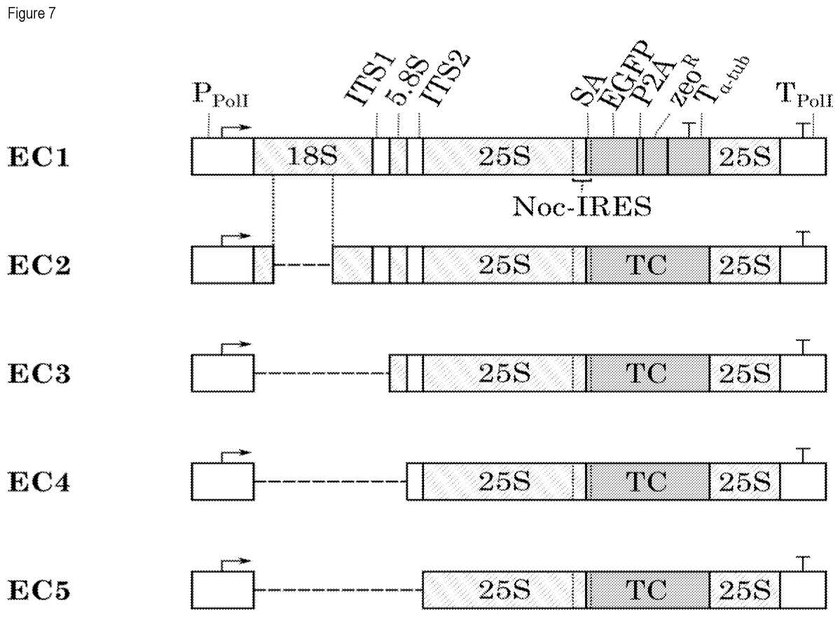

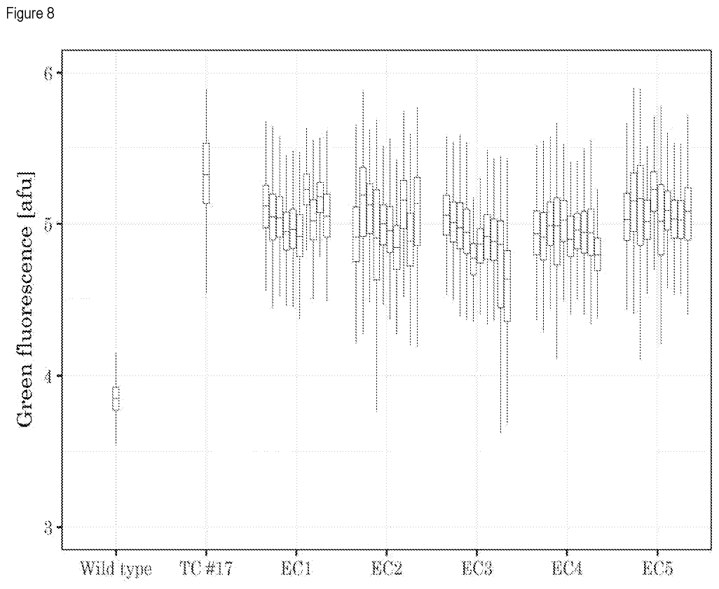

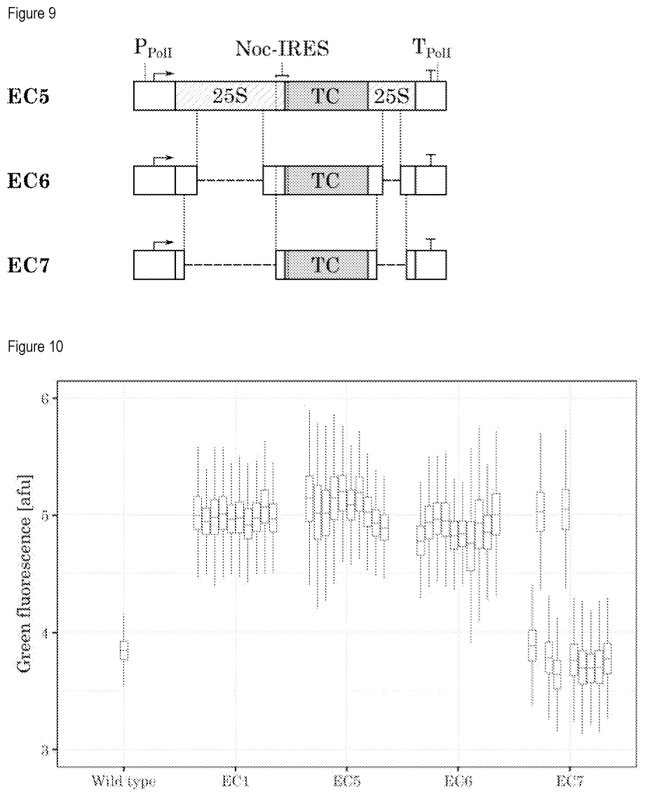

To ascertain which elements of the rDNA cistron are important for the fuRNA to escape the rigorous nucleolar RNA surveillance machinery and reach the cytosol in N. oceanica we designed a number of expression constructs (ECs) based on EC1 with a succession of missing elements from the 18S rDNA up to the ITS2 (internal transcribed spacer 2) as illustrated in FIG. 7. EC2 is missing internal parts of the 18S rDNA sequence whereas the ends of the gene were kept intact, which is attributed to the fact that the terminal sequences of rDNA elements serve a pivotal role in the maturation process of rRNAs. For instance, the U3 and U8 snoRNA molecules have nucleotide sequence complementarity to the 5′ ends of the 18S and 28S rDNA respectively and they are essential for rRNA maturation as guides for ribosomal binding factors that endonucleolytically cleave the nascent pre-rRNA molecule in metazoans (Hughes, 1996, Journal of Molecular Biology, 259 (4), 645-654; Peculis, 1997, Molecular and Cellular Biology, 17 (7), 3702-3713). Removing the terminal parts of an rRNA gene may therefore have deleterious consequences for fuRNA processing whereas the inner parts may be dispensable. (Musters et al., 1989, Molecular and Cellular Biology, 9 (2), 551-559) have shown that modified 25S rRNA molecules carrying an 18 nucleotide insertion in the middle part of the rRNA gene are not degraded but they are instead assembled into functional ribosomal in S. cerevisiae, supporting this notion. All constructs were able to yield viable transformant strains. After screening a minimum of 20 colonies per EC we found that all transformants exhibited fluorescence emission similar to that observed for EC1 (FIG. 8). This implies that not all erroneous rRNA transcripts are subject to nuclear degradation which is in good agreement with the findings of (Musters et al., 1989, Molecular and Cellular Biology, 9 (2), 551-559), who showed that deletion of the majority of the 18S rDNA did not affect the post transcriptional processing of the 5.8S and 25S rRNA transcribed from the same cistron in S. cerevisiae. Notably, (Musters et al., 1989, Molecular and Cellular Biology, 9 (2), 551-559) have also shown that deleting conserved regions of the 25S rDNA interfered with processing of the erroneous transcript beyond the level of 29S rRNA, indicating that not only the aberrant 25S rRNA but also the intact 5.8S rRNA were not considered for ribosomal assembly. Despite this, we found that EC4-5 carrying transformant strains were strongly fluorescent, although they are missing the 5.8S rDNA. This leads to the conclusion that either N. oceanica has a different mechanism than S. cerevisiae for detecting erroneous pre-rRNAs or that not all steps of regular rRNA processing are required for maturation and export of the fuRNA. In fact, it can be hypothesized that rRNA-like post transcriptional processing of the fuRNA may be detrimental for translation of the transgenes. Presumably, association of ribosomal proteins with the fuRNA would prevent binding of functional ribosomes and formation of the translation pre-initiation complex. To further understand the molecular mechanisms behind fuRNA processing we next modified EC5 by omitting different parts of the 25S rRNA gene, as depicted in FIG. 9. At least 10 transformant strains per construct were subjected to a GFP screen (FIG. 10).

### Example 7: Only Gene Insertion in the NOR Facilitates Strong Transgene Expression

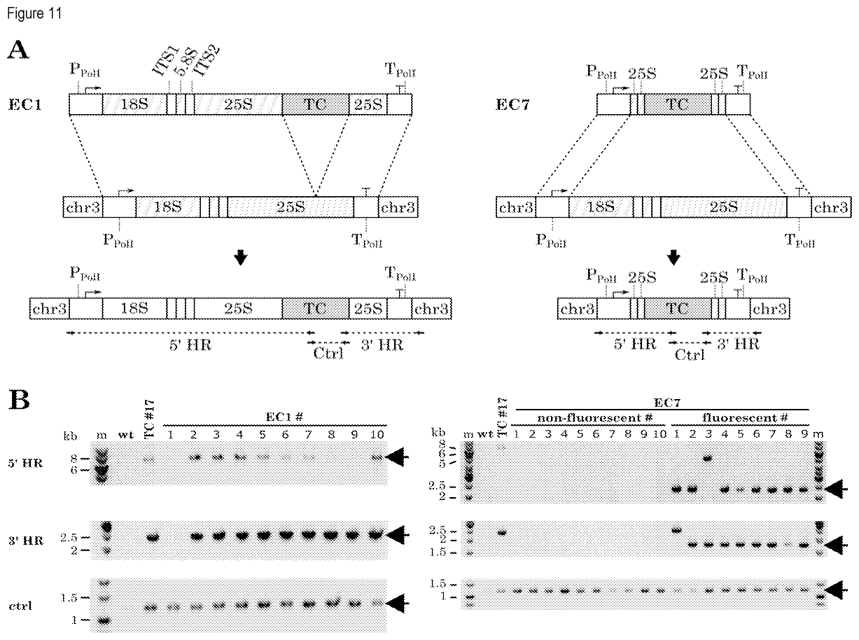

Construct EC7 had the shortest 25S sequences and was fully functional, but yielded high fluorescence only in a low fraction of transformants whereas almost all EC1-6 strains were highly fluorescent. In genetic engineering, differences in transgene expression between transformant strains are often related to epigenetic effects such as RNA interference or to positional effects based on the insertion site of the EC. The binary phenotype of EC7 strains i.e. the absence of intermediately fluorescent strains suggests that these effects were unusually strong for this EC compared to control constructs. As indicated above, in Nannochloropsis species foreign DNA is commonly integrated into the genome at random positions via non-homologous end joining (NHEJ). However, homologous recombination (HR) has been observed for this genus and it has been reported that targeted gene insertion based on CRISPR-Cas technique utilising 1000 bp of homology flanks is possible (Kilian et al., 2011, Proceedings of the National Academy of Sciences of the United States of America, 108 (52), 21265-21269; Naduthodi et al., 2019, Biotechnology for Biofuels, 12 (1), 66). The length of homologous sequences is generally accepted to affect the efficiency of HR (Elliott et al., 1998, Molecular and Cellular Biology, 18 (1), 93-101; Zhang et al., 2017, Genome Biology, 18 (1)). Compared to the other ECs, EC7 had significantly shorter sequences with complementarity to the 25S rDNA, suggesting that HR between the cassette and the rDNA cistrons would be less likely for this construct. Consequently, we tested whether occurrence of HR-mediated insertion may be linked to EGFP fluorescence levels of transformants. Therefore, we genetically characterized transformant strains of different ECs using PCR with primers directed to the rDNA cistron on chromosome 3 (FIG. 11A). The characterization results for EC1 and EC7 strains are shown in FIG. 11b. If available, a selection of strongly and weakly fluorescent strains were screened.

We found that cassette insertion had almost exclusively happened in the rDNA cistron on chromosome 3 in all highly fluorescent transformant lines, regardless of the type of EC. For weakly fluorescing strains on the other hand, the cassette was inserted in a different location. This observation corroborates our hypothesis that the strong expression levels of transgenes in TC #17 were caused by Pol I-based transcription because this enzyme complex is restricted to the nucleolus (Leger-Silvestre et al., 1999, Chromosoma, 108 (2), 103-113). To achieve high levels of Pol I activity it seems to be essential to incorporate the construct within the NOR, i.e. those parts of the genome that contain rDNA cistrons and any additional sequences which form the DNA constituent of the nucleolus.

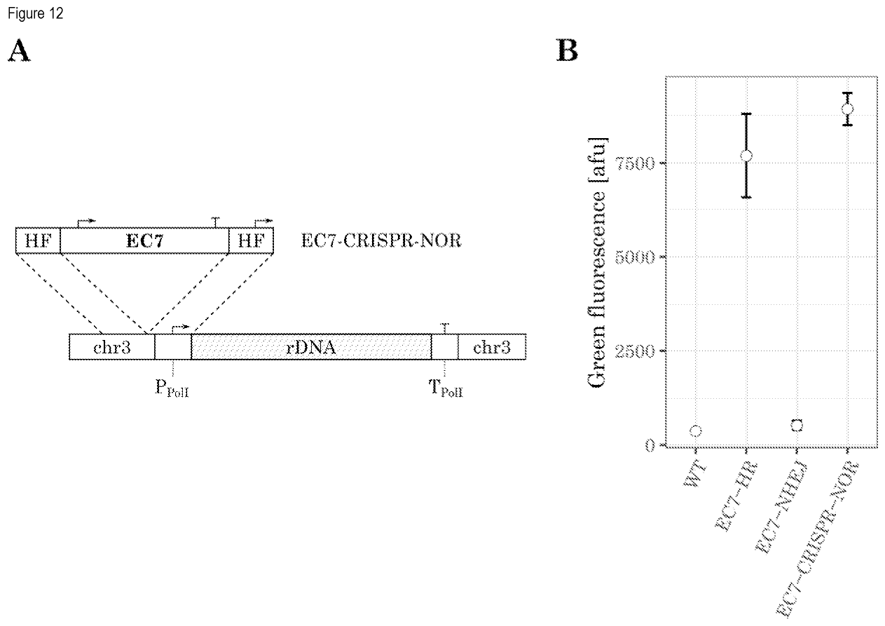

The genome architecture regarding NORs is comparable between different eukaryotic organisms. NORs usually exist as clusters of rDNA tandem repeats which are distributed typically over 1-6 chromosomes (McStay & Grummt, 2008, Annual Review of Cell and Developmental Biology, 24 (1), 131-157). The number of cistron copies in a single tandem repeat however varies significantly between different species and usually ranges from 70-140 (McStay & Grummt, 2008, Annual Review of Cell and Developmental Biology, 24 (1), 131-157; Petes, 1979, Proceedings of the National Academy of Sciences of the United States of America, 76 (1), 410-414; Sáez-Vásquez & Gadal, 2010, Molecular Plant, 3 (4), 678-690). In this regard, N. oceanica is unusual because all 4 rDNA loci contain single cistrons instead of tandem repeats (Gong et al., 2020, The Plant Journal, tpj. 15025; Vieler et al., 2012, PLOS Genetics, 8 (11), e1003064). It is generally accepted that not all rRNA genes are transcriptionally active in eukaryotic cells. Instead, ˜50% of all rDNA cistrons in mammalian cells are in a heterochromatic, inactive state (Conconi et al., 1989, Cell, 57 (5), 753-761; McStay & Grummt, 2008, Annual Review of Cell and Developmental Biology, 24 (1), 131-157). It is unknown if there are any preferentially active NORs in N. oceanica but based on the observed transgene expression levels of EC1-7 transformant strains, the NOR on chromosome 3 seems to regularly partake in the formation of the nucleolus and in rRNA gene expression. To further substantiate the connection between integration in a NOR and Pol I-based gene expression and to test whether we could employ the rRNA transcription machinery without knocking out an rDNA cistron, we decided to insert EC7 as the shortest functional construct in a targeted way adjacent to the rDNA cistron, i.e. presumably on the border of this NOR using CRISPR-Cas technology (FIG. 12A). We found similarly strong fluorescence emission of an EC7-CRISPR transformant strain that had EC7 inserted adjacent to the rDNA cistron (EC7-CRISPR-NOR), compared to strains where EC7 had replaced the rDNA cistron (EC7-HR, FIG. 12B). This shows that the transgene expression also occurs when the EC is inserted into other loci than the rDNA cistron on chromosome 3 as long as they are still part of a NOR.

### Example 8: Development of a Screening Pipeline for N. oceanica Transformant Strains with Strong Gene Expression

Consistent and strong expression of transgenes is often linked to insertion of an EC into specific genomic loci. This is perhaps best illustrated by the discovery of the adeno-associated virus integration site 1 locus of the human chromosome 19, as a “safe harbor” for robust transgene expression (Luo et al., 2014, STEM CELLS Translational Medicine, 3 (7), 821-835; Smith et al., 2008, Stem Cells, 26 (2), 496-504). For the Pol I-based expression system in N. oceanica, the rDNA cistron of chromosome 3 had proven to be a safe harbor in our previous experiments. Therefore, we created a system to easily select algal transformant strains with EC integration at this locus. An ideal stage for this selection is right after antibiotic selection, on agar plates. There are different ways to identify recombinant microbial colonies on agar plates using non-invasive methods, such as the blue-white screening (Ullmann et al., 1967, Journal of Molecular Biology, 24 (2), 339-343; Vieira & Messing, 1982, Gene, 19 (3), 259-268), or fluorescence screening through epifluorescence imaging (Kondo & Yumura, 2019; Wong & Truong, 2010, PLOS ONE, 5 (12), 1-5). To our knowledge, however, on-plate screenings have never been reported for microalgae. To investigate whether an on-plate fluorescence-based selection system is possible in N. oceanica, we created the strain EC7-tdTomato-S1 that carries the fluorescent reporter gene tdTomato, polycistronically linked to zeo^{R} in the rDNA locus of chromosome 3. Expression of the reporter gene is driven by the Pol I promoter and Noc-IRES. We found that, likely due to the strong and consistent gene expression at this locus, coupled to the intense brightness of tdTomato (Shaner et al., 2004, Nature Biotechnology, 22 (12), 1567-1572), this strain emits reporter fluorescence that is easily discernible on agar plates.

Using EC7-tdTomato-S1 as a parental strain, we were able to develop a tool for straightforward selection of daughter strains with EC integration in the safe harbor locus on chromosome 3. This was achieved by transforming EC7-tdTomato-S1 with an EC carrying a GFP-P2A-Blast^{R} cassette and homology arms complementary to the Pol I promoter and terminator plus flanking regions of chromosome 3. Transformants were selected on blasticidin S-containing media, and colonies that showed no detectable on-plate tdTomato fluorescence were selected for further analysis. We found that 10/10 of these colonies had no detectable levels of tdTomato fluorescence in flow cytometry analysis and GFP fluorescence at levels that were expected for integration at the safe harbor locus. Using PCR analysis, we were able to confirm that in 3/3 colonies the tdTomato-P2A-zeo^{R} cassette of the parental strain had been replaced with the GFP-P2A-Blast^{R} cassette of the new EC. In conclusion, this novel approach represents a streamlined process for straightforward screening and selection of N. oceanica strains that show highly efficient transgene expression, safeguarded by targeted EC insertion into the safe harbor of the rDNA cistron on chromosome 3.

### EXAMPLE 9: The Pol I Terminator is Dispensable for Transgene Expression

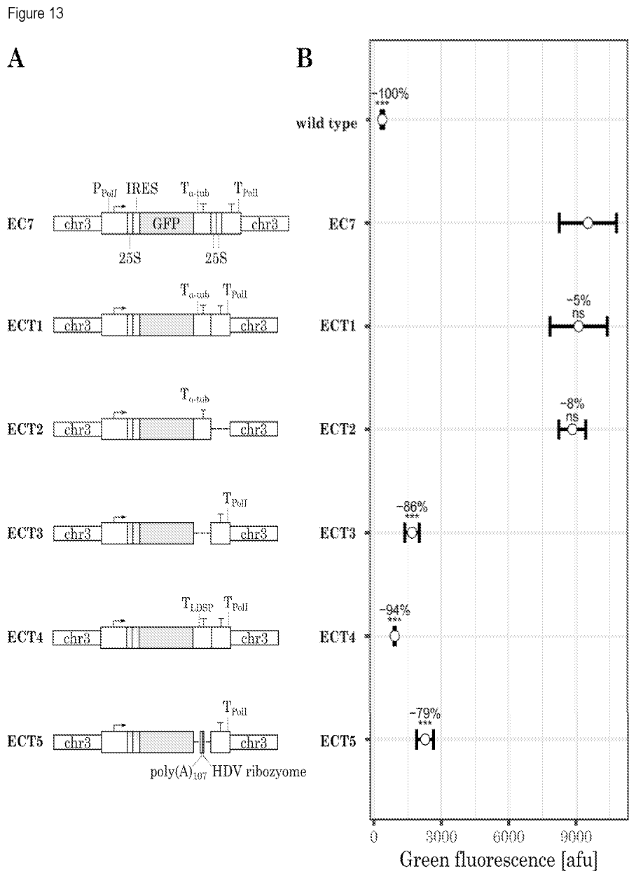

Based on the previous results we concluded that the integration of the EC within the NOR of the algal genome is essential to promote highly efficient transcription. We have further shown that the 255 nucleotides upstream of the transgene in the ECs contain a functional IRES element which enables translation of the uncapped fuRNA. To understand the relevance of the DNA elements on the 3′ side of the transgene we designed a series of cassettes and integrated them into the genome of N. oceanica at the safe harbor locus of the rDNA cistron on chromosome 3 (FIG. 13A). For this purpose, we employed the previously developed pipeline for selection of colonies with insertion at this locus, using EC7-tdTomato-S1 as a parental strain. The transformed ECs were based on EC7, but carried a Blast^{R} instead of zeo^{R} and homology flanks to facilitate HR-mediated insertion at the target site. All new ECs carried the Pol I promoter, the 5′-end of the 25S rDNA, and the Noc-IRES on the 5′-side of EGFP, but different sequences on the 3′-side of the new EGFP cassette.

ECT1 had the same elements as EC7 on the 3′ side of the EGFP cassette, with the exception of the 25S rDNA sequences, which were removed in ECT1. This deletion did not negatively affect fluorescence intensity in transformant lines (FIG. 13B). ECT2 was based on ECT1, but the Pol I terminator sequence was deleted in this construct. Fluorescence intensity in ECT2 transformant lines was comparable to EC7 and ECT1 transformants, indicating that the Pol I terminator is dispensable for transgene expression. This finding suggests that either the x-tubulin terminator can act as a transcriptional terminator not only for Pol II but also in the context of Poll transcription, or transcription terminates downstream of the EC in adjacent genomic DNA sequences in ECT2 transformants. In line with the former assumption, a previous study has shown that yeast Pol I can arrest and possibly terminate at a prokaryotic p-factor-independent transcriptional terminator motif in vitro (Clarke et al., 2018, Proceedings of the National Academy of Sciences of the United States of America, 115 (50), E11633-E11641). However, this transcriptional arrest was shown to involve a poly (T) tract, which is not present in the α-tubulin terminator sequence of N. oceanica.

### Example 10: Translation is Enhanced Through a Cis-Acting Element in the α-Tubulin Terminator Sequence

Pol II terminators contain the DNA elements that are involved in cleavage and polyadenylation of nascent pre-mRNA molecules. In the context of Pol II transcription, transcript cleavage triggers transcriptional termination (Proudfoot, 2016), which is similar to the termination process of Poll transcription (Prescott et al., 2004, Proceedings of the National Academy of Sciences of the United States of America, 101 (16), 6068-6073). Poly (A) tails are generally considered to be necessary for mRNA transport to the cytosol and cytosolic mRNA stability and they play a key role in translation initiation (Sachs, 1990, Current Opinion in Cell Biology, 2 (6), 1092-1098 However, it is unclear if Pol II poly (A) signals can induce polyadenylation in the context of Pol I transcription and no reports exist that would substantiate this notion. In fact, studies have shown that the C-terminal domain (CTD) of the Pol II enzyme complex plays an essential role in polyadenylation (Dantonel et al., 1997, Nature, 389 (6649), 399-402; Hirose & Manley, 1998, Nature, 395 (6697), 93-96). Because Pol I does not contain the CTD, the question arises which role Pol II transcriptional terminators play in the context of Pol I-based gene expression. We investigated whether the Pol II terminator present in our constructs was relevant for gene expression in N. oceanica by transforming the alga with a series of modified constructs.

First, we constructed ECT3 by removing the α-tubulin terminator sequence from ECT1 (FIG. 13A). Remarkably, microalgal transformant strains carrying ECT3 on average showed 86% decreased fluorescence compared to strains carrying the full length EC7 construct (FIG. 13B). To test whether this decrease was related to the deletion of the poly (A) signal in the terminator, we designed 2 additional cassettes. In ECT4 we inserted the transcriptional terminator from another endogenous Pol II-transcribed gene (LDSP) of N. oceanica between the EGFP cassette and the Pol I terminator sequence (FIG. 13A). This cassette was not able to induce the high fluorescence phenotype observed in EC7 or ECT1 strains. Instead, transformant strains displayed only 6% fluorescence compared to levels observed for EC7 transformants. To exclude the possibility that the LDSP terminator was unable to induce sufficient polyadenylation due to a weaker poly (A) signal compared to the x-tubulin terminator, we further constructed ECT5, by directly inserting a poly (A) coding tract between the EGFP cassette and the Pol I terminator (FIG. 13A). To facilitate formation of transcripts with free poly (A) tails instead of internal poly (A) tracts, we inserted an HDV-like self-cleaving ribozyme between the poly (A) coding sequence and the Pol I terminator. This ribozyme sequence promotes endoribonucleolytic transcript cleavage at its 5′-boundary upon transcription (Gao & Zhao, 2014, Journal of Integrative Plant Biology, 56 (4), 343-349). ECT5 transformant strains showed an increase in fluorescence compared to ECT3 and ECT4 transformants, but fluorescence intensity was only 21% compared to EC7 strains.

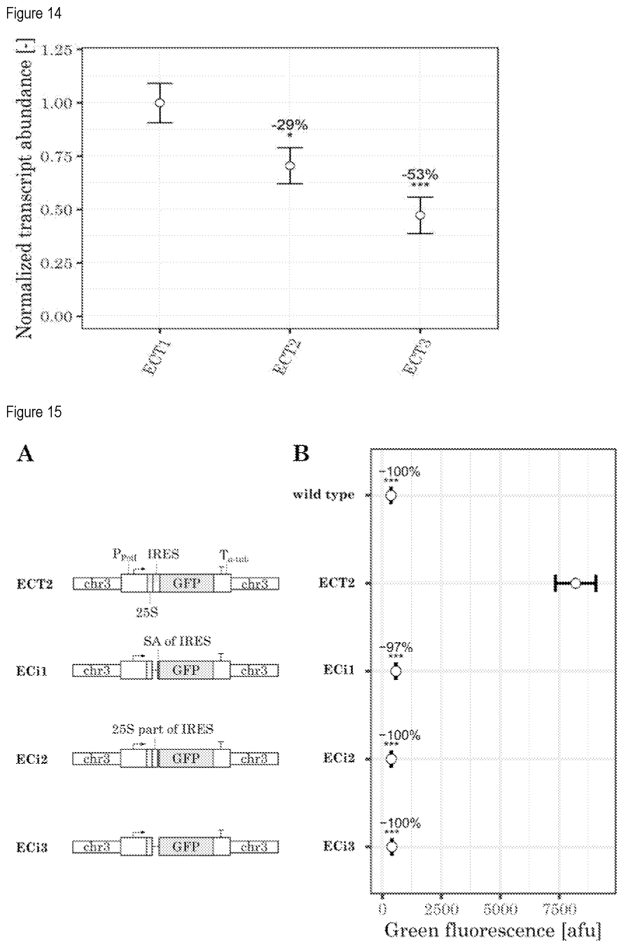

Concluding, removal of the α-tubulin terminator sequence resulted in a substantially decreased gene expression in ECT3 transformant strains compared to ECT1 and ECT2 strains. Gene expression levels were not restored to ECT1 levels by substituting the α-tubulin terminator with either the LDSP terminator or a poly (A) tail. These results suggest that the decreased transgene expression in ECT3 strains compared to ECT1 strains is related to neither transcriptional termination nor the presence or absence of a poly (A) tail in the fuRNA. It is well known that 3′-UTRs of mRNAs can contain cis-acting elements that are involved in regulation of post-transcriptional processing, transcript degradation, nucleocytoplasmic transport and translation initiation through interaction with specific RNA binding proteins (RBPs) (Matoulkova et al., 2012; Moore & Lindern, 2018). Prominent examples of cis-regulatory elements include the poly (A) tail itself and elements that control its length by governing cytoplasmic polyadenylation or deadenylation. Other, less generic 3′-UTR elements are directly involved in translation by affecting initiation or elongation rates (Hussey et al., 2011, Molecular Cell, 41 (4), 419-431; Kapasi et al., 2007, Molecular Cell, 25 (1), 113-126; Moore & Lindern, 2018; Nakamura et al., 2004, Developmental Cell, 6 (1), 69-78). The α-tubulin terminator sequence appears to contain one or multiple cis-acting elements that could be relevant for either stability, post-transcriptional processing or translation of the fuRNA. To test whether these elements increase transcript processing or stability, we quantified EGFP transcript abundance in representative ECT1, ECT2 and ECT3 strains through RTq-PCR (FIG. 14). EGFP transcript levels in the ECT3 strain were decreased by 53% (p<0.001) compared to the ECT1 strain but only by 33% (p=0.039) compared to the ECT2 strain. EGFP fluorescence levels of the ECT3 strain, however, were decreased by 85% (p<0.001) and 84% (p<0.001) relative to ECT1 and ECT2. Consequently, the decrease in transcript abundance alone cannot explain the decreased fluorescence levels of the ECT3 strain. The main effect of the α-tubulin terminator on gene expression thus appears to be related to either nucleoplasmic transport, or translation of the fuRNA.

Whereas a lot of cis-acting 3′-UTR elements are known that inhibit translation, far less reports exist about 3′-UTR elements that stimulate it. By far the most well understood 3′-UTR elements that have a stimulating effect on translation are 3′-cap-independent translation enhancers (3′-CITEs). These structurally and functionally highly diverse elements have thus far almost exclusively been found in positive strand RNA viruses of plants, where they substitute for the absence of a 5′-cap and poly (A) tail by recruiting either eukaryotic translation initiation factors (eIFs) or the ribosomal subunits to the viral genome (Gao et al., 2012, Journal of Virology, 86 (18), 9828-9842; Nicholson et al., 2010, Rna, 16 (7), 1402-1419; Stupina et al., 2008, Rna, 14 (11), 2379-2393; Wang et al., 2009, Journal of Biological Chemistry, 284 (21), 14189-14202). To induce translation initiation, the recruited trans-acting factors need to be brought into proximity with the 5′-UTR. In most viruses that contain 3′-CITE elements, this is achieved through circularization of the RNA molecule by long distance RNA-RNA interactions between complementary bases in the 5′-UTR and the 3′-UTR of the viral genome or through recruitment of a protein bridge by designated elements in both UTRs (Bradrick et al., 2006, Nucleic Acids Research, 34 (4), 1293-1303; Gazo et al., 2004, Journal of Biological Chemistry, 279 (14), 13584-13592; Souii et al., 2015, Current Microbiology, 71 (3), 387-395). Whereas 3′-CITE elements do require interaction between the 5′ and 3′-UTR of the RNA molecule, they do not rely on presence of an IRES in the 5′-UTR. We therefore decided to test if the α-tubulin terminator was capable of promoting fuRNA translation independently of the NocIRES, analogously to 3′-CITE elements of plant RNA viruses.

### Example 11: The Noc-IRES Requires 25S rRNA Sequence and the Poly (Y) Tract for Full Functionality

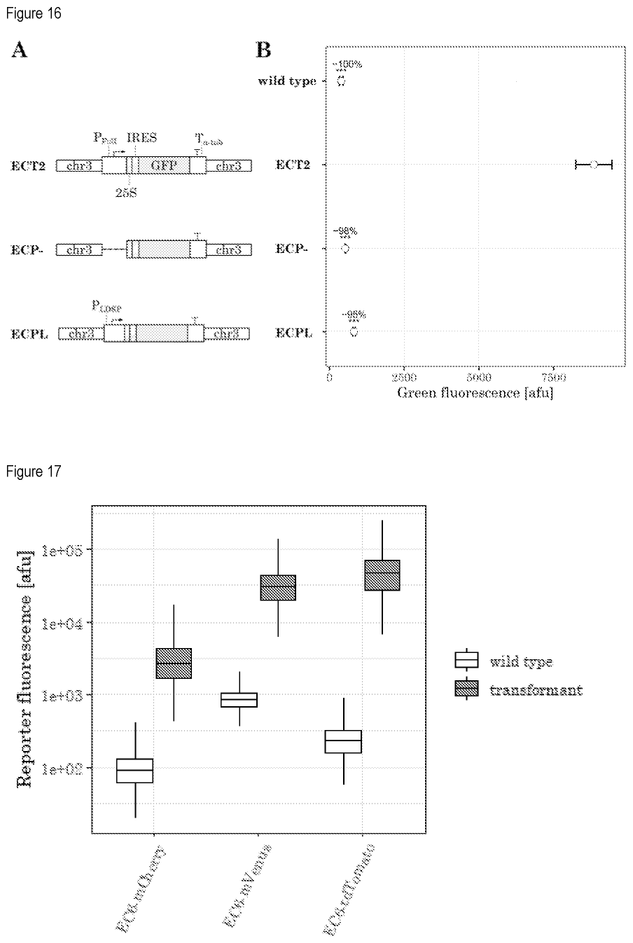

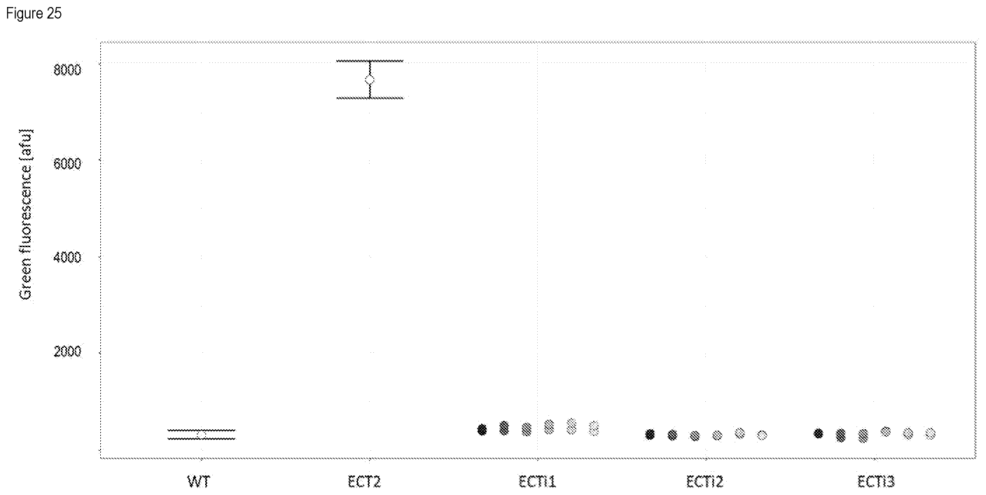

To understand more about the mode of translation initiation on the fuRNA and to test if the α-tubulin terminator could act analogously to plant RNA viral 3′-CITE elements, we tested the effect of partial and complete IRES deletions on construct ECT2, which was the shortest construct capable of driving high GFP expression (FIG. 15A). The construct ECi1 lacks the 200 5′-terminal nucleotides of the IRES, corresponding to the 25S rRNA nucleotides 2134-2336 of N. oceanica. In this construct the poly (Y) tract-containing SA sequence is directly connected to the 25S rRNA nucleotides 1-91, which had been included in all constructs to prevent fuRNA-degradation, as previously discussed. ECi2 instead lacks the 55 3′-terminal nucleotides of the IRES, corresponding to the SA sequence including the poly (Y) tract, which is a frequently found element in a variety of IRESs (Jang & Wimmer, 1990, Genes and Development, 4 (9), 1560-1572; Jaramillo-Mesa et al., 2018, Journal of Virology, 93 (5); Martinez-Salas et al., 2018). In ECi3, the entire 255 nucleotides of the IRES had been deleted. We inserted the ECs into the rDNA copy of chromosome 3, using the parental strain EC7-tdTomato-S1 and CRISPR/Cas technology as described above. All constructs, including ECi3, yielded viable transformants. Six colonies per EC were selected for loss of tdTomato fluorescence and subsequently subjected to EGFP fluorescence screening by flow cytometry (FIG. 24). Fluorescence levels of different colonies of the same construct were similar for all ECs. Correct HDR-mediated insertion was verified for three colonies per construct by PCR and sequencing and one representative colony was chosen for fluorescence quantification (FIG. 15B). Deletion of the 25S rRNA part of the IRES in ECi1 colonies had decreased EGFP fluorescence emission in transformants by 97% (p<0.001) compared to ECT2 strains. Although ECi2 and ECi3 colonies were viable, they showed no measurable EGFP fluorescence over wild type levels. The complete loss of fluorescence for these constructs suggests that the SA element contains a nucleotide sequence that is essential for activity of this IRES. Because ECi1 colonies retained a measurable level of fluorescence, the SA element alone appears to be sufficient for translation initiation on the fuRNA. As previously discussed, the poly (Y) tract of the SA is a candidate element for facilitating this translation initiation for instance by recruiting the ribosome through interaction with the known ITAF poly (Y) tract binding protein (PTB). Despite this, fluorescence emission levels were minute in ECi1 transformants compared to strains carrying the full length Noc-IRES, indicating that the 25S rRNA part of the Noc-IRES plays a major role in the high levels of activity of this IRES.

The absence of fluorescence in ECi3 strains indicates that the x-tubulin terminator is inefficient in promoting fuRNA translation independently of the Noc-IRES. The element thus appears to enhance gene expression by a different mechanism compared to plant virus 3′-CITE elements. Despite this, it is possible that the deletion of the IRES potentially interfered with long-distance RNA-RNA interactions between the α-tubulin terminator and the 5′-UTR, which could have prevented 3′-CITE-like activity of the element in ECi3 strains. However, it seems more likely that the α-tubulin terminator specifically stimulates IRES-mediated translation e.g. by recruiting trans-acting factors such as eIFs or ITAFs to the RNA. Similarly, translation of the foot-and-mouth disease virus RNA genome is known to involve an interaction between an IRES in its 5′-UTR and a 3′-UTR translation enhancer (TE). This interaction is not essential for gene expression in the virus but it has a stimulating effect on translation (García-Nuñez et al., 2014, Virology, 448, 303-313; Serrano et al., 2006, Journal of General Virology, 87 (10), 3013-3022). Alternatively, the TE element in the α-tubulin terminator may enhance translation not by recruiting trans-acting factors but by facilitating circularization of the fuRNA instead. This could help to guide the translation machinery back to the IRES after translation termination, analogously to models for translation re-inititation on circularized mRNAs (Alekhina et al., 2020, International Journal of Molecular Sciences, 21 (5), 1677). The exact mechanism by which the x-tubulin terminator sequence enhances translation of the fuRNA remains to be investigated. However, based on the results presented above, we can conclude that the α-tubulin terminator increases gene expression by a mechanism that appears to be related to translation. On top of that, the element further positively influences fuRNA abundance, which may be attributed to a positive effect on either transcription, post-transcriptional processing, or transcript stability.

### Example 12: The Pol I Promoter is Indispensable for Strong Transgene Expression

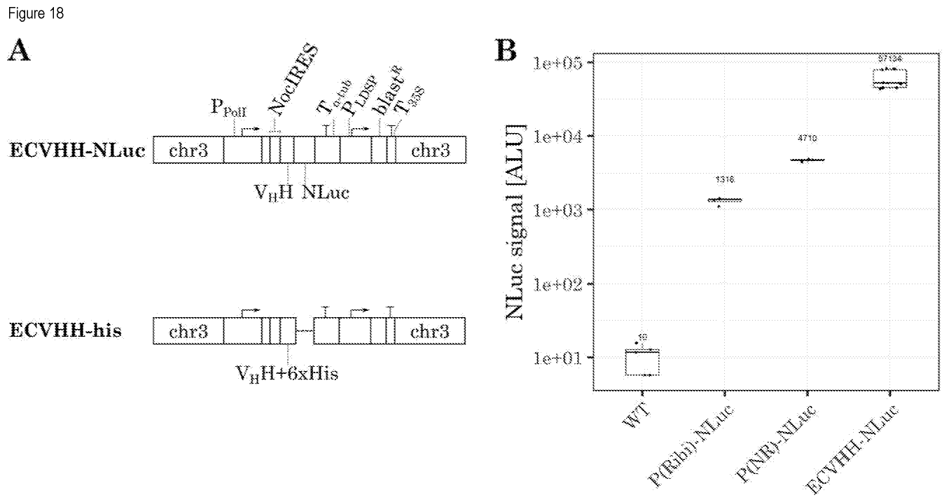

Based on the experiments discussed above, we concluded that insertion of an EC containing an IRES and TE element into the NOR of N. oceanica promotes highly efficient transgene expression. The high efficiency of this system is caused by an involvement of Pol I, producing extraordinarily high amount of transcript (FIG. 3b. The involvement of Pol I in this process is likely for several reasons: (i) the TC insertion was found within an rDNA cistron; (ii) transcription levels are substantially increased compared to a strong Pol II promoter (VCP); (iii) strong transgene expression occurs only when the EC is inserted in or adjacent to an rDNA cistron, which highlights that the nucleolar enzymatic machinery is required for transcription. Despite this strong evidence, we opted to further verify that transgenes are transcribed by Pol I and not by Pol II because previous studies have shown that Pol II can be active in the nucleolus of eukaryotic cells (Earley et al., 2010, Genes and Development, 24 (11), 1119-1132; Guo et al., 2015, Scientific Reports, 5 (1), 1-10; Song et al., 2019, BMC Biotechnology, 19 (1), 54). To this end, we designed 2 additional ECs, termed ECP- and ECPL, which were based on ECT2 as shortest functional construct. We removed the Pol I promoter sequence in ECP- and replaced it with the Pol II transcriptional promoter of the endogenous LDSP gene in ECPL (FIG. 16). The LDSP promoter had shown steady expression of fluorescent reporters in previous studies (data not shown). Both constructs were inserted into the rDNA copy of chromosome 3 through CRISPR/Cas system and in both cases we found viable N. oceanica transformants. We verified correct cassette insertion in transformants and screened them for EGFP fluorescence (FIG. 16). Removal of the Pol I promoter sequence abolished fluorescence in transformants, making this element indispensable for achieving high transgene expression levels. Transformant strains carrying ECPL in the NOR of chromosome 3 showed the same fluorescence as average fluorescence levels observed for transformant strains in which the construct had been randomly inserted into the genome (data not shown). Consequently, the integration of a cassette carrying a Pol II promoter within the NOR does not enhance transcription levels compared to nucleoplasmic insertion. This is in stark contrast to what we observed for cassettes carrying the Pol I promoter, which further substantiates that Poll mediates transgene expression in the highly fluorescent strains of this study.

### Example 13: Expression of Different Fluorescent Protein Genes

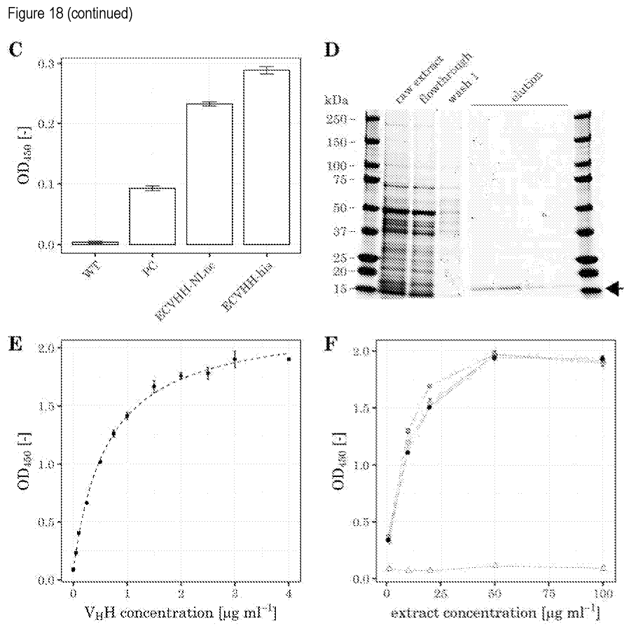

We successfully employed the presented gene expression system to heterologously express 3 fluorescent proteins other than EGFP in N. oceanica (FIG. 17). Whereas mVenus and mCherry are of similar size compared to EGFP, the 54.2 kDa DsRed variant tdTomato was chosen to test the robustness of the novel gene expression system for production of larger proteins. mCherry constructs induced slightly lower levels of fluorescence in transformant strains compared to EGFP while mVenus and tdTomato constructs yielded significantly improved fluorescence, likely because of the improved brightness of these proteins over EGFP.

### Example 14: Production of Therapeutic Proteins

We further tested the new gene expression system for the production of recombinant antibodies in N. oceanica. Recombinant antibodies are high-value biologics that are important scientific tools in biomedical research and powerful therapeutic agents for the treatment of infectious diseases and cancer (Lu et al., 2020). Conventional antibodies are immunoglobulin proteins that consist of multiple heavy and light peptide chains and bind to a target molecule with high specificity and affinity. With a size of ˜150 kDa, their application for the treatment of cancer is limited by tissue and tumor penetration (Baker et al., 2008, Clinical Cancer Research, 14 (7), 2171-2179; Beckman et al., 2007). Antibodies from camelid species are much smaller because they are devoid of light chains (Bannas et al., 2017). They contain a variable region, designated V_{H}H, which binds to antigens with specificity and affinity comparable to those of conventional antibodies. This VAH domain can be produced independently of the rest of the protein without losing its structural or functional properties. V_{H}H ‘nanobodies’ are extremely stable, highly soluble and they weigh only ˜15 kDa, which greatly improves their tissue penetration compared to human immunoglobulins.

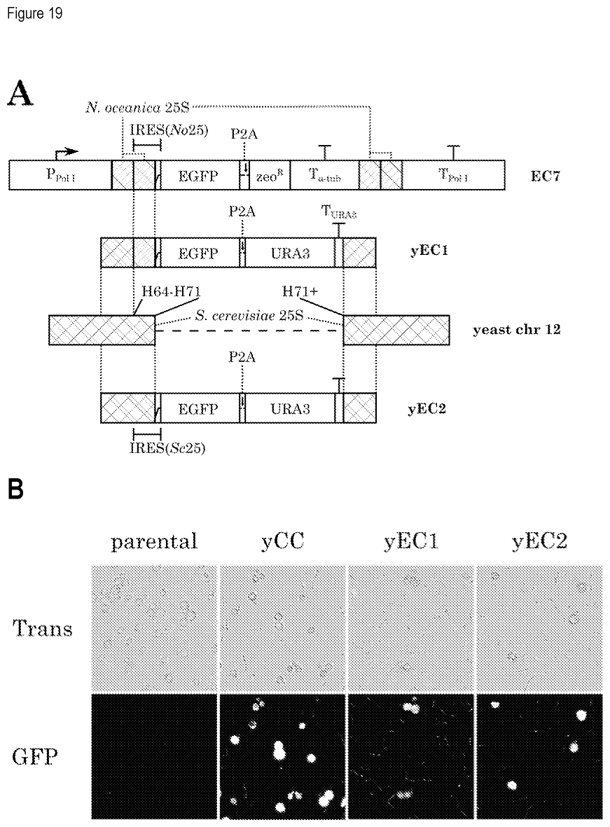

We engineered N. oceanica with the novel gene expression system to produce a V_{H}H that binds to GFP with high affinity and specificity (Rothbauer et al., 2008, Molecular and Cellular Proteomics, 7 (2), 282-289). The microalga was transformed with two different ECs, termed ECVHH-NLuc and ECVHH-his (FIG. 18A). The ECs were based on ECT1 with a few modifications. The EGFP-P2A-bleo^{R} cassette was replaced with a genetic V_{H}H-NLuc or V_{H}H-his-tag fusion in ECVHH-NLuc and ECVHH-his respectively. NLuc encodes Nanoluc luciferase which was included to ensure rapid screening of transformant strains (England et al., 2016). In ECVHH-his, a his-tag coding region was added to facilitate purification of the V_{H}H protein. On the 3′-side of the α-tubulin terminator sequence we inserted a second cistron, encoding a blasticidin antibiotic resistance gene under control of the Pol II LDSP promoter and the transcriptional terminator of cauliflower mosaic virus (35S). ECs were inserted into a parental strain carrying a modified ECT2 version in the NOR of chromosome 3, similarly to the procedure described above for straightforward screening and selection of transformant strains when using EC7-tdTomato-S1 as a parental strain for transformation. The modified ECT2 that was used to create the parental strain ECT2-tdTomato-S1 carried a tdTomato-P2A-zeo^{R} cassette instead of the GFP-P2A-Blast^{R} cassette present in ECT2. Upon transformation of ECT2-tdTomato-S1 with ECVHH cassettes, only strains with correct insertion of the ECVHH construct were subsequently screened for NLuc activity and presence of functional V_{H}H. NLuc signal in luminescence assays was 12-43× increased for ECVHH-NLuc transformants compared to levels observed for transformants carrying NLuc under control of two different Pol II promoters (FIG. 18B). In our screenings, all ECVHH-NLuc strains that carried the EC in the NOR showed high levels of NLuc activity, which matches our observations during earlier experiments on EGFP expression.

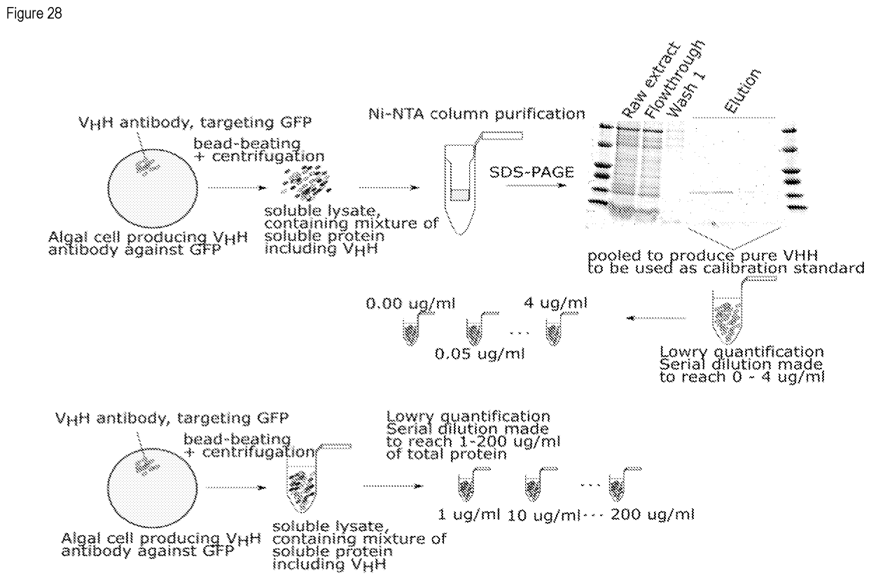

Subsequently we investigated whether the microalgal-produced V_{H}H was effectively binding GFP. Proteins were extracted from transformant strains and subjected to an indirect ELISA (schematic in FIG. 27). Protein extracts from strains carrying either version of ECVHH produced substantial amount of signal (FIG. 18C), which was high compared to that produced by a purified mammalian anti-GFP antibody (PC). Samples from strains expressing the his-tagged V_{H}H produced 24% higher signal compared to strains producing VAH-NLuc fusions. Hence, we considered only transformants carrying ECVHH-his for quantification of V_{H}H concentration.

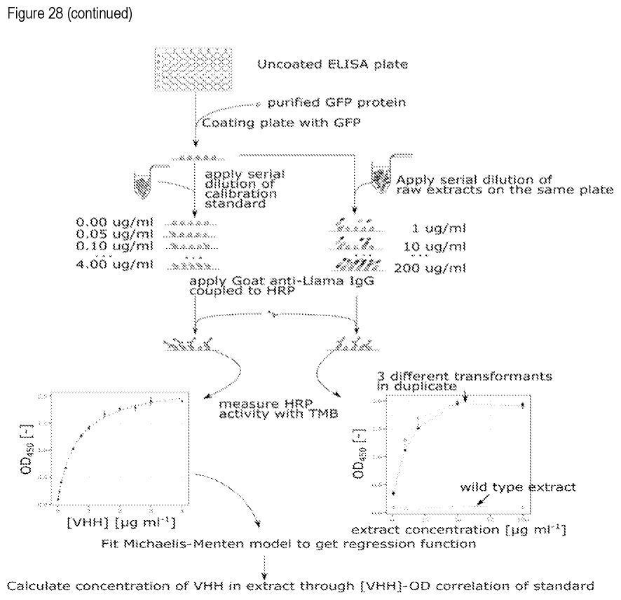

We quantified the fraction of V_{H}H per protein in the soluble extract by indirect ELISA (FIG. 28). First, we extracted proteins from a ECVHH-his transformant strain and purified the V_{H}H-his by immobilized metal affinity chromatography (FIG. 18D). Clean elution fractions were pooled and V_{H}H-his concentration was determined. The purified V_{H}H-his was then used as a calibration standard in an indirect ELISA (FIG. 18E). The relationship between OD₄₅₀ values and V_{H}H-his concentration was reliably described by a Michaelis-Menten model (R²>0.995). During this ELISA, we further analysed soluble extracts from three ECVHH-his transformant strains in serial dilution (FIG. 18F). Signal strength in extracts followed the same Michaelis-Menten dynamic as the calibration standard. Only the samples with a protein concentration of 1 μg ml⁻¹ produced a signal that fell into the almost-linear range of the calibration curve (FIG. 18E). With the data of these samples, we calculated the concentration of functional V_{H}H-his in the soluble extracts using the Michaelis-Menten model derived from the calibration standard. Fractions of V_{H}H-his per soluble protein were 8.55±0.02%, 9.63±0.08% and 7.98±0.48% for the three transformants. This highlights that the new gene expression system can be employed to produce high levels of functional protein in N. oceanica.

### Example 15: Application of the Gene Expression System in S. cerevisiae

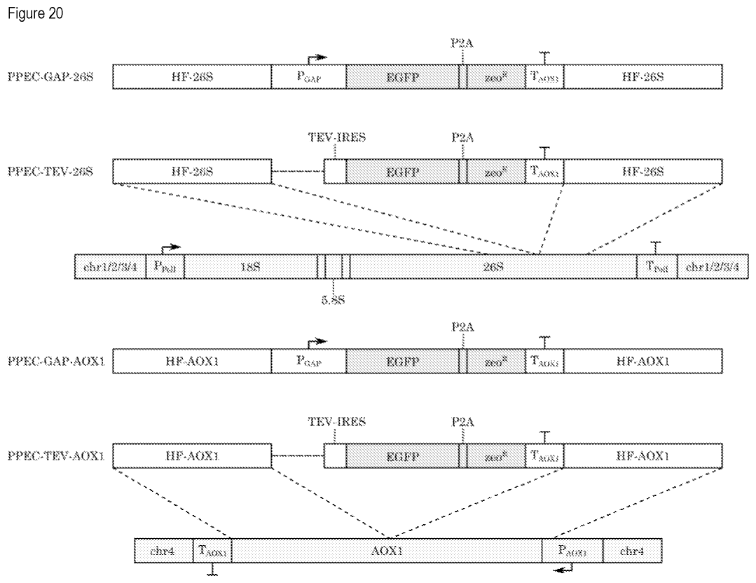

To investigate whether the nucleolar Pol I-based gene expression system can be transferred to other eukaryotic organisms, we transformed S. cerevisiae with a modified version of the EC. Because S. cerevisiae carries a substantially higher number of rDNA cistrons compared to N. oceanica (Petes, 1979, Proceedings of the National Academy of Sciences of the United States of America, 76 (1), 410-414) a single EC insertion may not be sufficient to drive strong gene expression. Therefore we designed promoterless yeast-specific ECs (yECs) with homology flanks for the 25S rRNA gene, possibly facilitating multiple insertions. Consequently we chose a control construct (yCC) that also facilitates multiple genomic integrations of the reporter gene under control of a strong Pol II promoter (TEF) (Maury et al., 2016, PLOS ONE, 11 (3), e0150394). yEC1 and yEC2 carry an EGFP-P2A-URA3 reporter gene fusion demarcated by the transcriptional URA3 terminator (FIG. 19A). Directly flanking EGFP on the 5′-side is the IRES as it was discovered in N. oceanica TC #17 i.e. a fusion of 25S rRNA helices H64-H71 and the SA element of the VCP1 gene intron 1. In yEC1 the nucleotide sequence encoding H64-H71 was identical to the N. oceanica version, whereas yEC2 carried the S. cerevisiae sequence. HR-mediated insertion was achieved by homology arms targeting the constructs directly to H71 of the 25S rRNA gene. Reporter fluorescence was seen in transformant strains of both yEC1 and yEC2 (FIG. 19B), proving that nucleolar Pol I-based gene expression can be achieved in other eukaryotic organisms. Interestingly, fluorescence emission levels were slightly higher for yEC2 compared to yEC1, despite a 93% nucleotide sequence identity between the helices 64-71 of N. oceanica and S. cerevisiae, suggesting that the high activity of the 25S rRNA-SA fusion IRES may vary somewhat depending on the organism and may involve high sequence similarity to the endogenous 25S rRNA. These results show that the nucleolar Pol I-based gene expression system can work in other lower eukaryotes as well. Presumably, transgene expression levels could further be improved by choosing an IRES with a strong translation initiation and/or by adding a TE element, as this greatly improved gene expression in N. oceanica.

### Example 16: Transfer of the Gene Expression System to Pichia pastoris

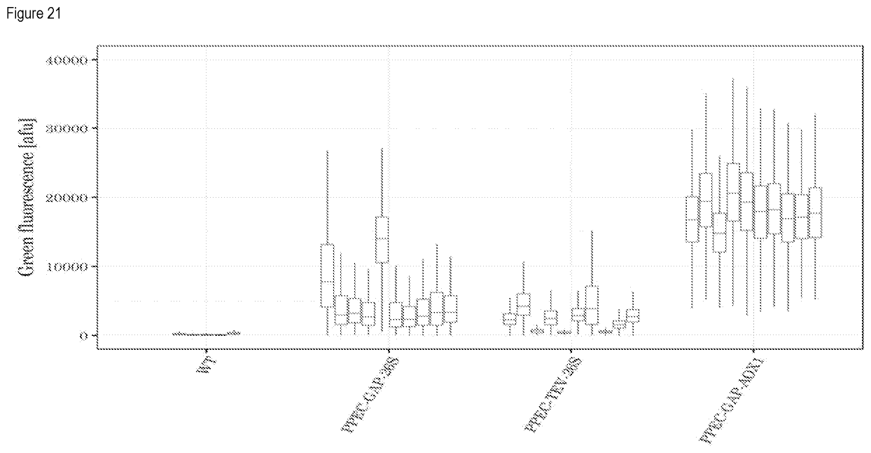

We chose to further test whether the novel gene expression system can be employed to express genes in P. pastoris. We chose P. pastoris because it is a widely used, industrially relevant platform for high efficiency production of recombinant protein (Cregg et al., 1993, Bio/Technology; Li et al., 2007). P. pastoris has an estimated 16 rDNA copies in total (De Schutter et al., 2009, Nature Biotechnology, 27 (6), 561-566), which is low compared to S. cerevisiae, but still higher than the four copies of N. oceanica. Because of this, a single EC insertion may not be sufficient to drive strong gene expression in P. pastoris. Therefore, we designed a promoterless P. pastoris-specific EC (PPEC-TEV-26S, FIG. 20) with homology flanks complementary to the 26S rRNA gene of P. pastoris, facilitating multiple genomic integrations and ensuring Pol I-mediated transcription of the transgenes. The reporter gene EGFP was polycistronically linked to zeo^{R} using a P2A peptide. On the 5′-side, flanking the GFP-P2A-zeo^{R} cassette in PPEC-TEV-26S was the tobacco etch virus (TEV) IRES that had previously shown strong IRES activity in P. pastoris (Huang et al., 2019, Biotechnology for Biofuels). Control cassettes (PPEC-GAP) contained the transcriptional promoter of the glyceraldehyde-3-phosphate dehydrogenase (GAP) gene instead of the IRES. The GAP promoter is one of the strongest constitutively active transcriptional promoters for Pol II in P. pastoris and used in numerous studies (Marx et al., 2009, FEMS Yeast Research, 9 (8), 1260-1270; Song et al., 2019, BMC Biotechnology, 19 (1), 54; Várnai et al., 2014, Microbial Cell Factories, 13 (1), 57; Zhang et al., 2009, Molecular Biology Reports, 36 (6), 1611-1619) as well as in commercial protein production systems. It was therefore an ideal reference for the strength of the novel expression system in P. pastoris. The control construct PPEC-GAP-26S carried the same homology flanks as PPEC-TEV-26S to allow multiple integrations. In all ECs, the reporter cassette was flanked on its 3′-side by the AOX1 transcriptional Pol II terminator, to safeguard correct gene expression from the constructs containing the GAP promoter. A second set of ECs was equipped with homology flanks complementary to the AOX1 locus, which is a well-studied, transcriptionally highly accessible region of the nucleoplasm that is not associated with Pol I transcription. Thus, it is a suitable locus to test dependency of EGFP expression from PPEC-TEV cassettes on NOR-specific insertion and to check for presence of a cryptic Pol II promoter in the PPEC-TEV construct.

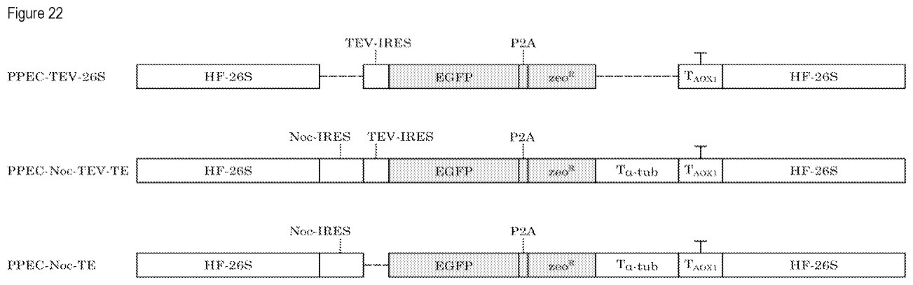

We transformed P. pastoris with all four constructs by electroporation. Transformation with PPEC-TEV-AOX1 did not yield any viable colonies, showing that the core of the PPEC-TEV cassettes does not contain a cryptic Pol II promoter and can therefore not cause reporter gene expression unless inserted into an actively transcribed gene. Viable transformants were found for PPEC-TEV-26S on the other hand, indicating that combination of Pol I promoter and the TEV IRES is capable of producing functional protein in P. pastoris. PPEC-GAP cassettes with both versions of the homology flanks produced viable transformant strains. Ten colonies were randomly picked for PPEC-TEV-26S, PPEC-GAP-26S and PPEC-GAP-AOX1 and analyzed for EGFP fluorescence by flow cytometry (FIG. 21). Six transformant strains per construct were further analyzed by PCR for insertion of the EC within a 26S rDNA sequence or the AOX1 locus. Insertion had occurred at the target site in all cases. EGFP fluorescence was consistently high for PPEC-GAP-AOX1 transformants (FIG. 21). Fluorescence of PPEC-GAP-26S strains was less consistent and overall lower than for PPEC-GAP-AOX1 strains, confirming that the AOX1 locus is a highly euchromatic region of the P. pastoris genome. PPEC-TEV-26S strains showed the highest variability in reporter expression and lowest average expression levels. Some colonies, however, had EGFP fluorescence levels comparable to the majority of PPEC-GAP-26S strains, proving that nucleolar gene expression using Pol I and an IRES can drive high levels of protein production in P. pastoris.

Despite this, reporter expression among PPEC-TEV-26S strains was highly variable. This is likely owed to the random insertion of the EC into any of the 16+rDNA cistrons of P. pastoris. It is well known that in human cells, rDNA cistrons can be in a transcriptionally active or inactive state, which is retained through cell division (Grob et al., 2014, Genes and Development, 28 (3), 220-230). The division into active and inactive states is also accepted for other eukaryotes, and it would be a possible explanation for the high variability in gene expression among different PPEC-TEV-26S transformants. It was further shown that in S. cerevisiae, transcriptional activity can vary even between different rDNA cistrons in the active state (Wittner et al., 2011, Cell, 145 (4), 543-554). Additionally, eukaryotic cells can contain partial rDNA cistrons and rRNA pseudogenes, which may be transcribed less efficiently or not at all (Ferretti et al., 2019, Chromosoma, 128 (2), 165-175; Floutsakou et al., 2013, Genome Research, 23 (12), 2003-2012). EC insertion into rRNA pseudogenes or rDNA cistrons with epigenetic modifications that affect affinity for Pol I may explain the high variability of reporter gene expression among different PPEC-TEV-26S transformants. Moreover, the high variability could be caused by multiple EC insertions or EC duplication events.

Although several PPEC-TEV-26S transformants showed gene expression levels comparable to the average of PPEC-GAP-26S transformants, best performers of PPEC-GAP-26S had higher expression levels (FIG. 21). Furthermore, reporter expression from Pol II control constructs inserted at the AOX1 locus was >3-fold increased for all colonies relative to the best PPEC-TEV-26S performers. The expression efficiency obtained with PPEC-TEV-26S in P. pastoris fall short compared to those obtained when transforming N. oceanica with similar constructs. Potential explanations for this include (i) different rDNA copy numbers of the different organisms (16 for P. pastoris versus 4 for N. oceanica); (ii) less control over the insertion site for PPEC-TEV-26S due to cistron-unspecific homology flanks; (iii) different IRES sequences; (iv) absence of a TE element for the P. pastoris cassettes. The last point seems particularly important because in our experiments with N. oceanica, the nucleotide sequence downstream of the reporter genes strongly modulated gene expression (FIG. 13B) and accounted for up to ˜17-fold changes in overall fluorescence levels. Analogously to our observations for N. oceanica, where insertion of the LDSP terminator into ECT3 constructs decreased gene expression of transformant strains by >50%, the presence of the AOX1 terminator may interfere with gene expression in PPEC-TEV-26S transformants. Unfortunately, little is known about functional interaction between IRESs and 3′-UTR elements, and no TE elements have been reported for P. pastoris. Furthermore, no element known for this species shows stronger IRES activity than the TEV IRES, limiting the options for short term improvements to the system.

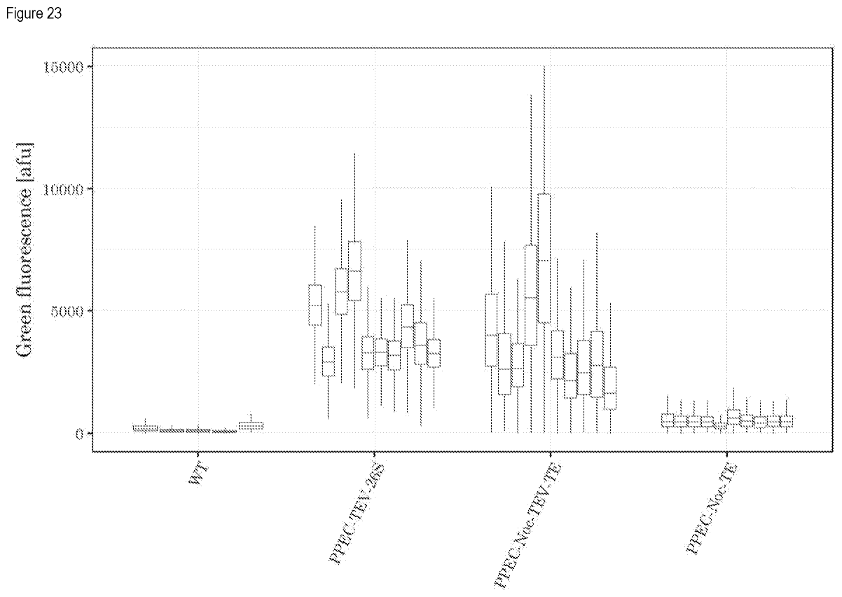

Due to the dearth of knowledge about suitable elements for improving gene expression P. pastoris, we tested whether the N. oceanica α-tubulin terminator can function as a TE element in the context of Pol I-based gene expression in yeast. We designed PPEC-Noc-TEV-TE that carried the N. oceanica α-tubulin terminator flanking zeo^{R} on the 3′-side (FIG. 22). Additionally, we added the IRES from N. oceanica 5′-adjacent to the TEV IRES in this construct, because it was unclear if the α-tubulin terminator depends on this element for activity. To assess whether the Noc-IRES element can drive cap-independent translation initiation not only in N. oceanica and S. cerevisiae but also in P. pastoris, we further constructed PPEC-Noc-TE by removing the TEV IRES from PPEC-Noc-TEV-TE.

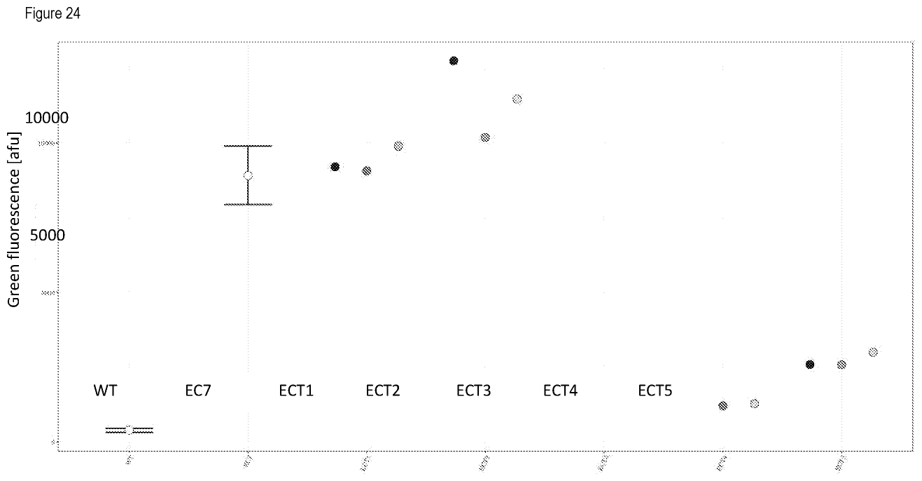

Reporter expression in PPEC-Noc-TEV-TE transformant strains was comparable to that of PPEC-TEV-TE strains (FIG. 23) indicating that addition of the x-tubulin terminator did not enhance gene expression. Consequently, this TE element may either be specific for N. oceanica, e.g. due to interaction with species-specific trans-acting factors, or it may specifically enhance cap-independent translation initiation at the Noc-IRES but not at other IRESs. Although PPEC-Noc-TE strains had substantially lower fluorescence levels than control strains, fluorescence was increased (p<0.001) compared to the wild type. Accordingly, the Noc-IRES is functionally active in P. pastoris. In our previous experiments we found that adapting the 25S rRNA sequence of the Noc-IRES to the endogenous S. cerevisiae rRNA sequence improved fluorescence of transformant cells. Analogously, Noc-IRES activity in P. pastoris might be improved in a similar fashion in future experiments. Furthermore, future studies should investigate whether IRES activity can be improved by changing the splice acceptor part of the Noc-IRES to an endogenous splice acceptor version, as this might aiding with recognition of the element by trans-acting factors such as PTB.

In conclusion, the novel transgene expression system is transferable to lower eukaryotes other than N. oceanica, such as S. cerevisiae and P. pastoris. To reach high levels of gene expression, NOR-localization is a crucial prerequisite, as we saw in our experiments with N. oceanica and P. pastoris. The choice of IRES and the elements present in the 3′-UTR can enhance the overall efficiency of the expression system. An artificially produced IRES, consisting of a combination of the highly conserved 25/26S rRNA helices 64-71 and a N. oceanica poly (Y) tract-containing splice acceptor element, was functional in all three organisms. The IRES was functionally complemented by a putative TE element in N. oceanica that greatly improved overall efficiency of the Pol I-based gene expression system and facilitated protein production at levels that are unparalleled by Pol II-based gene expression in this organism.

### Example 17: Pol I-Mediated Gene Expression in Mammalian Cells

We choose to further deliver a proof of concept for a functional nucleolar Pol I-based gene expression system in mammalian cells. Mammalian cells are industrially interesting especially for the production of complex therapeutic proteins such as antibodies, which require post-translational modifications such as glycosylation (Tripathi & Shrivastava, 2019, Frontiers in Bioengineering and Biotechnology, 7, 420). The NORs of human cell lines are well-studied, and they are located on the short arms of the acrocentric chromosomes 13, 14, 15, 21 and 22. As previously mentioned, it has been estimated that in human cells ˜50% of rDNA cistrons are transcriptionally inactivated by heterochromatinization due to lack of binding by HMG box protein UBF (McStay & Grummt, 2008, Annual Review of Cell and Developmental Biology, 24 (1), 131-157). Transcriptional inactivation of rDNA cistrons was also found in other metazoans, including yeast and plants (McStay, 2016 Genes & Development 30 (14), 1598-610). In A. thaliana, rDNA cistrons on chromosome 2 are in an inactive state whereas rDNA cistrons on chromosome 4 are active, suggesting that chromosomal context plays a role in regulating functional inactivation of NORs (Chandrasekhara et al., 2016, Genes and Development (30), 177-190). Consequently, it is important to identify the transcriptionally active NORs and rDNA cistrons to exploit Pol I for expression of transgenes in eukaryotic organisms.

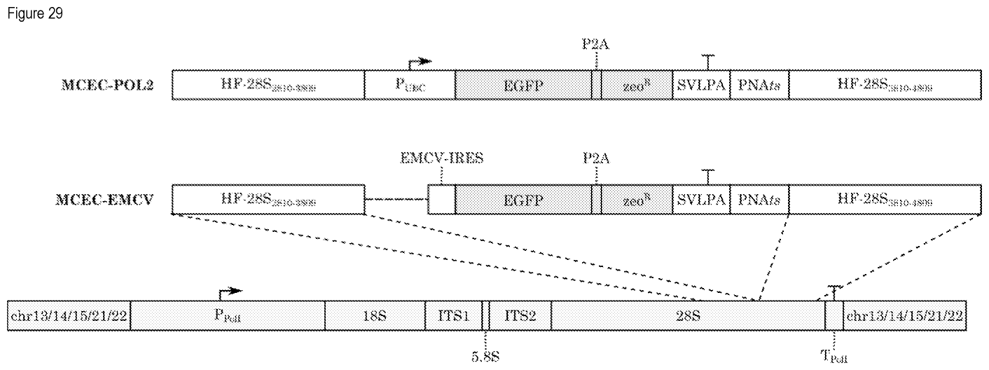

As an alternative to identifying and targeting non-silent NORs, we choose to design cassettes in a way that allows vector integration into any rDNA cistron, since this strategy was successful for producing transgenic P. pastoris strains. To this end, we design a DNA vector that can integrate into the 28S rDNA sequence of a mammalian cell line by homologous recombination or homology-directed repair (FIG. 29). This is facilitated by presence of homology flanks at the termini of a linear (e.g. PCR-produced) EC. The left and right flank are homologous to the human 28S rDNA bases 2810-3809 and 3810-4809, respectively (Kim et al., 2021, Scientific Reports 11 (1), 1-14), to ensure insertion of the vector at a similar rDNA position as identified in the 25S rDNA of N. oceanica TC #17, which proved to be an effective locus for cassette integration also in P. pastoris.

The EC carries EGFP as a reporter gene, polycistronically linked to a zeocin resistance gene via a P2A sequence that has been shown to be effective for transcriptional coupling of genes in several mammalian cell lines (Kim et al., 2011, PLOS ONE 6 (4), e18556). We choose Zeocin as one of the most promising selection agents for mammalian cell line developments (Lanza et al., 2013, Biotechnology Journal 8 (7), 811-821). In cassette MCEC-EMCV (SEQ ID. NO: 261), the reporter gene is flanked on the 5′-side by the encephalomyocarditis virus (EMCV) IRES as one of the strongest IRESs known for animal cells with activity conserved among a variety of different cell types. We choose the native preferred IRES, corresponding to viral bases 273-845, with the native A6 instead of the widely-used A7 bifurcation loop sequence (Bochkov & Palmenberg, 2006, BioTechniques 41 (3), 283-292). The reporter cassette is flanked on the 3′-side of the zeo^{R} STOP codon by the simian virus 40 late polyadenylation signal (SVLPA). Downstream of the SVLPA lies a peptide nucleic acid target site (PNAts) which is added to allow in vitro loading of ECs with complementary peptide nucleic acids (PNAs) that are equipped with a nucleolar localization sequence (NoLS), to improve trafficking of the PNA-loaded EC to the nucleolus of transfected cells. Homology flanks are attached to the construct to allow insertion of the EC by homologous recombination or homology directed repair. Upon faithful EC insertion into any rDNA cistron, the IRES and reporter genes will be oriented in frame with the 28S rRNA gene to allow transcription by Pol I. As we did for P. pastoris, we design a control construct MCEC-POL2 containing the constitutive endogenous ubiquitin C promoter (PUBc) sequence instead of the EMCV IRES. Due to the high gene copy number of rRNA genes in mammalian cells, multiple EC insertions are possible and expected to occur for both MCEC-EMCV and MCEC-POL2.

Unlike in yeast, where HR is often the predominant mechanism for integration of DNA into the genome, vector integrations in mammalian cells mostly occur by nonhomologous end joining (NHEJ). As vector integration outside of NORs is not desired, we attempt to improve homologous recombination of ECs with nucleolar rDNA cistrons by combining several strategies. (a) We load the PCR-synthesized MCECs in vitro with NoLS-equipped PNAs that bind to the PNAts of cassettes, thereby facilitating their trafficking to the nucleolus of transfected cells, to bring them into proximity with their target rDNA sequences. (b) We improve the occurrence of HR-mediated cassette insertion by co-delivery of Cas9 RNPs that cleave the 28S rDNA at position 3805-3806, thereby inducing HDR using the MCEC as repair template. Cas9 is previously expressed in bacterial cells as a fusion with a NoLS to safeguard localization of Cas9 RNPs to the nucleolus. Purified NoLS-Cas9 is loaded with chemically synthesized sgRNA to form cleavage-competent RNP, before co-delivery of this RNP and PNA-loaded MCECs during transfection. Faithful cassette insertion by HDR results in destruction of the sgRNA target site, preventing additional cleavages of rDNA loci upon correct EC insertion. (c) We improve the HR efficiency by chemically inducing cell cycle transitions of mammalian cells to the S/G2/M phase by application of Nocodazole (Zhang et al., 2017, Genome Biology 18 (1), 1-18). Combinations of strategies (a, a+b, a+c, a+b+c) are tested to improve the chance of obtaining transformants with faithful EC insertions.

After in vitro assembly of NoLS-Cas9 RNP with synthetic sgRNA, and in vitro loading of MCECs with NoLS-PNAs, we co-transfect mammalian cells with PNA-MCEC and with or without the preassembled RNP, using lipid nanoparticle delivery of DNA and RNP (FIG. 31). After a 48 hour recovery period, Zeocin selection agent is added to the transfected cell cultures to eliminate non-transfected cells. Polyclonal cultures are passaged twice over eight additional days, and then analysed for GFP fluorescence using flow cytometry and fluorescence microscopy. We find that both MCEC variants are able to generate viable transformants and cause GFP fluorescence in polyclonal cultures. Transfection controls without DNA addition are unable to generate viable cells. Fractions of GFP-positive (GFP+) transformants in MCEC-POL2-transfected cells is generally higher, likely due to the ability of PUBC to drive gene expression also upon NHEJ-mediated EC integration at nucleoplasmic loci. Fraction of GFP+ cells in transfected cultures increases through co-delivery of NoLS-Cas9, and also through application of chemicals that stimulate HR. Average GFP fluorescence in GFP+ cell populations is generally comparable or higher for MCEC-EMCV-transfected cultures compared to the control construct. This is likely attributed to the high translational capacity of the EMCV IRES coupled to the high transcriptional activity of Pol I, and exemplifies the potential of the nucleolar gene expression system for high levels of protein production in mammalian cells.

TABLE 1
Fraction of GFP-positive transformants in viable cells (average cellular
GFP intensity of GFP-positive viable cells in parentheses) in polyclonal
mammalian cell cultures 48 hours post-transfection. + and −
indicate higher and lower fractions/average fluorescence, respectively.

| | | | | NoLS-PNA + |
|---|---|---|---|---|
| | | NoLS-PNA + | NoLS-PNA + | NoLS-Cas9 + |
| Construct | NoLS-PNA | NoLS-Cas9 | Nocodazole | Nocodazole |
| No DNA | −−− | −−− | −−− | −−− |
| | (−−−) | (−−−) | (−−−) | (−−−) |
| MCEC-EMCV | −− | −/+ | −/+ | + |
| | (+) | (++) | (+) | (++) |
| MCEC-POL2 | + | ++ | ++ | ++ |
| | (−+) | (+) | (+) | (+) |

Further improvements to the expression levels will be achievable by modifying the ECs to contain one or multiple TE elements downstream of the gene cassette in the 3′-UTR of the fuRNA, to improve fuRNA stability or translation initiation at the EMCV IRES. This can easily be achieved by replacing the SVLPA element of the MCEC-EMCV by different 3′-UTRs or polyadenylation signal sequences obtained from cellular or viral genes. Alternatively, improving the activity of the EMCV IRES or substitution with a stronger IRES could directly translate into better gene expression rates. Furthermore, controlling the EC copy number might help to boost gene expression levels. Selection for multi-copy clonal lines could be possible by increasing antibiotic concentrations after the recovery step, and/or by destabilizing the selection marker protein shble, to endow a selective advantage onto cells with higher transgene expression levels. Destabilization of shble could for instance be achieved by addition of a destabilization domain to the protein's C-terminus, such as the PEST region of mouse ornithine decarboxylase (Kong Ng et al., 2007, Metabolic Engineering 9 (3), 304-316).

Further optimizations of a mammalian cell nucleolar gene expression system include constructing a minimum-size MCEC-EMCV construct that should feature (i) a fully functional Pol I promoter (including the core promoter, upstream control element, upstream enhancer elements, and possibly the upstream spacer terminator) as described by Goodfellow and Zomerdijk (2013, Sub-Cellular Biochemistry 61, 211-236); (ii) a strong IRES such as the EMCV-IRES; (iii) a GOI coding region coupled to a selection marker either by a 2A skipping peptide or by addition of a second, weaker IRES between the GOI and the selection marker gene; (iv) TE elements downstream of the GOI (in case of an EC with a second IRES between the GOI and the selection marker) or downstream of the selection marker (in case of a 2A-translational fusion of GOI and selection marker); (v) homology flanks that allow integration of the EC into the intergenic spacer sequences (IGS) of active NORs, to prevent a loss of functional ribosome building blocks, which might be a consequence of multiple EC insertions into rRNA coding regions.

### Example 18: Materials and Methods

### Media and Strains

N. oceanica IMET1 was kindly provided by prof. Jian Xu (Qingdao Institute for Bioenergy and Bioprocess Technology, Chinese Academy of Sciences). The organism was cultivated using artificial sea water (ASW) containing 419.23 mM NaCl, 22.53 mM Na₂SO₄, 5.42 mM CaCl₂), 4.88 mM K₂SO₄, 48.21 mM MgCl₂ and 20 mM HEPES at pH 8, supplemented with 2 ml/l of commercial nutribloom plus (Necton, Portugal) growth media (ASW-NB) in a HT Multitron Pro (Infors Benelux, Netherlands) orbital shaker unit operated at 25° C., 90 rpm, 0.2% CO₂ enriched air and an illuminated with warm-white fluorescent light bulbs with an intensity of 150 with a 16:8 h diurnal cycle.

S. cerevisiae W303 was kindly provided by Alex Kruis and cultivated in YPD media (Dymond, 2013, Methods in Enzymology, 533, 191-204) or minimal SC media lacking uracil for transformant selection (Dymond, 2013, Methods in Enzymology, 533, 191-204).

P. pastoris (re-named Komagataella phaffii) X-33 was purchased from Thermo Fisher Scientific (Invitrogen #C18000) and cultivated in YPD medium (10 g/l yeast extract, 20 g/l peptone and 20 g/l glucose) at 30° C. and 250 rpm. When selecting and screening transformants, 100 μg/ml zeocin was added to the medium.

A mammalian cell line is purchased from Thermo Fisher Scientific, and cultivated according to the manufacturer's instructions.

### Plasmid Construction

Plasmids were constructed using either restriction cloning or Gibson assembly technique (Gibson et al., 2009, Nature Methods, 6 (5), 343-345). For restriction cloning, different DNA elements were designed with terminal recognition sites for type IIS restriction enzyme Eco311. Fragments were amplified via PCR (Q5 polymerase, NEB #M0492) according to manufacturer instructions, column or gel-purified (Thermo Fisher Scientific #K0831) and 65 ng of the backbone was mixed with inserts in molar ratios of 1:2, supplemented with T4 DNA ligase (Thermo Fisher Scientific #EL0011) and the corresponding buffer as well as with Eco311 (Thermo Fisher Scientific #FD0293). The mixture was incubated for 6 cycles of 5 min, 37° C. and 5 min, 16° C. and finally for an additional 10 min, 37° C. and 5 min, 65° C. and transformed into chemically competent Escherichia coli TOP10. Competent cells were created using Mix & Go E. coli transformation kit (Zymo Research #T3001). For Gibson assemblies we used NEBuilder HiFi DNA Assembly mix (New England Biolabs) according to manufacturer instructions with 25 nucleotide overlap. The cloning vector employed for all experiments was pUC19 (GenBank accession number M77789.2). N. oceanica genomic sequences including promoters, terminators, SA, rDNA sequences and homology flanks were amplified from genomic DNA of N. oceanica IMET1 via PCR. The bleomycin resistance gene of Streptoalloteichus hindustanus (zeo^{R}, GenBank accession number A31898.1) was amplified from pPtPuc3 (addgene #62863) which was a kind gift from Hamilton Smith. The EGFP sequence was codon harmonized (Claassens et al., 2017, PLOS ONE, 12 (9), e0184355) and synthesized by Integrated DNA Technologies, Inc. (Coralville, USA). The viral P2A linker sequence used in microalgae expression constructs as described by (Poliner et al., 2017, Plant Biotechnology Journal) (29 amino acid version) was codon optimized and synthesized together with EGFP. tdTomato was amplified from pCSCMV: tdTomato (addgene #30530). mVenus was codon harmonized and synthesized by Integrated DNA Technologies, Inc. mCherry was amplified from pEF-mCherry-LSD which was a kind gift from Mihris Naduthodi. For the assembly of P. pastoris constructs, EGFP and P2A linker were amplified from yEC1 (assembled in this study). The selection marker zeo^{R} was amplified from EC5 (assembled in this study). The TEV IRES (Huang et al., 2019, Biotechnology for Biofuels) was synthesized by Integrated DNA Technologies. P. pastoris sequences including GAP promoter, AOX1 terminator and 26S rDNA and AOX1 homology flanks were PCR-amplified from genomic DNA extracted from P. pastoris X-33.

Yeast expression constructs were assembled using an EGFP sequence codon optimized for S. cerevisiae which was amplified from pYET1-TEF1-yeGFP as a kind gift from Alex Kruis. The auxotrophic selection marker URA3 and the URA3 terminator were amplified from the same plasmid. The P2A linker sequence used for yeast constructs as described by (Souza-Moreira et al., 2018, FEMS Yeast Research, 18 (5)) was codon optimized and included as a spacer in the 5′-overhangs of PCR primers. S. cerevisiae rDNA sequences were PCR amplified from genomic DNA extracted from S. cerevisiae W303. The yeast control construct pCfB2791 (addgene #63654) was a kind gift from Belén Adiego Pérez.

Genomic DNA was extracted from exponentially growing cultures using Phire Plant Direct PCR (Thermo Scientific #F160) dilution buffer. For N. oceanica, ˜1E7 cells were pelleted (15 min) and resuspended in 100 μl of dilution buffer, frozen at −20° C. for 20 min, boiled (90° C. for 10 min and 95° C. for 5 min) and pelleted (10 min) again. The supernatant was used as template for PCR. Genomic DNA was extracted from S. cerevisiae cells using a similar approach. Briefly, ˜1E7 cells were pelleted (3 min) and resuspended in 200 μl of dilution buffer, heated at 70° C. for 15 min and pelleted (1 min) again. The supernatant was used as template for PCR. Genomic DNA was extracted from P. pastoris cells in a similar fashion. Briefly, cells from liquid culture were pelleted (5 min) and resuspended in 100 μl of dilution buffer, heated at 70° C. for 15 min and pelleted (5 min) again. The supernatant was used as template for PCR.

Genomic DNA is extracted from mammalian cells using the DNeasy Blood and Tissue Kit (Qiagen #69504) according to manufacturer's instructions. The EMCV IRES for construct MCEC-EMCV is synthesized as a gene fragment according to the native preferred IRES sequence, including the A6 bifurcation loop, as described by Bochkov & Palmenberg (2006, BioTechniques, 41 (3), 283-292). The SVLPA is PCR-amplified from pGem2-UPAS nucleotides 2531-2729 (Wu & Alwine, 2004, Molecular and Cellular Biology 24 (7), 2789-2796). Homology flanks for MCEC cassettes are amplified from mammalian cell genomic DNA using Q5 polymerase. Humanized (codon-optimized) versions of EGFP (missing a STOP codon), the porcine teschovirus-1 self-cleaving 2A sequence P2A and the Zeocin-resistance gene shble are synthesized together as a single gene fragment. The individual elements together with a PCR-amplified pUC19-based vector backbone are used to assemble pMCEC-EMCV (Seq X1) by Gibson assembly technique as described above. For pMCEC-POL2, a mammalian ubiquitin C promoter sequence is amplified from genomic DNA and used to replace the EMCV IRES sequence in pMCEC-EMCV by Gibson assembly technique.

Transformation of N. oceanica

Transformation of N. oceanica was carried out using the electroporation protocol described by (Vieler et al., 2012, PLOS Genetics, 8 (11), e1003064). Briefly, exponentially growing culture with a cell density of ˜4E7 cells/ml was harvested at 4° C., washed twice with ice-cold 375 mM sorbitol and resuspended to 2.5E9 cells/ml. 200 μl of cell suspension were mixed with 20 μg of denatured salmon sperm DNA (10 g/ml) and 1-2 μg of linear DNA template (purified PCR product) and electroporated at 12 kV/cm, 600 12 shunt resistance and 50 μF capacitance in pre-cooled electroporation cuvettes. Immediately after the pulse application the suspension was transferred to 5 ml of 20° C. ASW-NB and recovered at 30 illumination without agitation for 24 h. Cells were pelleted and plated on ASW-NB agar (1%) plates, supplemented with 5 μg zeocin/ml for selection of zeocin-resistant cells. Plates were incubated at 25° C. and 80 for 3-4 weeks before transformant colonies were transferred to liquid media containing 5 μg/ml of zeocin. Depending on the desired analytical method, transformants were either cultivated in microplates (48 wells or 96 wells) or shake flasks for several days.

Transformation of N. oceanica Using Cas12a Ribonucleoprotein

Targeted gene insertion in the NOR of chromosome 3 (EC7-CRISPR-NOR) was achieved using CRISPR-Cas technique with a ribonucleoprotein (RNP)-based approach, as described by (Naduthodi et al., 2019, Biotechnology for Biofuels, 12 (1), 66). Purified FnCas12a and guide RNAs (CRISPR RNAs) were assembled in vitro and co-transformed with editing template to facilitate homology directed repair (HDR)-based insertion of the ECs. CRISPR RNAs (oCS235 and oCS236) were designed using CHOPCHOPv2 (Labun et al., 2016, Nucleic Acids Research, 44 (W1), W272-W276).

Transformation of S. cerevisiae

Transformation of S. cerevisiae was carried out according to the protocol described by (Gietz & Woods, 2002, Methods in Enzymology, 350, 87-96).

Transformation of P. pastoris

Transformation of P. pastoris was carried out by electroporation, according to the protocol described by the pPICZA, B, and C Pichia vectors kit (Invitrogen #V19020). Briefly, exponentially growing culture with a cell density of ˜7.5E7 cells/ml was harvested at 4° C., washed twice with ice-cold water, washed once with ice-cold 1 M sorbitol and resuspended to ˜1.5E10 cells/ml with ice-cold 1 M sorbitol. 80 μl of cell suspension was mixed with 2-5 μg of digested plasmid or 3 μg linear DNA template (purified PCR product), incubated 5 min on ice and electroporated at 7.5 kV/cm, 200 12 shunt resistance and 25 μF capacitance in pre-cooled electroporation cuvettes. Immediately after the pulse application, the suspension was transferred to 1 ml of ice-cold 1 M sorbitol and recovered for 2 h at 30° C. Cells were plated on YPD agar (2%) plates supplemented with 100 μg zeocin/ml for selection of zeocin-resistant cells. Plates were incubated at 30° C. for 3-4 d before transformant colonies were transferred to liquid media containing 100 μg/ml of zeocin.

Transfection of Mammalian Cells with and without RNP

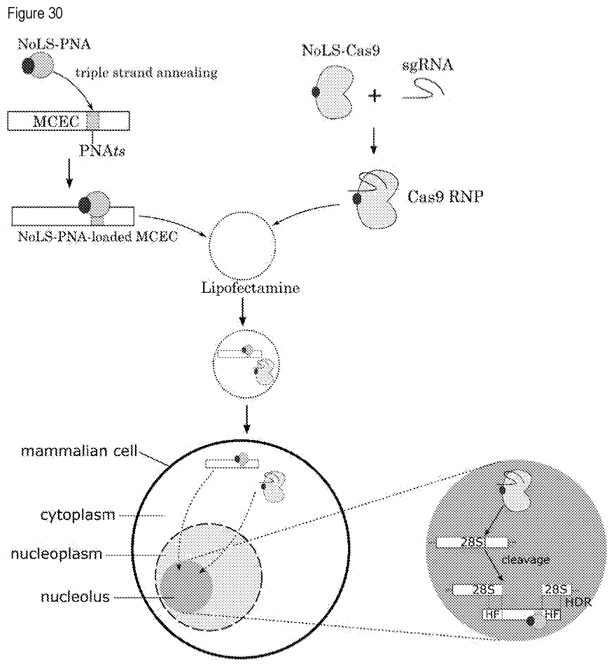

Transfection of mammalian cells is carried out by lipid nanoparticle delivery of linear NoLS-PNA-loaded MCECs with and without NoLS-Cas9 RNP (FIG. 30), on cells treated or not with chemical HR-inducers. NoLS-PNA with complementarity to the PNAts of MCECs and carrying the HIV-1 Rev protein nucleolar localization sequence (SEQ ID NO:264; Cochrane et al., 1990, Journal of Virology 64 (2), 881-885) is custom-made by PNA Bio Inc. The PNA and PNAts sequences of choice were previously described (Oprea et al., 2010, Molecular Biotechnology 45 (2), 171-179; Bigot et al., 2016, World patent WO2016016358A1), and the protocol for triple strand annealing of DNA template (PCR-amplified MCECs) and PNA was previously described by Oprea et al. (supra).

NoLS-Cas9 carrying the HIV-1 Rev protein NoLS is expressed in E. coli as described previously (Rajagopalan et al., 2018, Methods and Protocols 1 (2), 1-8), but using plasmid pET-NoLS-Cas9-6×His. This plasmid encodes the NoLS-Cas9 enzyme and is obtained via Gibson assembly technique by replacing the NLS encoded in the original plasmid pET-NLS-Cas9-6×His (addgene #62934) with the HIV-1 Rev NoLS coding sequence. Purified NoLS-Cas9 is assembled in vitro to form cleavage-competent RNP together with a synthetic sgRNA (SEQ ID NO:263) obtained from Integrated DNA Technologies, according to the sgRNA manufacturer's instructions.

NoLS-PNA-loaded MCECs and optionally NoLS-Cas9 RNP are packaged into lipid nanoparticles in vitro using CRISPRmax transfection reagent (Thermo Fisher Scientific #CMAX00008) according to manufacturer's instructions. Mammalian cells are plated in 48-well microplates and transfected using 0 or 5 pmol of RNP together with 15 pmol of DNA. Upon transfection, cells are grown for 48 hours before addition of Zeocin to the culture media for selection of transfected cells (350 μg/ml). Cells are grown for an additional 8 days in presence of Zeocin with sub-culturing after 2 and 6 days.

To improve HDR-mediated EC integration, a set of samples is treated with small molecules that control cell cycle progression (Zhang et al., 2017, Genome Biology 18 (1), 1-18). Such samples are treated with Nocodazole (100 ng/ml) 24 hours prior to transfection. Immediately before transfection, the small molecule is removed from the culture by washing with PBS and addition of the appropriate transfection media lacking Nocodazole.

### Analysis of Gene Expression

### Flow Cytometry Analysis

Expression of fluorescent reporter genes was quantified by measuring single cell fluorescence using flow cytometry analysis with different devices. Exponentially growing N. oceanica cultures were diluted to ˜4E6 cells/ml with ASW prior to analysis. Analyses for EC1-7 transformants shown in FIGS. 8-10 were carried out using an Attune NxT flow cytometer (Invitrogen, USA) equipped with lasers of 405 nm and 488 nm wavelengths. Singlet cells were selected by appropriate gating in the 488 nm forward and side scatter channels and only cells with a minimum chlorophyll a autofluorescence of 10,000 arbitrary fluorescence units (afu) in the red (detection at 710±25 nm, excitation at 405 nm) were considered for statistical analysis. EGFP signal was measured at 530±15 nm with blue excitation. Detector gains were set to 350 mV (forward and side scatter), 400 mV (710±25 nm) and 500 mV (530±15 nm). All other flow cytometry analyses were carried out using an SH800S (Sony Biotechnology, USA) instrument equipped with a 70 μm nozzle microfluidic chip and lasers of 488 nm and 561 nm wavelengths. Detector wavelengths for different channels were: 488 nm (forward scatter, gain 2); 488 nm (side scatter, gain 22%); 510±10 nm (EGFP, gain 45%); 525±25 nm (EGFP and mVenus, gain 45%), 585±15 nm (tdTomato, gain 45%), 617±15 nm (mCherry, gain 45%), 720±30 nm (chlorophyll a autofluorescence, gain 40%). A minimum of 50,000 events were screened per sample and only singlet events with a minimum chlorophyll a fluorescence of ˜7500 afu were considered for statistical analysis. Gating for singlet events was done using an automated gating pipeline that was written in R statistical computing software (R Core Team, 2018), using the flowcore (Ellis et al., 2009, Http://Bioconductor.org/), flowWorkspace (Finak, 2014, R Guide) and ggcyto (Phu et al., 2018, Bioinformatics) packages from the Bioconductor project (Gentleman et al., 2004, Genome Biology, 5 (10)).

Expression of EGFP in P. pastoris was quantified as follows. Liquid P. pastoris cultures were grown overnight in 12 or 48 wells plates, centrifuged and resuspended prior to analysis by vigorous pipetting to reduce aggregates. Flow cytometry analyses were carried out with the SH800S (Sony Biotechnology, USA) instrument using the same detector settings as for N. oceanica experiments. 30,000 events were screened per sample and only singlet events were considered for statistical analysis. The P. pastoris gating pipeline was similar to that developed for N. oceanica but did not contain a gate for chlorophyll a fluorescence.

Expression of EGFP in mammalian cells is quantified by harvesting of cultures 10 days after transfection through trypsin/EDTA treatment, washing once with PBS, and resuspending in PBS prior to single cell fluorescence analysis using the SH800S (Sony Biotechnology, USA) as described above for P. pastoris.

### RTq-PCR Analysis

Reporter gene transcript abundance was quantified using RTq-PCR. RNA was extracted from exponentially growing cultures using the E.Z.N.A. plant RNA kit (Omega Bio-tek #R6827) according to manufacturer instructions with following modifications. ˜3E8 cells were harvested by centrifugation (4000×g for 5 min at 4° C.), the pellet was snap-frozen in liquid N₂ and resuspended in 500 μl of RB buffer prepared with fresh 2-mercaptoethanol. The suspension was transferred to a bead beater tube (MP Biomedicals #116914500) and bead beat for 2×60 s at 4000 rpm with a 120 s pause between cycles with a Precellys 24 homogenizer (Bertin Technologies). After centrifugation (2500×g, 1 min) the supernatant was transferred to a Homogenizer Mini Column and processed according to the “Standard Protocol”. An on-column DNase I digestion step was conducted using RNase-free DNase Set I (Omega Bio-tek #E1091) for 25 min. After elution in nuclease-free (NF) H₂O, RNA concentration was measured with a NanoDrop device and integrity of RNA was monitored by separating 500 ng of RNA with agarose gel electrophoresis using a 1.25% agarose gel and an RNA denaturation step (10 min at 70° C. in a 66% (v/v) formamide solution) prior to gel loading. RNA was subjected to a second DNase I digestion using TURBO DNA-free Kit (Thermo Fisher Scientific #AM1907) with a 30 min digestion. Abscence of DNA was verified by using 50 ng of RNA as PCR template with Taq polymerase (Thermo Fisher Scientific #K1081).

cDNA was synthesized by subjecting 700 ng of total RNA to reverse transcription (New England Biolabs #M0253S) with random hexamer primers according to manufacturer instructions. cDNA libraries were diluted 1:48 with NF H₂O and subjected to qPCR on a CFX96 Real-Time PCR detection system (Bio-Rad laboratories) using SYBR Select Master Mix (Thermo Fisher Scientific #4472903) according to manufacturer instructions with 200 nM primer concentrations in technical triplicate. Primer efficiencies were calculated from a standard curve with purified PCR product at concentrations between 1E3-1E6 template copies/μl. GFP transcript abundance was quantified relative to Actin and VCP1 transcripts as reference genes.

### Western Blot Analysis

Protein presence and size was verified using western blot technique. Soluble protein was extracted from exponentially growing N. oceanica cultures. ˜1.5E9 cells were pelleted (2500× g, 5 min) and resuspended in 500 μl 0.075 mM Tris buffer (pH of 8). The suspension was bead beat 3× at 2500 rpm for 20 s with a 120 s pause between cycles, using Lysing Matrix E (#116914500, MP Biomedicals) with a Precellys 24 homogenizer (Bertin Technologies). Subsequently, the tubes were frozen at −20° C. for 90 min, thawed at 20° C. and pelleted (15000× g, 5 min). The protein-containing supernatant was transferred to fresh tubes and protein contents were quantified using a modified Lowry assay (DC Protein Assay, Biorad #5000116) with a BSA calibration standard (Lowry et al., 1951, The Journal of Biological Chemistry, 193 (1), 265-275). 45 μg of soluble protein was mixed with 5× Laemmli reagent (Laemmli, 1970, Nature, 227 (5259), 680-685), heated to 85° C. for 3 min and separated by SDS-PAGE on 4-15% TGX protein gels (Biorad #5678084) with TGS running buffer for 40 min at 200 V. Immediately after, proteins were transferred to PVDF membranes (Biorad #162-0177) using a Criterion Blotter (Biorad) at 50 V for 60 min with pre-cooled Towbin buffer (Towbin et al., 1979, Proceedings of the National Academy of Sciences of the United States of America, 76 (9), 4350-4354). Membranes were blocked with TBS-T+1% skim milk powder (Biorad #170-6404), incubated with a GFP antibody (500× diluted, Thermo Fisher Scientific #14-6674-82) on a rocking shaker for 1.5 h at RT and then overnight at 4° C., washed thrice with TBS-T, incubated for 2 h with an HRP-conjugated secondary antibody at RT (2000× diluted, Thermo Fisher Scientific #A10551) and washed thrice again. Chemiluminescence was detected using a ChemiDoc XRS+ system and enhanced chemiluminescence substrate (Thermo Fisher Scientific #34096). After detection, gels and membranes were Coomassie-stained (Meyer & Lamberts, 1965, BBA—General Subjects, 107 (1), 144-145) to confirm appropriate protein separation and equal blotting efficiencies across samples.

### Luminescence Assays

Nanoluc activity was determined using a modified version of the protocol reported by (Poliner et al., 2018, Plant Cell Reports). Briefly, NanoLuciferase substrate (Promega) was diluted 10,000× in ASW. Microalgal cultures were diluted in ASW to a concentration of 2E7 cells/ml and 100 μl of cell suspension was transferred to a 96 well microtiter plate. Only for bicistronic assays, tdTomato fluorescence was measured using a CLARIOstar Plus plate reader (BMG LABTECH GmbH), to verify that transgene transcription was similar in all transformant strains before addition of chemiluminescence substrate. Subsequently, 100 μl of substrate-containing ASW was added to each well, the plate was agitated for 20 s and luminescence was measured at 470±40 nm. In assays with ECVHH-NLuc transformant strains, the control strains carried either pNOC-superstacked-HygR-GFP_Nlux (P(Ribi)-NLuc) or pNOC-superstacked-dualux-NR (P(NR)-NLuc) (Poliner et al., 2020, Algal Research, 45, 101664).

### ELISA

Soluble protein was extracted from N. oceanica as follows. Approximately 6E8 exponentially growing cells were pelleted (3000× g, 10 min, 4° C.), resuspended in 500 μl TBS-T supplemented with 2 μl/ml of protease inhibitor (Merck #P9599, TBS-T-PI) and bead beat 3× at 2500 rpm for 20 s with a 120 s pause between cycles, using Lysing Matrix D (#116913500, MP Biomedicals). Debris was pelleted by centrifugation (15000×g, 10 min, 4° C.) and the supernatant was transferred to fresh tubes and kept on ice. Protein concentration was quantified using modified Lowry procedure as described above and soluble extract concentrations were adjusted to 1 mg/ml. Meanwhile, ELISA plates were prepared as follows. Recombinant purified GFP (Thermo Fisher Scientific #A42613) was resuspended in TBS (pH 8.0) at 2.5 μg/ml and 50 μl was transferred to each well of a MediSorp 96-well microtiter plate (Thermo Fisher Scientific #467320). Control wells were treated with TBS only. The plate was sealed and incubated at RT in the dark for 2 h. The solution was removed and the plate was blocked by addition of 170 μl of 1% skim milk powder (Biorad #170-6404) in TBS per well. After 1 h incubation at RT in the dark, blocking solution was removed and wells were washed several times with TBS-T. 50 μl of soluble microalgal extracts at 1 μg protein/ml were then added to each well. Control wells were supplemented with 50 μl of a commercial purified GFP antibody (Thermo Fisher Scientific #14-6674-82) that had been diluted to 1 μg/ml in TBS-T. The plate was sealed and incubated overnight at 4° C. Wells were then washed several times with TBS-T before 50 mul of secondary antibody conjugated to HRP was added. For algal extracts, a goat anti-llama antibody (Abcam #ab112786) was used (10,000× diluted in 0.5% skim milk powder in TBS-T). For control wells that had been treated with mouse IgG, a goat anti-mouse secondary antibody (Thermo Fisher Scientific #A10551) was used (500× diluted in 0.5% skim milk powder in TBS-T). The plate was sealed and incubated at RT in the dark for 1 h. Solutions were removed and wells were washed several times with TBS-T. Then, 90 μl of colorigenic TMB substrate (Thermo Fisher Scientific #10301494) was added to each well and incubated at RT in the dark for exactly 10 min. The reaction was stopped by addition of 90 μl of 0.16 M H₂SO₄ and optical density was measured at 450 nm.

For quantitative ELISA, V_{H}H Nanobody was purified from algal extracts as follows. 6E9 exponentially growing cells of an ECVHH-his transformant strain were harvested by centrifugation (3000×g, 10 min, 4° C.), resuspended in 500 μl of ice-cold TBS-T-PI and bead beated as described above. After pelleting of debris, the supernatant was transferred to a fresh vial and protein concentration was measured. His-tagged VAH was purified from the extract by Ni-NTA spin column purification (Thermo Fisher Scientific #88224) according to manufacturer instructions with following modifications. 5 wash steps were carried out using 700 μl of 25 mM imidazole in PBS and each time the flowthrough was collected in separate tubes. Then, 3 additional wash steps were conducted with 700 μl of 40, 50 and 70 mM imidazole in PBS and also collected separately. Lastly, the elution was done in 4 separate steps with 200, 200, 500 and 200 μl of 250 mM imidazole in PBS used in steps 1, 2, 3 and 4 respectively. The raw extract, and all flowthrough, wash and elution fractions were then analysed by SDS-PAGE. Briefly, samples were mixed with 5× Laemmli reagent, heated to 95° C. for 5 min and 30 μl were separated on a 4-15% TGX protein gel with TGS running buffer for 35 min at 200 V. The gel was stained with Coomassie Brilliant Blue R-250 (Biorad #1610436) overnight and destained in dH₂O with multiple changes over 1 d. Purification and SDS-PAGE analysis were done for 3 biological triplicates. Elution fractions that showed no visible bands other than the V_{H}H band after destaining and the elution buffer was replaced with TBS-T-PI using Amicon Ultra Centrifugal Filter Units (Merck #UFC500308) with 3 washing steps of 15 min centrifugation at 14000×g. All centrifugations were carried out at 4° C. Then, V_{H}H solutions of biological replicates were pooled, adjusted to 300 μl total volume and protein concentration was measured.

Purified VAH was diluted to 4.00, 3.00, 2.50, 2.00, 1.50, 1.00, 0.75, 0.50, 0.25, 0.10 and 0.05 g/ml in TBS-T-PI and used as a calibration standard in quantitative ELISA. For this, microtiter plates were covered with GFP and blocked with skim milk as described above. Soluble extracts from 3 ECVHH-his transformant strains were prepared as described above and diluted to 200, 100, 50, 20, 10 and 1 g protein/ml. 50 μl of all V_{H}H calibration standard dilutions and of all extract dilutions was transferred to separate wells in duplicates and incubated at RT for 1 h in the dark. Unbound molecules were washed off and secondary goat anti-llama antibody was applied at 10,000× dilution as described above. Unbound antibody molecules were removed by washing and then HRP activity was quantified by adding TMB substrate and measuring the OD₄₅₀ after 10 min. A dose-response curve was created for the calibration samples and fitted with a 3-parameter Michaelis-Menten regression model using the drc package (Ritz et al., 2015, PLOS ONE) of R statistical computing software (R Core Team, 2018). This model was used to calculate the V_{H}H concentration in algal extracts.

### Genome Walking Procedure

Genome walking was carried out for TC transformant strain #17 employing a modified version of procedures reported by (Goodman et al., 2009, Cell Host and Microbe, 6 (3), 279-289) and (Zhang et al., 2014, The Plant Cell, 26 (4), 1398-1409). The TC carries terminal recognition sites for type IIS restriction enzyme MmeI, which induces a 2 nucleotide staggered-end cut 20-21 nucleotides outside of its recognition site. The sequences were oriented in a way that the cut site was positioned in the flanking genomic DNA of transformant strains, in order to facilitate ‘excision’ of the ends of the TC together with 20-21 nucleotides of flanking sequences. For this, transformant genomic DNA was extracted from exponentially growing cultures. Briefly, ˜6E7 cells were pelleted (5000× g, 3 min) and resuspended in 150 μl of lysis buffer as described by (Daboussi et al., 2014, Nature Communications, 5, 98-106). The suspension was vortexed for 60 s at high speed, incubated overnight at RT, vortexed for 60 s again and centrifuged for 5 min. The supernatant was transferred to a fresh vial and purified using a FavorPrep column purification kit (Bio-Connect B.V. #FAGDC001) according to manufacturer instructions except that NF H₂O was used in the elution step. DNA concentration was determined using a NanoDrop device and 350 ng was digested with 0.16 μl MmeI (NEB #R0637) for 10 min. Integrity of genomic DNA and successful digestion were verified by agarose gel electrophoresis. Double stranded DNA adapters were assembled from oligos oCS091 and oCS092 by mixing 100 μM stock solutions, heating at 93° C. for 5 min and slowly (˜1 h) cooling the mixture until it reached RT. The bottom strand of the adapter was designed with a 5′-phosphate and 3′-NH₃ modification to increase ligation efficiency and to prevent unspecific amplification during the PCR steps. The top strand was designed to have a NN-3′ overhang after adapter formation to facilitate annealing to the NN-3′ overhang of MmeI-digested DNA fragments. 13 μg of digested DNA was mixed with 4.8 μl of 50 μM adapters and subjected to ligation using 2.5 U of T4 DNA ligase (Thermo Fisher Scientific #EL0014) in an 8 μl reaction for 60 min at 22° C. The reaction was stopped by heat inactivation of the enzyme and diluted 1:5 with NF H₂O. To amplify the target sequence over the genomic background, 1 μl of the solution was used as template for nested PCR (2 rounds total) with Taq polymerase using primers (concentration of 0.25 μM) specific for the adapter and for either the 5′-end or the 3′-end of the TC respectively (2 separate nested PCRs per transformant). After the first PCR iteration, PCR products were diluted 1:50 with NF H₂O and subjected to a second round of PCR with nested primers. For increased specificity, both PCR iterations were run with touchdown settings (first 7 cycles with an annealing temperature of 72° C. and then 32 or 28 cycles with the appropriate annealing temperature for the first and second iteration respectively). PCR products were purified and sequenced in order to reveal the nucleotide sequence surrounding the TC.

### Statistical Data Treatment

All data processing and statistical analysis was done using R statistical software (R Core Team, 2018). Significant main effects were evaluated by two-way ANOVA. Significant ANOVAs were followed by Tukey's HSD test to compare means of multiple groups. Differences were considered significant in case of p<0.05. For flow cytometry analysis, the median of a fluorescence distribution in a certain fluorescence channel was considered as average single cell fluorescence of a sample. Means of median single cell fluorescence were calculated and used to compare fluorescence intensity between different groups.

### Sequences

N. oceanica Polymerase I promoter, chromosome 3
(SEQ ID NO. 1)
GTTCGGAAACTATCGATAGGGTTTTGGCAGTTCAACTCCTCATAAGTTTTTCTTTCACCGTGGGCAAAATCGG
CTGTATTGCTGCCCATCACAAGACGCCCTGGATTCCTCCCTTGTCCTCACTTCATGAGAAGGAGATGAGTGT
GTGGGAGCACAGCATTTCTGCTTCGTCATGACCACTGAGAACGAATTGTTCTGCATGAATGTTTGCAATACAA
TGCGTTCTTGGAGATGAGAGCTTCTCGACATCTCCTGCCATAAACACATGCGCAGTTGACAAGAGAGCACCA
GGGGCTGAGGACGGAATGGTACGTGTGACCGAGAGATCGAATACGCATCCAGATCCCCACTCAGTTCTCCT
GTCGAAGACTTGCTTCGATCAGTGAAGATCACCTGGCCCTTTTTCTTTGTAAAAGGCAAGGCCCAGGGCGGT
CGGAAGGTGTTGTATTGGTACGCTTTGACGTAGCCTTCTCGTTGATACTTTGATTGGCATACGAAACTTGGTG
GGACGGCTTGATAACCGGCTGTTGCAACAAGGACTATGCATCGAAGGTGCACACCCTGGAAGCCCCGAGTC
CTCTGTTCGAGATACTGCTCCATCACACGAGCAGGACTCACAACGAGAGTTCAATCAATCGGGTCAGCTTCG
TATGAGTGATGCACCTGGTGAAGTTGCAGAGAGGCATCGGAAGAAGGTGCTTTTTGACACTGTCACGAGCG
TGTGTCCATGAGTCATGGCACATCTGATCTCGTGACCACCTTTTCATTCTTACGGAGAATGAGAAACGTCTCT
TGAAAAAATTTGCTGTCCAATCAGTGGCGATGCGGTGTTCTTGAACACGCTTGCCCTGACTGAACCATTGAA
GTTGTGTGATGTGTGAAGATGAATGTCTTCACCATAACTCAACAACAATAACGATAGCA
N. oceanica Polymerase I promoter, chromosome 9
(SEQ ID NO. 2)
CTTTTCTGTACCCATCGAGTCTGCATCAGCGACAGTTCAATTCCTCAAAAGGTTTTCATGGAACCCGATCAAG
TCGGCTGTATTGCTGTCCATCACAAGACGCCCTGGATTCCTCCATTGTTCTTTCTTTATGAGAAGCAGATAAG
TTTGTGTGAGCACAGCAGTTCTGCCTCGTCATGACCACTGAGAAGGACTTGTTTTGCATGCTTTTATGCGATA
CAATGCGTTCTTGGAGATGAGAGCTTCTCGACATCTCCTGCGATAAACACACGCGCAGTTGACAAGAGAGCA
CCAGGGGCTGAGGACGGAATGGTACGTGTGACCGAGAGATCGAATACGCATCCAGAACCCCACTCAGTTCT
CCTGTCGAAGACTTGCTTCGATCAGTGAAGATCACCTGGCCCTTTTTCTTTGTAAAAGGCAAGGCCCAGGGC
GGTCGGAAGGTGTTGTATTGGTACGCTTTGACGTAGCCTTCTCGTTGATACTTTGATTGGCATACGAAACTTG
GTGTGACGGCTGGATAACCGGCTGTTGCAACAAGGACTATGCATCGAAGGTGCACACCCTGGAAGCCCCGA
GTCCTCTGTTCGAGATACTGCTCCATCACACGAGCAGGACTCACAATGAGAGTTCATTCAATCGGGTCAGCT
TCGTATGAGTGATGCACCTGGTGAAGTTGCAGAGAGGCATCGGAAGAAGGTGCTTTTTGACACTGTCACGA
GCGTGTGTCCATGAGTCATGGCACATCTGATCACGTGACCACCTTTTCATTCTTACGGAGAATGAGAAACGT
CCCTTGAAAAAAATTTGCTGTCCAATCAGTGGCGATGCGGTGTTCCTGAACACGCTTGCCCTGACTGAACCA
TTGAAGTTGTGTGATGTGTGGAGATGCATGTCTTCACCGTAACTCATCTACAATAACGATAGCA
N. oceanica Polymerase I promoter, chromosome 15-1
(SEQ ID NO. 3)
GTTGTCTATTTAAATAGGAATTCTCACGGCAGTTCAACTCCCATTACGTTCTTTCTGCGATTGAGATGCTGGC
TGTATTGCTGCCCATCACAAGGCGCCCTGGATTCCTCCCTTGTCCTTTCTTCATGAGAAGCAGAAAACTGTGT
GTGAGCACAGCAGTTCTGTTTCGTCATGACATCTTAGAAGGAATTGTTTTGCATGTTTGTTTGCAATACAATG
CATTCTTGGAGATGAGAGCTTCTCGACATCTCCTGCCATAAACACATGCGCAGTTGACAAGAGAGCACCAGG
GGCTGAGGACGGAATGGTACGTGTGACCGAGAGATCGAATACGCATCCAGATCCCCACTCAGTTCTCCTGT
CGAAGACTTGCTTCGATCAGTGAAGATCACCTGGCCCTTTTTCTTTGTAAAAGGCAAGGCCCAGGGCGGTCG
GAAGGTGTTGTATTGGTACGCTTTGACGTAGCCTTCTCGTTGATACTTTGATTGGCATACGAAACTTGGTGTG
ACGGTTGGATAACCGGCTGTTGCAACAAGGACTATGCATCGAAGGTGTGCACCCTGGAAGCCCCGAGACCT
CTGTTCGAGTTACTGCTCCATCACACGAGCAGGACTCACAATGAGAGTTCAATCAATCGGGTCAGCTTCGTA
TGAGTGATGCACCTGGTGAAGTTGCAGAGAGGCATCGGAAGAAGGTGCTTTTTGACACTGTCACGAGCGTG
TGTCCATGAGTCATGGCACATCTGGTCTCGTGACCACCTTTTCATTCTTACGGAGAATGAGAAACGTCCCTTA
AAACATTTGCTGTCCAATCAGTGGCGATGCGGCGTTCCCGAACACGCTTGCCCTGACTGAACCATTGAAGTT
GTGTGATGTGTGGAGATGCATGTCTTCACCGTAACTCAACAACAATTACGATAGCA
N. oceanica Polymerase I promoter, chromosome 15-2
(SEQ ID NO. 4)
GTTGTCTATTTAAATAGGAATTCTCACGGCAGTTCAACTCCCATTACGTTCTTTCTGCCGTTGAGATGCTGGC
TGTATTGCTGCCCATCACAAGGCGCCCTGGATTCCTCCCTTGTCCTTTCTTCATGAGAAGCAGAAAACTGTGT
GTGAGCACAGCAGTTCTGTTTCGTCATGACATCTTAGAAGGAATTGTTTTGCATGTTTGTTTGCAATACAATG
CATTCTTGGAGATGAGAGCTTCTCGACATCTCCTGCCATAAACACATGCGCAGTTGACAAGAGAGCACCAGG
GGCTGAGGACGGAATGGTACGTGTGACCGAGAGATCGAATACGCATCCAGATCCCCACTCAGTTCTCCTGT
CGAAGACTTGCTTCGATCAGTGAAGATCACCTGGCCCTTTTTCTTTGTAAAAGGCAAGGCCCAGGGCGGTCG
GAAGGTGTTGTATTGGTACGCTTTGACGTAGCCTTCTCGTTGATACTTTGATTGGCATACGAAACTTGGTGTG
ACGGTTGGATAACCGGCTGTTGCAACAAGGACTATGCATCGAAGGTGCGCACCCTGGAAGCCCCGAGACCT
CTGTTCGAGATACTGCTCCATCACACGAGCAGGACTCACAATGAGAGTTCAATCAATCGGGTCAGCTTCGTG
TGAGTGATGCACCTGGTGAAGTTGCAGAGAGGCATCGGAAGAAGGTGCTTTTTGACACAGTCACGAGCGTG
TGTCCATGAGTCATGGCACATCTGATCTCGTGACCACCTTTTCATTCTTACGGAGAATGAGAAACGTCCCTTA
AAATATTTGCTGTCCAATCAGTGGCGATGCGGTGTTCCTGAACACGCTTGCCCTGACTGAACCATTGAAGTT
GTGTGATGTGTGGAGATACATGTCTTCACCGTAACTCAACAACAATTACGATAGCA
N. oceanica (NOC) IRES
1-203: 25S rDNA, partial (3′ 28S rDNA before cassette)
209-255: splice acceptor of NO25G00860 intron 1 (VCP intron)
(SEQ ID NO. 5)
ACAACCAGCTCAGAACTGGAGCGGACAAGGGGAATCCGACTGTTTAATTAAAACAAAGCATTGCGATGGTCG
GAGACGATGTTGACGCAATGTGATTTCTGCCCAGTGCTCTGAATGTCAAAGTGAAGGAATTCAACCAAGCGC
GGGTAAACGGCGGGAGTAACTATGACTCTCTTAAGGTAGCCAAATGCCTCGTCATCTAATTGGAATGCCCCT
CCCCCCTCCCTCCTTCCCTTCATCCTCCCCTCCGAGCAG
N. oceanica alpha tubulin terminator
(SEQ ID NO. 6)
TGGCCAGGGATCAGGAGGAGGGAGTGAAGAGGAGAAGGGATCTGGTTTCAGAGATCCCCACTTCTGCCGT
CGTCTTTCGGCCTTCCTTCCTTTTAGGTGTCATGCCTTAGGTCCTTCAAGTCCTCACCTGTCGTCGTCATGTG
TGTGTGTGCCCGTCATACAAGTCACTCGATCCAATTCACGCATCGGTTCAATCAAAATAAGACTAGACCCCG
AGGGAAGAAGGGCAGAAGGAAATCGAAGGGGTGGGATGTGTGTGAGAGAGGGAAGGAGAAATGAAAGAAG
TGAACAATGTCATGGTAGCCAGTAAGGAGAGAGTAGAAGCGAAGAAAGCAAAAGCACTGTTGTGAAGAAAC
GAAATGGAAGATGGTCATCGCTCCTGGCTCTACTTGTGGTTTTTCTATCTTTAATTTCAGGCGTCCTGGaCTC
GTTACATCAGCTCCCTTATCTCATTGGTTTATCCCCTACTCTACTGCTGCTTCTTCCTTCCATCCGTGACTGTA
TAACAACGAATTGTAGTACCGCAGATAGACAATAGAAAAATGCCAAAAAAGGCATCATTGATTTGCTCCTCCC
CATTAAGTCACTGTACGCCACCATCGCCACTACCCTCCCTT
N. oceanica Polymerase I terminator, proximal, chromosome 3
(SEQ ID NO. 7)
TGAAACCACTCCTAGTGAGATTGCGGAAAACCACACAACCGTGGACCTTCCTCGCAATTCACGCCCCCCCAT
CATACCTGACTCCCCAATCAACCTTTCTCCTCACAAAAGTGGGCTGAAGAGAGTCCAAGGGACGAAGGCATA
TGCTCATAACACTTCGTCACCGCCTATTAGAGGATCGGCGGTGAAGAAATGTCTCCCTCTTTGAAACTCCGG
GTAGCTTTTTGTTAATGTCAGATGTTTCCTGGACTTGACCTATTCCGAGGGCGCTTTCTTTCCCTCCCTACCC
CCGTAAATCCGGGTAGCTTTTTATTAAGGAGACGCAAGCGCTGTTTGCGCACATGCCGGTTACGAGACATCT
GCTTCCCTCGGGGCAATATCTAGGAGTCACATGTTTCTTTTTTGTTTCCTATGATGATACATGCTTTGGACGA
TTTTTCACAGGGACGAATGCCATACACACTTTTCATCTGTCCCTAAGAGGATGCACGGTAAAGAAAAAAAAAA
AAGTTCTCCGCCCTCCAATCTCCGGGTATCTTTTATGTTTGGCAGATGTTTCCTGGACTTGCCTGACGCCCCT
CAAACATACCTTCGAGTCTACGAACCATATCTTCACCAACCCCTTCTATTAAGAGGCTATTAAGAGGAAAGAC
GGGTTTGTTTAATATTTCGGGTGCCCTGGACTCATTCC
N. oceanica Polymerase I terminator, proximal, chromosome 9
(SEQ ID NO. 8)
TGAAACCACTCCTAGTGAGATTGCGGAAACCACACAACCGTGGACCTTCCTTGCAATTCACGCCCCCCATCA
TACCTGACTCCCCAAACAACCTTTCTCCTCACAAAAGTGGGCTGAAGAGAGAGTCAAAGGGACGAAAAGGCA
TATACTCATAACACTTCGTCACCGCCTCCTCTACTTCATTAGACTATCGGCGGTGAAGAAATGTCTCCCTCAG
GCCAAAATCCGGGTAGCGTTTTATTAATGTCAGATGTTTCCTGGACTTGGCCTATTTCGACTGCGCTTTCTTT
CTCTCCATCCTCCGTAATCCGGGTAGCTTTTTATTAATGTCAGATGTTTCCTGGACTTGGCCTATTTCGACTG
CGCTTTCTTTCTCTCCATCCTCCGTAATCCGGGTAGCTTTTTATTAATGTCATGTTTCCTGGACTTGGCCTATT
TCGACTGCGCTTTCTTTCTCTCCATCCTCCGTAATCCGGGTAGCTTTTTATTAATGTCATGTTTCCTGGACGG
CCTATTCAGAGGGCGCTTTCTTTCCCTCCCTTAACCCCCGTAAATCCGGGTAGCTTTTTATGAAGGAGACGC
AAGCGCTGTTTGCGCACATGCCGGTTACGAGACATCTGCTTCCCTCGGGGCAATATCTAGGAGTCACATGTT
TCTTTTTTGTTTCCTATGATGATACATGGTTTGGACGATTTTTCATAGGGACGAATGCCATACACACTTTTCAT
CTGTCCCTAAGAGGATGCACGGTAAAGAAAAAGTGCTGCGCCCTCCAATCTCCGGATATCTTTTATATTTGG
CAGATGTTTCCTGGACTTGCCTGACGCCCCCAAAACATACCTTCGAGTCTACGAACCATATCTTCACCAACC
CCTTCTATTAAGAGGCTATTAAGAGGAAAGACGGGTTTGTTTAATATTTCGGGTGCCCTGGACTCGCTCCCA
ACCGCCGCTCCCCAAGGGAAGAGATGTTACCTTGTTTTTT
N. oceanica Polymerase I terminator, proximal, chromosome 15-1
(SEQ ID NO. 9)
TGAAACCACTCCTAGTGAGATTGCGGAAACCACACAACCGTGGACCTTCCTCGCAATTCACGCCCCCCCATC
ATACCTGACTCCCCAAACAACCTTTCTCCTCACAAAAGTGGGCTGAAGAGAGAGTCAAAGGGACGAAAAGGC
ATATACTCATAACACTTCGTCACCGCCTCCTCTACTTCATTAGACTATCGGCGGTGAAGAAATGTCTCCCTCA
GGCCAAAATCCGGGTAGCTTTTTATTAATGTCAGATGTTTCCTGGACTTGGCCTATTTCGACTGCGCTTTCTT
TGTCTCCCTCTTCCAAACTCCGGGTAGCTTTTTATCAAATGTCAGATGTTTCCTGGACTTGGCCTATTTCGAC
TGCGCTTCCTTTCTCTCCATTCTCCGGAAATCCGGGTAGCTTTTTATTAATGTCAGATGTTTCCTTGACTTGAC
CTATTCCGAGTGCGCTCTCTTTCCCTTCCCTTAACTCCCGTAAATCCGGGTAGCTTTTTATGAAGGAGACGCA
AGCGCTGTTTGCGCACATGCCGGTCACGAGAGATCTGCTTCCCTCGGGGCAATATCTAGGAGTCACATGTTT
CTTTTTTGTTTCCTATGATGATACATGCTTTGGACGATTTTTCATAGGGACGAATGCCATACACACTTTTCATC
TGTCCCTAAGAGGATGCACGGTAAAGAAAAAGTGCTGCGCCCTCCAATCTCCGGGTATCTTTTATATTTGGC
AGATGTTTCCTGGACCTGACGCCCCCCAACATACCTTCGAGTCTACGAACCATATCTTCACCAACCCCTTCTA
TTAAGAGACTATTAAGAGGAAAGACGGGTTTGTCTAATATTTCGGGTGCCTTGGACTCGCCCCCAACCGCCG
CTCCCCCACAACCACACCTTCCTCTACCTATGG
N. oceanica Polymerase I terminator, proximal, chromosome 15-2
(SEQ ID NO. 10)
TGAAACCACTCCTAGTGAGATTGCGGAAACCACACAACCGTGGACCTTCCTCGCAATTCACGCCCCCCCATC
ATACCTGACTCCCCAAACAACCTTTCTCCTCACAAAAGTGGGCTGAAGAGAGAGTCAAAGGGACGAAAAGGC
ATATGCTCATAACACTTCGTCACCGCCTCCTCTACTTGATAGAGGATGGGCGGTGAAGAAATGTCTCCCTCTT
CCAAACTCCGGGTAGCTTTTTATCAAATGTCAGATGTTTCCTGGACTTGGCCTATTTCGACTGCGCTTCCTTT
CTCTCCATTCTCCGGAAATCCGGGTAGCTTTTTATAAAGGAGACGCAAGTGCTGTTTGCGCACATGCCGGTT
ACGAGACATCTGCTTCCCTCGGGGCAATATCTAGGAGTCACATGTTTCTTTTTTGTTTCCTATGATGATACAT
GCTTTGGACGATTTTTCATAGGGACGAATGCCATACACACTTTTCATCTGTCCCTAAGAGGATGCACGGTAAA
GAAAAAGTGCTGCGCCCTCCAATCTCCGGGTATCTTTTATATTTGGCAGATGTTTCCTGGACCTGACGCCCC
CCAACATACCTTCGAGTCTACGAACCATATCTTCACCAACCCCTTCTATTAAGAGACTATTAAGAGGAAAGAC
GGGTTTGTCTAATATTTCGGGTGCCTTGGACTCGCCCCCAACCGCCGCTCCCCCACAACCACACCTTCCTCT
ACCTATGG
EC7
1-924: Pol I promoter from Nannochloropsis oceanica, chromosome 3
925-1124: ITS2 + 25S rDNA, bases 1-91, Nannochloropsis oceanica
1125-1379: Noc-IRES
1125-1327: 25S rDNA, bases 2134-2336, Nannochloropsis oceanica
1328-1332: MmeI_recognition-site_truncated (complement)
1333-1379: VCP1_intron1_splice-acceptor
1380-2093: EGFP coding region (Codon harmonized for Nannochloropsis)
(encoding SEQ ID NO. 12)
2094-2180: 2A linker peptide sequence (P2A)
2181-2555: Bleomycin/Zeocin resistance gene from Steptoalloteichus
hindustanus
2556-3172: Alpha-tubulin_terminator from Nannochloropsis oceanica
3173-3178: MmeI_recognition-site
3179-3378: 25S rDNA, bases 2334-2533, Nannochloropsis oceanica
3379-3578: 25S rDNA, bases 3191-3390, Nannochloropsis oceanica
3579-4265: Pol I terminator from Nannochloropsis oceanica chromosome 3
(SEQ ID NO.11)
GTTCGGAAACTATCGATAGGGTTTTGGCAGTTCAACTCCTCATAAGTTTTTCTTTCACCGTGGGCAAAATCGG
CTGTATTGCTGCCCATCACAAGACGCCCTGGATTCCTCCCTTGTCCTCACTTCATGAGAAGGAGATGAGTGT
GTGGGAGCACAGCATTTCTGCTTCGTCATGACCACTGAGAACGAATTGTTCTGCATGAATGTTTGCAATACAA
TGCGTTCTTGGAGATGAGAGCTTCTCGACATCTCCTGCCATAAACACATGCGCAGTTGACAAGAGAGCACCA
GGGGCTGAGGACGGAATGGTACGTGTGACCGAGAGATCGAATACGCATCCAGATCCCCACTCAGTTCTCCT
GTCGAAGACTTGCTTCGATCAGTGAAGATCACCTGGCCCTTTTTCTTTGTAAAAGGCAAGGCCCAGGGCGGT
CGGAAGGTGTTGTATTGGTACGCTTTGACGTAGCCTTCTCGTTGATACTTTGATTGGCATACGAAACTTGGTG
GGACGGCTTGATAACCGGCTGTTGCAACAAGGACTATGCATCGAAGGTGCACACCCTGGAAGCCCCGAGTC
CTCTGTTCGAGATACTGCTCCATCACACGAGCAGGACTCACAACGAGAGTTCAATCAATCGGGTCAGCTTCG
TATGAGTGATGCACCTGGTGAAGTTGCAGAGAGGCATCGGAAGAAGGTGCTTTTTGACACTGTCACGAGCG
TGTGTCCATGAGTCATGGCACATCTGATCTCGTGACCACCTTTTCATTCTTACGGAGAATGAGAAACGTCTCT
TGAAAAAATTTGCTGTCCAATCAGTGGCGATGCGGTGTTCTTGAACACGCTTGCCCTGACTGAACCATTGAA
GTTGTGTGATGTGTGAAGATGAATGTCTTCACCATAACTCAACAACAATAACGATAGCAACACCAAACAGTTT
CGACTTGGCGGCATCTTCTCGGTGCCATAACAAACACTGAGAAAGCCTTTGGACTGATCCTGGCACTCGTTG
CCGTGTCATTCCATCTCCAATTCGGACCTCCAATCAAGCAAGGCTACCCGCTGAATTTAAGCATATAACTAAG
CGGAGGAAAAGAAACTAACCAGGATTCCCCTAGTAACGGCGACAACCAGCTCAGAACTGGAGCGGACAAGG
GGAATCCGACTGTTTAATTAAAACAAAGCATTGCGATGGTCGGAGACGATGTTGACGCAATGTGATTTCTGC
CCAGTGCTCTGAATGTCAAAGTGAAGGAATTCAACCAAGCGCGGGTAAACGGCGGGAGTAACTATGACTCT
CTTAAGGTAGCCAAATGCCTCGTCATCTAATTGGAATGCCCCTCCCCCCTCCCTCCTTCCCTTCATCCTCCCC
TCCGAGCAGATGAGCAAGGGGGAGGAGTTGTTCACGGGGGTGGTCCCCATCTTGGTGGAGTTGGACGGGG
ACGTGAACGGGCATAAGTTTAGCGTCAGTGGGGAGGGGGAGGGGGACGCCACGTATGGGAAGTTGACATT
GAAGTTTATCTGCACGACGGGGAAGTTGCCCGTGCCCTGGCCCACATTGGTCACGACGTTGACGTACGGGG
TGCAGTGCTTTAGCCGGTATCCCGACCACATGAAGCAGCACGATTTTTTCAAGAGCGCAATGCCCGAGGGG
TACGTGCAGGAGCGGACGATCTTTTTCAAGGACGATGGGAATTATAAGACACGGGCCGAGGTCAAGTTTGA
GGGGGACACATTGGTGAACCGGATTGAGTTGAAGGGGATCGACTTTAAGGAGGACGGGAATATCTTGGGGC
ATAAGTTGGAGTATAATTACAATAGCCATAACGTGTATATTATGGCCGATAAGCAGAAGAACGGGATTAAGGT
GAATTTCAAGATCCGGCATAATATCGAGGACGGGAGCGTGCAGTTGGCCGATCACTACCAGCAGAACACGC
CCATCGGGGACGGGCCCGTCTTGTTGCCCGATAATCACTATTTGAGTACGCAGAGCGCATTGAGTAAGGAC
CCCAATGAGAAGCGGGATCATATGGTCTTGTTGGAGTTTGTGACGGCCGCCGGGATCACACACGGGATCGA
CGAGTTGTATAAGATGACGACCCTGTCCTTCCAGGGGCCGGGGGCGACGAACTTCTCCTTGCTGAAGCAGG
CCGGGGACGTGGAGGAGAACCCGGGGCCCATGGCCAAGTTGACCAGTGCCGTTCCGGTGCTCACCGCGC
GCGACGTCGCCGGAGCGGTCGAGTTCTGGACCGACCGGCTCGGGTTCTCCCGGGACTTCGTGGAGGACG
ACTTCGCCGGTGTGGTCCGGGACGACGTGACCCTGTTCATCAGCGCGGTCCAGGACCAGGTGGTGCCGGA
CAACACCCTGGCCTGGGTGTGGGTGCGCGGCCTGGACGAGCTGTACGCCGAGTGGTCGGAGGTCGTGTC
CACGAACTTCCGGGACGCCTCCGGGCCGGCCATGACCGAGATCGGCGAGCAGCCGTGGGGGGGGGAGTT
CGCCCTGCGCGACCCGGCCGGCAACTGCGTGCACTTCGTGGCCGAGGAGCAGGACTGATGGCCAGGGAT
CAGGAGGAGGGAGTGAAGAGGAGAAGGGATCTGGTTTCAGAGATCCCCACTTCTGCCGTCGTCTTTCGGCC
TTCCTTCCTTTTAGGTGTCATGCCTTAGGTCCTTCAAGTCCTCACCTGTCGTCGTCATGTGTGTGTGTGCCCG
TCATACAAGTCACTCGATCCAATTCACGCATCGGTTCAATCAAAATAAGACTAGACCCCGAGGGAAGAAGGG
CAGAAGGAAATCGAAGGGGTGGGATGTGTGTGAGAGAGGGAAGGAGAAATGAAAGAAGTGAACAATGTCAT
GGTAGCCAGTAAGGAGAGAGTAGAAGCGAAGAAAGCAAAAGCACTGTTGTGAAGAAACGAAATGGAAGATG
GTCATCGCTCCTGGCTCTACTTGTGGTTTTTCTATCTTTAATTTCAGGCGTCCTGGACTCGTTACATCAGCTC
CCTTATCTCATTGGTTTATCCCCTACTCTACTGCTGCTTCTTCCTTCCATCCGTGACTGTATAACAACGAATTG
TAGTACCGCAGATAGACAATAGAAAAATGCCAAAAAAGGCATCATTGATTTGCTCCTCCCCATTAAGTCACTG
TACGCCACCATCGCCACTACCCTCCCTTTCCAACTAATTAGTGACGCGCATGAATGGATTAATGAGATTCCCA
CTGTCCCTATCTGCCATCTAGCGAACCCACAGCCAAGGGAACGGGCTTGGAATAATCAGCGGGGAAAGAAG
ACCCTGTTGAGCTTGACTCTAGTCCGACGTTGTGAAATGACTTATGAGGTGTAGCATAAGTGGGAGCTCCGG
CGACGATGAAATACCACTCGACGATATTCACCTCTTCTGATGCTACGCGTATCGCGATAGGCTTCGGCCCAG
CATCATACGTCATCATCATTTCAATCATTAGAAGAGATAAATCCTCTGTAGACGACTTTGATATGGAACGGGG
TATTGTAAGTGTGAGAGTAGGCTTTGTCCTACGATCCACTGAGATTCAGCCTCTTGTTCCGTCGATTTGTTGT
TGAAACCACTCCTAGTGAGATTGCGGAAAACCACACAACCGTGGACCTTCCTCGCAATTCACGCCCCCCCAT
CATACCTGACTCCCCAATCAACCTTTCTCCTCACAAAAGTGGGCTGAAGAGAGTCCAAGGGACGAAGGCATA
TGCTCATAACACTTCGTCACCGCCTATTAGAGGATCGGCGGTGAAGAAATGTCTCCCTCTTTGAAACTCCGG
GTAGCTTTTTGTTAATGTCAGATGTTTCCTGGACTTGACCTATTCCGAGGGCGCTTTCTTTCCCTCCCTACCC
CCGTAAATCCGGGTAGCTTTTTATTAAGGAGACGCAAGCGCTGTTTGCGCACATGCCGGTTACGAGACATCT
GCTTCCCTCGGGGCAATATCTAGGAGTCACATGTTTCTTTTTTGTTTCCTATGATGATACATGCTTTGGACGA
TTTTTCACAGGGACGAATGCCATACACACTTTTCATCTGTCCCTAAGAGGATGCACGGTAAAGAAAAAAAAAA
AAGTTCTCCGCCCTCCAATCTCCGGGTATCTTTTATGTTTGGCAGATGTTTCCTGGACTTGCCTGACGCCCCT
CAAACATACCTTCGAGTCTACGAACCATATCTTCACCAACCCCTTCTATTAAGAGGCTATTAAGAGGAAAGAC
GGGTTTGTTTAATATTTCGGGTGCCCTGGACTCA
EGFP coding region (Codon harmonized for Nannochloropsis)
(SEQ ID NO. 12)
MSKGEELFTGWPILVELDGDVNGHKFSVSGEGEGDATYGKLTLKFICTTGKLPVPWPTLVTTLTYGVQCFSRYP
DHMKQHDFFKSAMPEGYVQERTIFFKDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHYLSTQSALSKDPNEKRDHMVLLEFVT
AAGITHGIDELYK
EC7-tdTomato
1-924: Pol I promoter from Nannochloropsis oceanica, chromosome 3
925-1124: ITS2 + 25S rDNA, bases 1-91, Nannochloropsis oceanica
1125-1379: Noc-IRES
1125-1327: 25S rDNA, bases 2134-2336, Nannochloropsis oceanica
1328-1332: MmeI_recognition-site_truncated (complement)
1333-1379: VCP1_intron1_splice-acceptor
1380-2807: coding sequence for the fluorescent reporter tdTomato
(encoding SEQ ID NO. 14)
2808-2894: 2A linker peptide sequence (P2A)
2895-3269: Bleomycin/Zeocin resistance gene from Streptoalloteichus
hindustanus
3270-3886: Alpha-tubulin terminator from Nannochloropsis oceanica
3887-3892: MmeI_recognition-site
3893-4092: 25S rDNA, bases 2334-2533, Nannochloropsis oceanica
4093-4292: 25S rDNA, bases 3191-3390, Nannochloropsis oceanica
4293-4979: Pol I_terminator from Nannochloropsis oceanica chromosome 3
(SEQ ID. NO. 13)
GTTCGGAAACTATCGATAGGGTTTTGGCAGTTCAACTCCTCATAAGTTTTTCTTTCACCGTGGGCAAAATCGG
CTGTATTGCTGCCCATCACAAGACGCCCTGGATTCCTCCCTTGTCCTCACTTCATGAGAAGGAGATGAGTGT
GTGGGAGCACAGCATTTCTGCTTCGTCATGACCACTGAGAACGAATTGTTCTGCATGAATGTTTGCAATACAA
TGCGTTCTTGGAGATGAGAGCTTCTCGACATCTCCTGCCATAAACACATGCGCAGTTGACAAGAGAGCACCA
GGGGCTGAGGACGGAATGGTACGTGTGACCGAGAGATCGAATACGCATCCAGATCCCCACTCAGTTCTCCT
GTCGAAGACTTGCTTCGATCAGTGAAGATCACCTGGCCCTTTTTCTTTGTAAAAGGCAAGGCCCAGGGCGGT
CGGAAGGTGTTGTATTGGTACGCTTTGACGTAGCCTTCTCGTTGATACTTTGATTGGCATACGAAACTTGGTG
GGACGGCTTGATAACCGGCTGTTGCAACAAGGACTATGCATCGAAGGTGCACACCCTGGAAGCCCCGAGTC
CTCTGTTCGAGATACTGCTCCATCACACGAGCAGGACTCACAACGAGAGTTCAATCAATCGGGTCAGCTTCG
TATGAGTGATGCACCTGGTGAAGTTGCAGAGAGGCATCGGAAGAAGGTGCTTTTTGACACTGTCACGAGCG
TGTGTCCATGAGTCATGGCACATCTGATCTCGTGACCACCTTTTCATTCTTACGGAGAATGAGAAACGTCTCT
TGAAAAAATTTGCTGTCCAATCAGTGGCGATGCGGTGTTCTTGAACACGCTTGCCCTGACTGAACCATTGAA
GTTGTGTGATGTGTGAAGATGAATGTCTTCACCATAACTCAACAACAATAACGATAGCAACACCAAACAGTTT
CGACTTGGCGGCATCTTCTCGGTGCCATAACAAACACTGAGAAAGCCTTTGGACTGATCCTGGCACTCGTTG
CCGTGTCATTCCATCTCCAATTCGGACCTCCAATCAAGCAAGGCTACCCGCTGAATTTAAGCATATAACTAAG
CGGAGGAAAAGAAACTAACCAGGATTCCCCTAGTAACGGCGACAACCAGCTCAGAACTGGAGCGGACAAGG
GGAATCCGACTGTTTAATTAAAACAAAGCATTGCGATGGTCGGAGACGATGTTGACGCAATGTGATTTCTGC
CCAGTGCTCTGAATGTCAAAGTGAAGGAATTCAACCAAGCGCGGGTAAACGGCGGGAGTAACTATGACTCT
CTTAAGGTAGCCAAATGCCTCGTCATCTAATTGGAATGCCCCTCCCCCCTCCCTCCTTCCCTTCATCCTCCCC
TCCGAGCAGATGGTGAGCAAGGGCGAGGAGGTCATCAAAGAGTTCATGCGCTTCAAGGTGCGCATGGAGG
GCTCCATGAACGGCCACGAGTTCGAGATCGAGGGCGAGGGCGAGGGCCGCCCCTACGAGGGCACCCAGA
CCGCCAAGCTGAAGGTGACCAAGGGCGGCCCCCTGCCCTTCGCCTGGGACATCCTGTCCCCCCAGTTCAT
GTACGGCTCCAAGGCGTACGTGAAGCACCCCGCCGACATCCCCGATTACAAGAAGCTGTCCTTCCCCGAGG
GCTTCAAGTGGGAGCGCGTGATGAACTTCGAGGACGGCGGTCTGGTGACCGTGACCCAGGACTCCTCCCT
GCAGGACGGCACGCTGATCTACAAGGTGAAGATGCGCGGCACCAACTTCCCCCCCGACGGCCCCGTAATG
CAGAAGAAGACCATGGGCTGGGAGGCCTCCACCGAGCGCCTGTACCCCCGCGACGGCGTGCTGAAGGGC
GAGATCCACCAGGCCCTGAAGCTGAAGGACGGCGGCCACTACCTGGTGGAGTTCAAGACCATCTACATGGC
CAAGAAGCCCGTGCAACTGCCCGGCTACTACTACGTGGACACCAAGCTGGACATCACCTCCCACAACGAGG
ACTACACCATCGTGGAACAGTACGAGCGCTCCGAGGGCCGCCACCACCTGTTCCTGGGGCATGGCACCGG
CAGCACCGGCAGCGGCAGCTCCGGCACCGCCTCCTCCGAGGACAACAACATGGCCGTCATCAAAGAGTTC
ATGCGCTTCAAGGTGCGCATGGAGGGCTCCATGAACGGCCACGAGTTCGAGATCGAGGGCGAGGGCGAGG
GCCGCCCCTACGAGGGCACCCAGACCGCCAAGCTGAAGGTGACCAAGGGCGGCCCCCTGCCCTTCGCCTG
GGACATCCTGTCCCCCCAGTTCATGTACGGCTCCAAGGCGTACGTGAAGCACCCCGCCGACATCCCCGATT
ACAAGAAGCTGTCCTTCCCCGAGGGCTTCAAGTGGGAGCGCGTGATGAACTTCGAGGACGGCGGTCTGGT
GACCGTGACCCAGGACTCCTCCCTGCAGGACGGCACGCTGATCTACAAGGTGAAGATGCGCGGCACCAAC
TTCCCCCCCGACGGCCCCGTAATGCAGAAGAAGACCATGGGCTGGGAGGCCTCCACCGAGCGCCTGTACC
CCCGCGACGGCGTGCTGAAGGGCGAGATCCACCAGGCCCTGAAGCTGAAGGACGGCGGCCGCTACCTGG
TGGAGTTCAAGACCATCTACATGGCCAAGAAGCCCGTGCAACTGCCCGGCTACTACTACGTGGACACCAAG
CTGGACATCACCTCCCACAACGAGGACTACACCATCGTGGAACAGTACGAGCGCTCCGAGGGCCGCCACCA
CCTGTTCCTGTACGGCATGGACGAGCTGTACAAGATGACGACCCTGTCCTTCCAGGGGCCGGGGGCGACG
AACTTCTCCTTGCTGAAGCAGGCCGGGGACGTGGAGGAGAACCCGGGGCCCATGGCCAAGTTGACCAGTG
CCGTTCCGGTGCTCACCGCGCGCGACGTCGCCGGAGCGGTCGAGTTCTGGACCGACCGGCTCGGGTTCTC
CCGGGACTTCGTGGAGGACGACTTCGCCGGTGTGGTCCGGGACGACGTGACCCTGTTCATCAGCGCGGTC
CAGGACCAGGTGGTGCCGGACAACACCCTGGCCTGGGTGTGGGTGCGCGGCCTGGACGAGCTGTACGCC
GAGTGGTCGGAGGTCGTGTCCACGAACTTCCGGGACGCCTCCGGGCCGGCCATGACCGAGATCGGCGAG
CAGCCGTGGGGGGGGGAGTTCGCCCTGCGCGACCCGGCCGGCAACTGCGTGCACTTCGTGGCCGAGGAG
CAGGACTGATGGCCAGGGATCAGGAGGAGGGAGTGAAGAGGAGAAGGGATCTGGTTTCAGAGATCCCCAC
TTCTGCCGTCGTCTTTCGGCCTTCCTTCCTTTTAGGTGTCATGCCTTAGGTCCTTCAAGTCCTCACCTGTCGT
CGTCATGTGTGTGTGTGCCCGTCATACAAGTCACTCGATCCAATTCACGCATCGGTTCAATCAAAATAAGACT
AGACCCCGAGGGAAGAAGGGCAGAAGGAAATCGAAGGGGTGGGATGTGTGTGAGAGAGGGAAGGAGAAAT
GAAAGAAGTGAACAATGTCATGGTAGCCAGTAAGGAGAGAGTAGAAGCGAAGAAAGCAAAAGCACTGTTGT
GAAGAAACGAAATGGAAGATGGTCATCGCTCCTGGCTCTACTTGTGGTTTTTCTATCTTTAATTTCAGGCGTC
CTGGACTCGTTACATCAGCTCCCTTATCTCATTGGTTTATCCCCTACTCTACTGCTGCTTCTTCCTTCCATCCG
TGACTGTATAACAACGAATTGTAGTACCGCAGATAGACAATAGAAAAATGCCAAAAAAGGCATCATTGATTTG
CTCCTCCCCATTAAGTCACTGTACGCCACCATCGCCACTACCCTCCCTTTCCAACTAATTAGTGACGCGCAT
GAATGGATTAATGAGATTCCCACTGTCCCTATCTGCCATCTAGCGAACCCACAGCCAAGGGAACGGGCTTG
GAATAATCAGCGGGGAAAGAAGACCCTGTTGAGCTTGACTCTAGTCCGACGTTGTGAAATGACTTATGAGGT
GTAGCATAAGTGGGAGCTCCGGCGACGATGAAATACCACTCGACGATATTCACCTCTTCTGATGCTACGCGT
ATCGCGATAGGCTTCGGCCCAGCATCATACGTCATCATCATTTCAATCATTAGAAGAGATAAATCCTCTGTAG
ACGACTTTGATATGGAACGGGGTATTGTAAGTGTGAGAGTAGGCTTTGTCCTACGATCCACTGAGATTCAGC
CTCTTGTTCCGTCGATTTGTTGTTGAAACCACTCCTAGTGAGATTGCGGAAAACCACACAACCGTGGACCTTC
CTCGCAATTCACGCCCCCCCATCATACCTGACTCCCCAATCAACCTTTCTCCTCACAAAAGTGGGCTGAAGA
GAGTCCAAGGGACGAAGGCATATGCTCATAACACTTCGTCACCGCCTATTAGAGGATCGGCGGTGAAGAAA
TGTCTCCCTCTTTGAAACTCCGGGTAGCTTTTTGTTAATGTCAGATGTTTCCTGGACTTGACCTATTCCGAGG
GCGCTTTCTTTCCCTCCCTACCCCCGTAAATCCGGGTAGCTTTTTATTAAGGAGACGCAAGCGCTGTTTGCG
CACATGCCGGTTACGAGACATCTGCTTCCCTCGGGGCAATATCTAGGAGTCACATGTTTCTTTTTTGTTTCCT
ATGATGATACATGCTTTGGACGATTTTTCACAGGGACGAATGCCATACACACTTTTCATCTGTCCCTAAGAGG
ATGCACGGTAAAGAAAAAAAAAAAAGTTCTCCGCCCTCCAATCTCCGGGTATCTTTTATGTTTGGCAGATGTT
TCCTGGACTTGCCTGACGCCCCTCAAACATACCTTCGAGTCTACGAACCATATCTTCACCAACCCCTTCTATT
AAGAGGCTATTAAGAGGAAAGACGGGTTTGTTTAATATTTCGGGTGCCCTGGACTCA
tdTomato fluorescent reporter
(SEQ ID NO. 14)
MVSKGEEVIKEFMRFKVRMEGSMNGHEFEIEGEGEGRPYEGTQTAKLKVTKGGPLPFAWDILSPQFMYGSKAYV
KHPADIPDYKKLSFPEGFKWERVMNFEDGGLVTVTQDSSLQDGTLIYKVKMRGTNFPPDGPVMQKKTMGWEASTE
RLYPRDGVLKGEIHQALKLKDGGHYLVEFKTIYMAKKPVQLPGYYYVDTKLDITSHNEDYTIVEQYERSEGRHH
LFLGHGTGSTGSGSSGTASSEDNNMAVIKEFMRFKVRMEGSMNGHEFEIEGEGEGRPYEGTQTAKLKVTKGGP
LPFAWDILSPQFMYGSKAYVKHPADIPDYKKLSFPEGFKWERVMNFEDGGLVTVTQDSSLQDGTLIYKVKMRGT
NFPPDGPVMQKKTMGWEASTERLYPRDGVLKGEIHQALKLKDGGRYLVEFKTIYMAKKPVQLPGYYYVDTKLDIT
SHNEDYTIVEQYERSEGRHHLFLYGMDELYK
ECT2-tdTomato
1-1143: Left homology flank, targeting the EC to the NOR on chromosome 3
of Nannochloropsis oceanica, replacing the endogenous rDNA cistron
(when transforming wild type)
1144-2067: Pol I promoter from Nannochloropsis oceanica, chromosome 3
2068-2267: ITS2, partial + 25S rDNA, bases 1-91 from N. oceanica
2268-2522: Noc-IRES
2268-2470: 25S rDNA, bases 2134-2336 from Nannochloropsis oceanica
2471-2475: MmeI_recognition-site_truncated
2476-2522: VCP1_intron1_splice-acceptor
2523-3950: Encodes the fluorescent reporter tdTomato
(encoding SEQ ID NO. 14)
3951-4037: 2A linker peptide sequence
4038-4412: Bleomycin/Zeocin resistance gene from Streptoalloteichus
hindustanus
4413-5029: Alpha-tubulin_terminator from Nannochloropsis oceanica
5030-5035: MmeI_recognition-site
5036-6248: Right homology flank, targeting the EC to the NOR on
chromosome 3 of Nannochloropsis oceanica, replacing the endogenous rDNA
cistron (when transforming wild type)
(SEQ ID NO. 15)
AGGTAAGGAGTAGGGAGGGGGAGAGAGAAATAGGAGAGCGAGAATGAGCGGAAGGTGAGCAAGAGGAAG
TAGTTATACGACAGCATTAGAAGCAGCGAGAGGCAACCGAGAAATTGCTGGGGGGAGCGGCAAGGTTCTAA
GAAATTGGTAAGAGAACCAAAATCAATAGGGCAACGCATTGTCCTGAAGAAGAAATAAGAACCAAACCCTGC
GACAGCAATGCTTTGTTTTGAAAAACAATTGCTTGTACCTGCTGACACTCCACTTAAGCCTACCCATGCTGCC
ACTGCTATTGCTACCGCCACTGCAACCATCCCGACTACGGTTGCCCTTTTCTCCTCGGCAACGAGCCATAAG
AGGGCACCGCCAACTCAACAACTCAAAAACAGACCCCTTGACCCACCCTCCCAACCCAATCCCACTTGAGG
GCCAGGGCGGTGCGGAGCTTTTGACTTTTACCCTCCGAGTCCTCACCGTCTGCGTGGCCTTCCGGAGGATG
GATCCAACGGCACGCGCGATAAGTGCCCCCCTTCCACCAGCCCGCATGGCGGCGACGGCATGGCGGGCCT
TGACGAGGAATGAGGCCTGACTCGATGATAAGCAGAGGCAGGGCGAGTGCTGCGACTTACACCAGCTTGAC
TTGCCACGGCATCAGGAAGCACCGAAGTGCTAGGTGGAGACGCGATGGCCGTGGTCGTGAGGTTCATGCC
GCAGAAGGAGCGGAGGCGCTACTCCTGTTGCAGCGCTATCACTGTTGCAGCACTATATGTAACAACTTATTG
ATTCCATCGTTCTGTACAAAGTGCATCTTTGGTGTTCAATTGGGAAAGGAACGTGAGACAAGGCCACATCGT
TCTCACAGGCCTGCATGACGAAAACTATGCAGGCGGCGGAAAAAGAGCAAGGGAGGCTGCGTAACACAAC
GAGAACAAAACAAAAAGTGTGACTGGCATTTTTGTGTGTGTTAACAGCTGGTCGCAGTGATGGGAATCAAAA
TGCAAAAAGGAAAAAGGGAGAAGAACGAAAGTACGCAGGCCTACAACGTGTGAGCGTCTTCATTTCCACGA
CGTCTCATCACACGAATTGTCCATCATGTATTCTAGCGATACTCGAAAAGAATTCAAGAGAAGAAGAAAGAGC
TTAGTTCGGAAACTATCGATAGGGTTTTGGCAGTTCAACTCCTCATAAGTTTTTCTTTCACCGTGGGCAAAAT
CGGCTGTATTGCTGCCCATCACAAGACGCCCTGGATTCCTCCCTTGTCCTCACTTCATGAGAAGGAGATGAG
TGTGTGGGAGCACAGCATTTCTGCTTCGTCATGACCACTGAGAACGAATTGTTCTGCATGAATGTTTGCAATA
CAATGCGTTCTTGGAGATGAGAGCTTCTCGACATCTCCTGCCATAAACACATGCGCAGTTGACAAGAGAGCA
CCAGGGGCTGAGGACGGAATGGTACGTGTGACCGAGAGATCGAATACGCATCCAGATCCCCACTCAGTTCT
CCTGTCGAAGACTTGCTTCGATCAGTGAAGATCACCTGGCCCTTTTTCTTTGTAAAAGGCAAGGCCCAGGGC
GGTCGGAAGGTGTTGTATTGGTACGCTTTGACGTAGCCTTCTCGTTGATACTTTGATTGGCATACGAAACTTG
GTGGGACGGCTTGATAACCGGCTGTTGCAACAAGGACTATGCATCGAAGGTGCACACCCTGGAAGCCCCGA
GTCCTCTGTTCGAGATACTGCTCCATCACACGAGCAGGACTCACAACGAGAGTTCAATCAATCGGGTCAGCT
TCGTATGAGTGATGCACCTGGTGAAGTTGCAGAGAGGCATCGGAAGAAGGTGCTTTTTGACACTGTCACGA
GCGTGTGTCCATGAGTCATGGCACATCTGATCTCGTGACCACCTTTTCATTCTTACGGAGAATGAGAAACGT
CTCTTGAAAAAATTTGCTGTCCAATCAGTGGCGATGCGGTGTTCTTGAACACGCTTGCCCTGACTGAACCATT
GAAGTTGTGTGATGTGTGAAGATGAATGTCTTCACCATAACTCAACAACAATAACGATAGCAACACCAAACAG
TTTCGACTTGGCGGCATCTTCTCGGTGCCATAACAAACACTGAGAAAGCCTTTGGACTGATCCTGGCACTCG
TTGCCGTGTCATTCCATCTCCAATTCGGACCTCCAATCAAGCAAGGCTACCCGCTGAATTTAAGCATATAACT
AAGCGGAGGAAAAGAAACTAACCAGGATTCCCCTAGTAACGGCGACAACCAGCTCAGAACTGGAGCGGACA
AGGGGAATCCGACTGTTTAATTAAAACAAAGCATTGCGATGGTCGGAGACGATGTTGACGCAATGTGATTTC
TGCCCAGTGCTCTGAATGTCAAAGTGAAGGAATTCAACCAAGCGCGGGTAAACGGCGGGAGTAACTATGAC
TCTCTTAAGGTAGCCAAATGCCTCGTCATCTAATTGGAATGCCCCTCCCCCCTCCCTCCTTCCCTTCATCCTC
CCCTCCGAGCAGATGGTGAGCAAGGGCGAGGAGGTCATCAAAGAGTTCATGCGCTTCAAGGTGCGCATGG
AGGGCTCCATGAACGGCCACGAGTTCGAGATCGAGGGCGAGGGCGAGGGCCGCCCCTACGAGGGCACCC
AGACCGCCAAGCTGAAGGTGACCAAGGGCGGCCCCCTGCCCTTCGCCTGGGACATCCTGTCCCCCCAGTT
CATGTACGGCTCCAAGGCGTACGTGAAGCACCCCGCCGACATCCCCGATTACAAGAAGCTGTCCTTCCCCG
AGGGCTTCAAGTGGGAGCGCGTGATGAACTTCGAGGACGGCGGTCTGGTGACCGTGACCCAGGACTCCTC
CCTGCAGGACGGCACGCTGATCTACAAGGTGAAGATGCGCGGCACCAACTTCCCCCCCGACGGCCCCGTA
ATGCAGAAGAAGACCATGGGCTGGGAGGCCTCCACCGAGCGCCTGTACCCCCGCGACGGCGTGCTGAAGG
GCGAGATCCACCAGGCCCTGAAGCTGAAGGACGGCGGCCACTACCTGGTGGAGTTCAAGACCATCTACATG
GCCAAGAAGCCCGTGCAACTGCCCGGCTACTACTACGTGGACACCAAGCTGGACATCACCTCCCACAACGA
GGACTACACCATCGTGGAACAGTACGAGCGCTCCGAGGGCCGCCACCACCTGTTCCTGGGGCATGGCACC
GGCAGCACCGGCAGCGGCAGCTCCGGCACCGCCTCCTCCGAGGACAACAACATGGCCGTCATCAAAGAGT
TCATGCGCTTCAAGGTGCGCATGGAGGGCTCCATGAACGGCCACGAGTTCGAGATCGAGGGCGAGGGCGA
GGGCCGCCCCTACGAGGGCACCCAGACCGCCAAGCTGAAGGTGACCAAGGGCGGCCCCCTGCCCTTCGC
CTGGGACATCCTGTCCCCCCAGTTCATGTACGGCTCCAAGGCGTACGTGAAGCACCCCGCCGACATCCCCG
ATTACAAGAAGCTGTCCTTCCCCGAGGGCTTCAAGTGGGAGCGCGTGATGAACTTCGAGGACGGCGGTCTG
GTGACCGTGACCCAGGACTCCTCCCTGCAGGACGGCACGCTGATCTACAAGGTGAAGATGCGCGGCACCA
ACTTCCCCCCCGACGGCCCCGTAATGCAGAAGAAGACCATGGGCTGGGAGGCCTCCACCGAGCGCCTGTA
CCCCCGCGACGGCGTGCTGAAGGGCGAGATCCACCAGGCCCTGAAGCTGAAGGACGGCGGCCGCTACCT
GGTGGAGTTCAAGACCATCTACATGGCCAAGAAGCCCGTGCAACTGCCCGGCTACTACTACGTGGACACCA
AGCTGGACATCACCTCCCACAACGAGGACTACACCATCGTGGAACAGTACGAGCGCTCCGAGGGCCGCCA
CCACCTGTTCCTGTACGGCATGGACGAGCTGTACAAGATGACGACCCTGTCCTTCCAGGGGCCGGGGGCG
ACGAACTTCTCCTTGCTGAAGCAGGCCGGGGACGTGGAGGAGAACCCGGGGCCCATGGCCAAGTTGACCA
GTGCCGTTCCGGTGCTCACCGCGCGCGACGTCGCCGGAGCGGTCGAGTTCTGGACCGACCGGCTCGGGT
TCTCCCGGGACTTCGTGGAGGACGACTTCGCCGGTGTGGTCCGGGACGACGTGACCCTGTTCATCAGCGC
GGTCCAGGACCAGGTGGTGCCGGACAACACCCTGGCCTGGGTGTGGGTGCGCGGCCTGGACGAGCTGTA
CGCCGAGTGGTCGGAGGTCGTGTCCACGAACTTCCGGGACGCCTCCGGGCCGGCCATGACCGAGATCGG
CGAGCAGCCGTGGGGGGGGGAGTTCGCCCTGCGCGACCCGGCCGGCAACTGCGTGCACTTCGTGGCCGA
GGAGCAGGACTGATGGCCAGGGATCAGGAGGAGGGAGTGAAGAGGAGAAGGGATCTGGTTTCAGAGATCC
CCACTTCTGCCGTCGTCTTTCGGCCTTCCTTCCTTTTAGGTGTCATGCCTTAGGTCCTTCAAGTCCTCACCTG
TCGTCGTCATGTGTGTGTGTGCCCGTCATACAAGTCACTCGATCCAATTCACGCATCGGTTCAATCAAAATAA
GACTAGACCCCGAGGGAAGAAGGGCAGAAGGAAATCGAAGGGGTGGGATGTGTGTGAGAGAGGGAAGGA
GAAATGAAAGAAGTGAACAATGTCATGGTAGCCAGTAAGGAGAGAGTAGAAGCGAAGAAAGCAAAAGCACT
GTTGTGAAGAAACGAAATGGAAGATGGTCATCGCTCCTGGCTCTACTTGTGGTTTTTCTATCTTTAATTTCAG
GCGTCCTGGACTCGTTACATCAGCTCCCTTATCTCATTGGTTTATCCCCTACTCTACTGCTGCTTCTTCCTTC
CATCCGTGACTGTATAACAACGAATTGTAGTACCGCAGATAGACAATAGAAAAATGCCAAAAAAGGCATCATT
GATTTGCTCCTCCCCATTAAGTCACTGTACGCCACCATCGCCACTACCCTCCCTTTCCAACTCATCGTCATCG
TCCTTTCCCGTACGAGAAATTTAAAAGCTAGACCTCTACTTACCCCCTACTATTAACCCGCTCATCCTGACCT
CTCACTCCTCTTCCTCATCTTCCTCCACCACCGGCACACCCTCCACTTCTCCTTCGTGCCGTACAGGTAACAA
AGCCAACCTCTCGAGCACTTCTAACGCTGGGCCCTTGGGGTTGCCATGGACCAGGGCCTCTATCTGTCGAC
GTTGAGAGCGTGTCATGGATAGCTCAAAATCAATTATCACATCTACTACCGACAGGGCATTCTTCCAGCTTTC
ACTAATTAGCAAACTCTCTAGCAATGTCACGAGAATATCCGCCCCAGCGACATCATCTCGATCTCGCATCTCT
TGCAGCAAGCCCGCAACCCGATCCAGGCGACCTCGATCCACGTGACAGAGCGCTGCTACTGCTGCAGGGT
CCCCGCCCTGAAATGCTGGAGGCAAATTGTGATATACATCAAATGCCTTCTCAATCTGCTTTGGATTCGCCG
CCCAGCATCCACGGAGCAAAGACCCATAGAACCCTCGGTCCGCCTTCCACCCCTGTTCCTCCACCAGGCCG
AGGATGGTTTCAGCTGCGGCCAGGTCAGGCGGATCTAGCTTGGTGCAATAGACAAACGTGGCCCACCGGTA
AGTGTCATAACTTGTGTTTGGCTTCAAGACCAAGTCCCCCAGCAGATCTCTCGCCCGGTCCGACTGGCCAAC
GCGTAAGGAGGCGAGAATCGCAGGGACACCTAAGCGATCATGTTTTACCAGCATCTCCCGCGGTAAGGTAG
CAGCCACGTCCGCCAGCTCCTGGAAGCGACATGCTTGCTTCAGACACTTGAAAAGCCGGCGGTAGGTCAGT
GGGTTCGGCATCGAGGATGGCCCATGGCTGAGCATTTCGTGATATAGACGCAAAGCCCTCTCCGACATGCC
CGTCGGGTCATCACGCACCAAACGGTAGGTCACGTACATCGCCTCATTGTACATGATCGTGTTGGGGGTAC
GCCCACTCGCTTTGAATGTCTCAAGCAATTCTAAGGCCTCACGTCGGCTTTTCACATCTGCCTTGGCCAACG
CATGCATGAATGCACCGAACATGATATCATTCGAAGCCTCTTCCGGATCGCCCGCCAACGTTCTGAAGCGCG
TCAGAAGGCCATTCGCCTGCGTGCGATTCCTCAAATCCGCCGTCACACTAATCA
EC-VHH
1-1143: Left homology flank, targeting the EC to the NOR on chromosome
3 of Nannochloropsis oceanica, replacing the endogenous rDNA cistron
(when transforming wild type) or EC7-tdTomato (when transforming
EC7-tdTomato-S1) or ECT2-tdTomato (when transforming ECT2-tdTomato-S1)
1144-2067: Pol I promoter from Nannochloropsis oceanica, chromosome 3
2068-2267: ITS2 + 25S rDNA, bases 1-91, Nannochloropsis oceanica
2268-2522: Noc-IRES
2268-2470: 25S rDNA, bases 2134-2336, Nannochloropsis oceanica 2471-2475:
MmeI_recognition-site_truncated (complement)
2476-2522:VCP1_intron1_splice-acceptor
2523-2870: GFP-binding fragment of a single-chain camelid antibody
(Rothbauer et al., 2008) (encoding SEQ ID NO. 17)
2874-2900: Human influenca hemagglutinin-tag (HA-tag)
2904-2921: 6xHis affinity tag (6xHis tag)/
2925-3541: Alpha-tubulin_terminator from Nannochloropsis oceanica
3542-3547: MmeI recognition site
3548-4317: Transcriptional promoter (RNA Polymerase II) of the
Nannochloropsis oceanica LDSP gene (LDSP promoter)
4318-4713: Blasticidin resistance gene
4714-4950: Cauliflower mosaic virus transcriptional terminator
(RNA polymerase II) (CaMV 35S terminator)
4951-6163: Right homology flank, targeting the EC to the NOR on
chromosome 3 of Nannochloropsis oceanica, replacing the endogenous rDNA
cistron (when transforming wild type) or EC7-tdTomato (when transforming
EC7-tdTomato-S1) or ECT2-tdTomato (when transforming ECT2-tdTomato-S1)
(SEQ ID NO. 16)
AGGTAAGGAGTAGGGAGGGGGAGAGAGAAATAGGAGAGCGAGAATGAGCGGAAGGTGAGCAAGAGGAAG
TAGTTATACGACAGCATTAGAAGCAGCGAGAGGCAACCGAGAAATTGCTGGGGGGAGCGGCAAGGTTCTAA
GAAATTGGTAAGAGAACCAAAATCAATAGGGCAACGCATTGTCCTGAAGAAGAAATAAGAACCAAACCCTGC
GACAGCAATGCTTTGTTTTGAAAAACAATTGCTTGTACCTGCTGACACTCCACTTAAGCCTACCCATGCTGCC
ACTGCTATTGCTACCGCCACTGCAACCATCCCGACTACGGTTGCCCTTTTCTCCTCGGCAACGAGCCATAAG
AGGGCACCGCCAACTCAACAACTCAAAAACAGACCCCTTGACCCACCCTCCCAACCCAATCCCACTTGAGG
GCCAGGGCGGTGCGGAGCTTTTGACTTTTACCCTCCGAGTCCTCACCGTCTGCGTGGCCTTCCGGAGGATG
GATCCAACGGCACGCGCGATAAGTGCCCCCCTTCCACCAGCCCGCATGGCGGCGACGGCATGGGGGGCCT
TGACGAGGAATGAGGCCTGACTCGATGATAAGCAGAGGCAGGGCGAGTGCTGCGACTTACACCAGCTTGAC
TTGCCACGGCATCAGGAAGCACCGAAGTGCTAGGTGGAGACGCGATGGCCGTGGTCGTGAGGTTCATGCC
GCAGAAGGAGCGGAGGCGCTACTCCTGTTGCAGCGCTATCACTGTTGCAGCACTATATGTAACAACTTATTG
ATTCCATCGTTCTGTACAAAGTGCATCTTTGGTGTTCAATTGGGAAAGGAACGTGAGACAAGGCCACATCGT
TCTCACAGGCCTGCATGACGAAAACTATGCAGGCGGCGGAAAAAGAGCAAGGGAGGCTGCGTAACACAAC
GAGAACAAAACAAAAAGTGTGACTGGCATTTTTGTGTGTGTTAACAGCTGGTCGCAGTGATGGGAATCAAAA
TGCAAAAAGGAAAAAGGGAGAAGAACGAAAGTACGCAGGCCTACAACGTGTGAGCGTCTTCATTTCCACGA
CGTCTCATCACACGAATTGTCCATCATGTATTCTAGCGATACTCGAAAAGAATTCAAGAGAAGAAGAAAGAGC
TTAGTTCGGAAACTATCGATAGGGTTTTGGCAGTTCAACTCCTCATAAGTTTTTCTTTCACCGTGGGCAAAAT
CGGCTGTATTGCTGCCCATCACAAGACGCCCTGGATTCCTCCCTTGTCCTCACTTCATGAGAAGGAGATGAG
TGTGTGGGAGCACAGCATTTCTGCTTCGTCATGACCACTGAGAACGAATTGTTCTGCATGAATGTTTGCAATA
CAATGCGTTCTTGGAGATGAGAGCTTCTCGACATCTCCTGCCATAAACACATGCGCAGTTGACAAGAGAGCA
CCAGGGGCTGAGGACGGAATGGTACGTGTGACCGAGAGATCGAATACGCATCCAGATCCCCACTCAGTTCT
CCTGTCGAAGACTTGCTTCGATCAGTGAAGATCACCTGGCCCTTTTTCTTTGTAAAAGGCAAGGCCCAGGGC
GGTCGGAAGGTGTTGTATTGGTACGCTTTGACGTAGCCTTCTCGTTGATACTTTGATTGGCATACGAAACTTG
GTGGGACGGCTTGATAACCGGCTGTTGCAACAAGGACTATGCATCGAAGGTGCACACCCTGGAAGCCCCGA
GTCCTCTGTTCGAGATACTGCTCCATCACACGAGCAGGACTCACAACGAGAGTTCAATCAATCGGGTCAGCT
TCGTATGAGTGATGCACCTGGTGAAGTTGCAGAGAGGCATCGGAAGAAGGTGCTTTTTGACACTGTCACGA
GCGTGTGTCCATGAGTCATGGCACATCTGATCTCGTGACCACCTTTTCATTCTTACGGAGAATGAGAAACGT
CTCTTGAAAAAATTTGCTGTCCAATCAGTGGCGATGCGGTGTTCTTGAACACGCTTGCCCTGACTGAACCATT
GAAGTTGTGTGATGTGTGAAGATGAATGTCTTCACCATAACTCAACAACAATAACGATAGCAACACCAAACAG
TTTCGACTTGGCGGCATCTTCTCGGTGCCATAACAAACACTGAGAAAGCCTTTGGACTGATCCTGGCACTCG
TTGCCGTGTCATTCCATCTCCAATTCGGACCTCCAATCAAGCAAGGCTACCCGCTGAATTTAAGCATATAACT
AAGCGGAGGAAAAGAAACTAACCAGGATTCCCCTAGTAACGGCGACAACCAGCTCAGAACTGGAGCGGACA
AGGGGAATCCGACTGTTTAATTAAAACAAAGCATTGCGATGGTCGGAGACGATGTTGACGCAATGTGATTTC
TGCCCAGTGCTCTGAATGTCAAAGTGAAGGAATTCAACCAAGCGCGGGTAAACGGCGGGAGTAACTATGAC
TCTCTTAAGGTAGCCAAATGCCTCGTCATCTAATTGGAATGCCCCTCCCCCCTCCCTCCTTCCCTTCATCCTC
CCCTCCGAGCAGATGCAGGTCCAGTTGGTCGAGAGCGGCGGCGCATTGGTCCAGCCAGGCGGCAGCTTGC
GGTTGAGCTGTGCAGCAAGCGGCTTTCCCGTCAATCGGTACAGCATGCGGTGGTACCGGCAGGCACCCGG
CAAGGAGCGGGAGTGGGTCGCAGGCATGAGCTCTGCGGGCGACCGGAGCAGCTACGAGGACAGCGTCAA
GGGCCGGTTTACGATTAGCCGGGACGACGCACGGAATACGGTCTACTTGCAAATGAATAGCTTGAAGCCCG
AGGACACGGCAGTGTACTACTGCAATGTCAACGTGGGGTTTGAGTACTGGGGCCAGGGGACGCAGGTCAC
GGTCAGCAGCGGCTACCCCTATGACGTGCCCGACTACGCCGGCCATCACCATCACCATCACTGATGGCCAG
GGATCAGGAGGAGGGAGTGAAGAGGAGAAGGGATCTGGTTTCAGAGATCCCCACTTCTGCCGTCGTCTTTC
GGCCTTCCTTCCTTTTAGGTGTCATGCCTTAGGTCCTTCAAGTCCTCACCTGTCGTCGTCATGTGTGTGTGTG
CCCGTCATACAAGTCACTCGATCCAATTCACGCATCGGTTCAATCAAAATAAGACTAGACCCCGAGGGAAGA
AGGGCAGAAGGAAATCGAAGGGGTGGGATGTGTGTGAGAGAGGGAAGGAGAAATGAAAGAAGTGAACAAT
GTCATGGTAGCCAGTAAGGAGAGAGTAGAAGCGAAGAAAGCAAAAGCACTGTTGTGAAGAAACGAAATGGA
AGATGGTCATCGCTCCTGGCTCTACTTGTGGTTTTTCTATCTTTAATTTCAGGCGTCCTGGACTCGTTACATC
AGCTCCCTTATCTCATTGGTTTATCCCCTACTCTACTGCTGCTTCTTCCTTCCATCCGTGACTGTATAACAACG
AATTGTAGTACCGCAGATAGACAATAGAAAAATGCCAAAAAAGGCATCATTGATTTGCTCCTCCCCATTAAGT
CACTGTACGCCACCATCGCCACTACCCTCCCTTTCCAACGATGGAGTGGATGGAGGAGGAGGCGAGCGTAG
CAGCAAGCGTGAGTTATACAGCCAGGCACATGTCGCAATCCTTCGGTCTCGGGCTTAAAATCCACGCACTAA
TCACGCTGGGCCATGCAAAGAGCAATGCCGAGGCCCACCACACAAAACGCTGTGTCGCGCGTTGCGGCCT
GAAGCTTCATACTTCTTAGTCGCCGCCAAAAGGGCTCGAGAGACGAGACCCGTTGGCATGACCGATGTTGT
TCGACGCGGTTTGCTTCGTCACAGTCGACGTGATTCAGGAATCTGGAGCCTGCAGATCATTTTTTTCAGCCT
GATATCGTTCTTTTCCACTGAGAACCATCAGACCACCTTTTCTTCCATTGTGTGAAGGAGTAGGAGTTGCCGT
GCTGCTTTGTGGGAGACATCTGCGATGGTGACCAGCCTCCCGTCGTCTGGTCGACGTGACGAGCCTCTTCA
CTGTTCTTCGACGGAGAGACGCAAGCGAGACGGCTCTAGACCTTTTGGACACGCATTCTGTGTGTGAACTA
GTGGACAGTGATACCACGTCTGAAAGCTCACCACTGCCCATGGTGCAGCTACTTGTCACAAAGTTTTGACTC
CGTCGGTATCACCATTCGCGCTCGTGTGCCTGGTTGTTCCGCCACGCCGGCCTGCCCCGGGGCGGGGCAA
TATTCTAAAATCTCACGCAAAACACCGCACTTACCCCTCACACATATTCGTGATAGACCACCACCAATCTCAG
CCCGCATCAACACAGGAGGGCCCATGCCCCTCTCGCAGGAGGAGAGCACGCTGATCGAGAGGGCTACGGC
GACGATTAACTCGATTCCTATCTCCGAGGATTACTCGGTGGCTTCCGCTGCTCTGTCGTCCGACGGACGGAT
CTTCACGGGGGTCAACGTGTACCATTTTACGGGGGGGCCTTGCGCGGAGCTGGTGGTCCTGGGGACCGCC
GCTGCTGCCGCGGCTGGGAACCTGACGTGCATTGTCGCCATCGGGAACGAGAACCGCGGGATTCTGAGCC
CCTGCGGGCGGTGCCGGCAGGTCCTGCTCGACCTCCACCCTGGGATCAAGGCGATTGTGAAGGATTCCGA
CGGGCAGCCTACGGCGGTCGGTATCAGAGAACTTCTCCCTTCGGGCTACGTGTGGGAGAGCGCTTGAACTA
GTCAGGTCACTGGATTTTGGTTTTAGGAATTAGAAATTTTATTGATAGAAGTATTTTACAAATACAAATACATA
CTAAGGGTTTCTTATATGCTCAACACATGAGCGAAACCCTATAAGAACCCTAATTCCCTTATCTGGGAACTAC
TCACACATTATTCTGGAGAAAAATAGAGAGAGATAGATTTGTAGAGAGAGACTGGTGATTTTTGCGGACTCC
GGTCGGCATCTACTTCATCGTCATCGTCCTTTCCCGTACGAGAAATTTAAAAGCTAGACCTCTACTTACCCCC
TACTATTAACCCGCTCATCCTGACCTCTCACTCCTCTTCCTCATCTTCCTCCACCACCGGCACACCCTCCACT
TCTCCTTCGTGCCGTACAGGTAACAAAGCCAACCTCTCGAGCACTTCTAACGCTGGGCCCTTGGGGTTGCCA
TGGACCAGGGCCTCTATCTGTCGACGTTGAGAGCGTGTCATGGATAGCTCAAAATCAATTATCACATCTACT
ACCGACAGGGCATTCTTCCAGCTTTCACTAATTAGCAAACTCTCTAGCAATGTCACGAGAATATCCGCCCCA
GCGACATCATCTCGATCTCGCATCTCTTGCAGCAAGCCCGCAACCCGATCCAGGCGACCTCGATCCACGTG
ACAGAGCGCTGCTACTGCTGCAGGGTCCCCGCCCTGAAATGCTGGAGGCAAATTGTGATATACATCAAATG
CCTTCTCAATCTGCTTTGGATTCGCCGCCCAGCATCCACGGAGCAAAGACCCATAGAACCCTCGGTCCGCCT
TCCACCCCTGTTCCTCCACCAGGCCGAGGATGGTTTCAGCTGCGGCCAGGTCAGGCGGATCTAGCTTGGTG
CAATAGACAAACGTGGCCCACCGGTAAGTGTCATAACTTGTGTTTGGCTTCAAGACCAAGTCCCCCAGCAGA
TCTCTCGCCCGGTCCGACTGGCCAACGCGTAAGGAGGCGAGAATCGCAGGGACACCTAAGCGATCATGTTT
TACCAGCATCTCCCGCGGTAAGGTAGCAGCCACGTCCGCCAGCTCCTGGAAGCGACATGCTTGCTTCAGAC
ACTTGAAAAGCCGGCGGTAGGTCAGTGGGTTCGGCATCGAGGATGGCCCATGGCTGAGCATTTCGTGATAT
AGACGCAAAGCCCTCTCCGACATGCCCGTCGGGTCATCACGCACCAAACGGTAGGTCACGTACATCGCCTC
ATTGTACATGATCGTGTTGGGGGTACGCCCACTCGCTTTGAATGTCTCAAGCAATTCTAAGGCCTCACGTCG
GCTTTTCACATCTGCCTTGGCCAACGCATGCATGAATGCACCGAACATGATATCATTCGAAGCCTCTTCCGG
ATCGCCCGCCAACGTTCTGAAGCGCGTCAGAAGGCCATTCGCCTGCGTGCGATTCCTCAAATCCGCCGTCA
CACTAATCA
GFP-binding fragment of a single-chain camelid antibody
(SEQ ID NO. 17)
MQVQLVESGGALVQPGGSLRLSCAASGFPVNRYSMRWYRQAPGKEREWVAGMSSAGDRSSYEDSVKGRFTIS
RDDARNTVYLQMNSLKPEDTAVYYCNVNVGFEYWGQGTQVTVSS
PPEC-TEV-26S
1-1000: Left homology flank, targeting the EC to the 26S gene of
Pichia pastoris
1001-1146: Internal Ribosome Entry Site from Tobacco Etch Virus (TEV IRES)
1147-1860: yEGFP coding region (codons optimized for translation in
S. cerevisiae and C. albicans) (encoding SEQ ID NO. 12)
1861-1917: 2A peptide from porcine teschovirus-1 polyprotein (P2A)
1918-2292: Bleomycin/Zeocin resistance gene from Streptoalloteichus
hindustanus
2293-2539: Transcriptional terminator (RNA polymerase II) of the AOX1
gene of Pichia pastoris (AOX1 terminator)
2540-3539: Left homology flank, targeting the EC to the 26S gene of
Pichia pastoris
(SEQ ID NO 18)
GACCCCCTCAGTGGGCCATTTTTGGTAAGCAGAACTGGCGATGCGGGATGAACCGAACGCAGGGTTAAGGT
GCCGGAAGCACGCTCAAAGACACCACAAAAGGTGTTGGTTCATCTGGACAGCCGGACGGTGGCCATGGAA
GTCGGAATCCGCCAAGGAGTGTGTAACAACTCACCGGCCGAATGAACTAGCCCTGAAAATGGATGGCGCTA
AGCGTGCTACCTATACCCTGCCGGAGAACAGAAAGCTCTCCGAGTAGGCAGGCGTGGGGGTGGTGTGGAA
GGGTGCTTGTGAAGGTGCCTGGAACCGCCCCTAGTGCAGATCTTGGTGGCAGTAGCAATATTCAACGGAGC
CGTTGAAGACCGAAGTGGGGAAAGGTTCCACGGGAAGGGAGATCCTCCGTGGGTGAGACGGTCCTAAGGG
CGCGCGTACGGTAGCGTCCGAAAGGGAAGACAGTCAAGATTCTGTCTCCGGGGGAATGAGTGGTGACACG
AGACGGCCAAAGACGGTGGCGGAAGGCGTAGGAGGAGTTTTCTTTTCTTCTTAAGGGCGTTGTGCCTGGAT
GGTTTAGCCGGCGAAGGGCAGTAAAACCCGAAGAGCGTGCCCCTAGTTGGTGCGTCTACGCATCTCCGACA
TCCCGTGAAAATTTGGTTTATTTATGTTATTCCCCCGCCCGTACTGACAACCGCAGCAGGTCTCCAAGGTGAA
CAGCCTCTGGTGATAGAACAATGTAGATAAGGGAAGTCGGCAAATTGGATCCGTAACCTCGGGACAAGGATT
GGCTCTGGGGGTCGCTTTTATCGACCAACCCAGAACTGGTACGGACAAAGGGAATCTGACTGTCTAATTAAA
ACATAGGGTGGTGCGAGTCCCAACGGATGTGCACACCACCTGATTTCTGCCCAGTGCTCTGAATGTCAAAGT
GAAGAAATTCATCCAAGCGCGGGTAAACGGGGGGAGTAACTATGACTCTCTTAAGGTAGCCAAATGCCTCGT
CATCAAATAACAAATCTCAACACAACATATACAAAACAAACGAATCTCAAGCAATCAAGCATTCTACTTCTATT
GCAGCAATTTAAATCATTTCTTTTAAAGCAAAAGCAATTTTCTGAAAATTTTCACCATTTACGAACGATAGCAAT
GATGTCTAAAGGTGAAGAATTATTCACTGGTGTTGTCCCAATTTTGGTTGAATTAGATGGTGATGTTAATGGT
CACAAATTTTCTGTCTCCGGTGAAGGTGAAGGTGATGCTACTTACGGTAAATTGACCTTAAAATTTATTTGTAC
TACTGGTAAATTGCCAGTTCCATGGCCAACCTTAGTCACTACTTTAACTTATGGTGTTCAATGTTTTTCTAGAT
ACCCAGATCATATGAAACAACATGACTTTTTCAAGTCTGCCATGCCAGAAGGTTATGTTCAAGAAAGAACTAT
TTTTTTCAAAGATGACGGTAACTACAAGACCAGAGCTGAAGTCAAGTTTGAAGGTGATACCTTAGTTAATAGA
ATCGAATTAAAAGGTATTGATTTTAAAGAAGATGGTAACATTTTAGGTCACAAATTGGAATACAACTATAACTC
TCACAATGTTTACATCATGGCTGACAAACAAAAGAATGGTATCAAAGTTAACTTCAAAATTAGACACAACATTG
AAGATGGTTCTGTTCAATTAGCTGACCATTATCAACAAAATACTCCAATTGGTGATGGTCCAGTCTTGTTACC
AGACAACCATTACTTATCCACTCAATCTGCCTTATCCAAAGATCCAAACGAAAAGAGAGACCACATGGTCTTG
TTAGAATTTGTTACTGCTGCTGGTATTACCCATGGTATTGATGAATTGTACAAAGCTACTAATTTTTCTTTGTTA
AAACAAGCTGGTGATGTTGAAGAAAATCCTGGTCCAATGGCCAAGTTGACCAGTGCCGTTCCGGTGCTCACC
GCGCGCGACGTCGCCGGAGCGGTCGAGTTCTGGACCGACCGGCTCGGGTTCTCCCGGGACTTCGTGGAG
GACGACTTCGCCGGTGTGGTCCGGGACGACGTGACCCTGTTCATCAGCGCGGTCCAGGACCAGGTGGTGC
CGGACAACACCCTGGCCTGGGTGTGGGTGCGCGGCCTGGACGAGCTGTACGCCGAGTGGTCGGAGGTCG
TGTCCACGAACTTCCGGGACGCCTCCGGGCCGGCCATGACCGAGATCGGCGAGCAGCCGTGGGGGGGG
AGTTCGCCCTGCGCGACCCGGCCGGCAACTGCGTGCACTTCGTGGCCGAGGAGCAGGACTGATCAAGAGG
ATGTCAGAATGCCATTTGCCTGAGAGATGCAGGCTTCATTTTTGATACTTTTTTATTTGTAACCTATATAGTAT
AGGATTTTTTTTGTCATTTTGTTTCTTCTCGTACGAGCTTGCTCCTGATCAGCCTATCTCGCAGCTGATGAATA
TCTTGTGGTAGGGGTTTGGGAAAATCATTCGAGTTTGATGTTTTTCTTGGTATTTCCCACTCCTCTTCAGAGT
ACAGAAGATTAAGTGAGATAATTAGTGACGCGCATGAATGGATTAACGAGATTCCCACTGTCCCTATCTACTA
TCTAGCGAAACCACAGCCAAGGGAACGGGCTTGGCGCGTCAGCGGGGAAAGAAGACCCTGTTGAGCTTGA
CTCTAGTTTGGGCCGTGGGGGAACATGGGGGGTGTAGTATAGGGGGGAGCCGTTTCGGCGGCGCCCGTGA
AATACCCCCACCCCTATAGTTCCTCCACTAATCGCAGAGAAACCTCCAGGCGGGGAGTTTGGCTGGGGCGG
CACATCTGTTAAACCATAACGCAGATGTCCCAAGGGGGGCTCATGAAGAACAGAAATCTTCAGCCGAGCAAA
AGGGCAAATGCCCCCTTGATTTTGATTTTCAGTGTGAATACAAACCATGAAAGTGTGGCCTATCGATCCTTTA
CACCCTCAGACCCTGAGGCTAGAGGTGCCAGAAAAGTTACCACAGGGATAACTGGCTTGTGGCAGTCAAGC
GTTCATAGCGACATTGCTTTTTGATTCTTCGATGTCGGCTCTTCCTATCATACCGAAGCAGAATTCGGTAAGC
GTTGGATTGTTCACCCACTAATAGGGAACGTGAGCTGGGTTTAGACCGTCGTGAGACAGGTTAGTTTTACCC
TACTGATGAGCCCCACGGCAGTAATTGAACTTAGTACGAGAGGAACCGTTCATTCGGACATTTGGTTTTTGG
GGCTGCCTGACAAAGCAGCCCCGACGCTACGTCCGTCAGATCATGGCTGAACGCCTCTAAGTCAGAATCTG
TGCTGGGGTGGGGAGGAGTGTACAAGAAGGATATATTTCATAGCAGCATTCCACAATCTCCCGCCCACGAC
TTGACCCCGACAGGGTATTGTACGCAGAAGAGTGGCCTTGCCGTCACGATCTGCTGAGATTAAGCCCCGGT
CGAGCGATTGGAACTTCCATACCGAAGGAAACACACCGAGCGCAGCGAGGTTGTGTTTCCGAAGACGTAAT
CCCACCAAGCGGTACCA
Tobacco etch virus (TEV) IRES
(SEQ ID NO. 19)
GCCAAGCTTGCATGCGAGTCCCCGGAAATAACAAATCTCAACACAACATATACAAAACAAACGAATCTCAAG
CAATCAAGCATTCTACTTCTATTGCAGCAATTTAAATCATTTCTTTTAAAGCAAAAGCAATTTTCTGAAAATTTT
CACCATTTACGAACGATAGCAATGATGTCTAAAGGTGAAGAATTATTCA
yEC2
1-300: Homology flank, directing yEC2 to the 25S rDNA of Saccharomyces
cerevisiae
301-555: Putative IRES-encoding element analogous to Noc-IRES of
Nannochloropsis oceanica, but with Saccharomyces cerevisiae 25S rDNA
sequence
301-503: Part of IRES(Sc25), homologous to the 25S rDNA bases 2134-2336 of
Nannochloropsis oceanica.
504-508) MmeI_recognition-site_truncated (complement)
509-555: VCP1_intron1_splice-acceptor
556-1269: yEGFP (codons optimized for translation in S. cerevisiae and
C. albicans) (SEQ ID NO. 12)
1270-1326: 2A peptide from porcine teschovirus-1 polyprotein
1327-2130: S. cerevisiae orotidine-5′-phosphate decarboxylase, required
for uracil biosynthesis (URA3)
2131-2208: URA3 RNA polymerase Il transcriptional terminator
2209-2508: Homology flank, directing yEC2 to the 25S rDNA of
Saccharomyces cerevisiae
(SEQ ID NO. 20)
GTGAAAATCCACAGGAAGGAATAGTTTTCATGCCAGGTCGTACTGATAACCGCAGCAGGTCTCCAAGGTGAA
CAGCCTCTAGTTGATAGAATAATGTAGATAAGGGAAGTCGGCAAAATAGATCCGTAACTTCGGGATAAGGAT
TGGCTCTAAGGGTCGGGTAGTGAGGGCCTTGGTCAGACGCAGCGGGCGTGCTTGTGGACTGCTTGGTGGG
GCTTGCTCTGCTAGGCGGACTACTTGCGTGCCTTGTTGTAGACGGCCTTGGTAGGTCTCTTGTAGACCGTC
GCTTGCTACAATTAACGATCAACTTAGAACTGGTACGGACAAGGGGAATCTGACTGTCTAATTAAAACATAGC
ATTGCGATGGTCAGAAAGTGATGTTGACGCAATGTGATTTCTGCCCAGTGCTCTGAATGTCAAAGTGAAGAA
ATTCAACCAAGCGCGGGTAAACGGCGGGAGTAACTATGACTCTCTTAAGGTAGCCAAATGCCTCGTCATCTA
ATTGGAATGCCCCTCCCCCCTCCCTCCTTCCCTTCATCCTCCCCTCCGAGCAGATGTCTAAAGGTGAAGAAT
TATTCACTGGTGTTGTCCCAATTTTGGTTGAATTAGATGGTGATGTTAATGGTCACAAATTTTCTGTCTCCGGT
GAAGGTGAAGGTGATGCTACTTACGGTAAATTGACCTTAAAATTTATTTGTACTACTGGTAAATTGCCAGTTC
CATGGCCAACCTTAGTCACTACTTTAACTTATGGTGTTCAATGTTTTTCTAGATACCCAGATCATATGAAACAA
CATGACTTTTTCAAGTCTGCCATGCCAGAAGGTTATGTTCAAGAAAGAACTATTTTTTTCAAAGATGACGGTAA
CTACAAGACCAGAGCTGAAGTCAAGTTTGAAGGTGATACCTTAGTTAATAGAATCGAATTAAAAGGTATTGAT
TTTAAAGAAGATGGTAACATTTTAGGTCACAAATTGGAATACAACTATAACTCTCACAATGTTTACATCATGGC
TGACAAACAAAAGAATGGTATCAAAGTTAACTTCAAAATTAGACACAACATTGAAGATGGTTCTGTTCAATTAG
CTGACCATTATCAACAAAATACTCCAATTGGTGATGGTCCAGTCTTGTTACCAGACAACCATTACTTATCCACT
CAATCTGCCTTATCCAAAGATCCAAACGAAAAGAGAGACCACATGGTCTTGTTAGAATTTGTTACTGCTGCTG
GTATTACCCATGGTATTGATGAATTGTACAAAGCTACTAATTTTTCTTTGTTAAAACAAGCTGGTGATGTTGAA
GAAAATCCTGGTCCAATGTCGAAAGCTACATATAAGGAACGTGCTGCTACTCATCCTAGTCCTGTTGCTGCC
AAGCTATTTAATATCATGCACGAAAAGCAAACAAACTTGTGTGCTTCATTGGATGTTCGTACCACCAAGGAAT
TACTGGAGTTAGTTGAAGCATTAGGTCCCAAAATTTGTTTACTAAAAACACATGTGGATATCTTGACTGATTTT
TCCATGGAGGGCACAGTTAAGCCGCTAAAGGCATTATCCGCCAAGTACAATTTTTTACTCTTCGAAGACAGA
AAATTTGCTGACATTGGTAATACAGTCAAATTGCAGTACTCTGCGGGTGTATACAGAATAGCAGAATGGGCA
GACATTACGAATGCACACGGTGTGGTGGGCCCAGGTATTGTTAGCGGTTTGAAGCAGGCGGCAGAAGAAGT
AACAAAGGAACCTAGAGGCCTTTTGATGTTAGCAGAATTGTCATGCAAGGGCTCCCTATCTACTGGAGAATA
TACTAAGGGTACTGTTGACATTGCGAAGAGCGACAAAGATTTTGTTATCGGCTTTATTGCTCAAAGAGACATG
GGTGGAAGAGATGAAGGTTACGATTGGTTGATTATGACACCCGGTGTGGGTTTAGATGACAAGGGAGACGC
ATTGGGTCAACAGTATAGAACCGTGGATGATGTGGTCTCTACAGGATCTGACATTATTATTGTTGGAAGAGG
ACTATTTGCAAAGGGAAGGGATGCTAAGGTAGAGGGTGAACGTTACAGAAAAGCAGGCTGGGAAGCATATT
TGAGAAGATGCGGCCAGCAAAACTAAAAAACTGTATTATAAGTAAATGCATGTATACTAAACTCACAAATTAG
AGCTTCAATTTAATTATATCAGTTATTACCCTAATTAGTGACGCGCATGAATGGATTAACGAGATTCCCACTGT
CCCTATCTACTATCTAGCGAAACCACAGCCAAGGGAACGGGCTTGGCAGAATCAGCGGGGAAAGAAGACCC
TGTTGAGCTTGACTCTAGTTTGACATTGTGAAGAGACATAGAGGGTGTAGAATAAGTGGGAGCTTCGGCGCC
AGTGAAATACCACTACCTTTATAGTTTCTTTACTTATTCAATGAAGCGGAGCTGGAATTCATTTTCCACGTTCT
AGCATTCAAGGTCCCATTCGGGGCTGATCCGGGTTGAAGA
Sce-IRES
1-203: S. cerevisiae RDN25-1, partial
209-255: splice acceptor of N. oceanica NO25G00860 intron 1 (VCP intron)
(SEQ ID NO. 21)
CGATCAACTTAGAACTGGTACGGACAAGGGGAATCTGACTGTCTAATTAAAACATAGCATTGCGATGGTCAG
AAAGTGATGTTGACGCAATGTGATTTCTGCCCAGTGCTCTGAATGTCAAAGTGAAGAAATTCAACCAAGCGC
GGGTAAACGGCGGGAGTAACTATGACTCTCTTAAGGTAGCCAAATGCCTCGTCATCTAATTGGAATGCCCCT
CCCCCCTCCCTCCTTCCCTTCATCCTCCCCTCCGAGCAG
EGFP codon harmonized for N. oceanica used for construction of pCS-CC
(SEQ ID NO 22)
CTTCATCCTCCCCTCCGAGCAGATGAGCAAGGGGGAGGAGTTGTTCACGGGGGTGGTCCCCATCTTGGTG
GAGTTGGACGGGGACGTGAACGGGCATAAGTTTAGCGTCAGTGGGGAGGGGGAGGGGGACGCCACGTAT
GGGAAGTTGACATTGAAGTTTATCTGCACGACGGGGAAGTTGCCCGTGCCCTGGCCCACATTGGTCACGAC
GTTGACGTACGGGGTGCAGTGCTTTAGCCGGTATCCCGACCACATGAAGCAGCACGATTTTTTCAAGAGCG
CAATGCCCGAGGGGTACGTGCAGGAGCGGACGATCTTTTTCAAGGACGATGGGAATTATAAGACACGGGCC
GAGGTCAAGTTTGAGGGGGACACATTGGTGAACCGGATTGAGTTGAAGGGGATCGACTTTAAGGAGGACGG
GAATATCTTGGGGCATAAGTTGGAGTATAATTACAATAGCCATAACGTGTATATTATGGCCGATAAGCAGAAG
AACGGGATTAAGGTGAATTTCAAGATCCGGCATAATATCGAGGACGGGAGCGTGCAGTTGGCCGATCACTA
CCAGCAGAACACGCCCATCGGGGACGGGCCCGTCTTGTTGCCCGATAATCACTATTTGAGTACGCAGAGCG
CATTGAGTAAGGACCCCAATGAGAAGCGGGATCATATGGTCTTGTTGGAGTTTGTGACGGCCGCCGGGATC
ACACACGGGATCGACGAGTTGTATAAGATGACGACCCTGTCCTTCCAGGGGCCGGGGGCGACGAACTTCTC
CTTGCTGAAGC
mVenus codon harmonized for N. oceanica used for construction of
pCS-EC6-mVenus
(SEQ ID NO. 23)
TTCCCTTCATCCTCCCCTCCGAGCAGATGGTGAGCAAGGGGGAGGAGTTGTTTACGGGCGTGGTGCCCATC
TTGGTCGAGTTGGACGGGGACGTTAACGGGCACAAGTTTAGCGTGTCCGGGGAGGGGGAGGGGGATGCCA
CGTACGGGAAGTTGACGTTGAAGCTTATCTGCACGACGGGGAAGTTGCCCGTGCCCTGGCCCACGCTTGTG
ACGACGCTTGGGTACGGGTTGCAGTGCTTTGCCCGCTACCCCGACCACATGAAGCAGCACGACTTTTTTAA
GTCCGCCATGCCCGAGGGGTACGTCCAGGAGCGCACGATCTTTTTTAAGGACGACGGGAACTACAAGACGC
GCGCCGAGGTGAAGTTTGAGGGGGACACGTTGGTGAACCGCATCGAGTTGAAGGGGATCGACTTTAAGGA
GGACGGGAACATCTTGGGCCACAAGTTGGAGTACAACTACAACAGCCACAACGTCTATATCACGGCCGACA
AGCAGAAGAACGGGATCAAGGCCAACTTTAAGATCCGCCACAACATCGAGGACGGGGGGGTGCAGCTTGC
CGACCACTACCAGCAGAACACGCCCATCGGGGACGGGCCCGTGTTGTTGCCCGACAACCACTACTTGAGCT
ACCAGTCCAAGTTGAGCAAGGACCCCAACGAGAAGCGCGATCACATGGTCTTGTTGGAGTTTGTGACGGCC
GCCGGCATCACCCTTGGGATGGACGAGTTGTACAAG

TABLE 2
Oligonucleotides used in this study.

| ID | Sequence | Application |
|---|---|---|
| oCS001 | ATGATTACGCCAAGCTTGCATGCGA | Amplification of P_{VCP1} from No IMET1 gDNA for |
| | GTCCCCGGGTTGGAGGGGTCGTTT | construction of |
| | ATTTCTCTGCC | pCS-CC |
| oCS002 | CTGCTCGGAGGGGAGGATG | Amplification of P_{VCP1} from No IMET 1 gDNA for |
| | | construction of pCS-CC |
| oCS003 | ACGAACTTCTCCTTGCTGAAGCAGG | Amplification of zeo^{R} from pPtPuc3 for construction of |
| | CCGGGGACGTGGAGGAGAACCCG | pCS-CC |
| | GGGCCCATGGCCAAGTTGACCAGT | |
| | G | |
| oCS004 | CCTGATCCCTGGCCATCAGTCCTG | Amplification of zeo^{R} from pPtPuc3 for construction of |
| | CTCCTCGGC | pCS-CC |
| oCS005 | GGAGCAGGACTGATGGCCAGGGAT | Amplification of T_{αtub} from No IMET1 gDNA for |
| | CAGGAGGAGGGAG | construction of pCS-CC |
| oCS006 | AAGACTATTTGCTAAGGGAGGGTA | Amplification of T_{αtub} from No IMET1 gDNA for |
| | GTGGCGATG | construction of pCS-CC |
| oCS007 | ACTACCCTCCCTTAGCAAATAGTCT | Amplification of T_{CLPP} from No IMET 1 gDNA for |
| | TTATTTAGAACACAGAAGAAG | construction of pCS-CC |
| oCS008 | CTCGGTACCCGGGGATCCTCTAGA | Amplification of T_{CLPP} from No IMET 1 gDNA for |
| | GGAGTCTGGTTGTTGGAGAGTGAG | construction of pCS-CC |
| | GGGGAGGGGGAG | |
| oCS009 | TCCAACAACCAGACTCCTCTAGAGGA | Amplification of backbone from pUC19 for |
| | TCCCCGGG | construction of pCS-CC |
| oCS010 | TCCAACCCGGGGACTCGCATGCAAG | Amplification of backbone from pUC19 for |
| | CTTGGCGTAATC | construction of pCS-CC |
| oCS011 | TAGGTCTCAGTCACGCGGTATCATTG | Amplification of backbone from pCS-CC for |
| | CAGCA | construction of pCS-TC |
| oCS012 | TAGGTCTCTGCATTCCAACCCGGGG | Amplification of backbone from pCS-CC for |
| | ACTCGCAT | construction of pCS-TC |
| oCS013 | ATGGTCTCACCTTTCCAACAACCAGA | Amplification of backbone from pCS-CC for |
| | CTCCTCT | construction of pCS-TC |
| oCS014 | ATGGTCTCTTGACCCACGCTCACCG | Amplification of backbone from pCS-CC for |
| | GCTCCAG | construction of pCS-TC |
| oCS015 | TAGGTCTCTATGCCCCTCCCCCCTCC | Amplification of insert 1 from pCS-CC for construction |
| | CTCCTT | of pCS-TC |
| oCS016 | TAGGTCTCTTCCAGGACGCCTGAAAT | Amplification of insert 1 from pCS-CC for construction |
| | TAAAG | of pCS-TC |
| oCS017 | TAGGTCTCATGGACTCGTTACATCAG | Amplification of insert 2 from pCS-CC for construction |
| | CTCCCT | of pCS-TC |
| oCS018 | TAGGTCTCTAAGGGAGGGTAGTGGC | Amplification of insert 2 from pCS-CC for construction |
| | GATG | of pCS-TC |
| oCS019 | GTTGGAATGCCCCTCCCC | Amplification of TC from pCS-TC |
| oCS020 | GTTGGAAAGGGAGGGTAGTGG | Amplification of TC from pCS-TC |
| oCS021 | GTTGGAGGGGTCGTTTATTTC | Amplification of CC from pCS-CC |
| oCS022 | GTTGGAAAGGGAGGGTAGTGGCGAT | Amplification of CC from pCS-CC. Amplification of |
| | | insert 2 from pCS-ECT2 for construction of pCS- |
| | | ECVHH-his |
| oCS023 | GTTCGGAAACTATCGATAGGGTTTT | Amplification of rDNA cistron from chromosome 3 of |
| | | No IMET1 TC #17 amplification of EC1-7 and |
| | | derivatives |
| oCS024 | CTTTCAGTATATATTGGCAGCGCT | Amplification of rDNA cistron from chromosome 3 of |
| | | No IMET1 TC #17 |
| oCS025 | GAGTCCAGGGCACCCGAAAT | Amplification of EC1-7 and derivatives |
| oCS026 | AACCAGACTCCTCTAGAGGATCC | Amplification of backbone for construction of |
| | | intermediate cloning vector pCS-CVO from pCS-TC |
| oCS027 | CCGGGGACTCGCATGCAAGCTTG | Amplification of backbone for pCS-CVO from pCS-TC |
| oCS028 | ATGAGCAAGGGGGAGGAGTTGTTC | Amplification of insert 2 from pCS-TC for construction |
| | | of pCS-CV0 |
| oCS029 | AAGGGAGGGTAGTGGCGATGGTG | Amplification of insert 2 from pCS-TC for construction |
| | | of pCS-CV0 |
| oCS030 | GCCAAGCTTGCATGCGAGTCCCCGG | Amplification of insert 1 from No IMET 1 gDNA for |
| | GTTCGGAAACTATCGATAGGGTTTTG | construction of pCS-CV0 |
| oCS031 | TGAACAACTCCTCCCCCTTGCTCATT | Amplification of insert 1 from No IMET 1 gDNA for |
| | GCTATCGTTATTGTTGTTGAGTTATG | construction of pCS-CV0 |
| oCS032 | GCCACCATCGCCACTACCCTCCCTTT | Amplification of insert 3 from No IMET 1 gDNA for |
| | TGTCCTACGATCCACTGAGATTC | construction of pCS-CV0 |
| oCS033 | CCCGGGGATCCTCTAGAGGAGTCTG | Amplification of insert 3 from No IMET 1 gDNA for |
| | GTTGAGTCCAGGGCACCCGAAATATT | construction of pCS-CV0 |
| | AAAC | |
| oCS034 | TGAAACCACTCCTAGTGAGATTG | Amplification of backbone from pCS-CVO for |
| | | construction of pCS-EC1-7; Amplification of insert 1 |
| | | from No IMET1 gDNA for construction of pCS-ECT1 |
| oCS035 | TGCTATCGTTATTGTTGTTGAG | Amplification of backbone from pCS-CVO for |
| | | construction of pCS-EC1-7; Amplification of insert 1 |
| | | from No IMET1 gDNA for construction of pCS-CV1 |
| oCS036 | TAACTCAACAACAATAACGATAGCAA | Amplification of insert 1 for construction of pCS-EC5 |
| | CACCAAACAGTTTCGACTTGGC | pCS-EC6 & pCS-EC7 from No IMET1 TC #17 gDNA; |
| | | Amplification of insert 2 from pCS-EC7 for |
| | | construction of pCS-CV1 |
| oCS037 | CTGAGCTGGTTGTCGCCGTTACTAG | Amplification of insert 1 for construction of pCS-EC7 |
| | GGGAATCCTGG | from No IMET1 TC #17 gDNA |
| oCS038 | CCTAGTAACGGCGACAACCAGCTCA | Amplification of insert 2 for construction of pCS-EC7 |
| | GAACTGGAGCG | from No IMET1 TC #17 gDNA |
| oCS039 | GTGAATATCGTCGAGTGGTATTTCAT | Amplification of insert 2 for construction of pCS-EC7 |
| | CGTCGCCGGAG | from No IMET1 TC #17 gDNA |
| oCS040 | ATGAAATACCACTCGACGATATTCAC | Amplification of insert 3 for construction of pCS-EC7 |
| | CTCTTCTGATG | from No IMET1 TC #17 gDNA; Amplification of insert |
| | | 4 from No IMET1 gDNA for construction of pCS-CV1 |
| oCS041 | CGCAATCTCACTAGGAGTGGTTTCAA | Amplification of insert 1 for construction of pCS-EC3 |
| | CAACAAATCGACGGAACAAGAG | pCS-EC4 & pCS-EC5; Amplification of insert 3 for |
| | | construction of pCS-EC6 & pCS-EC7; all from No |
| | | IMET1 TC #17 gDNA |
| oCS042 | TAACTCAACAACAATAACGATAGCAG | Amplification of insert 1 for construction of pCS-EC4 |
| | CCAAGCAGTCGGTGGACGTTAC | from No IMET1 TC #17 gDNA |
| oCS043 | TAACTCAACAACAATAACGATAGCAA | Amplification of insert 1 for construction of pCS-EC3 |
| | GTCTTCGGACGGAAAAAGCCTG | from No IMET1 TC #17 gDNA |
| oCS044 | TAACTCAACAACAATAACGATAGCAA | Amplification of insert 1 for construction of pCS-EC2 |
| | TCTGGTTGATTCTGCCAGTAGTC | from No IMET1 TC #17 gDNA |
| oCS045 | TAAGTTTCCCCGTTTGAATGATTCGT | Amplification of insert 1 for construction of pCS-EC2 |
| | CGCTGGCAAAAG | from No IMET1 TC #17 gDNA |
| oCS046 | ACGAATCATTCAAACGGGGAAACTTA | Amplification of insert 2 for construction of pCS-EC2 |
| | CCAGGTCCAG | from No IMET1 TC #17 gDNA |
| oCS047 | CGCAATCTCACTAGGAGTGGTTTCAA | Amplification of insert 2 for construction of pCS-EC2 |
| | CAACAAATCGACGGAACAAGAGG | from No IMET1 TC #17 gDNA |
| oCS048 | GGGTTTTCAAGGGCCGACTCTCTTTT | Amplification of insert 1 for construction of pCS-EC6 |
| | CAAAGTTCTTTGCATC | from No IMET1 TC #17 gDNA |
| oCS049 | TTGAAAAGAGAGTCGGCCCTTGAAAA | Amplification of insert 2 for construction of pCS-EC6 |
| | CCCGGTGGCG | from No IMET1 TC #17 gDNA |
| oCS050 | TGTCGCGACAACAGTTTAACAGATGT | Amplification of insert 2 for construction of pCS-EC6 |
| | GCCGCCCCAGCC | from No IMET1 TC #17 gDNA |
| oCS051 | ACATCTGTTAAACTGTTGTCGCGACA | Amplification of insert 3 for construction of pCS-EC6 |
| | GTAATACAAC | from No IMET1 TC #17 gDNA |
| oCS052 | AAAACCCTGGCGTTACCCAAC | Amplification of backbone for construction of pCS- |
| | | EC7-CRISPR-NOR & intermediate cloning vectors |
| | | pCS-CV1 and pCS-CV3 from pCS-CV0 |
| oCS053 | AAACCGCTCACAATTCCACAC | Amplification of backbone for construction of pCS- |
| | | EC7-CRISPR-NOR & pCS-CV1 and pCS-CV3 from |
| | | pCS-CV0 |
| oCS054 | TTCCTTGCCACTACAATTGTATCTAA | Amplification of insert 2 for construction of pCS-EC7- |
| | GTTCGGAAACTATCGATAGGGTTTTG | CRISPR-NOR & pCS-CV3 from pCS-EC7 |
| oCS055 | AGAGGGCTGAAATTGACGGTCTACAA | Amplification of insert 2 for construction of pCS-EC7- |
| | GAGTCCAGGGCACCCGAAATATTAAA | CRISPR-NOR & pCS-CV3 from pCS-EC7 |
| | C | |
| oCS056 | GTATGTTGTGTGGAATTGTGAGCGGT | Amplification of insert 1 for construction of pCS-EC7- |
| | TTAAATCTCTCTCTCCCTCTCTC | CRISPR-NOR from No IMET1 gDNA |
| oCS057 | TTAGATACAATTGTAGTGGCAAGGAA | Amplification of insert 1 for construction of pCS-EC7- |
| | CACAAAATGCCAGTCACACTTTTTG | CRISPR-NOR from No IMET1 gDNA |
| oCS058 | TTGTAGACCGTCAATTTCAGCCCTCT | Amplification of insert 3 for construction of pCS-EC7- |
| | TATCTTGTGTGTGTTAACAGCTGGTC | CRISPR-NOR from No IMET1 gDNA |
| oCS059 | TTAAGTTGGGTAACGCCAGGGTTTTA | Amplification of insert 3 for construction of pCS-EC7- |
| | AAGTATCAACGAGAAGGCTAC | CRISPR-NOR from No IMET1 gDNA |
| oCS060 | GTTGTGTGGAATTGTGAGCGGTTTAA | Amplification of insert 1 for construction of pCS-CV3 |
| | ATGATCTCAGTCAGCCTCCG | from No IMET1 gDNA |
| oCS061 | TTAGATACAATTGTAGTGGCAAGGAA | Amplification of insert 1 for construction of pCS-CV3 |
| | CACAAAGTGGGAAGGAAAGGGAAGT | from No IMET1 gDNA |
| | G | |
| oCS062 | TTGTAGACCGTCAATTTCAGCCCTCT | Amplification of insert 3 for construction of pCS-CV3 |
| | TATCTCCGTCCACTCGTGATGCCTG | from No IMET1 gDNA |
| oCS063 | TTAAGTTGGGTAACGCCAGGGTTTTA | Amplification of insert 3 for construction of pCS-CV3 |
| | AATTTGATAGCAGGAATATGGGAACA | from No IMET1 gDNA |
| | GCAG | |
| oCS064 | TTGTGAAGTGAGCGAAGGT | Amplification of EC7-CRISPR-NOR from pCS-EC7- |
| | | CRISPR-NOR |
| oCS065 | AAAGTATCAACGAGAAGGCTACG | Amplification of EC7-CRISPR-NOR from pCS-EC7- |
| | | CRISPR-NOR |
| oCS066 | ATGACGACCCTGTCCTTCCAGG | Amplification of backbone from pCS-EC6 for |
| | | construction of pCS-EC6-mVenus/mCherry/tdTomato |
| oCS067 | CTGCTCGGAGGGGAGGATG | Amplification of backbone from pCS-EC6 for |
| | | construction of pCS-EC6-mVenus/mCherry/tdTomato; |
| | | Amplification of insert 2 from pCS-EV7 for |
| | | construction of pCS-CV1; Amplification of backbone |
| | | from pCS-CV2 for construction of pCS-ECT2; |
| | | Amplification of backbone from pCS-ECT2 for |
| | | construction of pCS-ECVHH-his |
| oCS068 | TCCCTTCATCCTCCCCTCCGAGCAGA | Amplification of insert from pCSCMV:tdTomato |
| | TGGTGAGCAAGGGCGAG | (addgene #30530) for construction of pCS-EC6- |
| | | tdTomato; Amplification of insert from |
| | | pEFmCherryLSD for construction of pCS-EC6- |
| | | mCherry |
| oCS069 | GCCCCTGGAAGGACAGGGTCGTCAT | Amplification of insert from pCSCMV:tdTomato |
| | CTTGTACAGCTCGTCCATGC | (addgene #30530) for construction of pCS-EC6- |
| | | tdTomato; Amplification of insert from |
| | | pEFmCherryLSD for construction of pCS-EC6- |
| | | mCherry |
| oCS070 | CCCTTGGGATGGACGAGTTGTACAA | Amplification of backbone from pCS-CC for |
| | GATGACGACCCTGTCCTTCCAG | construction of pCS-CC-mVenus |
| oCS071 | CTGCTCGGAGGGGAGGATG | Amplification of backbone from pCS-CC for |
| | | construction of pCS-CC-mVenus |
| oCS072 | CTCCTTCCCTTCATCCTCCC | Amplification of insert from pCS-CC-mVenus for |
| | | construction of pCS-EC6-mVenus |
| oCS073 | CTGGAAGGACAGGGTCGT | Amplification of insert from pCS-CC-mVenus for |
| | | construction of pCS-EC6-mVenus |
| oCS074 | AACCAGACTCCTCTAGAGGATCC | Amplification of backbone from pCS-EC6 for |
| | | construction of pCS-yEC1 & pCS-yEC2 |
| oCS075 | CCGGGGACTCGCATGCAAGCTTG | Amplification of backbone from pCS-EC6 for |
| | | construction of pCS-yEC1 & pCS-yEC2 |
| oCS076 | GCCAAGCTTGCATGCGAGTCCCCGG | Amplification of insert 1 from Sc W303 gDNA for |
| | GTGAAAATCCACAGGAAG | construction of pCS-yEC1 & pCS-yEC2 |
| oCS077 | CTGAGCTGGTTGTTTAATTGTAGCAA | Amplification of insert 1 from Sc W303 gDNA for |
| | GCGAC | construction of pCS-yEC1 |
| oCS078 | CACCTTTAGACATCTGCTCGGAGGG | Amplification of insert 1 from Sc W303 gDNA for |
| | GAGGATGAAGGGAAGGAGGGAGGG | construction of pCS-yEC2 |
| | GGGAGGGGCATTCCAATTAGATGAC | |
| | GAGGCATTTGGC | |
| oCS079 | TGCTACAATTAAACAACCAGCTCAGA | Amplification of insert 2 from Sc W303 gDNA for |
| | ACTGGAG | construction of pCS-yEC1 |
| oCS080 | CACCTTTAGACATCTGCTCGGAGGG | Amplification of insert 2 from Sc W303 gDNA for |
| | GAGGATG | construction of pCS-yEC1 |
| oCS081 | CCCTCCGAGCAGATGTCTAAAGGTG | Amplification of EGFP from pYET1-TEF1-yeGFP for |
| | AAGAATTATTC | construction of pCS-yEC1 & pCS-yEC2 |
| oCS082 | GTTTTAACAAAGAAAAATTAGTAGCT | Amplification of EGFP from pYET1-TEF1-yeGFP for |
| | TTGTACAATTCATCAATACCATG | construction of pCS-yEC1 & pCS-yEC2 |
| oCS083 | GCTACTAATTTTTCTTTGTTAAAACA | Amplification of URA3 and T_{URA3} from pYET1-TEF1- |
| | AGCTGGTGATGTTGAAGAAAATCCTG | yeGFP for construction of pCS-yEC1 & pCS-yEC2 |
| | GTCCAATGTCGAAAGCTACATATAAG | |
| oCS084 | CGTCACTAATTAGGGTAATAACTGAT | Amplification of URA3 and T_{URA3} from pYET1-TEF1- |
| | ATAATTAAATTGAAG | yeGFP for construction of pCS-yEC1 & pCS-yEC2 |
| oCS085 | TCAGTTATTACCCTAATTAGTGACGC | Amplification of insert 5 for construction of pCS-yEC1; |
| | GCATGAATG | Amplification of insert 4 for construction of pCS-yEC2; |
| | | from Sc W303 gDNA |
| oCS086 | GGGGATCCTCTAGAGGAGTCTGGTT | Amplification of insert 5 for construction of pCS-yEC1; |
| | TCTTCAACCCGGATCAGC | Amplification of insert 4 for construction of pCS-yEC2; |
| | | from Sc W303 gDNA |
| oCS087 | GTGAAAATCCACAGGAAGGAATAGTT | Amplification of yEC1 from pCS-yEC1 and |
| | | amplification of yEC2 from pCS-yEC2 |
| oCS088 | TCTTCAACCOGGATCAGC | Amplification of yEC1 from pCS-yEC1 and |
| | | amplification of yEC2 from pCS-yEC2 |
| oCS089 | TAGGCAGGCGGGCACCTCGCGTTAG | Genome walking adapter top oligo |
| | TGGCTGGGTCTAGGCGCTCTGGGCG | |
| | GGTAACGTGGAGNN | |
| oCS090 | /5PHOS/CTCCACGTTACCC/ | Genome walking adapter bottom oligo |
| | 3AMMO/ | |
| oCS091 | TAGGCAGGCGGGCACCTCGCGTTAG | Genome walking procedure both sides nested PCR |
| | TG | cycle 1 |
| oCS092 | CCCCGTGAACAACTCCTCCC | Genome walking procedure 5′ side nested PCR cycle |
| | | 1 |
| oCS093 | ATGGTCATCGCTCCTGGCTCTACTTG | Genome walking procedure 3′ side nested PCR cycle |
| | TGG | 1 |
| oCS094 | GTTAGTGGCTGGGTCTAGGCGCTCT | Genome walking procedure both sides nested PCR |
| | GG | cycle 2 |
| oCS095 | TGAACAACTCCTCCCCCTTG | Genome walking procedure 5′ side nested PCR cycle |
| | | 2 & sequencing |
| oCS096 | GCTGCTTCTTCCTTCCATCCGTGACT | Genome walking procedure 3′ side nested PCR cycle |
| | GTAT | 2 & sequencing |
| oCS097 | CGGTTGGTTGTAGGGGACGTGAGAA | PCR amplification of rDNA cistron locus on scaffold |
| | | 127 for finding insertion site in No IMET1 TC #17 |
| oCS098 | AGTGTCGATTTCGGCTGTGGGTAA | PCR amplification of rDNA cistron locus on scaffold |
| | | 127 for finding insertion site in No IMET1 TC #17 |
| oCS099 | GATTGGTTTCGGCCATCTTCCCT | PCR amplification of rDNA cistron locus on scaffold |
| | | 127 for finding insertion site in No IMET1 TC #17 |
| oCS100 | GGGGTCGTGGCAGAAATCTATAAGT | PCR amplification of rDNA cistron locus on scaffold |
| | G | 267 for finding insertion site in No IMET1 TC #17 |
| oCS101 | ATGGGCAGAGAGAGCAAGGGAAA | PCR amplification of rDNA cistron locus on scaffold |
| | | 267 for finding insertion site in No IMET1 TC #17 |
| oCS102 | GATTGGTTTCGGCCATCTTCCCT | PCR amplification of rDNA cistron locus on scaffold |
| | | 267 for finding insertion site in No IMET1 TC #17 |
| oCS103 | CGTCTCATCACACGAATTGTCCATCA | PCR amplification of rDNA cistron locus on scaffold |
| | | 341 for finding insertion site in No IMET1 TC #17 |
| oCS104 | GTTAGAAGTGCTCGAGAGGTTGGC | PCR amplification of rDNA cistron locus on scaffold |
| | | 341 for finding insertion site in No IMET1 TC #17 |
| oCS105 | GATTGGTTTCGGCCATCTTCCCT | PCR amplification of rDNA cistron locus on scaffold |
| | | 341 for finding insertion site in No IMET1 TC #17 |
| oCS106 | CGTCTCATCACACGAATTGTCCATCA | Checking NOR specific insertion in EC1 & EC7 |
| | | transformants 5′ side specific reaction |
| oCS107 | GGAACGGCACTGGTCAACTT | Checking NOR specific insertion in EC1 & EC7 |
| | | transformants 5′ side specific reaction |
| oCS108 | ATGGTCATCGCTCCTGGCTCTACTTG | Checking NOR specific insertion in EC1 & EC7 |
| | TGG | transformants 3′ side specific reaction |
| oCS109 | GTTAGAAGTGCTCGAGAGGTTGGC | Checking NOR specific insertion in EC1 & EC7 |
| | | transformants 3′ side specific reaction |
| oCS110 | ATTGAGTAAGGACCCCAAT | Checking NOR specific insertion in EC1 & EC7 |
| | | transformants control reaction |
| oCS111 | GTTGGAAAGGGAGGGTAG | Checking NOR specific insertion in EC1 & EC7 |
| | | transformants control reaction |
| oCS112 | CTCTCTGACGGAGGAGCTCTA | Verifying correct HDR insertion in EC7-CRISPR-NOR |
| | | transformants |
| oCS113 | TGAGGTTTAGATAACTTCTCACGCT | Verifying correct HDR insertion in EC7-CRISPR-NOR |
| | | transformants |
| oCS114 | GGAACGGCACTGGTCAACTT | Verifying correct HDR insertion in EC7-CRISPR-NOR |
| | | transformants 5′ side specific reaction |
| oCS115 | ACAACGAATTGTAGTACCGCAGA | Verifying correct HDR insertion in EC7-CRISPR-NOR |
| | | transformants 3′ side specific reaction |
| oCS116 | TGTGTTCCTTGCCACTACAAT | Verifying correct HDR insertion in EC7-CRISPR-NOR |
| | | transformants control reaction |
| oCS117 | AGAGGGCTGAAATTGACGGT | Verifying correct HDR insertion in EC7-CRISPR-NOR |
| | | transformants control reaction |
| oCS118 | GGATAACAATTTCACACAGG | Sequencing of pCS-CC |
| oCS119 | GGCACCTTATCAGAAAGA | Sequencing of pCS-CC |
| oCS120 | CCTGATCCCTGGCCATCAGTCCTGCT | Sequencing of pCS-CC |
| | CCTCGGC | |
| oCS121 | GTCGTGACTGGGAAAACCCTGGCG | Sequencing of pCS-CC pCS-TC |
| oCS122 | TGATTACGCCAAGCTTGCAT | Sequencing of pCS-TC |
| oCS123 | AAACGACGGCCAGTGAAT | Sequencing of pCS-TC |
| oCS124 | GGAACGGCACTGGTCAACTT | Sequencing of pCS-TC |
| oCS125 | GGTCTTGTTGGAGTTTGTGACG | Sequencing of pCS-TC |
| oCS126 | GTTGGAATGCCCCTCCCC | Sequencing of pCS-EC2-pCS-EC7 |
| oCS127 | CCATTGAAGTTGTGTGATG | Sequencing of pCS-EC2-pCS-EC7 |
| oCS128 | GGCGTGAATTGCGAGGAA | Sequencing of pCS-EC2-pCS-EC7 |
| oCS129 | ATGACGACCCTGTCCTTCCAGG | Sequencing of pCS-EC2-pCS-EC7 |
| oCS130 | TGATTACGCCAAGCTTGCAT | Sequencing of pCS-EC2-pCS-EC7 pCS-yEC1 pCS- |
| | | yEC2 |
| oCS131 | TAACTCAACAACAATAACGATAGCAG | Sequencing of pCS-EC2-pCS-EC7 |
| | CCAAGCAGTCGGTGGACGTTAC | |
| oCS132 | GCGTGAGAAGTTATCTAAAC | Sequencing of pCS-EC2-pCS-EC7 |
| oCS133 | CTGCTCGGAGGGGAGGATG | Sequencing of pCS-EC2-pCS-EC7 |
| oCS134 | TAGGCACCCCAGGCTTTAC | Sequencing of pCS-EC2-pCS-CVO and derivatives |
| | | pCS-yEC1 & pCS-yEC2 |
| oCS135 | ATGACGACCCTGTCCTTC | Sequencing of pCS-EC2-pCS-CV0 |
| oCS136 | GATTGGCATACGAAACTTGGTGGGA | Sequencing of pCS-CV0 |
| oCS137 | ATGAGCAAGGGGGAGGAG | Sequencing of pCS-CV0 |
| oCS138 | AAACGACGGCCAGTGAAT | Sequencing of pCS-CV0 pCS-yEC1 pCS-yEC2 |
| oCS139 | TGCTATCGTTATTGTTGTTGAG | Sequencing of pCS-EC7-CRISPR; Amplification of |
| | | insert 1 for assembly of pCS-CV1 |
| oCS140 | TCAGTCCTGCTCCTCGGCCACGA | Sequencing of pCS-EC7-CRISPR |
| oCS141 | ATGGTCATCGCTCCTGGCTCTACTTG | Sequencing of pCS-EC7-CRISPR |
| | TGG | |
| oCS142 | TGAAACCACTCCTAGTGAGATTG | Sequencing of pCS-EC7-CRISPR |
| oCS143 | TTGTAGACCGTCAATTTCAGCCCTCT | Sequencing of pCS-EC7-CRISPR |
| | TATCTTGTGTGTGTTAACAGCTGGTC | |
| oCS144 | TTGTAGACCGTCAATTTCAGCCCTCT | Sequencing of pCS-EC7-CRISPR |
| | TATCTCCGTCCACTCGTGATGCCTG | |
| oCS145 | GGAACGGCACTGGTCAACTT | Sequencing of pCS-EC6-derivatives |
| oCS146 | GCTGCTTCTTCCTTCCATCCGTGACT | Sequencing of pCS-EC6-derivatives |
| | GTAT | |
| oCS147 | GATTGGTTTCGGCCATCTTCCCT | Sequencing of pCS-EC6-derivatives |
| oCS148 | TAGCCAAATGCCTCGTCA | Sequencing of pCS-yEC1-pCS-yEC2; Amplification |
| | | of insert 3 from pCS-EV6-tdTomato for construction of |
| | | pCS-CV1 |
| oCS149 | CGTCACTAATTAGGGTAATAACTGAT | Sequencing of pCS-yEC1-pCS-yEC2 |
| | ATAATTAAATTGAAG | |
| oCS150 | GCATCAGAAGAGGTGAATATCGTCGA | Amplification of insert 3 from pCS-EV6-tdTomato for |
| | GTGGTATTTCATCGTCGCCGGA | construction of |
| | | pCS-CV1 |
| oCS151 | TTAAGTTGGGTAACGCCAGGGTTTTA | Amplification of insert 4 from No IMET1 gDNA for |
| | AAGCGGATGCGTATGTGTTG | construction of pCS-CV1 |
| oCS152 | ATCGCCACTACCCTCCCTTTCCAACT | Amplification of backbone from pCS-CV1 for 1- |
| | CATCGTCATCGTCCTTT | fragment ligation construction of pCS-CV2 |
| oCS153 | GTTGGAAAGGGAGGGTAG | Amplification of backbone from pCS-CV1 for 1- |
| | | fragment ligation construction of pCS-CV2 |
| oCS154 | TGGCCAGGGATCAGGAGG | Amplification of backbone from pCS-CV2 for |
| | | construction of pCS-ECT2; Amplification of insert 2 |
| | | from pCS-ECT2 for construction of pCS-ECVHH-his |
| oCS155 | TCCCTTCATCCTCCCCTCCGAGCAGA | Amplification of insert 1 from EC6 for construction of |
| | TGAGCAAGGGGGAGGAGTTG | pCS-ECT2 |
| oCS156 | CGAGAGGGGCATGGGCCCCGGGTT | Amplification of insert 1 from EC6 for construction of |
| | CTCCTC | pCS-ECT2 |
| oCS157 | GAACCCGGGGCCCATGCCCCTCTCG | Amplification of insert 2 from pNOC-ARS-CRISPR- |
| | CAGGAG | BlastR for construction of pCS-ECT2 |
| oCS158 | ACTCCCTCCTCCTGATCCCTGGCCAT | Amplification of insert 2 from pNOC-ARS-CRISPR- |
| | CAAGCGCTCTCCCACAC | BlastR for construction of pCS-ECT2 |
| oCS159 | ACACCAAACAGTTTCGAC | Amplification of backbone from pCS-ECT2 for |
| | | construction of pCS-ECPL via GA and for |
| | | construction of pCS-ECP- via 1-fragment ligation |
| oCS160 | TAAGCTCTTTCTTCTTCTCTTG | Amplification of backbone from pCS-ECT2 for |
| | | construction of pCS-ECPL via GA and for |
| | | construction of pCS-ECP-via 1-fragment ligation |
| oCS161 | ATTCAAGAGAAGAAGAAAGAGCTTAG | Amplification of insert 1 from No IMET1 gDNA for |
| | ATGGAGTGGATGGAGGAG | construction of pCS-ECPL |
| oCS162 | CCGCCAAGTCGAAACTGTTTGGTGTT | Amplification of insert 1 from No IMET1 gDNA for |
| | GTTGATGCGGGCTGAGATTG | construction of pCS-ECPL |
| oCS163 | TCATCGTCATCGTCCTTT | Amplification of backbone from pCS-ECT2 for GA |
| | | construction of pCS-ECT1 & pCS-ECT4 & pCS- |
| | | ECT5; Amplification of backbone from pCS-ECT2 for |
| | | 1-fragment ligation construction of pCS-ECT3 |
| oCS164 | CGCAATCTCACTAGGAGTGGTTTCAA | Amplification of backbone from pCS-ECT2 for |
| | AGGGAGGGTAGTGGCGATG | construction of pCS-ECT1 |
| oCS165 | GTACGGGAAAGGACGATGACGATGA | Amplification of insert 1 from No IMET1 gDNA for |
| | TGAGTCCAGGGCACCCGA | construction of pCS-ECT1 |
| oCS166 | CGCAATCTCACTAGGAGTGGTTTCAT | Amplification of backbone from pCS-ECT2 for 1- |
| | CAAGCGCTCTCCCACAC | fragment ligation construction of pCS-ECT3 & pCS- |
| | | ECT5 |
| oCS167 | TCTACTCGTCTCTCTTGGATCTTTCT | Amplification of backbone from pCS-ECT2 for |
| | CAAGCGCTCTCCCACAC | construction of pCS-ECT4 |
| oCS168 | GAAAGATCCAAGAGAGACG | Amplification of insert 1 from pNOC-ARS-CRISPR- |
| | | BlastR for construction of pCS-ECT4 |
| oCS169 | CGCAATCTCACTAGGAGTGGTTTCAT | Amplification of insert 1 from pNOC-ARS-CRISPR- |
| | AGTGGATCAGCTTGCATG | BlastR for construction of pCS-ECT4 |
| oCS170 | ACGTGTGGGAGAGCGCTTGAAAAAAA | Ultramer containing poly(A) coding tract for |
| | AAAAAAAAAAAAAAAAAAAAAAAAAA | construction of pCS-ECT5 |
| | AAAAAAAAAAAAAAAAAAAAAAAAAA | |
| | AAAAAAAAAAAAAAAAAAAAAAAAAA | |
| | AAAAAAAAAAAAAAAAAAAAAAAGGC | |
| | CGGCATGGTCCCAGCCTCCTCGCTGG | |
| | CGCCGGCTGGGCAACATGCTTCGGCA | |
| | TGGCGAATGGGACTCCTG | |
| oCS171 | ATCAGAGAACTTCTCCCTTCGGGCTA | Amplification of insert 1 from poly(A)-containing |
| | CGTGTGGGAGAGCGCTTG | ultramer for construction of pCS-ECT5 |
| oCS172 | CGCAATCTCACTAGGAGTGGTTTCAC | Amplification of insert 1 from poly(A)-containing |
| | AGGAGTCCCATTCGCCATG | ultramer for construction of pCS-ECT5 |
| oCS173 | TCATCGTCATCGTCCTTTCCC | Amplification of backbone from pCS-ECT2 for |
| | | construction of pCS-ECVHH-his |
| oCS174 | ATCGCCACTACCCTCCCTTTCCAACG | Amplification of insert 3 from pNOC-ARS-CRISPR- |
| | ATGGAGTGGATGGAGGAG | BlastR for construction of pCS-ECVHH-his |
| oCS175 | GTACGGGAAAGGACGATGACGATGA | Amplification of insert 3 from pNOC-ARS-CRISPR- |
| | AGTAGATGCCGACCGGAG | BlastR for construction of pCS-ECVHH-his |
| oCS176 | TGGCCAGGGATCAGGAGG | Amplification of backbone from pCS-ECVHH-his for |
| | | construction of pCS-ECVHH-NLuc |
| oCS177 | TCCTCGAGAGTAAACACCATGCTGCT | Amplification of backbone from pCS-ECVHH-his for |
| | GACCGTGACCTG | construction of pCS-ECVHH-NLuc |
| oCS178 | ATGGTGTTTACTCTCGAGGACTTCG | Amplification of insert 1 from pCS-EC-BRA-Noc-IRES |
| | | for construction of pCS-ECVHH-NLuc |
| oCS179 | CTCCTCCTGATCCCTGGCCATCATCC | Amplification of insert 1 from pCS-EC-BRA-Noc-IRES |
| | GGCCAGGATGCG | for construction of pCS-ECVHH-NLuc |
| oCS180 | TGGCCAGGGATCAGGAGG | Amplification of backbone from pCS-CV3 for |
| | | construction of pCS-EC-BRA vectors |
| oCS181 | TTAGATACAATTGTAGTGGCAAGGAA | Amplification of backbone from pCS-CV3 for |
| | CAC | construction of pCS-EC-BRA vectors |
| oCS182 | TCCTTGCCACTACAATTGTATCTAAG | Amplification of insert 1 from pNOC-ARS-CRISPR- |
| | ATGGAGTGGATGGAGGAG | BlastR for construction of pCS-EC-BRA vectors |
| oCS183 | TGACCTCCTCGCCCTTGCTCACCATT | Amplification of insert 1 from pNOC-ARS-CRISPR- |
| | GTTGATGCGGGCTGAGATTG | BlastR for construction of pCS-EC-BRA vectors |
| oCS184 | ATGGTGAGCAAGGGCGAGGA | Amplification of insert 2 from pCS-EC6-tdTomato for |
| | | construction of pCS-EC-BRA vectors |
| oCS185 | TCAGTCCTGCTCCTCGGC | Amplification of insert 2 from pCS-EC6-tdTomato for |
| | | construction of pCS-EC-BRA vectors except pCS-EC- |
| | | BRA-NC |
| oCS186 | CGAAGTCCTCGAGAGTAAACACCATT | Amplification of insert 2 from pCS-EC6-tdTomato for |
| | TAGCTATCAGTCCTGCTCCTCGGC | construction of pCS-EC-BRA-NC |
| oCS187 | ATGGTGTTTACTCTCGAGGACTTCG | Amplification of insert 3 from pNOC-ARS-CRISPR- |
| | | BlastR for construction of pCS-EC-BRA vectors |
| oCS188 | ACTCCCTCCTCCTGATCCCTGGCCAT | Amplification of insert 3 from pNOC-ARS-CRISPR- |
| | CACGCGTAGTCGGGCACGTC | BlastR for construction of pCS-EC-BRA vectors |
| oCS189 | CTTCGTGGCCGAGGAGCAGGACTGA | Amplification of insert 4 from pCS-EC6-tdTomato for |
| | TAGCTAAACAACCAGCTCAGAACTGG | construction of pCS-EC-BRA-Noc-IRES. Other pCS- |
| | AG | EC-BRA vectors assembled with gblock fragments |
| | | instead |
| oCS190 | CGAAGTCCTCGAGAGTAAACACCATC | Amplification of insert 4 from pCS-EC6-tdTomato for |
| | TGCTCGGAGGGGAGGATG | construction of pCS-EC-BRA-Noc-IRES. Other pCS- |
| | | EC-BRA vectors assembled with gblock fragments |
| | | instead |
| oCS191 | CCGGGGACTCGCATGCAA | Amplification of backbone from pCS-yEC1 for |
| | | construction of intermediate cloning vectors pCS- |
| | | PPEC-TEV0 & pCS-PPEC-GAP0 and vectors pCS- |
| | | PPEC-TEV-26S & pCS-PPEC-TEV-AOX1 & pCS- |
| | | PPEC-GAP-26S & pCS-PPEC-GAP-AOX1 |
| oCS192 | GCCAAGCTTGCATGCGAGTCCCCGG | Amplification of GAP promoter from Pp X-33 gDNA |
| | TTTTTGTAGAAATGTCTTGGTG | for construction of the intermediate cloning vector |
| | | pCS-PPEC-GAPO |
| oCS193 | TGAATAATTCTTCACCTTTAGACATA | Amplification of GAP promoter from Pp X-33 gDNA |
| | TAGTTGTTCAATTGATTGAAATAG | for construction of the intermediate cloning vector |
| | | pCS-PPEC-GAP0 and sequencing of pCS-PPEC- |
| | | GAP derivatives |
| oCS194 | ATGTCTAAAGGTGAAGAATTATTC | Amplification of EGFP from pCS-yEC1 for |
| | | construction of intermediate cloning vectors pCS- |
| | | PPEC-TEV0 & pCS-PPEC-GAP0 and sequencing of |
| | | pCS-PPEC-Noc-TE |
| oCS195 | GAACGGCACTGGTCAACTTGGCCATT | Amplification of EGFP from pCS-yEC1 for construction |
| | GGACCAGGATTTTCTTC | of intermediate cloning vectors pCS- |
| | | PPEC-TEV0 & pCS-PPEC-GAP0 and sequencing of |
| | | pCS-PPEC-TEV derivatives |
| oCS196 | ATGGCCAAGTTGACCAGTG | Amplification of zeoR from EC5 for construction of |
| | | intermediate cloning vectors pCS-PPEC-TEV0 & |
| | | pCS-PPEC-GAP0 and sequencing of pCS-PPEC- |
| | | TEV derivatives & pCS-PPEC-GAP derivatives& |
| | | pCS-PPEC-Noc-TE |
| oCS197 | TCAGTCCTGCTCCTCGGC | Amplification of zeoR from EC5 for construction of |
| | | intermediate cloning vectors pCS-PPEC-TEV0 & |
| | | pCS-PPEC-GAP0 and sequencing of pCS-PPEC- |
| | | TEV derivatives & pCS-PPEC-GAP derivatives |
| oCS198 | CTTCGTGGCCGAGGAGCAGGACTGA | Amplification of AOX1 terminator from Pp X-33 gDNA |
| | TCAAGAGGATGTCAGAATG | for construction of intermediate cloning vectors pCS- |
| | | PPEC-TEV0 & pCS-PPEC-GAPO |
| oCS199 | GGGGATCCTCTAGAGGAGTCTGGTT | Amplification of AOX1 terminator from Pp X-33 gDNA |
| | TCTCACTTAATCTTCTGTACTC | for construction of intermediate cloning vectors pCS- |
| | | PPEC-TEV0 & pCS-PPEC-GAPO |
| oCS200 | TTTTTGTAGAAATGTCTTGGTG | Amplification of insert 2 from the intermediate cloning |
| | | vector pCS-PPEC-GAP0 for construction of pCS- |
| | | PPEC-GAP-26S & pCS-PPEC-GAP-AOX1 |
| oCS201 | TCTCACTTAATCTTCTGTACTC | Amplification of insert 2 from the intermediate cloning |
| | | vector pCS-PPEC-TEV0 for construction of pCS- |
| | | PPEC-TEV-26S and pCS-PPEC-TEV-AOX1 & |
| | | amplification of insert 2 from the intermediate cloning |
| | | vector pCS-PPEC-GAP0 for construction of pCS- |
| | | PPEC-GAP-26S & pCS-PPEC-GAP-AOX1 |
| oCS202 | AAATAACAAATCTCAACACAAC | Amplification of insert 2 from the intermediate cloning |
| | | vector pCS-PPEC-TEV0 for construction of pCS- |
| | | PPEC-TEV-26S & pCS-PPEC-TEV-AOX1 |
| oCS203 | GCCAAGCTTGCATGCGAGTCCCCGG | Amplification of insert 1 from Pp X-33 gDNA for |
| | AGTACTGACCCCCTCAGTGGGCCA | construction of pCS-PPEC-TEV-26S & pCS-PPEC- |
| | | GAP-26S |
| oCS204 | GGACACCAAGACATTTCTACAAAAAG | Amplification of insert 1 from Pp X-33 gDNA for |
| | ATGACGAGGCATTTGGCTACCTTAAG | construction of pCS-PPEC-GAP-26S |
| oCS205 | TCAGAGTACAGAAGATTAAGTGAGAT | Amplification of insert 3 from Pp X-33 gDNA for |
| | AATTAGTGACGCGCATGAATGG | construction of pCS-PPEC-TEV-26S & pCS-PPEC- |
| | | GAP-26S |
| oCS206 | GGGGATCCTCTAGAGGAGTCTGGTT | Amplification of insert 3 from Pp X-33 gDNA for |
| | AGTACTTGGTACCGCTTGGTGGGATT | construction of pCS-PPEC-TEV-26S & pCS-PPEC- |
| | AC | GAP-26S |
| oCS207 | TATGTTGTGTTGAGATTTGTTATTTG | Amplification of insert 1 from Pp X-33 gDNA for |
| | ATGACGAGGCATTTGGCTACCTTAAG | construction of pCS-PPEC-TEV-26S |
| oCS208 | GCCAAGCTTGCATGCGAGTCCCCGG | Amplification of insert 1 from Pp X-33 gDNA for |
| | AGTACTCTAGCAAGACCGGTCTTC | construction of pCS-PPEC-TEV-AOX1 & pCS-PPEC- |
| | | GAP-AOX1 |
| oCS209 | GGACACCAAGACATTTCTACAAAAAT | Amplification of insert 1 from Pp X-33 gDNA for |
| | CCGTGGTGATGCTGAGATTC | construction of pCS-PPEC-GAP-AOX1 |
| oCS210 | TCAGAGTACAGAAGATTAAGTGAGAC | Amplification of insert 3 from Pp X-33 gDNA for |
| | GAAGTCATCGAAAGACTC | construction of pCS-PPEC-TEV-AOX1 & pCS-PPEC- |
| | | GAP-AOX1 |
| oCS211 | GGGGATCCTCTAGAGGAGTCTGGTT | Amplification of insert 3 from Pp X-33 gDNA for |
| | AGTACTTAATTATTCGAAACGATGGC | construction of pCS-PPEC-TEV-AOX1 & pCS-PPEC- |
| | | GAP-AOX1 and sequencing of pCS-PPEC-TEV- |
| | | AOX1 |
| oCS212 | TATGTTGTGTTGAGATTTGTTATTTT | Amplification of insert 1 from Pp X-33 gDNA for |
| | CCGTGGTGATGCTGAGATTC | construction of pCS-PPEC-TEV-AOX1 |
| oCS213 | GATGACGAGGCATTTGGC | Amplification of backbone from pCS-PPEC-TEV-26S |
| | | for construction of pCS-PPEC-Noc-TEV-TE |
| oCS214 | TCAAGAGGATGTCAGAATGC | Amplification of backbone from pCS-PPEC-TEV-26S |
| | | for construction of pCS-PPEC-Noc-TEV-TE |
| oCS215 | TAAGGTAGCCAAATGCCTCGTCATCA | Amplification of insert 1 from EC5 for construction of |
| | CAACCAGCTCAGAACTGGAG | pCS-PPEC-Noc-TEV-TE |
| oCS216 | TATGTTGTGTTGAGATTTGTTATTTT | Amplification of insert 1 from EC5 for construction of |
| | GATCATCTGCTCGGAGGGGAGGATG | pCS-PPEC-Noc-TEV-TE |
| oCS217 | AAATAACAAATCTCAACACAACA | Amplification of insert 2 from pCS-PPEC-TEV-26S for |
| | | construction of pCS-PPEC-Noc-TEV-TE |
| oCS218 | TGATCAGTCCTGCTCCTC | Amplification of insert 2 from pCS-PPEC-TEV-26S for |
| | | construction of pCS-PPEC-Noc-TEV-TE |
| oCS219 | CGTGGCCGAGGAGCAGGACTGATCA | Amplification of insert 3 from EC5 for construction of |
| | CAGTGGCCAGGGATCAGGAGG | pCS-PPEC-Noc-TEV-TE and sequencing of pCS- |
| | | PPEC-Noc-TEV-TE |
| oCS220 | AAATGGCATTCTGACATCCTCTTGAA | Amplification of insert 3 from EC5 for construction of |
| | AGGGAGGGTAGTGGCGATG | pCS-PPEC-Noc-TEV-TE |
| oCS221 | /5PHOS/ATGTCTAAAGGTGAAGAAT | Amplification of backbone from pCS-PPEC-Noc-TEV- |
| | TATTCACTGGT | TE for construction of pCS-PPEC-Noc-TE |
| oCS222 | CATCTGCTCGGAGGGGAGGAT | Sequencing of pCS-PPEC-Noc-TEV-TE |
| oCS223 | GATGTTGACGCAATGTGAT | Sequencing of pCS-PPEC-Noc-TEV-TE |
| oCS224 | CAAGCCCGTTCCCTTGGCT | Sequencing of pCS-PPEC-Noc-TEV-TE |
| oCS225 | GCCCAGTGCTCTGAATGTCA | Sequencing of pCS-PPEC-Noc-TE |
| oCS226 | TGGTCTTGTAGTTACCGTCA | Sequencing of pCS-PPEC-Noc-TE |
| oCS227 | GACCCCCTCAGTGGGCCAT | Amplification of constructs PPEC-TEV-26S & PPEC- |
| | | Noc-TE |
| oCS228 | TGGTACCGCTTGGTGGGATT | Amplification of constructs PPEC-TEV-26S & PPEC- |
| | | Noc-TE |
| oCS229 | TGCCGAAGTTTCCCTCAGGAT | Checking NOR 26S specific insertion in PPEC-TEV- |
| | | 26S & PPEC-GAP-26S transformants 5′side specific |
| | | reaction |
| oCS230 | TCACAATGCTGCAAATGACGCT | Checking AOX1 specific insertion in PPEC-GAP- |
| | | AOX1 transformants 5′side specific reaction |
| oCS231 | CTAAGGTTGGCCATGGAACTGG | Checking NOR and AOX1 specific insertion in PPEC- |
| | | TEV derivative & PPEC-GAP derivative transformants |
| | | 5′side specific reaction |
| oCS232 | ACGACGTGACCCTGTTCATCA | Checking NOR and AOX1 specific insertion in PPEC- |
| | | TEV derivative & PPEC-GAP derivative transformants |
| | | 3′side specific reaction |
| oCS233 | TTTTGGAAGTGGAGGTGTCACG | Checking NOR 26S specific insertion in PPEC-TEV- |
| | | 26S & PPEC-GAP-26S transformants 3′ side specific |
| | | reaction |
| oCS234 | GAAACACCCGCTTTTTGGATGA | Checking AOX1 specific insertion in PPEC-GAP- |
| | | AOX1 transformants 3′ side specific reaction |
| oCS235 | AAUUUCUACUGUUGUAGAUGUGUGU | crRNA with complemetarity to the NOR locus of N. |
| | GUUAACAGCUGGUCGCAG | oceanica on chromosome 3, adjacent to the rDNA |
| | | cistron. Used for targeted insertion of EC7-CRISPR- |
| | | NOR |
| oCS236 | AAUUUCUACUGUUGUAGAUAUGACG | crRNA with complementarity to the tdTomato gene. |
| | GCCAUGUUGUUGUCCUCG | Used for targeted insertion of different ECs into |
| | | the NOR on chromosome 3 of N. oceanica strains |
| | | carrying a tdTomato gene in that locus, such as |
| | | “landing site” strains EC7-tdTomato-S1 or ECT2- |
| | | tdTomato-S1 |

MCEC-EMCV (SEQ ID NO:261), including EMCV IRES, SVPLA, EGFP, P2A, shble, PNAts, and homology flanks to direct the cassette to the 28S rDNA of mammalian cells.
1-1000 Left HF, complementary to the 28S rDNA of a mammalian cell line, C989A
1001-1551 IRES of the EMCV
1552-2280 EGFP, humanized, with four additional N-terminal AAs to safeguard optimal EMCV IRES performance
2290-2346 2A peptide from porcine teschovirus-1 polyprotein
2347-2721 Zeocin resistance gene, codon optimized for H. sapiens
2722-2857 SVLPA
2858-2933 PNAts, contains binding site for NoLS-PNA to facilitate EC trafficking to the nucleolus
2873-2918 DNA triplex formation site upon PNA binding
2934-3933 Right HF, complementary to the 28S rDNA of a mammalian cell line

```
GAACAGCCTCTGGCATGTTGGAACAATGTAGGTAAGGGAAGTCGGCAAGCCGGATCCGTAACTTCGGG
ATAAGGATTGGCTCTAAGGGCTGGGTCGGTCGGGCTGGGGCGCGAAGCGGGGCTGGGCGCGCGCCGCG
GCTGGACGAGGCGCCGCCGCCCCCCCCACGCCCGGGGCACCCCCCTCGCGGCCCTCCCCCGCCCCACC
CCGCGCGCGCCGCTCGCTCCCTCCCCGCCCCGCGCCCTCTCTCTCTCTCTCTCCCCCGCTCCCCGTCC
TCCCCCCTCCCCGGGGGAGCGCCGCGTGGGGGCGGCGGCGGGGGGAGAAGGGTCGGGGCGGCAGGGGC
CGGCGGCGGCCCGCCGCGGGGCCCCGGCGGCGGGGGCACGGTCCCCCGCGAGGGGGGCCCGGGCACCC
GGGGGGCCGGCGGCGGCGGCGACTCTGGACGCGAGCCGGGCCCTTCCCGTGGATCGCCCCAGCTGCGG
CGGGCGTCGCGGCCGCCCCCGGGGAGCCCGGCGGGCGCCGGCGCGCCCCCCCCCACCCCCACCCCACG
TCTCGTCGCGCGCGCGTCCGCTGGGGGCGGGGAGCGGTCGGGCGGCGGCGGTCGGCGGGCGGCGGGGC
GGGGCGGTTCGTCCCCCCGCCCTACCCCCCCGGCCCCGTCCGCCCCCCGTTCCCCCCTCCTCCTCGGC
GCGCGGCGGCGGCGGCGGCGGCGGCAGGCGGCGGAGGGGCCGCGGGCCGGTCCCCCCCGCCGGGTCCG
CCCCCGGGGCCGCGGTTCCGCGCGGCGCCTCGCCTCGGCCGGCGCCTAGCAGCCGACTTAGAACTGGT
GCGGACCAGGGGAATCCGACTGTTTAATTAAAACAAAGCATCGCGAAGGCCCGCGGCGGGTGTTGACG
CGATGTGATTTCTGCCCAGTGCTCTGAATGTCAAAGTGAAGAAATTCAATGAAGCGCGGGTAAACGGC
GGGAGTAACTATGACTCTCTTAAGGTAGCCAAATGCATCGTCATCTAAACGTTACTGGCCGAAGCCGC
TTGGAATAAGGCCGGTGTGCGTTTGTCTATATGTTATTTTCCACCATATTGCCGTCTTTTGGCAATGT
GAGGGCCCGGAAACCTGGCCCTGTCTTCTTGACGAGCATTCCTAGGGGTCTTTCCCCTCTCGCCAAAG
GAATGCAAGGTCTGTTGAATGTCGTGAAGGAAGCAGTTCCTCTGGAAGCTTCTTGAAGACAAGCAACG
TCTGTAGCGACCCTTTGCAGGCAGCGGAACCCCCCACCTGGCGACAGGTGCCTCTGCGGCCAAAAGCC
ACGTGTATAAGATACACCTGCAAAGGCGGCACAACCCCAGTGCCACGTTGTGAGTAGGATAGTTGTGG
AAAGAGTCAAATGGCTCTCCTCAAGCGTATTCAACAAGGGGCTGAAGGATGCCCAGAAGGTACCCCAT
TGTATGGGATCTGATCTGGGGCCTCGGTGCACATGCTTTACATGTGTTTAGTCGAGGTTAAAAAACGT
CTAGGCCCCCCGAACCACGGGGACGTGGTTTTCCTTTGAAAAACACGATGATAATATGGCCACAACCA
TGGTCAGTAAGGGAGAGGAATTGTTCACTGGGGTAGTGCCTATATTGGTAGAACTCGACGGAGATGTG
AATGGCCACAAATTTTCTGTGTCAGGGGAGGGTGAGGGTGATGCAACCTACGGAAAATTGACGCTGAA
GTTTATTTGTACGACGGGCAAGCTGCCAGTTCCCTGGCCCACATTGGTAACAACCCTTACCTATGGAG
TACAGTGTTTCAGTCGATATCCAGATCACATGAAACAGCACGACTTTTTCAAAAGCGCGATGCCGGAA
GGATACGTTCAAGAAAGGACTATATTCTTCAAGGATGATGGCAACTACAAAACACGAGCAGAAGTAAA
GTTCGAGGGGGATACGCTCGTAAATAGGATCGAACTCAAGGGAATAGACTTCAAGGAGGACGGGAATA
TACTCGGACATAAGTTGGAGTACAACTATAATAGCCATAATGTATATATAATGGCCGACAAGCAGAAG
AATGGAATCAAAGTAAATTTCAAAATTCGCCACAACATCGAGGATGGCTCCGTCCAGCTTGCGGATCA
TTATCAGCAGAATACTCCAATCGGAGATGGACCTGTTTTGCTGCCGGATAATCATTACCTGTCTACAC
AGTCAGCCCTTTCAAAAGATCCGAACGAGAAGCGGGACCATATGGTACTTCTTGAGTTCGTCACCGCG
GCGGGAATAACACTTGGGATGGATGAACTCTACAAAGGAAGCGGAGCTACTAACTTCAGCCTGCTGAA
GCAGGCTGGAGACGTGGAGGAGAACCCTGGACCTATGGCTAAACTCACAAGTGCGGTCCCTGTTTTGA
CTGCAAGGGATGTTGCAGGAGCCGTTGAGTTCTGGACGGATCGATTGGGGTTTAGCCGAGATTTTGTC
GAAGATGACTTTGCTGGCGTTGTACGAGATGATGTCACATTGTTTATATCTGCTGTTCAGGATCAAGT
TGTACCGGACAACACATTGGCTTGGGTATGGGTGCGGGGTCTCGATGAGCTGTACGCAGAGTGGAGCG
AGGTTGTGAGTACGAATTTTAGAGATGCAAGCGGCCCTGCCATGACGGAGATCGGGGAACAACCATGG
GGCCGCGAATTTGCCTTGAGAGACCCTGCGGGTAATTGTGTGCACTTTGTTGCCGAAGAGCAGGATTG
AAAAAAATGCTTTATTTGTGAAATTTGTGATGCTATTGCTTTATTTGTAACCATTATAAGCTGCAATA
AACAAGTTAACAACAACAATTGCATTCATTTTATGTTTCAGGTTCAGGGGGAGGTGTGGGAGGTTTTT
TGTGATCCTAGCATTAAAGAAGAAAACTAAGAAGAAAACTAAGAAGAAAACTAAGAAGAAAAGTATCA
TGAACTGCCTTAGTGACGCGCATGAATGGATGAACGAGATTCCCACTGTCCCTACCTACTATCCAGCG
AAACCACAGCCAAGGGAACGGGCTTGGCGGAATCAGCGGGGAAAGAAGACCCTGTTGAGCTTGACTCT
AGTCTGGCACGGTGAAGAGACATGAGAGGTGTAGAATAAGTGGGAGGCCCCCGGCGCCCCCCCGGTGT
CCCCGCGAGGGGCCCGGGGCGGGGTCCGCCGGCCCTGCGGGCCGCCGGTGAAATACCACTACTCTGAT
CGTTTTTTCACTGACCCGGTGAGGCGGGGGGGCGAGCCCCGAGGGGCTCTCGCTTCTGGCGCCAAGCG
CCCGGCCGCGCGCCGGCCGGGCGCGACCCGCTCCGGGGACAGTGCCAGGTGGGGAGTTTGACTGGGGC
GGTACACCTGTCAAACGGTAACGCAGGTGTCCTAAGGCGAGCTCAGGGAGGACAGAAACCTCCCGTGG
AGCAGAAGGGCAAAAGCTCGCTTGATCTTGATTTTCAGTACGAATACAGACCGTGAAAGCGGGGCCTC
ACGATCCTTCTGACCTTTTGGGTTTTAAGCAGGAGGTGTCAGAAAAGTTACCACAGGGATAACTGGCT
TGTGGCGGCCAAGCGTTCATAGCGACGTCGCTTTTTGATCCTTCGATGTCGGCTCTTCCTATCATTGT
GAAGCAGAATTCACCAAGCGTTGGATTGTTCACCCACTAATAGGGAACGTGAGCTGGGTTTAGACCGT
CGTGAGACAGGTTAGTTTTACCCTACTGATGATGTGTTGTTGCCATGGTAATCCTGCTCAGTACGAGA
GGAACCGCAGGTTCAGACATTTGGTGTATGTGCTTGGCTGAGGAGCCAATGGGGCGAAGCTACCATCT
GTGGGATTATGACTGAACGCCTCTAAGTCAGAATCCCGCCCAGGCGGAACGATACGGCAGCGCCGCGG
AGCCTCGGTTGGCCTCGGATAGCCGGTCCCCCGCCTGTCCCCGCCGGGGGCCGCCC
Synthetic sgRNA sequence for assembly of NoLS-Cas9 RNP for 28S rDNA
cleavage in mammalian cells.
The spacer motif complementary to the 28S rDNA is underlined
  (SEQ ID NO. 263)
GCGUCACUAAUUAGAUGACGGUUUUAGAGCUAGAAAUAGCAAGUUAAAAUAAGGCUAGUCCGUUAUCA
ACUUGAAAAAGUGGCACCGAGUCGGUGCUUUU
HIV-1 Rev protein nucleolar localization sequence.
  (SEQ ID NO. 264)
RQARRNRRRRWRERQRQI
```

1. A method for expressing or producing one or more proteins of interest in a eukaryotic cell by
a. introducing into a eukaryotic cell a nucleic acid molecule comprising a polynucleotide encoding a protein of interest (POI),
wherein said nucleic acid molecule is targeted to the nucleolar DNA, preferably to a nucleolar organizer region (NOR), of said organism to insert or form upon integration of said nucleic acid molecule a chimeric gene comprising the following operably-linked elements:
i. a polymerase I promoter;
ii. a polynucleotide encoding an internal ribosomal entry site (IRES);
iii. said polynucleotide encoding said POI;
iv. optionally, a 3′ end region/transcription terminator.

2. The method of claim 1, wherein prior to introduction said nucleic acid molecule already comprises said polymerase I promoter, preferably wherein said nucleic acid molecule already comprises said chimeric gene.

3. The method of claim 1, wherein said nucleic acid molecule is flanked with one or more flanking sequences for allowing integration of said nucleic acid molecule at a predefined site in said nucleolar DNA by homologous recombination; and/or
wherein a DNA break is induced at a predefined site in said nucleolar DNA by a sequence specific nuclease (SSN), thereby allowing integration of said nucleic acid molecule at said predefined site.

4. The method of claim 1, wherein said chimeric gene further comprises a polynucleotide encoding a translational enhancer (TE) or a cap-independent translation enhancer (CITE).

5. The method of claim 1, wherein said chimeric gene further comprises a polynucleotide encoding a second IRES sequence (and optionally a second TE/CITE) operably-linked to a second polynucleotide encoding a second protein of interest (POI).

6. The method of claim 1, wherein expression of said POI is enhanced compared to expression driven by an average pol II promoter, preferably enhanced compared to a strong pol II promoter.

7. The method of claim 1, comprising the further step of isolating and optionally purifying said one or more produced POIs.

8. A chimeric gene for producing one or more proteins of interest (POI) comprising the following operably-linked elements:
i. a polymerase I promoter;
ii. a polynucleotide encoding an internal ribosomal entry site (IRES);
iii. said polynucleotide encoding said POI;
iv. optionally, a 3′ end region/transcription terminator.

9. A (transgenic/cis-genic) eukaryotic cell for expressing or producing one or more proteins of interest (POI) comprising the chimeric gene of claim 8,
wherein said chimeric gene has been integrated into the nucleolar DNA of said cell, preferably into a nucleolar organiser region (NOR).

10. The method of claim 1, wherein:
a) said chimeric gene is integrated in or in the vicinity of an rDNA cistron, preferably within 10 kb of an rDNA cistron; or
b) said chimeric gene is integrated outside an rDNA cistron.

11. The cell of claim 9, wherein:
a) said chimeric gene is integrated in or in the vicinity of an rDNA cistron, preferably within 10 kb of an rDNA cistron; or
b) said chimeric gene is integrated outside an rDNA cistron.

12. The method of claim 1, wherein:
a) said cell is selected from an animal cell, plant cell, a protist cell and fungal cell; or
b) said cell is a (unicellular) plant cell, algal cell or yeast cell, preferably wherein said cell is selected from a Nannochloropsis sp., a Saccharomyces sp. or Pichia sp.

13. The cell of claim 9, wherein:
a) said cell is selected from an animal cell, plant cell, a protist cell and fungal cell; or
b) said cell is a (unicellular) plant cell, algal cell or yeast cell, preferably wherein said cell is selected from a Nannochloropsis sp., a Saccharomyces sp. or Pichia sp.

14. The method of claim 1, wherein said cell is from a Nannochloropsis sp, preferably Nannochloropsis oceanica.

15. A method for producing a protein or polypeptide of interest (POI), comprising the steps of
a. providing the cell of claim 9; and optionally
b. isolating and/or purifying said protein or polypeptide

16. A nucleic acid molecule or vector for expressing one or more proteins of interest (POI) in a eukaryotic cell, said nucleic acid molecule or vector comprising a polynucleotide encoding said at least one (POI), wherein upon integration into the nucleolar DNA, preferably into a nucleolar organizer region (NOR), of said eukaryotic cell the chimeric gene of claim 8 is formed.

17. A kit for expressing one or more proteins of interest (POIs) in a eukaryotic cell, said kit comprising one or more containers comprising the vector or nucleic acid molecule of claim 16.

18. The nucleic acid molecule or vector or kit of claim 16, wherein said polynucleotide encoding said at least one (POI) is flanked with one or more flanking sequences that allow insertion of said polynucleotide encoding said POI into a predefined site into a nucleolar organizer region (NOR) of said eukaryotic cell by homologous recombination to insert or form said chimeric gene; and/or
wherein said nucleic acid molecule or vector or kit further comprises an expression cassette for expressing a sequence specific nuclease capable of inducing a DNA break at a predefined site in the nucleolar DNA (e.g. NOR) of said eukaryotic cell for allowing integration of said polynucleotide encoding said POI at said predefined site to insert or form said chimeric gene.

19. The nucleic acid molecule or vector or kit of claim 16, which comprises said polymerase I promoter, preferably which comprises said chimeric gene.

20. The cell of claim 9, wherein said cell is from a Nannochloropsis sp, preferably Nannochloropsis oceanica.


SEQUENCE LISTING (264 sequences)

SEQ ID NO: 1 - DNA, 924 bp, Nannochloropsis oceanica
gttcggaaactatcgatagggttttggcagttcaactcctcataagtttttctttcaccgtgggcaaaatcggctgtattgctgcccatcacaagacgccctggattcctcccttgtcctcacttcatgagaaggagatgagtgtgtgggagcacagcatttctgcttcgtcatgaccactgagaacgaattgttctgcatgaatgtttgcaatacaatgcgttcttggagatgagagcttctcgacatctcctgccataaacacatgcgcagttgacaagagagcaccaggggctgaggacggaatggtacgtgtgaccgagagatcgaatacgcatccagatccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgggacggcttgataaccggctgttgcaacaaggactatgcatcgaaggtgcacaccctggaagccccgagtcctctgttcgagatactgctccatcacacgagcaggactcacaacgagagttcaatcaatcgggtcagcttcgtatgagtgatgcacctggtgaagttgcagagaggcatcggaagaaggtgctttttgacactgtcacgagcgtgtgtccatgagtcatggcacatctgatctcgtgaccaccttttcattcttacggagaatgagaaacgtctcttgaaaaaatttgctgtccaatcagtggcgatgcggtgttcttgaacacgcttgccctgactgaaccattgaagttgtgtgatgtgtgaagatgaatgtcttcaccataactcaacaacaataacgatagca

SEQ ID NO: 2 - DNA, 929 bp, Nannochloropsis oceanica
cttttctgtacccatcgagtctgcatcagcgacagttcaattcctcaaaaggttttcatggaacccgatcaagtcggctgtattgctgtccatcacaagacgccctggattcctccattgttctttctttatgagaagcagataagtttgtgtgagcacagcagttctgcctcgtcatgaccactgagaaggacttgttttgcatgcttttatgcgatacaatgcgttcttggagatgagagcttctcgacatctcctgcgataaacacacgcgcagttgacaagagagcaccaggggctgaggacggaatggtacgtgtgaccgagagatcgaatacgcatccagaaccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgtgacggctggataaccggctgttgcaacaaggactatgcatcgaaggtgcacaccctggaagccccgagtcctctgttcgagatactgctccatcacacgagcaggactcacaatgagagttcattcaatcgggtcagcttcgtatgagtgatgcacctggtgaagttgcagagaggcatcggaagaaggtgctttttgacactgtcacgagcgtgtgtccatgagtcatggcacatctgatcacgtgaccaccttttcattcttacggagaatgagaaacgtcccttgaaaaaaatttgctgtccaatcagtggcgatgcggtgttcctgaacacgcttgccctgactgaaccattgaagttgtgtgatgtgtggagatgcatgtcttcaccgtaactcatctacaataacgatagca

SEQ ID NO: 3 - DNA, 922 bp, Nannochloropsis oceanica
gttgtctatttaaataggaattctcacggcagttcaactcccattacgttctttctgcgattgagatgctggctgtattgctgcccatcacaaggcgccctggattcctcccttgtcctttcttcatgagaagcagaaaactgtgtgtgagcacagcagttctgtttcgtcatgacatcttagaaggaattgttttgcatgtttgtttgcaatacaatgcattcttggagatgagagcttctcgacatctcctgccataaacacatgcgcagttgacaagagagcaccaggggctgaggacggaatggtacgtgtgaccgagagatcgaatacgcatccagatccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgtgacggttggataaccggctgttgcaacaaggactatgcatcgaaggtgtgcaccctggaagccccgagacctctgttcgagttactgctccatcacacgagcaggactcacaatgagagttcaatcaatcgggtcagcttcgtatgagtgatgcacctggtgaagttgcagagaggcatcggaagaaggtgctttttgacactgtcacgagcgtgtgtccatgagtcatggcacatctggtctcgtgaccaccttttcattcttacggagaatgagaaacgtcccttaaaacatttgctgtccaatcagtggcgatgcggcgttcccgaacacgcttgccctgactgaaccattgaagttgtgtgatgtgtggagatgcatgtcttcaccgtaactcaacaacaattacgatagca

SEQ ID NO: 4 - DNA, 922 bp, Nannochloropsis oceanica
gttgtctatttaaataggaattctcacggcagttcaactcccattacgttctttctgccgttgagatgctggctgtattgctgcccatcacaaggcgccctggattcctcccttgtcctttcttcatgagaagcagaaaactgtgtgtgagcacagcagttctgtttcgtcatgacatcttagaaggaattgttttgcatgtttgtttgcaatacaatgcattcttggagatgagagcttctcgacatctcctgccataaacacatgcgcagttgacaagagagcaccaggggctgaggacggaatggtacgtgtgaccgagagatcgaatacgcatccagatccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgtgacggttggataaccggctgttgcaacaaggactatgcatcgaaggtgcgcaccctggaagccccgagacctctgttcgagatactgctccatcacacgagcaggactcacaatgagagttcaatcaatcgggtcagcttcgtgtgagtgatgcacctggtgaagttgcagagaggcatcggaagaaggtgctttttgacacagtcacgagcgtgtgtccatgagtcatggcacatctgatctcgtgaccaccttttcattcttacggagaatgagaaacgtcccttaaaatatttgctgtccaatcagtggcgatgcggtgttcctgaacacgcttgccctgactgaaccattgaagttgtgtgatgtgtggagatacatgtcttcaccgtaactcaacaacaattacgatagca

SEQ ID NO: 5 - DNA, 255 bp, Artificial Sequence
  N. oceanica (NOC) IRES
  misc_feature (1)..(203): 25S rDNA, partial (3′ 28S rDNA before cassette)
  misc_feature (209)..(255): splice acceptor of NO25G00860 intron 1 (VCP intron)
acaaccagctcagaactggagcggacaaggggaatccgactgtttaattaaaacaaagcattgcgatggtcggagacgatgttgacgcaatgtgatttctgcccagtgctctgaatgtcaaagtgaaggaattcaaccaagcgcgggtaaacggcgggagtaactatgactctcttaaggtagccaaatgcctcgtcatctaattggaatgcccctcccccctccctccttcccttcatcctcccctccgagcag

SEQ ID NO: 6 - DNA, 617 bp, Nannochloropsis oceanica
tggccagggatcaggaggagggagtgaagaggagaagggatctggtttcagagatccccacttctgccgtcgtctttcggccttccttccttttaggtgtcatgccttaggtccttcaagtcctcacctgtcgtcgtcatgtgtgtgtgtgcccgtcatacaagtcactcgatccaattcacgcatcggttcaatcaaaataagactagaccccgagggaagaagggcagaaggaaatcgaaggggtgggatgtgtgtgagagagggaaggagaaatgaaagaagtgaacaatgtcatggtagccagtaaggagagagtagaagcgaagaaagcaaaagcactgttgtgaagaaacgaaatggaagatggtcatcgctcctggctctacttgtggtttttctatctttaatttcaggcgtcctggactcgttacatcagctcccttatctcattggtttatcccctactctactgctgcttcttccttccatccgtgactgtataacaacgaattgtagtaccgcagatagacaatagaaaaatgccaaaaaaggcatcattgatttgctcctccccattaagtcactgtacgccaccatcgccactaccctccctt

SEQ ID NO: 7 - DNA, 691 bp, Nannochloropsis oceanica
tgaaaccactcctagtgagattgcggaaaaccacacaaccgtggaccttcctcgcaattcacgcccccccatcatacctgactccccaatcaacctttctcctcacaaaagtgggctgaagagagtccaagggacgaaggcatatgctcataacacttcgtcaccgcctattagaggatcggcggtgaagaaatgtctccctctttgaaactccgggtagctttttgttaatgtcagatgtttcctggacttgacctattccgagggcgctttctttccctccctacccccgtaaatccgggtagctttttattaaggagacgcaagcgctgtttgcgcacatgccggttacgagacatctgcttccctcggggcaatatctaggagtcacatgtttcttttttgtttcctatgatgatacatgctttggacgatttttcacagggacgaatgccatacacacttttcatctgtccctaagaggatgcacggtaaagaaaaaaaaaaaagttctccgccctccaatctccgggtatcttttatgtttggcagatgtttcctggacttgcctgacgcccctcaaacataccttcgagtctacgaaccatatcttcaccaaccccttctattaagaggctattaagaggaaagacgggtttgtttaatatttcgggtgccctggactcattcc

SEQ ID NO: 8 - DNA, 984 bp, Nannochloropsis oceanica
tgaaaccactcctagtgagattgcggaaaccacacaaccgtggaccttccttgcaattcacgccccccatcatacctgactccccaaacaacctttctcctcacaaaagtgggctgaagagagagtcaaagggacgaaaaggcatatactcataacacttcgtcaccgcctcctctacttcattagactatcggcggtgaagaaatgtctccctcaggccaaaatccgggtagcgttttattaatgtcagatgtttcctggacttggcctatttcgactgcgctttctttctctccatcctccgtaatccgggtagctttttattaatgtcagatgtttcctggacttggcctatttcgactgcgctttctttctctccatcctccgtaatccgggtagctttttattaatgtcatgtttcctggacttggcctatttcgactgcgctttctttctctccatcctccgtaatccgggtagctttttattaatgtcatgtttcctggacggcctattcagagggcgctttctttccctcccttaacccccgtaaatccgggtagctttttatgaaggagacgcaagcgctgtttgcgcacatgccggttacgagacatctgcttccctcggggcaatatctaggagtcacatgtttcttttttgtttcctatgatgatacatggtttggacgatttttcatagggacgaatgccatacacacttttcatctgtccctaagaggatgcacggtaaagaaaaagtgctgcgccctccaatctccggatatcttttatatttggcagatgtttcctggacttgcctgacgcccccaaaacataccttcgagtctacgaaccatatcttcaccaaccccttctattaagaggctattaagaggaaagacgggtttgtttaatatttcgggtgccctggactcgctcccaaccgccgctccccaagggaagagatgttaccttgtttttt

SEQ ID NO: 9 - DNA, 906 bp, Nannochloropsis oceanica
tgaaaccactcctagtgagattgcggaaaccacacaaccgtggaccttcctcgcaattcacgcccccccatcatacctgactccccaaacaacctttctcctcacaaaagtgggctgaagagagagtcaaagggacgaaaaggcatatactcataacacttcgtcaccgcctcctctacttcattagactatcggcggtgaagaaatgtctccctcaggccaaaatccgggtagctttttattaatgtcagatgtttcctggacttggcctatttcgactgcgctttctttgtctccctcttccaaactccgggtagctttttatcaaatgtcagatgtttcctggacttggcctatttcgactgcgcttcctttctctccattctccggaaatccgggtagctttttattaatgtcagatgtttccttgacttgacctattccgagtgcgctctctttcccttcccttaactcccgtaaatccgggtagctttttatgaaggagacgcaagcgctgtttgcgcacatgccggtcacgagagatctgcttccctcggggcaatatctaggagtcacatgtttcttttttgtttcctatgatgatacatgctttggacgatttttcatagggacgaatgccatacacacttttcatctgtccctaagaggatgcacggtaaagaaaaagtgctgcgccctccaatctccgggtatcttttatatttggcagatgtttcctggacctgacgccccccaacataccttcgagtctacgaaccatatcttcaccaaccccttctattaagagactattaagaggaaagacgggtttgtctaatatttcgggtgccttggactcgcccccaaccgccgctcccccacaaccacaccttcctctacctatgg

SEQ ID NO: 10 - DNA, 733 bp, Nannochloropsis oceanica
tgaaaccactcctagtgagattgcggaaaccacacaaccgtggaccttcctcgcaattcacgcccccccatcatacctgactccccaaacaacctttctcctcacaaaagtgggctgaagagagagtcaaagggacgaaaaggcatatgctcataacacttcgtcaccgcctcctctacttgatagaggatgggcggtgaagaaatgtctccctcttccaaactccgggtagctttttatcaaatgtcagatgtttcctggacttggcctatttcgactgcgcttcctttctctccattctccggaaatccgggtagctttttataaaggagacgcaagtgctgtttgcgcacatgccggttacgagacatctgcttccctcggggcaatatctaggagtcacatgtttcttttttgtttcctatgatgatacatgctttggacgatttttcatagggacgaatgccatacacacttttcatctgtccctaagaggatgcacggtaaagaaaaagtgctgcgccctccaatctccgggtatcttttatatttggcagatgtttcctggacctgacgccccccaacataccttcgagtctacgaaccatatcttcaccaaccccttctattaagagactattaagaggaaagacgggtttgtctaatatttcgggtgccttggactcgcccccaaccgccgctcccccacaaccacaccttcctctacctatgg

SEQ ID NO: 11 - DNA, 4265 bp, Artificial Sequence
  EC7
  promoter (1)..(924): Pol I promoter from Nannochloropsis oceanica, chromosome 3
  misc_feature (925)..(1124): ITS2+25S rDNA, bases 1-91, Nannochloropsis oceanica
  misc_feature (1125)..(1379): Noc-IRES
  CDS (1380)..(2093): EGFP coding region (Codon harmonized for Nannochloropsis)
  misc_feature (2094)..(2180): 2A linker peptide sequence (P2A)
  gene (2181)..(2555): Bleomycin/Zeocin resistance gene from Steptoalloteichus hindustanus
  terminator (2556)..(3172): Alpha-tubulin_terminator from Nannochloropsis oceanica
  misc_feature (3173)..(3178): MmeI_recognition-site
  misc_feature (3179)..(3378): 25S rDNA, bases 2334-2533, Nannochloropsis oceanica
  misc_feature (3379)..(3578): 25S rDNA, bases 3191-3390, Nannochloropsis oceanica
  terminator (3579)..(4265): Pol I terminator from Nannochloropsis oceanica chromosome 3
gttcggaaactatcgatagggttttggcagttcaactcctcataagtttttctttcaccgtgggcaaaatcggctgtattgctgcccatcacaagacgccctggattcctcccttgtcctcacttcatgagaaggagatgagtgtgtgggagcacagcatttctgcttcgtcatgaccactgagaacgaattgttctgcatgaatgtttgcaatacaatgcgttcttggagatgagagcttctcgacatctcctgccataaacacatgcgcagttgacaagagagcaccaggggctgaggacggaatggtacgtgtgaccgagagatcgaatacgcatccagatccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgggacggcttgataaccggctgttgcaacaaggactatgcatcgaaggtgcacaccctggaagccccgagtcctctgttcgagatactgctccatcacacgagcaggactcacaacgagagttcaatcaatcgggtcagcttcgtatgagtgatgcacctggtgaagttgcagagaggcatcggaagaaggtgctttttgacactgtcacgagcgtgtgtccatgagtcatggcacatctgatctcgtgaccaccttttcattcttacggagaatgagaaacgtctcttgaaaaaatttgctgtccaatcagtggcgatgcggtgttcttgaacacgcttgccctgactgaaccattgaagttgtgtgatgtgtgaagatgaatgtcttcaccataactcaacaacaataacgatagcaacaccaaacagtttcgacttggcggcatcttctcggtgccataacaaacactgagaaagcctttggactgatcctggcactcgttgccgtgtcattccatctccaattcggacctccaatcaagcaaggctacccgctgaatttaagcatataactaagcggaggaaaagaaactaaccaggattcccctagtaacggcgacaaccagctcagaactggagcggacaaggggaatccgactgtttaattaaaacaaagcattgcgatggtcggagacgatgttgacgcaatgtgatttctgcccagtgctctgaatgtcaaagtgaaggaattcaaccaagcgcgggtaaacggcgggagtaactatgactctcttaaggtagccaaatgcctcgtcatctaattggaatgcccctcccccctccctccttcccttcatcctcccctccgagcagatgagcaagggggaggagttgttcacgggggtggtccccatcttggtggagttggacggggacgtgaacgggcataagtttagcgtcagtggggagggggagggggacgccacgtatgggaagttgacattgaagtttatctgcacgacggggaagttgcccgtgccctggcccacattggtcacgacgttgacgtacggggtgcagtgctttagccggtatcccgaccacatgaagcagcacgattttttcaagagcgcaatgcccgaggggtacgtgcaggagcggacgatctttttcaaggacgatgggaattataagacacgggccgaggtcaagtttgagggggacacattggtgaaccggattgagttgaaggggatcgactttaaggaggacgggaatatcttggggcataagttggagtataattacaatagccataacgtgtatattatggccgataagcagaagaacgggattaaggtgaatttcaagatccggcataatatcgaggacgggagcgtgcagttggccgatcactaccagcagaacacgcccatcggggacgggcccgtcttgttgcccgataatcactatttgagtacgcagagcgcattgagtaaggaccccaatgagaagcgggatcatatggtcttgttggagtttgtgacggccgccgggatcacacacgggatcgacgagttgtataagatgacgaccctgtccttccaggggccgggggcgacgaacttctccttgctgaagcaggccggggacgtggaggagaacccggggcccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatggccagggatcaggaggagggagtgaagaggagaagggatctggtttcagagatccccacttctgccgtcgtctttcggccttccttccttttaggtgtcatgccttaggtccttcaagtcctcacctgtcgtcgtcatgtgtgtgtgtgcccgtcatacaagtcactcgatccaattcacgcatcggttcaatcaaaataagactagaccccgagggaagaagggcagaaggaaatcgaaggggtgggatgtgtgtgagagagggaaggagaaatgaaagaagtgaacaatgtcatggtagccagtaaggagagagtagaagcgaagaaagcaaaagcactgttgtgaagaaacgaaatggaagatggtcatcgctcctggctctacttgtggtttttctatctttaatttcaggcgtcctggactcgttacatcagctcccttatctcattggtttatcccctactctactgctgcttcttccttccatccgtgactgtataacaacgaattgtagtaccgcagatagacaatagaaaaatgccaaaaaaggcatcattgatttgctcctccccattaagtcactgtacgccaccatcgccactaccctccctttccaactaattagtgacgcgcatgaatggattaatgagattcccactgtccctatctgccatctagcgaacccacagccaagggaacgggcttggaataatcagcggggaaagaagaccctgttgagcttgactctagtccgacgttgtgaaatgacttatgaggtgtagcataagtgggagctccggcgacgatgaaataccactcgacgatattcacctcttctgatgctacgcgtatcgcgataggcttcggcccagcatcatacgtcatcatcatttcaatcattagaagagataaatcctctgtagacgactttgatatggaacggggtattgtaagtgtgagagtaggctttgtcctacgatccactgagattcagcctcttgttccgtcgatttgttgttgaaaccactcctagtgagattgcggaaaaccacacaaccgtggaccttcctcgcaattcacgcccccccatcatacctgactccccaatcaacctttctcctcacaaaagtgggctgaagagagtccaagggacgaaggcatatgctcataacacttcgtcaccgcctattagaggatcggcggtgaagaaatgtctccctctttgaaactccgggtagctttttgttaatgtcagatgtttcctggacttgacctattccgagggcgctttctttccctccctacccccgtaaatccgggtagctttttattaaggagacgcaagcgctgtttgcgcacatgccggttacgagacatctgcttccctcggggcaatatctaggagtcacatgtttcttttttgtttcctatgatgatacatgctttggacgatttttcacagggacgaatgccatacacacttttcatctgtccctaagaggatgcacggtaaagaaaaaaaaaaaagttctccgccctccaatctccgggtatcttttatgtttggcagatgtttcctggacttgcctgacgcccctcaaacataccttcgagtctacgaaccatatcttcaccaaccccttctattaagaggctattaagaggaaagacgggtttgtttaatatttcgggtgccctggactca

SEQ ID NO: 12 - protein, 238 aa, Artificial Sequence
  Synthetic Construct
MSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGIDELYK

SEQ ID NO: 13 - DNA, 4979 bp, Artificial Sequence
  EC7-tdTomato
  promoter (1)..(924): Pol I promoter from Nannochloropsis oceanica, chromosome 3
  misc_feature (925)..(1124): ITS2+25S rDNA, bases 1-91, Nannochloropsis oceanica
  misc_feature (1125)..(1379): Noc-IRES
  CDS (1380)..(2807): coding sequence for the fluorescent reporter tdTomato
  misc_feature (2808)..(2894): 2A linker peptide sequence (P2A)
  gene (2895)..(3269): Bleomycin/Zeocin resistance gene from Streptoalloteichus hindustanus
  terminator (3270)..(3886): Alpha-tubulin terminator from Nannochloropsis oceanica
  misc_feature (3887)..(3892): MmeI_recognition-site
  misc_feature (3893)..(4092): 25S rDNA, bases 2334-2533, Nannochloropsis oceanica
  misc_feature (4093)..(4292): 25S rDNA, bases 3191-3390, Nannochloropsis oceanica
  terminator (4293)..(4979): Pol I_terminator from Nannochloropsis oceanica chromosome 3
gttcggaaactatcgatagggttttggcagttcaactcctcataagtttttctttcaccgtgggcaaaatcggctgtattgctgcccatcacaagacgccctggattcctcccttgtcctcacttcatgagaaggagatgagtgtgtgggagcacagcatttctgcttcgtcatgaccactgagaacgaattgttctgcatgaatgtttgcaatacaatgcgttcttggagatgagagcttctcgacatctcctgccataaacacatgcgcagttgacaagagagcaccaggggctgaggacggaatggtacgtgtgaccgagagatcgaatacgcatccagatccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgggacggcttgataaccggctgttgcaacaaggactatgcatcgaaggtgcacaccctggaagccccgagtcctctgttcgagatactgctccatcacacgagcaggactcacaacgagagttcaatcaatcgggtcagcttcgtatgagtgatgcacctggtgaagttgcagagaggcatcggaagaaggtgctttttgacactgtcacgagcgtgtgtccatgagtcatggcacatctgatctcgtgaccaccttttcattcttacggagaatgagaaacgtctcttgaaaaaatttgctgtccaatcagtggcgatgcggtgttcttgaacacgcttgccctgactgaaccattgaagttgtgtgatgtgtgaagatgaatgtcttcaccataactcaacaacaataacgatagcaacaccaaacagtttcgacttggcggcatcttctcggtgccataacaaacactgagaaagcctttggactgatcctggcactcgttgccgtgtcattccatctccaattcggacctccaatcaagcaaggctacccgctgaatttaagcatataactaagcggaggaaaagaaactaaccaggattcccctagtaacggcgacaaccagctcagaactggagcggacaaggggaatccgactgtttaattaaaacaaagcattgcgatggtcggagacgatgttgacgcaatgtgatttctgcccagtgctctgaatgtcaaagtgaaggaattcaaccaagcgcgggtaaacggcgggagtaactatgactctcttaaggtagccaaatgcctcgtcatctaattggaatgcccctcccccctccctccttcccttcatcctcccctccgagcagatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccgctacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagatgacgaccctgtccttccaggggccgggggcgacgaacttctccttgctgaagcaggccggggacgtggaggagaacccggggcccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatggccagggatcaggaggagggagtgaagaggagaagggatctggtttcagagatccccacttctgccgtcgtctttcggccttccttccttttaggtgtcatgccttaggtccttcaagtcctcacctgtcgtcgtcatgtgtgtgtgtgcccgtcatacaagtcactcgatccaattcacgcatcggttcaatcaaaataagactagaccccgagggaagaagggcagaaggaaatcgaaggggtgggatgtgtgtgagagagggaaggagaaatgaaagaagtgaacaatgtcatggtagccagtaaggagagagtagaagcgaagaaagcaaaagcactgttgtgaagaaacgaaatggaagatggtcatcgctcctggctctacttgtggtttttctatctttaatttcaggcgtcctggactcgttacatcagctcccttatctcattggtttatcccctactctactgctgcttcttccttccatccgtgactgtataacaacgaattgtagtaccgcagatagacaatagaaaaatgccaaaaaaggcatcattgatttgctcctccccattaagtcactgtacgccaccatcgccactaccctccctttccaactaattagtgacgcgcatgaatggattaatgagattcccactgtccctatctgccatctagcgaacccacagccaagggaacgggcttggaataatcagcggggaaagaagaccctgttgagcttgactctagtccgacgttgtgaaatgacttatgaggtgtagcataagtgggagctccggcgacgatgaaataccactcgacgatattcacctcttctgatgctacgcgtatcgcgataggcttcggcccagcatcatacgtcatcatcatttcaatcattagaagagataaatcctctgtagacgactttgatatggaacggggtattgtaagtgtgagagtaggctttgtcctacgatccactgagattcagcctcttgttccgtcgatttgttgttgaaaccactcctagtgagattgcggaaaaccacacaaccgtggaccttcctcgcaattcacgcccccccatcatacctgactccccaatcaacctttctcctcacaaaagtgggctgaagagagtccaagggacgaaggcatatgctcataacacttcgtcaccgcctattagaggatcggcggtgaagaaatgtctccctctttgaaactccgggtagctttttgttaatgtcagatgtttcctggacttgacctattccgagggcgctttctttccctccctacccccgtaaatccgggtagctttttattaaggagacgcaagcgctgtttgcgcacatgccggttacgagacatctgcttccctcggggcaatatctaggagtcacatgtttcttttttgtttcctatgatgatacatgctttggacgatttttcacagggacgaatgccatacacacttttcatctgtccctaagaggatgcacggtaaagaaaaaaaaaaaagttctccgccctccaatctccgggtatcttttatgtttggcagatgtttcctggacttgcctgacgcccctcaaacataccttcgagtctacgaaccatatcttcaccaaccccttctattaagaggctattaagaggaaagacgggtttgtttaatatttcgggtgccctggactca

SEQ ID NO: 14 - protein, 476 aa, Artificial Sequence
  Synthetic Construct
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGRYLVEFKTIYMAKKPVQLPGYYYVDTKLDITSHNEDYTIVEQYERSEGRHHLFLYGMDELYK

SEQ ID NO: 15 - DNA, 6248 bp, Artificial Sequence
  ECT2-tdTomato
  misc_feature (1)..(1143): Left homology flank, targeting the EC to the NOR on chromosome 3 of Nannochloropsis oceanica, replacing the endogenous rDNA cistron (when transforming wild type)
  promoter (1144)..(2067): Pol I promoter from Nannochloropsis oceanica, chromosome 3
  misc_feature (2068)..(2267): ITS2, partial + 25S rDNA, bases 1-91 from N. oceanica
  misc_feature (2268)..(2522): Noc-IRES
  misc_feature (2523)..(3950): coding sequence for the fluorescent reporter tdTomato
  misc_feature (3951)..(4037): 2A linker peptide sequence
  gene (4038)..(4412): Bleomycin/Zeocin resistance gene from Streptoalloteichus hindustanus
  terminator (4413)..(5029): Alpha-tubulin_terminator from Nannochloropsis oceanica
  misc_feature (5030)..(5035): MmeI_recognition-site
  misc_feature (5036)..(6248): Right homology flank, targeting the EC to the NOR on chromosome 3 of Nannochloropsis oceanica, replacing the endogenous rDNA cistron (when transforming wild type)
aggtaaggagtagggagggggagagagaaataggagagcgagaatgagcggaaggtgagcaagaggaagtagttatacgacagcattagaagcagcgagaggcaaccgagaaattgctggggggagcggcaaggttctaagaaattggtaagagaaccaaaatcaatagggcaacgcattgtcctgaagaagaaataagaaccaaaccctgcgacagcaatgctttgttttgaaaaacaattgcttgtacctgctgacactccacttaagcctacccatgctgccactgctattgctaccgccactgcaaccatcccgactacggttgcccttttctcctcggcaacgagccataagagggcaccgccaactcaacaactcaaaaacagaccccttgacccaccctcccaacccaatcccacttgagggccagggcggtgcggagcttttgacttttaccctccgagtcctcaccgtctgcgtggccttccggaggatggatccaacggcacgcgcgataagtgccccccttccaccagcccgcatggcggcgacggcatggcgggccttgacgaggaatgaggcctgactcgatgataagcagaggcagggcgagtgctgcgacttacaccagcttgacttgccacggcatcaggaagcaccgaagtgctaggtggagacgcgatggccgtggtcgtgaggttcatgccgcagaaggagcggaggcgctactcctgttgcagcgctatcactgttgcagcactatatgtaacaacttattgattccatcgttctgtacaaagtgcatctttggtgttcaattgggaaaggaacgtgagacaaggccacatcgttctcacaggcctgcatgacgaaaactatgcaggcggcggaaaaagagcaagggaggctgcgtaacacaacgagaacaaaacaaaaagtgtgactggcatttttgtgtgtgttaacagctggtcgcagtgatgggaatcaaaatgcaaaaaggaaaaagggagaagaacgaaagtacgcaggcctacaacgtgtgagcgtcttcatttccacgacgtctcatcacacgaattgtccatcatgtattctagcgatactcgaaaagaattcaagagaagaagaaagagcttagttcggaaactatcgatagggttttggcagttcaactcctcataagtttttctttcaccgtgggcaaaatcggctgtattgctgcccatcacaagacgccctggattcctcccttgtcctcacttcatgagaaggagatgagtgtgtgggagcacagcatttctgcttcgtcatgaccactgagaacgaattgttctgcatgaatgtttgcaatacaatgcgttcttggagatgagagcttctcgacatctcctgccataaacacatgcgcagttgacaagagagcaccaggggctgaggacggaatggtacgtgtgaccgagagatcgaatacgcatccagatccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgggacggcttgataaccggctgttgcaacaaggactatgcatcgaaggtgcacaccctggaagccccgagtcctctgttcgagatactgctccatcacacgagcaggactcacaacgagagttcaatcaatcgggtcagcttcgtatgagtgatgcacctggtgaagttgcagagaggcatcggaagaaggtgctttttgacactgtcacgagcgtgtgtccatgagtcatggcacatctgatctcgtgaccaccttttcattcttacggagaatgagaaacgtctcttgaaaaaatttgctgtccaatcagtggcgatgcggtgttcttgaacacgcttgccctgactgaaccattgaagttgtgtgatgtgtgaagatgaatgtcttcaccataactcaacaacaataacgatagcaacaccaaacagtttcgacttggcggcatcttctcggtgccataacaaacactgagaaagcctttggactgatcctggcactcgttgccgtgtcattccatctccaattcggacctccaatcaagcaaggctacccgctgaatttaagcatataactaagcggaggaaaagaaactaaccaggattcccctagtaacggcgacaaccagctcagaactggagcggacaaggggaatccgactgtttaattaaaacaaagcattgcgatggtcggagacgatgttgacgcaatgtgatttctgcccagtgctctgaatgtcaaagtgaaggaattcaaccaagcgcgggtaaacggcgggagtaactatgactctcttaaggtagccaaatgcctcgtcatctaattggaatgcccctcccccctccctccttcccttcatcctcccctccgagcagatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccgctacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagatgacgaccctgtccttccaggggccgggggcgacgaacttctccttgctgaagcaggccggggacgtggaggagaacccggggcccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatggccagggatcaggaggagggagtgaagaggagaagggatctggtttcagagatccccacttctgccgtcgtctttcggccttccttccttttaggtgtcatgccttaggtccttcaagtcctcacctgtcgtcgtcatgtgtgtgtgtgcccgtcatacaagtcactcgatccaattcacgcatcggttcaatcaaaataagactagaccccgagggaagaagggcagaaggaaatcgaaggggtgggatgtgtgtgagagagggaaggagaaatgaaagaagtgaacaatgtcatggtagccagtaaggagagagtagaagcgaagaaagcaaaagcactgttgtgaagaaacgaaatggaagatggtcatcgctcctggctctacttgtggtttttctatctttaatttcaggcgtcctggactcgttacatcagctcccttatctcattggtttatcccctactctactgctgcttcttccttccatccgtgactgtataacaacgaattgtagtaccgcagatagacaatagaaaaatgccaaaaaaggcatcattgatttgctcctccccattaagtcactgtacgccaccatcgccactaccctccctttccaactcatcgtcatcgtcctttcccgtacgagaaatttaaaagctagacctctacttaccccctactattaacccgctcatcctgacctctcactcctcttcctcatcttcctccaccaccggcacaccctccacttctccttcgtgccgtacaggtaacaaagccaacctctcgagcacttctaacgctgggcccttggggttgccatggaccagggcctctatctgtcgacgttgagagcgtgtcatggatagctcaaaatcaattatcacatctactaccgacagggcattcttccagctttcactaattagcaaactctctagcaatgtcacgagaatatccgccccagcgacatcatctcgatctcgcatctcttgcagcaagcccgcaacccgatccaggcgacctcgatccacgtgacagagcgctgctactgctgcagggtccccgccctgaaatgctggaggcaaattgtgatatacatcaaatgccttctcaatctgctttggattcgccgcccagcatccacggagcaaagacccatagaaccctcggtccgccttccacccctgttcctccaccaggccgaggatggtttcagctgcggccaggtcaggcggatctagcttggtgcaatagacaaacgtggcccaccggtaagtgtcataacttgtgtttggcttcaagaccaagtcccccagcagatctctcgcccggtccgactggccaacgcgtaaggaggcgagaatcgcagggacacctaagcgatcatgttttaccagcatctcccgcggtaaggtagcagccacgtccgccagctcctggaagcgacatgcttgcttcagacacttgaaaagccggcggtaggtcagtgggttcggcatcgaggatggcccatggctgagcatttcgtgatatagacgcaaagccctctccgacatgcccgtcgggtcatcacgcaccaaacggtaggtcacgtacatcgcctcattgtacatgatcgtgttgggggtacgcccactcgctttgaatgtctcaagcaattctaaggcctcacgtcggcttttcacatctgccttggccaacgcatgcatgaatgcaccgaacatgatatcattcgaagcctcttccggatcgcccgccaacgttctgaagcgcgtcagaaggccattcgcctgcgtgcgattcctcaaatccgccgtcacactaatca

SEQ ID NO: 16 - DNA, 6163 bp, Artificial Sequence
  EC-VHH
  misc_feature (1)..(1143): Left homology flank, targeting the EC to the NOR on chromosome 3 of Nannochloropsis oceanica, replacing the endogenous rDNA cistron (when transforming wt) or EC7-tdTomato (when transforming EC7-tdTomato-S1) or ECT2-tdTomato (when transforming
  promoter (1144)..(2067): Pol I promoter from Nannochloropsis oceanica, chromosome 3
  misc_feature (2068)..(2267): ITS2+25S rDNA, bases 1-91, Nannochloropsis oceanica
  misc_feature (2268)..(2522): Noc-IRES
  CDS (2523)..(2870): GFP-binding fragment of a single-chain camelid antibody
  misc_feature (2874)..(2900): Human influenca hemagglutinin-tag (HA-tag)
  misc_feature (2904)..(2921): 6xHis affinity tag (6xHis tag)
  terminator (2925)..(3541): Alpha-tubulin_terminator from Nannochloropsis oceanica
  misc_feature (3542)..(3547): MmeI recognition site
  promoter (3548)..(4317): Transcriptional promoter (RNA Polymerase II) of the Nannochloropsis oceanica LDSP gene (LDSP promoter)
  gene (4318)..(4713): Blasticidin resistance gene
  terminator (4714)..(4950): Cauliflower mosaic virus transcriptional terminator (RNA polymerase II) (CaMV 35S terminator)
  misc_feature (4951)..(6163): Right homology flank, targeting the EC to the NOR on chromosome 3 of Nannochloropsis oceanica, replacing the endogenous rDNA cistron (when transforming wt) or EC7-tdTomato (when transforming EC7-tdTomato-S1) or ECT2-tdTomato (when transforming
aggtaaggagtagggagggggagagagaaataggagagcgagaatgagcggaaggtgagcaagaggaagtagttatacgacagcattagaagcagcgagaggcaaccgagaaattgctggggggagcggcaaggttctaagaaattggtaagagaaccaaaatcaatagggcaacgcattgtcctgaagaagaaataagaaccaaaccctgcgacagcaatgctttgttttgaaaaacaattgcttgtacctgctgacactccacttaagcctacccatgctgccactgctattgctaccgccactgcaaccatcccgactacggttgcccttttctcctcggcaacgagccataagagggcaccgccaactcaacaactcaaaaacagaccccttgacccaccctcccaacccaatcccacttgagggccagggcggtgcggagcttttgacttttaccctccgagtcctcaccgtctgcgtggccttccggaggatggatccaacggcacgcgcgataagtgccccccttccaccagcccgcatggcggcgacggcatggcgggccttgacgaggaatgaggcctgactcgatgataagcagaggcagggcgagtgctgcgacttacaccagcttgacttgccacggcatcaggaagcaccgaagtgctaggtggagacgcgatggccgtggtcgtgaggttcatgccgcagaaggagcggaggcgctactcctgttgcagcgctatcactgttgcagcactatatgtaacaacttattgattccatcgttctgtacaaagtgcatctttggtgttcaattgggaaaggaacgtgagacaaggccacatcgttctcacaggcctgcatgacgaaaactatgcaggcggcggaaaaagagcaagggaggctgcgtaacacaacgagaacaaaacaaaaagtgtgactggcatttttgtgtgtgttaacagctggtcgcagtgatgggaatcaaaatgcaaaaaggaaaaagggagaagaacgaaagtacgcaggcctacaacgtgtgagcgtcttcatttccacgacgtctcatcacacgaattgtccatcatgtattctagcgatactcgaaaagaattcaagagaagaagaaagagcttagttcggaaactatcgatagggttttggcagttcaactcctcataagtttttctttcaccgtgggcaaaatcggctgtattgctgcccatcacaagacgccctggattcctcccttgtcctcacttcatgagaaggagatgagtgtgtgggagcacagcatttctgcttcgtcatgaccactgagaacgaattgttctgcatgaatgtttgcaatacaatgcgttcttggagatgagagcttctcgacatctcctgccataaacacatgcgcagttgacaagagagcaccaggggctgaggacggaatggtacgtgtgaccgagagatcgaatacgcatccagatccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgggacggcttgataaccggctgttgcaacaaggactatgcatcgaaggtgcacaccctggaagccccgagtcctctgttcgagatactgctccatcacacgagcaggactcacaacgagagttcaatcaatcgggtcagcttcgtatgagtgatgcacctggtgaagttgcagagaggcatcggaagaaggtgctttttgacactgtcacgagcgtgtgtccatgagtcatggcacatctgatctcgtgaccaccttttcattcttacggagaatgagaaacgtctcttgaaaaaatttgctgtccaatcagtggcgatgcggtgttcttgaacacgcttgccctgactgaaccattgaagttgtgtgatgtgtgaagatgaatgtcttcaccataactcaacaacaataacgatagcaacaccaaacagtttcgacttggcggcatcttctcggtgccataacaaacactgagaaagcctttggactgatcctggcactcgttgccgtgtcattccatctccaattcggacctccaatcaagcaaggctacccgctgaatttaagcatataactaagcggaggaaaagaaactaaccaggattcccctagtaacggcgacaaccagctcagaactggagcggacaaggggaatccgactgtttaattaaaacaaagcattgcgatggtcggagacgatgttgacgcaatgtgatttctgcccagtgctctgaatgtcaaagtgaaggaattcaaccaagcgcgggtaaacggcgggagtaactatgactctcttaaggtagccaaatgcctcgtcatctaattggaatgcccctcccccctccctccttcccttcatcctcccctccgagcagatgcaggtccagttggtcgagagcggcggcgcattggtccagccaggcggcagcttgcggttgagctgtgcagcaagcggctttcccgtcaatcggtacagcatgcggtggtaccggcaggcacccggcaaggagcgggagtgggtcgcaggcatgagctctgcgggcgaccggagcagctacgaggacagcgtcaagggccggtttacgattagccgggacgacgcacggaatacggtctacttgcaaatgaatagcttgaagcccgaggacacggcagtgtactactgcaatgtcaacgtggggtttgagtactggggccaggggacgcaggtcacggtcagcagcggctacccctatgacgtgcccgactacgccggccatcaccatcaccatcactgatggccagggatcaggaggagggagtgaagaggagaagggatctggtttcagagatccccacttctgccgtcgtctttcggccttccttccttttaggtgtcatgccttaggtccttcaagtcctcacctgtcgtcgtcatgtgtgtgtgtgcccgtcatacaagtcactcgatccaattcacgcatcggttcaatcaaaataagactagaccccgagggaagaagggcagaaggaaatcgaaggggtgggatgtgtgtgagagagggaaggagaaatgaaagaagtgaacaatgtcatggtagccagtaaggagagagtagaagcgaagaaagcaaaagcactgttgtgaagaaacgaaatggaagatggtcatcgctcctggctctacttgtggtttttctatctttaatttcaggcgtcctggactcgttacatcagctcccttatctcattggtttatcccctactctactgctgcttcttccttccatccgtgactgtataacaacgaattgtagtaccgcagatagacaatagaaaaatgccaaaaaaggcatcattgatttgctcctccccattaagtcactgtacgccaccatcgccactaccctccctttccaacgatggagtggatggaggaggaggcgagcgtagcagcaagcgtgagttatacagccaggcacatgtcgcaatccttcggtctcgggcttaaaatccacgcactaatcacgctgggccatgcaaagagcaatgccgaggcccaccacacaaaacgctgtgtcgcgcgttgcggcctgaagcttcatacttcttagtcgccgccaaaagggctcgagagacgagacccgttggcatgaccgatgttgttcgacgcggtttgcttcgtcacagtcgacgtgattcaggaatctggagcctgcagatcatttttttcagcctgatatcgttcttttccactgagaaccatcagaccaccttttcttccattgtgtgaaggagtaggagttgccgtgctgctttgtgggagacatctgcgatggtgaccagcctcccgtcgtctggtcgacgtgacgagcctcttcactgttcttcgacggagagacgcaagcgagacggctctagaccttttggacacgcattctgtgtgtgaactagtggacagtgataccacgtctgaaagctcaccactgcccatggtgcagctacttgtcacaaagttttgactccgtcggtatcaccattcgcgctcgtgtgcctggttgttccgccacgccggcctgccccggggcggggcaatattctaaaatctcacgcaaaacaccgcacttacccctcacacatattcgtgatagaccaccaccaatctcagcccgcatcaacacaggagggcccatgcccctctcgcaggaggagagcacgctgatcgagagggctacggcgacgattaactcgattcctatctccgaggattactcggtggcttccgctgctctgtcgtccgacggacggatcttcacgggggtcaacgtgtaccattttacgggggggccttgcgcggagctggtggtcctggggaccgccgctgctgccgcggctgggaacctgacgtgcattgtcgccatcgggaacgagaaccgcgggattctgagcccctgcgggcggtgccggcaggtcctgctcgacctccaccctgggatcaaggcgattgtgaaggattccgacgggcagcctacggcggtcggtatcagagaacttctcccttcgggctacgtgtgggagagcgcttgaactagtcaggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcggactccggtcggcatctacttcatcgtcatcgtcctttcccgtacgagaaatttaaaagctagacctctacttaccccctactattaacccgctcatcctgacctctcactcctcttcctcatcttcctccaccaccggcacaccctccacttctccttcgtgccgtacaggtaacaaagccaacctctcgagcacttctaacgctgggcccttggggttgccatggaccagggcctctatctgtcgacgttgagagcgtgtcatggatagctcaaaatcaattatcacatctactaccgacagggcattcttccagctttcactaattagcaaactctctagcaatgtcacgagaatatccgccccagcgacatcatctcgatctcgcatctcttgcagcaagcccgcaacccgatccaggcgacctcgatccacgtgacagagcgctgctactgctgcagggtccccgccctgaaatgctggaggcaaattgtgatatacatcaaatgccttctcaatctgctttggattcgccgcccagcatccacggagcaaagacccatagaaccctcggtccgccttccacccctgttcctccaccaggccgaggatggtttcagctgcggccaggtcaggcggatctagcttggtgcaatagacaaacgtggcccaccggtaagtgtcataacttgtgtttggcttcaagaccaagtcccccagcagatctctcgcccggtccgactggccaacgcgtaaggaggcgagaatcgcagggacacctaagcgatcatgttttaccagcatctcccgcggtaaggtagcagccacgtccgccagctcctggaagcgacatgcttgcttcagacacttgaaaagccggcggtaggtcagtgggttcggcatcgaggatggcccatggctgagcatttcgtgatatagacgcaaagccctctccgacatgcccgtcgggtcatcacgcaccaaacggtaggtcacgtacatcgcctcattgtacatgatcgtgttgggggtacgcccactcgctttgaatgtctcaagcaattctaaggcctcacgtcggcttttcacatctgccttggccaacgcatgcatgaatgcaccgaacatgatatcattcgaagcctcttccggatcgcccgccaacgttctgaagcgcgtcagaaggccattcgcctgcgtgcgattcctcaaatccgccgtcacactaatca

SEQ ID NO: 17 - protein, 116 aa, Artificial Sequence
  Synthetic Construct
MQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 18 - DNA, 3539 bp, Artificial Sequence
  PPEC-TEV-26S
  misc_feature (1)..(1000): Left homology flank, targeting the EC to the 26S gene of Pichia pastoris
  misc_feature (1001)..(1146): Internal Ribosome Entry Site from Tobacco Etch Virus (TEV IRES)
  misc_feature (1147)..(1860): yEGFP coding region (codons optimized for translation in S. cerevisiae and C. albicans)
  misc_feature (1861)..(1917): 2A peptide from porcine teschovirus-1 polyprotein (P2A)
  gene (1918)..(2292): Bleomycin/Zeocin resistance gene from Streptoalloteichus hindustanus
  terminator (2293)..(2539): Transcriptional terminator (RNA polymerase II) of the AOX1 gene of Pichia pastoris (AOX1 terminator)
  misc_feature (2540)..(3539): Left homology flank, targeting the EC to the 26S gene of Pichia pastoris
gaccccctcagtgggccatttttggtaagcagaactggcgatgcgggatgaaccgaacgcagggttaaggtgccggaagcacgctcaaagacaccacaaaaggtgttggttcatctggacagccggacggtggccatggaagtcggaatccgccaaggagtgtgtaacaactcaccggccgaatgaactagccctgaaaatggatggcgctaagcgtgctacctataccctgccggagaacagaaagctctccgagtaggcaggcgtgggggtggtgtggaagggtgcttgtgaaggtgcctggaaccgcccctagtgcagatcttggtggcagtagcaatattcaacggagccgttgaagaccgaagtggggaaaggttccacgggaagggagatcctccgtgggtgagacggtcctaagggcgcgcgtacggtagcgtccgaaagggaagacagtcaagattctgtctccgggggaatgagtggtgacacgagacggccaaagacggtggcggaaggcgtaggaggagttttcttttcttcttaagggcgttgtgcctggatggtttagccggcgaagggcagtaaaacccgaagagcgtgcccctagttggtgcgtctacgcatctccgacatcccgtgaaaatttggtttatttatgttattcccccgcccgtactgacaaccgcagcaggtctccaaggtgaacagcctctggtgatagaacaatgtagataagggaagtcggcaaattggatccgtaacctcgggacaaggattggctctgggggtcgcttttatcgaccaacccagaactggtacggacaaagggaatctgactgtctaattaaaacatagggtggtgcgagtcccaacggatgtgcacaccacctgatttctgcccagtgctctgaatgtcaaagtgaagaaattcatccaagcgcgggtaaacggcgggagtaactatgactctcttaaggtagccaaatgcctcgtcatcaaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagcaatgatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaacttatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtattgatgaattgtacaaagctactaatttttctttgttaaaacaagctggtgatgttgaagaaaatcctggtccaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagataattagtgacgcgcatgaatggattaacgagattcccactgtccctatctactatctagcgaaaccacagccaagggaacgggcttggcgcgtcagcggggaaagaagaccctgttgagcttgactctagtttgggccgtgggggaacatggggggtgtagtataggggggagccgtttcggcggcgcccgtgaaatacccccacccctatagttcctccactaatcgcagagaaacctccaggcggggagtttggctggggcggcacatctgttaaaccataacgcagatgtcccaaggggggctcatgaagaacagaaatcttcagccgagcaaaagggcaaatgcccccttgattttgattttcagtgtgaatacaaaccatgaaagtgtggcctatcgatcctttacaccctcagaccctgaggctagaggtgccagaaaagttaccacagggataactggcttgtggcagtcaagcgttcatagcgacattgctttttgattcttcgatgtcggctcttcctatcataccgaagcagaattcggtaagcgttggattgttcacccactaatagggaacgtgagctgggtttagaccgtcgtgagacaggttagttttaccctactgatgagccccacggcagtaattgaacttagtacgagaggaaccgttcattcggacatttggtttttggggctgcctgacaaagcagccccgacgctacgtccgtcagatcatggctgaacgcctctaagtcagaatctgtgctggggtggggaggagtgtacaagaaggatatatttcatagcagcattccacaatctcccgcccacgacttgaccccgacagggtattgtacgcagaagagtggccttgccgtcacgatctgctgagattaagccccggtcgagcgattggaacttccataccgaaggaaacacaccgagcgcagcgaggttgtgtttccgaagacgtaatcccaccaagcggtacca

SEQ ID NO: 19 - DNA, 196 bp, Tobacco etch virus
gccaagcttgcatgcgagtccccggaaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagcaatgatgtctaaaggtgaagaattattca

SEQ ID NO: 20 - DNA, 2508 bp, Artificial Sequence
  yEC2
  misc_feature (1)..(300): Homology flank, directing yEC2 to the 25S rDNA of Saccharomyces cerevisiae
  misc_feature (301)..(555): Putative IRES-encoding element analogous to Noc-IRES of Nannochloropsis oceanica, but with Saccharomyces cerevisiae 25S rDNA sequence
  misc_feature (556)..(1269): yEGFP (codons optimized for translation in S. cerevisiae and C. albicans)
  misc_feature (1270)..(1326): 2A peptide from porcine teschovirus-1 polyprotein
  gene (1327)..(2130): S. cerevisiae orotidine-5′-phosphate decarboxylase, required for uracil biosynthesis (URA3)
  terminator (2131)..(2208): URA3 RNA polymerase II transcriptional terminator
  misc_feature (2209)..(2508): Homology flank, directing yEC2 to the 25S rDNA of Saccharomyces cerevisiae
gtgaaaatccacaggaaggaatagttttcatgccaggtcgtactgataaccgcagcaggtctccaaggtgaacagcctctagttgatagaataatgtagataagggaagtcggcaaaatagatccgtaacttcgggataaggattggctctaagggtcgggtagtgagggccttggtcagacgcagcgggcgtgcttgtggactgcttggtggggcttgctctgctaggcggactacttgcgtgccttgttgtagacggccttggtaggtctcttgtagaccgtcgcttgctacaattaacgatcaacttagaactggtacggacaaggggaatctgactgtctaattaaaacatagcattgcgatggtcagaaagtgatgttgacgcaatgtgatttctgcccagtgctctgaatgtcaaagtgaagaaattcaaccaagcgcgggtaaacggcgggagtaactatgactctcttaaggtagccaaatgcctcgtcatctaattggaatgcccctcccccctccctccttcccttcatcctcccctccgagcagatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaacttatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtattgatgaattgtacaaagctactaatttttctttgttaaaacaagctggtgatgttgaagaaaatcctggtccaatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaagagacatagagggtgtagaataagtgggagcttcggcgccagtgaaataccactacctttatagtttctttacttattcaatgaagcggagctggaattcattttccacgttctagcattcaaggtcccattcggggctgatccgggttgaaga

SEQ ID NO: 21 - DNA, 255 bp, Artificial Sequence
  Sce-IRES
  misc_feature (1)..(203): S. cerevisiae RDN25-1, partial
  misc_feature (209)..(255): splice acceptor of N. oceanica NO25G00860 intron 1 (VCP intron)
cgatcaacttagaactggtacggacaaggggaatctgactgtctaattaaaacatagcattgcgatggtcagaaagtgatgttgacgcaatgtgatttctgcccagtgctctgaatgtcaaagtgaagaaattcaaccaagcgcgggtaaacggcgggagtaactatgactctcttaaggtagccaaatgcctcgtcatctaattggaatgcccctcccccctccctccttcccttcatcctcccctccgagcag

SEQ ID NO: 22 - DNA, 791 bp, Artificial Sequence
  EGFP codon harmonized for N. oceanica
cttcatcctcccctccgagcagatgagcaagggggaggagttgttcacgggggtggtccccatcttggtggagttggacggggacgtgaacgggcataagtttagcgtcagtggggagggggagggggacgccacgtatgggaagttgacattgaagtttatctgcacgacggggaagttgcccgtgccctggcccacattggtcacgacgttgacgtacggggtgcagtgctttagccggtatcccgaccacatgaagcagcacgattttttcaagagcgcaatgcccgaggggtacgtgcaggagcggacgatctttttcaaggacgatgggaattataagacacgggccgaggtcaagtttgagggggacacattggtgaaccggattgagttgaaggggatcgactttaaggaggacgggaatatcttggggcataagttggagtataattacaatagccataacgtgtatattatggccgataagcagaagaacgggattaaggtgaatttcaagatccggcataatatcgaggacgggagcgtgcagttggccgatcactaccagcagaacacgcccatcggggacgggcccgtcttgttgcccgataatcactatttgagtacgcagagcgcattgagtaaggaccccaatgagaagcgggatcatatggtcttgttggagtttgtgacggccgccgggatcacacacgggatcgacgagttgtataagatgacgaccctgtccttccaggggccgggggcgacgaacttctccttgctgaagc

SEQ ID NO: 23 - DNA, 743 bp, Artificial Sequence
  mVenus codon harmonized for N. oceanica
ttcccttcatcctcccctccgagcagatggtgagcaagggggaggagttgtttacgggcgtggtgcccatcttggtcgagttggacggggacgttaacgggcacaagtttagcgtgtccggggagggggagggggatgccacgtacgggaagttgacgttgaagcttatctgcacgacggggaagttgcccgtgccctggcccacgcttgtgacgacgcttgggtacgggttgcagtgctttgcccgctaccccgaccacatgaagcagcacgacttttttaagtccgccatgcccgaggggtacgtccaggagcgcacgatcttttttaaggacgacgggaactacaagacgcgcgccgaggtgaagtttgagggggacacgttggtgaaccgcatcgagttgaaggggatcgactttaaggaggacgggaacatcttgggccacaagttggagtacaactacaacagccacaacgtctatatcacggccgacaagcagaagaacgggatcaaggccaactttaagatccgccacaacatcgaggacgggggggtgcagcttgccgaccactaccagcagaacacgcccatcggggacgggcccgtgttgttgcccgacaaccactacttgagctaccagtccaagttgagcaaggaccccaacgagaagcgcgatcacatggtcttgttggagtttgtgacggccgccggcatcacccttgggatggacgagttgtacaag

SEQ ID NO: 24 - DNA, 60 bp, Artificial Sequence
  oCS001
atgattacgccaagcttgcatgcgagtccccgggttggaggggtcgtttatttctctgcc

SEQ ID NO: 25 - DNA, 19 bp, Artificial Sequence
  oCS002
ctgctcggaggggaggatg

SEQ ID NO: 26 - DNA, 73 bp, Artificial Sequence
  oCS003
acgaacttctccttgctgaagcaggccggggacgtggaggagaacccggggcccatggccaagttgaccagtg

SEQ ID NO: 27 - DNA, 33 bp, Artificial Sequence
  oCS004
cctgatccctggccatcagtcctgctcctcggc

SEQ ID NO: 28 - DNA, 37 bp, Artificial Sequence
  oCS005
ggagcaggactgatggccagggatcaggaggagggag

SEQ ID NO: 29 - DNA, 33 bp, Artificial Sequence
  oCS006
aagactatttgctaagggagggtagtggcgatg

SEQ ID NO: 30 - DNA, 46 bp, Artificial Sequence
  oCS007
actaccctcccttagcaaatagtctttatttagaacacagaagaag

SEQ ID NO: 31 - DNA, 60 bp, Artificial Sequence
  oCS008
ctcggtacccggggatcctctagaggagtctggttgttggagagtgagggggagggggag

SEQ ID NO: 32 - DNA, 34 bp, Artificial Sequence
  oCS009
tccaacaaccagactcctctagaggatccccggg

SEQ ID NO: 33 - DNA, 37 bp, Artificial Sequence
  oCS010
tccaacccggggactcgcatgcaagcttggcgtaatc

SEQ ID NO: 34 - DNA, 31 bp, Artificial Sequence
  oCS011
taggtctcagtcacgcggtatcattgcagca

SEQ ID NO: 35 - DNA, 33 bp, Artificial Sequence
  oCS012
taggtctctgcattccaacccggggactcgcat

SEQ ID NO: 36 - DNA, 33 bp, Artificial Sequence
  oCS013
atggtctcacctttccaacaaccagactcctct

SEQ ID NO: 37 - DNA, 32 bp, Artificial Sequence
  oCS014
atggtctcttgacccacgctcaccggctccag

SEQ ID NO: 38 - DNA, 32 bp, Artificial Sequence
  oCS015
taggtctctatgcccctcccccctccctcctt

SEQ ID NO: 39 - DNA, 31 bp, Artificial Sequence
  oCS016
taggtctcttccaggacgcctgaaattaaag

SEQ ID NO: 40 - DNA, 32 bp, Artificial Sequence
  oCS017
taggtctcatggactcgttacatcagctccct

SEQ ID NO: 41 - DNA, 29 bp, Artificial Sequence
  oCS018
taggtctctaagggagggtagtggcgatg

SEQ ID NO: 42 - DNA, 18 bp, Artificial Sequence
  oCS019
gttggaatgcccctcccc

SEQ ID NO: 43 - DNA, 21 bp, Artificial Sequence
  oCS020
gttggaaagggagggtagtgg

SEQ ID NO: 44 - DNA, 21 bp, Artificial Sequence
  oCS021
gttggaggggtcgtttatttc

SEQ ID NO: 45 - DNA, 25 bp, Artificial Sequence
  oCS022
gttggaaagggagggtagtggcgat

SEQ ID NO: 46 - DNA, 25 bp, Artificial Sequence
  oCS023
gttcggaaactatcgatagggtttt

SEQ ID NO: 47 - DNA, 24 bp, Artificial Sequence
  oCS024
ctttcagtatatattggcagcgct

SEQ ID NO: 48 - DNA, 20 bp, Artificial Sequence
  oCS025
gagtccagggcacccgaaat

SEQ ID NO: 49 - DNA, 23 bp, Artificial Sequence
  oCS026
aaccagactcctctagaggatcc

SEQ ID NO: 50 - DNA, 23 bp, Artificial Sequence
  oCS027
ccggggactcgcatgcaagcttg

SEQ ID NO: 51 - DNA, 24 bp, Artificial Sequence
  oCS028
atgagcaagggggaggagttgttc

SEQ ID NO: 52 - DNA, 23 bp, Artificial Sequence
  oCS029
aagggagggtagtggcgatggtg

SEQ ID NO: 53 - DNA, 51 bp, Artificial Sequence
  oCS030
gccaagcttgcatgcgagtccccgggttcggaaactatcgatagggttttg

SEQ ID NO: 54 - DNA, 52 bp, Artificial Sequence
  oCS031
tgaacaactcctcccccttgctcattgctatcgttattgttgttgagttatg

SEQ ID NO: 55 - DNA, 49 bp, Artificial Sequence
  oCS032
gccaccatcgccactaccctcccttttgtcctacgatccactgagattc

SEQ ID NO: 56 - DNA, 55 bp, Artificial Sequence
  oCS033
cccggggatcctctagaggagtctggttgagtccagggcacccgaaatattaaac

SEQ ID NO: 57 - DNA, 23 bp, Artificial Sequence
  oCS034
tgaaaccactcctagtgagattg

SEQ ID NO: 58 - DNA, 22 bp, Artificial Sequence
  oCS035
tgctatcgttattgttgttgag

SEQ ID NO: 59 - DNA, 48 bp, Artificial Sequence
  oCS036
taactcaacaacaataacgatagcaacaccaaacagtttcgacttggc

SEQ ID NO: 60 - DNA, 36 bp, Artificial Sequence
  oCS037
ctgagctggttgtcgccgttactaggggaatcctgg

SEQ ID NO: 61 - DNA, 36 bp, Artificial Sequence
  oCS038
cctagtaacggcgacaaccagctcagaactggagcg

SEQ ID NO: 62 - DNA, 37 bp, Artificial Sequence
  oCS039
gtgaatatcgtcgagtggtatttcatcgtcgccggag

SEQ ID NO: 63 - DNA, 37 bp, Artificial Sequence
  oCS040
atgaaataccactcgacgatattcacctcttctgatg

SEQ ID NO: 64 - DNA, 48 bp, Artificial Sequence
  oCS041
cgcaatctcactaggagtggtttcaacaacaaatcgacggaacaagag

SEQ ID NO: 65 - DNA, 48 bp, Artificial Sequence
  oCS042
taactcaacaacaataacgatagcagccaagcagtcggtggacgttac

SEQ ID NO: 66 - DNA, 48 bp, Artificial Sequence
  oCS043
taactcaacaacaataacgatagcaagtcttcggacggaaaaagcctg

SEQ ID NO: 67 - DNA, 49 bp, Artificial Sequence
  oCS044
taactcaacaacaataacgatagcaatctggttgattctgccagtagtc

SEQ ID NO: 68 - DNA, 38 bp, Artificial Sequence
  oCS045
taagtttccccgtttgaatgattcgtcgctggcaaaag

SEQ ID NO: 69 - DNA, 36 bp, Artificial Sequence
  oCS046
acgaatcattcaaacggggaaacttaccaggtccag

SEQ ID NO: 70 - DNA, 49 bp, Artificial Sequence
  oCS047
cgcaatctcactaggagtggtttcaacaacaaatcgacggaacaagagg

SEQ ID NO: 71 - DNA, 42 bp, Artificial Sequence
  oCS048
gggttttcaagggccgactctcttttcaaagttctttgcatc

SEQ ID NO: 72 - DNA, 36 bp, Artificial Sequence
  oCS049
ttgaaaagagagtcggcccttgaaaacccggtggcg

SEQ ID NO: 73 - DNA, 38 bp, Artificial Sequence
  oCS050
tgtcgcgacaacagtttaacagatgtgccgccccagcc

SEQ ID NO: 74 - DNA, 36 bp, Artificial Sequence
  oCS051
acatctgttaaactgttgtcgcgacagtaatacaac

SEQ ID NO: 75 - DNA, 21 bp, Artificial Sequence
  oCS052
aaaaccctggcgttacccaac

SEQ ID NO: 76 - DNA, 21 bp, Artificial Sequence
  oCS053
aaaccgctcacaattccacac

SEQ ID NO: 77 - DNA, 52 bp, Artificial Sequence
  oCS054
ttccttgccactacaattgtatctaagttcggaaactatcgatagggttttg

SEQ ID NO: 78 - DNA, 53 bp, Artificial Sequence
  oCS055
agagggctgaaattgacggtctacaagagtccagggcacccgaaatattaaac

SEQ ID NO: 79 - DNA, 49 bp, Artificial Sequence
  oCS056
gtatgttgtgtggaattgtgagcggtttaaatctctctctccctctctc

SEQ ID NO: 80 - DNA, 51 bp, Artificial Sequence
  oCS057
ttagatacaattgtagtggcaaggaacacaaaatgccagtcacactttttg

SEQ ID NO: 81 - DNA, 52 bp, Artificial Sequence
  oCS058
ttgtagaccgtcaatttcagccctcttatcttgtgtgtgttaacagctggtc

SEQ ID NO: 82 - DNA, 47 bp, Artificial Sequence
  oCS059
ttaagttgggtaacgccagggttttaaagtatcaacgagaaggctac

SEQ ID NO: 83 - DNA, 46 bp, Artificial Sequence
  oCS060
gttgtgtggaattgtgagcggtttaaatgatctcagtcagcctccg

SEQ ID NO: 84 - DNA, 52 bp, Artificial Sequence
  oCS061
ttagatacaattgtagtggcaaggaacacaaagtgggaaggaaagggaagtg

SEQ ID NO: 85 - DNA, 51 bp, Artificial Sequence
  oCS062
ttgtagaccgtcaatttcagccctcttatctccgtccactcgtgatgcctg

SEQ ID NO: 86 - DNA, 56 bp, Artificial Sequence
  oCS063
ttaagttgggtaacgccagggttttaaatttgatagcaggaatatgggaacagcag

SEQ ID NO: 87 - DNA, 19 bp, Artificial Sequence
  oCS064
ttgtgaagtgagcgaaggt

SEQ ID NO: 88 - DNA, 23 bp, Artificial Sequence
  oCS065
aaagtatcaacgagaaggctacg

SEQ ID NO: 89 - DNA, 22 bp, Artificial Sequence
  oCS066
atgacgaccctgtccttccagg

SEQ ID NO: 90 - DNA, 19 bp, Artificial Sequence
  oCS067
ctgctcggaggggaggatg

SEQ ID NO: 91 - DNA, 43 bp, Artificial Sequence
  oCS068
tcccttcatcctcccctccgagcagatggtgagcaagggcgag

SEQ ID NO: 92 - DNA, 45 bp, Artificial Sequence
  oCS069
gcccctggaaggacagggtcgtcatcttgtacagctcgtccatgc

SEQ ID NO: 93 - DNA, 47 bp, Artificial Sequence
  oCS070
cccttgggatggacgagttgtacaagatgacgaccctgtccttccag

SEQ ID NO: 94 - DNA, 19 bp, Artificial Sequence
  oCS071
ctgctcggaggggaggatg

SEQ ID NO: 95 - DNA, 20 bp, Artificial Sequence
  oCS072
ctccttcccttcatcctccc

SEQ ID NO: 96 - DNA, 18 bp, Artificial Sequence
  oCS073
ctggaaggacagggtcgt

SEQ ID NO: 97 - DNA, 23 bp, Artificial Sequence
  oCS074
aaccagactcctctagaggatcc

SEQ ID NO: 98 - DNA, 23 bp, Artificial Sequence
  oCS075
ccggggactcgcatgcaagcttg

SEQ ID NO: 99 - DNA, 43 bp, Artificial Sequence
  oCS076
gccaagcttgcatgcgagtccccgggtgaaaatccacaggaag

SEQ ID NO: 100 - DNA, 31 bp, Artificial Sequence
  oCS077
ctgagctggttgtttaattgtagcaagcgac

SEQ ID NO: 101 - DNA, 86 bp, Artificial Sequence
  oCS078
cacctttagacatctgctcggaggggaggatgaagggaaggagggaggggggaggggcattccaattagatgacgaggcatttggc

SEQ ID NO: 102 - DNA, 33 bp, Artificial Sequence
  oCS079
tgctacaattaaacaaccagctcagaactggag

SEQ ID NO: 103 - DNA, 32 bp, Artificial Sequence
  oCS080
cacctttagacatctgctcggaggggaggatg

SEQ ID NO: 104 - DNA, 36 bp, Artificial Sequence
  oCS081
ccctccgagcagatgtctaaaggtgaagaattattc

SEQ ID NO: 105 - DNA, 49 bp, Artificial Sequence
  oCS082
gttttaacaaagaaaaattagtagctttgtacaattcatcaataccatg

SEQ ID NO: 106 - DNA, 78 bp, Artificial Sequence
  oCS083
gctactaatttttctttgttaaaacaagctggtgatgttgaagaaaatcctggtccaatgtcgaaagctacatataag

SEQ ID NO: 107 - DNA, 41 bp, Artificial Sequence
  oCS084
cgtcactaattagggtaataactgatataattaaattgaag

SEQ ID NO: 108 - DNA, 35 bp, Artificial Sequence
  oCS085
tcagttattaccctaattagtgacgcgcatgaatg

SEQ ID NO: 109 - DNA, 43 bp, Artificial Sequence
  oCS086
ggggatcctctagaggagtctggtttcttcaacccggatcagc

SEQ ID NO: 110 - DNA, 26 bp, Artificial Sequence
  oCS087
gtgaaaatccacaggaaggaatagtt

SEQ ID NO: 111 - DNA, 18 bp, Artificial Sequence
  oCS088
tcttcaacccggatcagc

SEQ ID NO: 112 - DNA, 64 bp, Artificial Sequence
  oCS089
  misc_feature (63)..(64): n is a, c, g, or t
taggcaggcgggcacctcgcgttagtggctgggtctaggcgctctgggcgggtaacgtggagnn

SEQ ID NO: 113 - DNA, 13 bp, Artificial Sequence
  oCS090
  misc_feature (1)..(1): 5 PHOS
  misc_feature (13)..(13): 3 AMMO
ctccacgttaccc

SEQ ID NO: 114 - DNA, 27 bp, Artificial Sequence
  oCS091
taggcaggcgggcacctcgcgttagtg

SEQ ID NO: 115 - DNA, 20 bp, Artificial Sequence
  oCS092
ccccgtgaacaactcctccc

SEQ ID NO: 116 - DNA, 29 bp, Artificial Sequence
  oCS093
atggtcatcgctcctggctctacttgtgg

SEQ ID NO: 117 - DNA, 27 bp, Artificial Sequence
  oCS094
gttagtggctgggtctaggcgctctgg

SEQ ID NO: 118 - DNA, 20 bp, Artificial Sequence
  oCS095
tgaacaactcctcccccttg

SEQ ID NO: 119 - DNA, 30 bp, Artificial Sequence
  oCS096
gctgcttcttccttccatccgtgactgtat

SEQ ID NO: 120 - DNA, 25 bp, Artificial Sequence
  oCS097
cggttggttgtaggggacgtgagaa

SEQ ID NO: 121 - DNA, 24 bp, Artificial Sequence
  oCS098
agtgtcgatttcggctgtgggtaa

SEQ ID NO: 122 - DNA, 23 bp, Artificial Sequence
  oCS099
gattggtttcggccatcttccct

SEQ ID NO: 123 - DNA, 26 bp, Artificial Sequence
  oCS100
ggggtcgtggcagaaatctataagtg

SEQ ID NO: 124 - DNA, 23 bp, Artificial Sequence
  oCS101
atgggcagagagagcaagggaaa

SEQ ID NO: 125 - DNA, 23 bp, Artificial Sequence
  oCS102
gattggtttcggccatcttccct

SEQ ID NO: 126 - DNA, 26 bp, Artificial Sequence
  oCS103
cgtctcatcacacgaattgtccatca

SEQ ID NO: 127 - DNA, 24 bp, Artificial Sequence
  oCS104
gttagaagtgctcgagaggttggc

SEQ ID NO: 128 - DNA, 23 bp, Artificial Sequence
  oCS105
gattggtttcggccatcttccct

SEQ ID NO: 129 - DNA, 26 bp, Artificial Sequence
  oCS106
cgtctcatcacacgaattgtccatca

SEQ ID NO: 130 - DNA, 20 bp, Artificial Sequence
  oCS107
ggaacggcactggtcaactt

SEQ ID NO: 131 - DNA, 29 bp, Artificial Sequence
  oCS108
atggtcatcgctcctggctctacttgtgg

SEQ ID NO: 132 - DNA, 24 bp, Artificial Sequence
  oCS109
gttagaagtgctcgagaggttggc

SEQ ID NO: 133 - DNA, 19 bp, Artificial Sequence
  oCS110
attgagtaaggaccccaat

SEQ ID NO: 134 - DNA, 18 bp, Artificial Sequence
  oCS111
gttggaaagggagggtag

SEQ ID NO: 135 - DNA, 21 bp, Artificial Sequence
  oCS112
ctctctgacggaggagctcta

SEQ ID NO: 136 - DNA, 25 bp, Artificial Sequence
  oCS113
tgaggtttagataacttctcacgct

SEQ ID NO: 137 - DNA, 20 bp, Artificial Sequence
  oCS114
ggaacggcactggtcaactt

SEQ ID NO: 138 - DNA, 23 bp, Artificial Sequence
  oCS115
acaacgaattgtagtaccgcaga

SEQ ID NO: 139 - DNA, 21 bp, Artificial Sequence
  oCS116
tgtgttccttgccactacaat

SEQ ID NO: 140 - DNA, 20 bp, Artificial Sequence
  oCS117
agagggctgaaattgacggt

SEQ ID NO: 141 - DNA, 20 bp, Artificial Sequence
  oCS118
ggataacaatttcacacagg

SEQ ID NO: 142 - DNA, 18 bp, Artificial Sequence
  oCS119
ggcaccttatcagaaaga

SEQ ID NO: 143 - DNA, 33 bp, Artificial Sequence; identical to SEQ ID NO: 27
  oCS120

SEQ ID NO: 144 - DNA, 24 bp, Artificial Sequence
  oCS121
gtcgtgactgggaaaaccctggcg

SEQ ID NO: 145 - DNA, 20 bp, Artificial Sequence
  oCS122
tgattacgccaagcttgcat

SEQ ID NO: 146 - DNA, 18 bp, Artificial Sequence
  oCS123
aaacgacggccagtgaat

SEQ ID NO: 147 - DNA, 20 bp, Artificial Sequence
  oCS124
ggaacggcactggtcaactt

SEQ ID NO: 148 - DNA, 22 bp, Artificial Sequence
  oCS125
ggtcttgttggagtttgtgacg

SEQ ID NO: 149 - DNA, 18 bp, Artificial Sequence
  oCS126
gttggaatgcccctcccc

SEQ ID NO: 150 - DNA, 19 bp, Artificial Sequence
  oCS127
ccattgaagttgtgtgatg

SEQ ID NO: 151 - DNA, 18 bp, Artificial Sequence
  oCS128
ggcgtgaattgcgaggaa

SEQ ID NO: 152 - DNA, 22 bp, Artificial Sequence
  oCS129
atgacgaccctgtccttccagg

SEQ ID NO: 153 - DNA, 20 bp, Artificial Sequence
  oCS130
tgattacgccaagcttgcat

SEQ ID NO: 154 - DNA, 48 bp, Artificial Sequence; identical to SEQ ID NO: 65
  oCS131

SEQ ID NO: 155 - DNA, 20 bp, Artificial Sequence
  oCS132
gcgtgagaagttatctaaac

SEQ ID NO: 156 - DNA, 19 bp, Artificial Sequence
  oCS133
ctgctcggaggggaggatg

SEQ ID NO: 157 - DNA, 19 bp, Artificial Sequence
  oCS134
taggcaccccaggctttac

SEQ ID NO: 158 - DNA, 18 bp, Artificial Sequence
  oCS135
atgacgaccctgtccttc

SEQ ID NO: 159 - DNA, 25 bp, Artificial Sequence
  oCS136
gattggcatacgaaacttggtggga

SEQ ID NO: 160 - DNA, 18 bp, Artificial Sequence
  oCS137
atgagcaagggggaggag

SEQ ID NO: 161 - DNA, 18 bp, Artificial Sequence
  oCS138
aaacgacggccagtgaat

SEQ ID NO: 162 - DNA, 22 bp, Artificial Sequence
  oCS139
tgctatcgttattgttgttgag

SEQ ID NO: 163 - DNA, 23 bp, Artificial Sequence
  oCS140
tcagtcctgctcctcggccacga

SEQ ID NO: 164 - DNA, 29 bp, Artificial Sequence
  oCS141
atggtcatcgctcctggctctacttgtgg

SEQ ID NO: 165 - DNA, 23 bp, Artificial Sequence
  oCS142
tgaaaccactcctagtgagattg

SEQ ID NO: 166 - DNA, 52 bp, Artificial Sequence; identical to SEQ ID NO: 81
  oCS143

SEQ ID NO: 167 - DNA, 51 bp, Artificial Sequence; identical to SEQ ID NO: 85
  oCS144

SEQ ID NO: 168 - DNA, 20 bp, Artificial Sequence
  oCS145
ggaacggcactggtcaactt

SEQ ID NO: 169 - DNA, 30 bp, Artificial Sequence; identical to SEQ ID NO: 119
  oCS146

SEQ ID NO: 170 - DNA, 23 bp, Artificial Sequence
  oCS147
gattggtttcggccatcttccct

SEQ ID NO: 171 - DNA, 18 bp, Artificial Sequence
  oCS148
tagccaaatgcctcgtca

SEQ ID NO: 172 - DNA, 41 bp, Artificial Sequence; identical to SEQ ID NO: 107
  oCS149

SEQ ID NO: 173 - DNA, 48 bp, Artificial Sequence
  oCS150
gcatcagaagaggtgaatatcgtcgagtggtatttcatcgtcgccgga

SEQ ID NO: 174 - DNA, 46 bp, Artificial Sequence
  oCS151
ttaagttgggtaacgccagggttttaaagcggatgcgtatgtgttg

SEQ ID NO: 175 - DNA, 43 bp, Artificial Sequence
  oCS152
atcgccactaccctccctttccaactcatcgtcatcgtccttt

SEQ ID NO: 176 - DNA, 18 bp, Artificial Sequence
  oCS153
gttggaaagggagggtag

SEQ ID NO: 177 - DNA, 18 bp, Artificial Sequence
  oCS154
tggccagggatcaggagg

SEQ ID NO: 178 - DNA, 46 bp, Artificial Sequence
  oCS155
tcccttcatcctcccctccgagcagatgagcaagggggaggagttg

SEQ ID NO: 179 - DNA, 30 bp, Artificial Sequence
  oCS156
cgagaggggcatgggccccgggttctcctc

SEQ ID NO: 180 - DNA, 31 bp, Artificial Sequence
  oCS157
gaacccggggcccatgcccctctcgcaggag

SEQ ID NO: 181 - DNA, 43 bp, Artificial Sequence
  oCS158
actccctcctcctgatccctggccatcaagcgctctcccacac

SEQ ID NO: 182 - DNA, 18 bp, Artificial Sequence
  oCS159
acaccaaacagtttcgac

SEQ ID NO: 183 - DNA, 22 bp, Artificial Sequence
  oCS160
taagctctttcttcttctcttg

SEQ ID NO: 184 - DNA, 44 bp, Artificial Sequence
  oCS161
attcaagagaagaagaaagagcttagatggagtggatggaggag

SEQ ID NO: 185 - DNA, 46 bp, Artificial Sequence
  oCS162
ccgccaagtcgaaactgtttggtgttgttgatgcgggctgagattg

SEQ ID NO: 186 - DNA, 18 bp, Artificial Sequence
  oCS163
tcatcgtcatcgtccttt

SEQ ID NO: 187 - DNA, 45 bp, Artificial Sequence
  oCS164
cgcaatctcactaggagtggtttcaaagggagggtagtggcgatg

SEQ ID NO: 188 - DNA, 43 bp, Artificial Sequence
  oCS165
gtacgggaaaggacgatgacgatgatgagtccagggcacccga

SEQ ID NO: 189 - DNA, 43 bp, Artificial Sequence
  oCS166
cgcaatctcactaggagtggtttcatcaagcgctctcccacac

SEQ ID NO: 190 - DNA, 43 bp, Artificial Sequence
  oCS167
tctactcgtctctcttggatctttctcaagcgctctcccacac

SEQ ID NO: 191 - DNA, 19 bp, Artificial Sequence
  oCS168
gaaagatccaagagagacg

SEQ ID NO: 192 - DNA, 44 bp, Artificial Sequence
  oCS169
cgcaatctcactaggagtggtttcatagtggatcagcttgcatg

SEQ ID NO: 193 - DNA, 200 bp, Artificial Sequence
  oCS170
acgtgtgggagagcgcttgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggccggcatggtcccagcctcctcgctggcgccggctgggcaacatgcttcggcatggcgaatgggactcctg

SEQ ID NO: 194 - DNA, 44 bp, Artificial Sequence
  oCS171
atcagagaacttctcccttcgggctacgtgtgggagagcgcttg

SEQ ID NO: 195 - DNA, 45 bp, Artificial Sequence
  oCS172
cgcaatctcactaggagtggtttcacaggagtcccattcgccatg

SEQ ID NO: 196 - DNA, 21 bp, Artificial Sequence
  oCS173
tcatcgtcatcgtcctttccc

SEQ ID NO: 197 - DNA, 44 bp, Artificial Sequence
  oCS174
atcgccactaccctccctttccaacgatggagtggatggaggag

SEQ ID NO: 198 - DNA, 43 bp, Artificial Sequence
  oCS175
gtacgggaaaggacgatgacgatgaagtagatgccgaccggag

SEQ ID NO: 199 - DNA, 18 bp, Artificial Sequence
  oCS176
tggccagggatcaggagg

SEQ ID NO: 200 - DNA, 38 bp, Artificial Sequence
  oCS177
tcctcgagagtaaacaccatgctgctgaccgtgacctg

SEQ ID NO: 201 - DNA, 25 bp, Artificial Sequence
  oCS178
atggtgtttactctcgaggacttcg

SEQ ID NO: 202 - DNA, 38 bp, Artificial Sequence
  oCS179
ctcctcctgatccctggccatcatccggccaggatgcg

SEQ ID NO: 203 - DNA, 18 bp, Artificial Sequence
  oCS180
tggccagggatcaggagg

SEQ ID NO: 204 - DNA, 29 bp, Artificial Sequence
  oCS181
ttagatacaattgtagtggcaaggaacac

SEQ ID NO: 205 - DNA, 44 bp, Artificial Sequence
  oCS182
tccttgccactacaattgtatctaagatggagtggatggaggag

SEQ ID NO: 206 - DNA, 46 bp, Artificial Sequence
  oCS183
tgacctcctcgcccttgctcaccattgttgatgcgggctgagattg

SEQ ID NO: 207 - DNA, 20 bp, Artificial Sequence
  oCS184
atggtgagcaagggcgagga

SEQ ID NO: 208 - DNA, 18 bp, Artificial Sequence
  oCS185
tcagtcctgctcctcggc

SEQ ID NO: 209 - DNA, 50 bp, Artificial Sequence
  oCS186
cgaagtcctcgagagtaaacaccatttagctatcagtcctgctcctcggc

SEQ ID NO: 210 - DNA, 25 bp, Artificial Sequence
  oCS187
atggtgtttactctcgaggacttcg

SEQ ID NO: 211 - DNA, 46 bp, Artificial Sequence
  oCS188
actccctcctcctgatccctggccatcacgcgtagtcgggcacgtc

SEQ ID NO: 212 - DNA, 53 bp, Artificial Sequence
  oCS189
cttcgtggccgaggagcaggactgatagctaaacaaccagctcagaactggag

SEQ ID NO: 213 - DNA, 44 bp, Artificial Sequence
  oCS190
cgaagtcctcgagagtaaacaccatctgctcggaggggaggatg

SEQ ID NO: 214 - DNA, 18 bp, Artificial Sequence
  oCS191
ccggggactcgcatgcaa

SEQ ID NO: 215 - DNA, 47 bp, Artificial Sequence
  oCS192
gccaagcttgcatgcgagtccccggtttttgtagaaatgtcttggtg

SEQ ID NO: 216 - DNA, 50 bp, Artificial Sequence
  oCS193
tgaataattcttcacctttagacatatagttgttcaattgattgaaatag

SEQ ID NO: 217 - DNA, 24 bp, Artificial Sequence
  oCS194
atgtctaaaggtgaagaattattc

SEQ ID NO: 218 - DNA, 43 bp, Artificial Sequence
  oCS195
gaacggcactggtcaacttggccattggaccaggattttcttc

SEQ ID NO: 219 - DNA, 19 bp, Artificial Sequence
  oCS196
atggccaagttgaccagtg

SEQ ID NO: 220 - DNA, 18 bp, Artificial Sequence
  oCS197
tcagtcctgctcctcggc

SEQ ID NO: 221 - DNA, 44 bp, Artificial Sequence
  oCS198
cttcgtggccgaggagcaggactgatcaagaggatgtcagaatg

SEQ ID NO: 222 - DNA, 47 bp, Artificial Sequence
  oCS199
ggggatcctctagaggagtctggtttctcacttaatcttctgtactc

SEQ ID NO: 223 - DNA, 22 bp, Artificial Sequence
  oCS200
tttttgtagaaatgtcttggtg

SEQ ID NO: 224 - DNA, 22 bp, Artificial Sequence
  oCS201
tctcacttaatcttctgtactc

SEQ ID NO: 225 - DNA, 22 bp, Artificial Sequence
  oCS202
aaataacaaatctcaacacaac

SEQ ID NO: 226 - DNA, 49 bp, Artificial Sequence
  oCS203
gccaagcttgcatgcgagtccccggagtactgaccccctcagtgggcca

SEQ ID NO: 227 - DNA, 52 bp, Artificial Sequence
  oCS204
ggacaccaagacatttctacaaaaagatgacgaggcatttggctaccttaag

SEQ ID NO: 228 - DNA, 48 bp, Artificial Sequence
  oCS205
tcagagtacagaagattaagtgagataattagtgacgcgcatgaatgg

SEQ ID NO: 229 - DNA, 53 bp, Artificial Sequence
  oCS206
ggggatcctctagaggagtctggttagtacttggtaccgcttggtgggattac

SEQ ID NO: 230 - DNA, 52 bp, Artificial Sequence
  oCS207
tatgttgtgttgagatttgttatttgatgacgaggcatttggctaccttaag

SEQ ID NO: 231 - DNA, 49 bp, Artificial Sequence
  oCS208
gccaagcttgcatgcgagtccccggagtactctagcaagaccggtcttc

SEQ ID NO: 232 - DNA, 46 bp, Artificial Sequence
  oCS209
ggacaccaagacatttctacaaaaatccgtggtgatgctgagattc

SEQ ID NO: 233 - DNA, 44 bp, Artificial Sequence
  oCS210
tcagagtacagaagattaagtgagacgaagtcatcgaaagactc

SEQ ID NO: 234 - DNA, 51 bp, Artificial Sequence
  oCS211
ggggatcctctagaggagtctggttagtacttaattattcgaaacgatggc

SEQ ID NO: 235 - DNA, 46 bp, Artificial Sequence
  oCS212
tatgttgtgttgagatttgttattttccgtggtgatgctgagattc

SEQ ID NO: 236 - DNA, 18 bp, Artificial Sequence
  oCS213
gatgacgaggcatttggc

SEQ ID NO: 237 - DNA, 20 bp, Artificial Sequence
  oCS214
tcaagaggatgtcagaatgc

SEQ ID NO: 238 - DNA, 46 bp, Artificial Sequence
  oCS215
taaggtagccaaatgcctcgtcatcacaaccagctcagaactggag

SEQ ID NO: 239 - DNA, 51 bp, Artificial Sequence
  oCS216
tatgttgtgttgagatttgttattttgatcatctgctcggaggggaggatg

SEQ ID NO: 240 - DNA, 23 bp, Artificial Sequence
  oCS217
aaataacaaatctcaacacaaca

SEQ ID NO: 241 - DNA, 18 bp, Artificial Sequence
  oCS218
tgatcagtcctgctcctc

SEQ ID NO: 242 - DNA, 46 bp, Artificial Sequence
  oCS219
cgtggccgaggagcaggactgatcacagtggccagggatcaggagg

SEQ ID NO: 243 - DNA, 45 bp, Artificial Sequence
  oCS220
aaatggcattctgacatcctcttgaaagggagggtagtggcgatg

SEQ ID NO: 244 - DNA, 30 bp, Artificial Sequence
  oCS221
  misc_feature (1)..(1): 5 PHOS
atgtctaaaggtgaagaattattcactggt

SEQ ID NO: 245 - DNA, 21 bp, Artificial Sequence
  oCS222
catctgctcggaggggaggat

SEQ ID NO: 246 - DNA, 19 bp, Artificial Sequence
  oCS223
gatgttgacgcaatgtgat

SEQ ID NO: 247 - DNA, 19 bp, Artificial Sequence
  oCS224
caagcccgttcccttggct

SEQ ID NO: 248 - DNA, 20 bp, Artificial Sequence
  oCS225
gcccagtgctctgaatgtca

SEQ ID NO: 249 - DNA, 20 bp, Artificial Sequence
  oCS226
tggtcttgtagttaccgtca

SEQ ID NO: 250 - DNA, 19 bp, Artificial Sequence
  oCS227
gaccccctcagtgggccat

SEQ ID NO: 251 - DNA, 20 bp, Artificial Sequence
  oCS228
tggtaccgcttggtgggatt

SEQ ID NO: 252 - DNA, 21 bp, Artificial Sequence
  oCS229
tgccgaagtttccctcaggat

SEQ ID NO: 253 - DNA, 22 bp, Artificial Sequence
  oCS230
tcacaatgctgcaaatgacgct

SEQ ID NO: 254 - DNA, 22 bp, Artificial Sequence
  oCS231
ctaaggttggccatggaactgg

SEQ ID NO: 255 - DNA, 21 bp, Artificial Sequence
  oCS232
acgacgtgaccctgttcatca

SEQ ID NO: 256 - DNA, 22 bp, Artificial Sequence
  oCS233
ttttggaagtggaggtgtcacg

SEQ ID NO: 257 - DNA, 22 bp, Artificial Sequence
  oCS234
gaaacacccgctttttggatga

SEQ ID NO: 258 - RNA, 43 nt, Artificial Sequence
  oCS235
aauuucuacuguuguagauguguguguuaacagcuggucgcag

SEQ ID NO: 259 - RNA, 43 nt, Artificial Sequence
  oCS236
aauuucuacuguuguagauaugacggccauguuguuguccucg

SEQ ID NO: 260 - RNA, 471 nt, Saccharomyces cerevisiae
agaauaauguagauaagggaagucggcaaaauagauccguaacuucgggauaaggauuggcucuaagggucggguagugagggccuuggucagacgcagcgggcgugcuuguggacugcuugguggggcuugcucugcuaggcggacuacuugcgugccuuguuguagacggccuugguaggucucuuguagaccgucgcuugcuacaauuaacgaucaacuuagaacugguacggacaaggggaaucugacugucuaauuaaaacauagcauugcgauggucagaaagugauguugacgcaaugugauuucugcccagugcucugaaugucaaagugaagaaauucaaccaagcgcggguaaacggcgggaguaacuaugacucucuuaagguagccaaaugccucgucaucuaauuagugacgcgcaugaauggauuaacgagauucccacugucccuaucuacuaucu

SEQ ID NO: 261 - DNA, 3933 bp, Artificial Sequence
  MCEC-EMCV
  misc_feature (1)..(1000): Left HF, complementary to the 28S rDNA of a mammalian cell line, C989A
  misc_feature (1001)..(1551): IRES of the EMCV
  misc_feature (1552)..(2280): EGFP, humanized, with four additional N-terminal AAs to safeguard optimal EMCV IRES performance
  misc_feature (2290)..(2346): 2A peptide from porcine teschovirus-1 polyprotein
  gene (2347)..(2721): Zeocin resistance gene, codon optimized for H. sapiens
  misc_feature (2722)..(2857): SVLPA
  misc_feature (2858)..(2933): PNAts, contains binding site for NoLS-PNA to facilitate EC trafficking to the nucleolus
  misc_feature (2873)..(2918): DNA triplex formation site upon PNA binding
  misc_feature (2934)..(3933): Right HF, complementary to the 28S rDNA of a mammalian cell line
gaacagcctctggcatgttggaacaatgtaggtaagggaagtcggcaagccggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccgcgcgcgccgctcgctccctccccgccccgcgccctctctctctctctctcccccgctccccgtcctcccccctccccgggggagcgccgcgtgggggcggcggcggggggagaagggtcggggcggcaggggccggcggcggcccgccgcggggccccggcggcgggggcacggtcccccgcgaggggggcccgggcacccggggggccggcggcggcggcgactctggacgcgagccgggcccttcccgtggatcgccccagctgcggcgggcgtcgcggccgcccccggggagcccggcgggcgccggcgcgcccccccccacccccaccccacgtctcgtcgcgcgcgcgtccgctgggggcggggagcggtcgggcggcggcggtcggcgggcggcggggcggggcggttcgtccccccgccctacccccccggccccgtccgccccccgttcccccctcctcctcggcgcgcggcggcggcggcggcggcggcaggcggcggaggggccgcgggccggtcccccccgccgggtccgcccccggggccgcggttccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcatcgtcatctaaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaagcaacgtctgtagcgaccctttgcaggcagcggaaccccccacctggcgacaggtgcctctgcggccaaaagccacgtgtataagatacacctgcaaaggcggcacaaccccagtgccacgttgtgagtaggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtcagtaagggagaggaattgttcactggggtagtgcctatattggtagaactcgacggagatgtgaatggccacaaattttctgtgtcaggggagggtgagggtgatgcaacctacggaaaattgacgctgaagtttatttgtacgacgggcaagctgccagttccctggcccacattggtaacaacccttacctatggagtacagtgtttcagtcgatatccagatcacatgaaacagcacgactttttcaaaagcgcgatgccggaaggatacgttcaagaaaggactatattcttcaaggatgatggcaactacaaaacacgagcagaagtaaagttcgagggggatacgctcgtaaataggatcgaactcaagggaatagacttcaaggaggacgggaatatactcggacataagttggagtacaactataatagccataatgtatatataatggccgacaagcagaagaatggaatcaaagtaaatttcaaaattcgccacaacatcgaggatggctccgtccagcttgcggatcattatcagcagaatactccaatcggagatggacctgttttgctgccggataatcattacctgtctacacagtcagccctttcaaaagatccgaacgagaagcgggaccatatggtacttcttgagttcgtcaccgcggcgggaataacacttgggatggatgaactctacaaaggaagcggagctactaacttcagcctgctgaagcaggctggagacgtggaggagaaccctggacctatggctaaactcacaagtgcggtccctgttttgactgcaagggatgttgcaggagccgttgagttctggacggatcgattggggtttagccgagattttgtcgaagatgactttgctggcgttgtacgagatgatgtcacattgtttatatctgctgttcaggatcaagttgtaccggacaacacattggcttgggtatgggtgcggggtctcgatgagctgtacgcagagtggagcgaggttgtgagtacgaattttagagatgcaagcggccctgccatgacggagatcggggaacaaccatggggccgcgaatttgccttgagagaccctgcgggtaattgtgtgcactttgttgccgaagagcaggattgaaaaaaatgctttatttgtgaaatttgtgatgctattgctttatttgtaaccattataagctgcaataaacaagttaacaacaacaattgcattcattttatgtttcaggttcagggggaggtgtgggaggttttttgtgatcctagcattaaagaagaaaactaagaagaaaactaagaagaaaactaagaagaaaagtatcatgaactgccttagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgccggccctgcgggccgccggtgaaataccactactctgatcgttttttcactgacccggtgaggcgggggggcgagccccgaggggctctcgcttctggcgccaagcgcccggccgcgcgccggccgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcaccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggctgaggagccaatggggcgaagctaccatctgtgggattatgactgaacgcctctaagtcagaatcccgcccaggcggaacgatacggcagcgccgcggagcctcggttggcctcggatagccggtcccccgcctgtccccgccggcgggccgccc

SEQ ID NO: 262 - skipped

SEQ ID NO: 263 - RNA, 100 nt, Artificial Sequence
  Synthetic sgRNA sequence for assembly of NoLS-Cas9 RNP for 28S rDNA cleavage in mammalian cells
  misc_feature (1)..(20): spacer motif complementary to the 28S rDNA
gcgucacuaauuagaugacgguuuuagagcuagaaauagcaaguuaaaauaaggcuaguccguuaucaacuugaaaaaguggcaccgagucggugcuuuu

SEQ ID NO: 264 - protein, 18 aa, Artificial Sequence
  HIV-1 Rev protein nucleolar localization sequence
RQARRNRRRRWRERQRQI